(19) United States
(12) Patent Application Publication
(10) Pub. No.: US 2003/0180953 A1
(43) Pub. Date: Sep. 25, 2003

(54) Gene disruption methodologies for drug target discovery

(75) Inventors: Terry Roemer, Montreal (CA); Bo Jiang, Montreal (CA); Charles Boone, Toronto (CA); Howard Bussey, Westmount (CA); Kari L. Ohlsen, San Diego, CA (US)

Correspondence Address: PENNIE AND EDMONDS, 1155 AVENUE OF THE AMERICAS, NEW YORK, NY, 100362711

(73) Assignee: Elitra Pharmaceuticals, Inc.

(21) Appl. No.: 10/032,585

(22) Filed: Dec. 20, 2001

Related U.S. Application Data
(63) Continuation-in-part of application No. 09/792,024, filed on Feb. 20, 2001.
(60) Provisional application No. 60/259,128, filed on Dec. 29, 2000.
(60) Provisional application No. 60/314,050, filed on Aug. 22, 2001.

(51) Int. Cl. C12N 1/18; C12N 1/16; C12N 15/74

(52) U.S. Cl. 435/483; 435/484; 435/254.2

(57) ABSTRACT

The present invention provides methods and compositions that enable the experimental determination as to whether any gene in the genome of a diploid pathogenic organism is essential, and whether it is required for virulence or pathogenicity. The methods involve the construction of genetic mutants in which one allele of a specific gene is inactivated while the other allele of the gene is placed under conditional expression. The identification of essential genes and those genes critical to the development of virulent infections, provides a basis for the development of screens for new drugs against such pathogenic organisms.

The present invention further provides Candida albicans genes that are demonstrated to be essential and are potential targets for drug screening. The nucleotide sequence of the target genes can be used for various drug discovery purposes, such as expression of the recombinant protein, hybridization assay and construction of nucleic acid arrays. The uses of proteins encoded by the essential genes, and genetically engineered cells comprising modified alleles of essential genes in various screening methods are also encompassed by the invention.

This application claims priority to the U.S. provisional application serial No. 60/259,128, filed Dec. 29, 2000, U.S. non-provisional application Ser. No. 09/792,024, filed Feb. 20, 2001; and U.S. provisional application serial No. 60/314,050, filed Aug. 22, 2001, which are all incorporated herein by reference in their entirety.

### 1. INTRODUCTION

The present invention is directed toward (1) methods for constructing strains useful for identification and validation of gene products as effective targets for therapeutic intervention, (2) methods for identifying and validating gene products as effective targets for therapeutic intervention, (3) a collection of identified essential genes, and (4) screening methods and assay procedures for the discovery of new drugs.

### 2. BACKGROUND OF THE INVENTION

Validation of a cellular target for drug screening purposes generally involves an experimental demonstration that inactivation of that gene product leaves the cell inviable. Accordingly, a drug active against the same essential gene product expressed, for example, by a pathogenic fungus, would be predicted to be an effective therapeutic agent. Similarly, a gene product required for fungal pathogenicity and virulence is also expected to provide a suitable target for drug screening programs. Target validation in this instance is based upon a demonstration that inactivation of the gene encoding the virulence factor creates a fungal strain that is shown to be either less pathogenic or, ideally, avirulent, in animal model studies. Identification and validation of drug targets are critical issues for detection and discovery of new drugs because these targets form the basis for high throughput screens within the pharmaceutical industry.

Target discovery has traditionally been a costly, time-consuming process, in which newly-identified genes and gene products have been individually analyzed as potentially-suitable drug targets. DNA sequence analysis of entire genomes has markedly accelerated the gene discovery process. Consequently, new methods and tools are required to analyze this information, first to identify all of the genes of the organism, and then, to discern which genes encode products that will be suitable targets for the discovery of effective, non-toxic drugs. Gene discovery through sequence analysis alone does not validate either known or novel genes as drug targets. Elucidation of the function of a gene from the underlying and a determination of whether or not that gene is essential still present substantial obstacles to the identification of appropriate drug targets. These obstacles are especially pronounced in diploid organisms.

C. albicans is a major fungal pathogen of humans. An absence of identified specific, sensitive, and unique drug targets in this organism has hampered the development of effective, non-toxic compounds for clinical use. The recent completion of the DNA sequence analysis of the entire C. albicans genome has rejuvenated efforts to identify new antifungal drug targets. Nevertheless, two primary obstacles to the exploitation of this information for the development of useful drug targets remain: the paucity of suitable markers for genetic manipulations in C. albicans and the inherent difficulty in establishing, in this diploid organism, whether a specific gene encodes an essential product. Co-pending provisional patent application, filed Feb. 18, 2000, discloses the identification of dominant selectable markers, and the construction of two genes encoding those markers, which are suitable for transformation and gene disruption in C. albicans.

Current methods for gene disruption in C. albicans (FIG. 1) typically involve a multistep process employing a “URA blaster” gene cassette which is recombined into the genome, displacing the target gene of interest. The URA blaster cassette comprises the CaURA3 marker which is selectable in the corresponding auxotrophic host and which is flanked by direct repeats of the Salmonella typhimurium HisG gene. The URA blaster cassette also carries flanking sequences corresponding to the gene to be replaced, which facilitate precise replacement of that gene by homologous recombination. Putative heterozygous transformants, which have had one allele of the target gene deleted, are selected as uracil prototrophs, and their identity and chromosomal structure confirmed by Southern blot and PCR analyses. Isolates within which intrachromosomal recombination events have occurred between HisG repeats, leading to excision of the CaURA3 gene and loss of the integrated cassette, are selected on 5-fluoroorotic acid (5-FOA) containing media. This allows a repetition of the entire process, including reuse of the Ura-blaster cassette, for disruption of the second allele of the target gene. In those instances in which the target gene is nonessential, homozygous gene disruptions are produced in the second round gene replacement and identified by Southern blot and PCR analyses.

However, homozygous deletion strains, which lack both alleles of a gene that is essential will not be viable. Accordingly, the Ura blaster method will not provide an unequivocal result, establishing the essential nature of the target gene since alternative explanations, including poor growth of a viable mutant strain, may be equally likely for the negative results obtained. More recent approaches for identification of essential genes, including those disclosed by Wilson, R. B., Davis, D., Mitchell, A. P. (1999) J. Bacteriol. 181:1868-74, employ multiple auxotrophic markers and a PCR-based gene disruption strategy. Although such methods effectively overcome the need to use the Ura Blaster cassette, determination of whether a given gene is essential, and therefore, a potentially useful target, remains labor-intensive and unsuitable for genome-wide analyses. Substantial effort is required to support a statistically valid conclusion that a given gene is essential when using either the Ura blaster cassette or multiple auxotrophic marker-based methods for gene disruption in Candida albicans. Typically, between 30 and 40 second round transformants must all be confirmed as reconstructed heterozygous strains (using PCR or Southern blot analysis) resulting from homologous recombination between the disruption fragment and previously constructed disruption allele, before statistical support to the claim that the gene is essential can be made. Moreover, since secondary mutations may be selected in either the transformation step or 5-FOA counterselection (if the Ura blaster cassette is reused), two independently constructed heterozygous strains are preferably examined during the attempted disruption of the second allele. In addition, demonstration that a particular phenotype is linked to the homozygous mutation of the target gene (and not a secondary mutation) requires complementation of the defect by transforming a wild type copy of the gene back into the disruption strain.

Finally, the Ura blaster method precludes direct demonstration of gene essentiality. Therefore, one is unable to critically evaluate the terminal phenotype characteristic of essential target genes. Consequently, establishing whether inactivation of a validated drug target gene results in cell death (i.e., a cidal terminal phenotype) versus growth inhibition (i.e., a static terminal phenotype) is not possible with current approaches, despite the value such information would provide in prioritizing drug targets for suitability in drug development.

Clearly, since current gene disruption methods are labor intensive and largely refractile to a high throughput strategy for target validation, there is a need for effective methods and tools for unambiguous, rapid, and accurate identification of essential genes in diploid, pathogenic fungi, and particularly, in Candida albicans. The present invention overcomes these limitations in current drug discovery approaches by enabling high throughput strategies that provide rapid identification, validation, and prioritization of drug targets, and consequently, accelerate drug screening.

### 3. SUMMARY OF THE INVENTION

The present invention provides effective and efficient methods that enable, for each gene in the genome of an organism, the experimental determination as to whether that gene is essential, and for a pathogenic organism, in addition, whether it is required for virulence or pathogenicity. The identification and validation of essential genes and those genes critical to the development of virulent infections, provides a basis for the development of high-throughput screens for new drugs against the pathogenic organism.

The present invention can be practiced with any organism independent of ploidy, and in particular, pathogenic fungi. Preferably, the pathogenic fungi are diploid pathogenic fungi, including but not limited to Candida albicans, Aspergillus fumigatus, Cryptococcus neoformans and the like.

In one embodiment, the present invention is directed toward a method for constructing a diploid fungal strain in which one allele of a gene is modified by insertion of or replacement by a cassette comprising an expressible dominant selectable marker. This cassette is introduced into the chromosome by recombination, thereby providing a heterozygous strain in which the first allele of the gene is inactivated.

The other allele of the gene is modified by the introduction, by recombination, of a promoter replacement fragment comprising a heterologous promoter, such that the expression of the second allele of the gene is regulated by the heterologous promoter. Expression from the heterologous promoter can be regulated by the presence of a transactivator protein comprising a DNA-binding domain and transcription-activation domain. The DNA-binding domain of this transactivator protein recognizes and binds to a sequence in the heterologous promoter and increases transcription of that promoter. The transactivator protein can be produced in the cell by expressing a nucleotide sequence encoding the protein.

This method for the construction of a diploid fungus having both alleles of a gene modified, is carried out, in parallel, with each and every gene of the organism, thereby allowing the assembly a collection of diploid fungal cells each of which comprises the modified alleles of a gene. This collection, therefore, comprises modified alleles of substantially all of the genes of the diploid organism. As used herein, the term “substantially all” includes at least 60%, 70%, 80%, 90%, 95% or 99% of the total. Preferably, every gene in the genome of the diploid organism is represented in the collection.

The present invention also encompasses diploid organisms, such as diploid pathogenic fungal strains, comprising modified alleles of a gene, where the first allele of a gene is inactivated by insertion of or replacement by a nucleotide sequence encoding an expressible dominant selectable marker; and where the second allele of the gene has also been modified so that expression of the second allele is regulated by a heterologous promoter. In one aspect of the present invention, the alleles modified in the mutant diploid fungal strain correspond to an essential gene, which is required for growth, viability and survival of the strain. In another aspect of the present invention, the modified alleles correspond to a gene required for the virulence and pathogenicity of the diploid pathogenic fungal strain against a host organism. In both cases, the essential gene and the virulence/pathogenicity gene are potential drug targets.

Accordingly, the present invention encompasses collections of mutant diploid fungal strains wherein each collection comprises a plurality of strains, each strain containing the modified alleles of a different gene. The collections of strains of the invention include modified alleles for substantially all the different essential genes in the genome of a fungus or substantially all the different virulence genes in the genome of a pathogenic fungus.

In another embodiment, the present invention is directed to nucleic acid microarrays which comprise a plurality of defined nucleotide sequences disposed at identifiable positions in an array on a substrate. The defined nucleotide sequences can comprise oligonucleotides complementary to, and capable of hybridizing with, the nucleotide sequences of the essential genes of the diploid pathogenic organism that are required for the growth and survival of the diploid pathogenic organism, the nucleotide sequences of genes contributing to the pathogenicity or virulence of the organism, and/or the unique molecular tags employed to mark each of the mutant strains.

The present invention is also directed to methods for the identification of genes essential to the survival of a diploid organism, and of genes that contribute to the virulence and/or pathogenicity of the diploid pathogenic organism. First, the invention provides mutants of diploid organisms, such as mutant fungal cells, having one allele of a gene inactivated by insertion of or replacement with a disruption cassette, and the other allele modified by a nucleic acid molecule comprising a heterologous regulated promoter, such that expression of that second allele is under the control of the heterologous promoter. Second, such mutant cells are cultured under conditions where the second allele of the modified gene is substantially not expressed. The viability or pathogenicity of the cells are then determined. The resulting loss of viability or exhibition of a severe growth defect indicates that the gene that is modified in the mutant cells is essential to the survival of a pathogenic fungus. Similarly, the resulting loss of virulence and/or pathogenicity of the mutant cells indicates that the gene that is modified contributes to the virulence and/or pathogenicity of the pathogenic fungus.

In yet another embodiment of the present invention, the mutant pathogenic fungal strains constructed according to the methods disclosed are used for the detection of antifungal agents effective against pathogenic fungi. Mutant cells of the invention are cultured under differential growth conditions in the presence or absence of a test compound. The growth rates are then compared to indicate whether or not the compound is active against a target gene product. The second allele of the target gene may be substantially underexpressed to provide cells with enhanced sensitivity to compounds active against the gene product expressed by the modified allele. Alternatively, the second allele may be substantially overexpressed to provide cells with increased resistance to compounds active against the gene product expressed by the modified allele of the target gene.

In yet another embodiment of the present invention, the strains constructed according to the methods disclosed are used for the screening of therapeutic agents effective for the treatment of non-fungal infectious diseases in a plant or an animal, such as a human. As a consequence of the similarity of a target's amino acid sequence with a plant or animal counterpart, or the lack of sequence similarity, active compounds so identified may have therapeutic applications for the treatment of diseases in the plant or animal, in particular, human diseases, such as cancers and immune disorders.

The present invention, in other embodiments, further encompasses the use of transcriptional profiling and proteomics techniques to analyze the expression of essential and/or virulence genes under a variety of conditions, including in the presence of known drugs. The information yielded from such studies can be used to uncover the target and mechanism of known drugs, to discover new drugs that act in a similar fashion to known drugs, and to delineate the interactions between gene products that are essential to growth and survival of the organism and that are instrumental to virulence and pathogenicity of the organism.

In a further embodiment of the present invention, a set of genes of a pathogenic organism are identified as potential targets for drug screening. Such genes comprise, genes that have been determined, using the methods and criteria disclosed herein, to be essential for survival of a pathogenic fungus and/or for the virulence and/or pathogenicity of the pathogenic fungus. The polynucleotides of the essential genes or virulence genes of a pathogenic organism (i.e., the target genes) provided by the present invention can be used by various drug discovery purposes. Without limitation, the polynucleotides can be used to express recombinant protein for characterization, screening or therapeutic use; as markers for host tissues in which the pathogenic organisms invade or reside (either permanently or at a particular stage of development or in a disease states); to compare with DNA sequences of other related or distant pathogenic organisms to identify potential orthologous essential or virulence genes; for selecting and making oligomers for attachment to a nucleic acid array for examination of expression patterns; to raise anti-protein antibodies using DNA immunization techniques; as an antigen to raise anti-DNA antibodies or elicit another immune response; and as a therapeutic agent (e.g., antisense). Where the polynucleotide encodes a protein which binds or potentially binds to another protein (such as, for example, in a receptor-ligand interaction), the polynucleotide can also be used in assays to identify polynucleotides encoding the other protein with which binding occurs or to identify inhibitors of the binding interaction.

The polypeptides or proteins encoded by the essential genes and virulence genes (i.e. the target gene products) provided by the present invention can also be used in assays to determine biological activity, including its uses as a member in a panel or an array of multiple proteins for high-throughput screening; to raise antibodies or to elicit immune response; as a reagent (including the labeled reagent) in assays designed to quantitatively determine levels of the protein (or its receptor) in biological fluids; as a marker for host tissues in which the pathogenic organisms invade or reside (either permanently or at a particular stage of development or in a disease states); and, of course, to isolate correlative receptors or ligands (also referred to as binding partners) especially in the case of virulence factors. Where the protein binds or potentially binds to another protein (such as, for example, in a receptor-ligand interaction), the protein can be used to identify the other protein with which binding occurs or to identify inhibitors of the binding interaction. Proteins involved in these binding interactions can also be used to screen for peptide or small molecule inhibitors or agonists of the binding interaction, such as those involved in invasiveness, and pathogenicity of the pathogenic organism.

Any or all of these drug discovery utilities are capable of being developed into a kit for commercialization as research products. The kits may comprise polynucleotides and/or polypeptides corresponding to a plurality of essential genes and virulence genes of the invention, antibodies, and/or other reagents.

### 4. BRIEF DESCRIPTION OF THE DRAWINGS

FIG. 1 depicts the URA blaster method for gene disruption in Candida albicans.

FIG. 2 depicts the GRACE method for constructing a gene disruption of one allele of a gene (CaKRE9), and promoter replacement of the second allele of the target gene, placing the second allele under conditional, regulated control by a heterologous promoter.

FIG. 3 presents conditional gene expression, using GRACE technology, with KRE1, KRE5, KRE6 and KRE9.

FIG. 4 presents conditional gene expression using GRACE technology with CaKRE1, CaTUB1, CaALG7, CaAUR1, CaFKS1 and CaSAT2.

FIG. 5 presents a Northern Blot Analysis of CaHIS3, CaALR1, CaCDC24 and CaKRE9 mRNA isolated from GRACE strains to illustrate elevated expression under non-repressing conditions.

FIG. 6 presents growth of a CaHIS3 heterozygote strain and a tetracycline promoter-regulated CaHIS3 GRACE strain compared to growth of a wild-type diploid CaHIS3 strain in the presence and absence of 3-aminotriazole (3-AT).

FIG. 6A depicts growth of a wild-type strain and a CaHIS3 heterozygote strain as compared with a CaHIS3 GRACE strain constitutively expressing the tetracycline promoter-regulated imidazoleglycerol phosphate dehydratase, in the presence of inhibitory levels of 3-aminotriazole.

FIG. 6B depicts growth of a wild-type strain, a haploinsufficient CaHIS3 heterozygote strain, and a CaHIS3 GRACE strain constitutively expressing the tetracycline promoter-regulated imidazoleglycerol phosphate dehydratase, in the presence of an intermediate level of 3-aminotriazole.

FIG. 6C depicts growth of a wild-type strain, a haploinsufficient CaHIS3 heterozygote strain, and a CaHIS3 GRACE strain minimally expressing the tetracycline promoter-regulated imidazoleglycerol phosphate dehydratase, in the presence of an intermediate level of 3-aminotriazole.

FIG. 6D demonstrates the hypersensitivity of the CaHIS3 GRACE strain minimally expressing the tetracycline promoter-regulated imidazoleglycerol phosphate dehydratase, in the presence of an intermediate level of 3-aminotriazole.

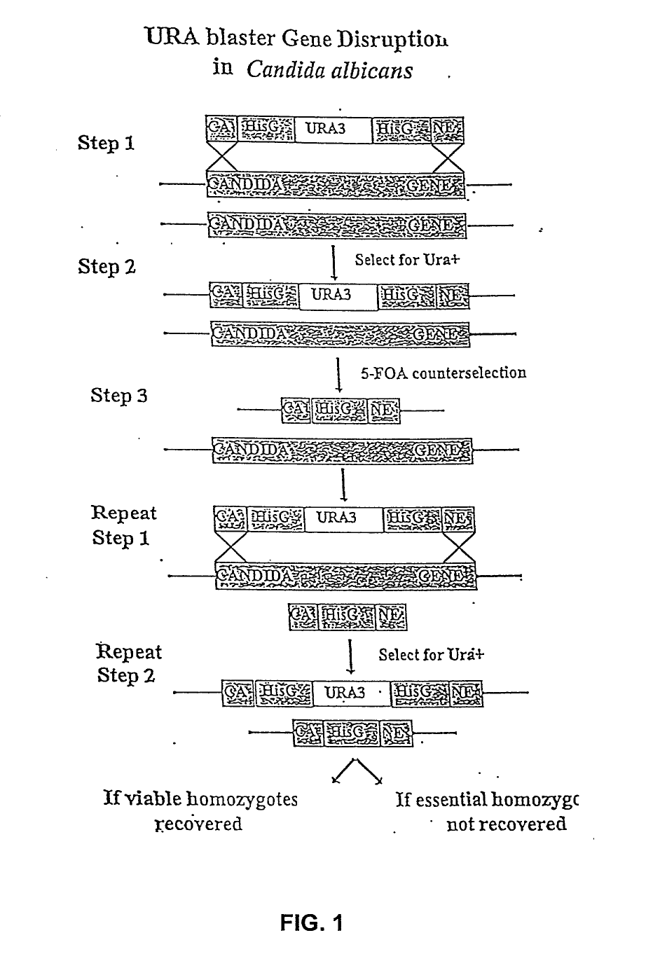

### 5. DETAILED DESCRIPTION OF THE INVENTION

5.1 Gene Disruption and Drug Target Discovery

The present invention provides a systematic and efficient method for drug target identification and validation. The approach is based on genomics information as well as the biological function of individual genes.

The methods of the invention generates a collection of genetic mutants in which the dosage of specific genes can be modulated, such that their functions in growth, survival, and/or pathogenicity can be investigated. The information accrued from such investigations allows the identification of individual gene products as potential drug targets. The present invention further provides methods of use of the genetic mutants either individually or as a collection in drug screening and for investigating the mechanisms of drug action.

Generally, in gene disruption experiments, the observation that homozygous deletions cannot be generated for both alleles of a gene in a diploid organism, cannot, per se, support the conclusion that the gene is an essential gene. Rather, a direct demonstration of expression of the gene in question that is coupled with viability of the cell carrying that gene, is required for the unambiguous confirmation that the gene in question is essential.

A direct demonstration that a given gene is essential for survival of a cell can be established by disrupting its expression in diploid organisms which have a haploid stage. For example, in Saccharomyces cerevisiae, this is achieved by complete removal of the gene product through gene disruption methods in a diploid cell type, followed by sporulation and tetrad dissection of the meiotic progeny to enable direct comparison of haploid yeast strains possessing single mutational differences. However, such an approach is not applicable to asexual yeast strains, which include most diploid pathogenic cell types, and alternative methods are required for eliminating expression of a putative essential gene.

In one embodiment, the invention provides a method for creating a diploid mutant cell of an organism in which the dosage of a specific gene can be modulated. By this method of the invention, one allele of a target gene in a diploid cell of an organism is disrupted while the second allele is modified by having its promoter replaced by a regulated promoter of heterologous origin. A strain constructed in this manner is said to comprise a modified allelic pair, i.e., a gene wherein both alleles are modified as described above. Where the genomic DNA sequence of the organism is available, this process may be repeated with each and every gene of the organism, thereby constructing a collection of mutant organisms each harboring a disrupted allele and an allele which can be conditionally expressed. This gene disruption strategy, therefore, provides a substantially complete set of potential drug target genes for that organism. This collection of mutant organisms, comprising a substantially complete set of modified allelic pairs, forms the basis for the development of high throughput drug screening assays. A collection of such mutant organisms can be made even when the genomic sequences of an organism are not completely sequenced. It is contemplated that a smaller collection of mutant organisms can be made, wherein in each mutant organism, one allele of a desired subset of gene is disrupted, and the other allele of the genes in this subset is placed under conditional expression. The method of the invention employed for the construction of such strains is referred to herein as the GRACE method, where the acronym is derived from the phrase gene replacement and conditional expression.

The GRACE method, which involves disruption of one allele coupled with conditional expression of the other allele, overcomes limitations relying upon repeated cycles of disruption with the URA blaster cassette followed by counterselection for its loss. The GRACE method permits large scale target validation in a diploid pathogenic microorganism, such as a pathogenic fungus.

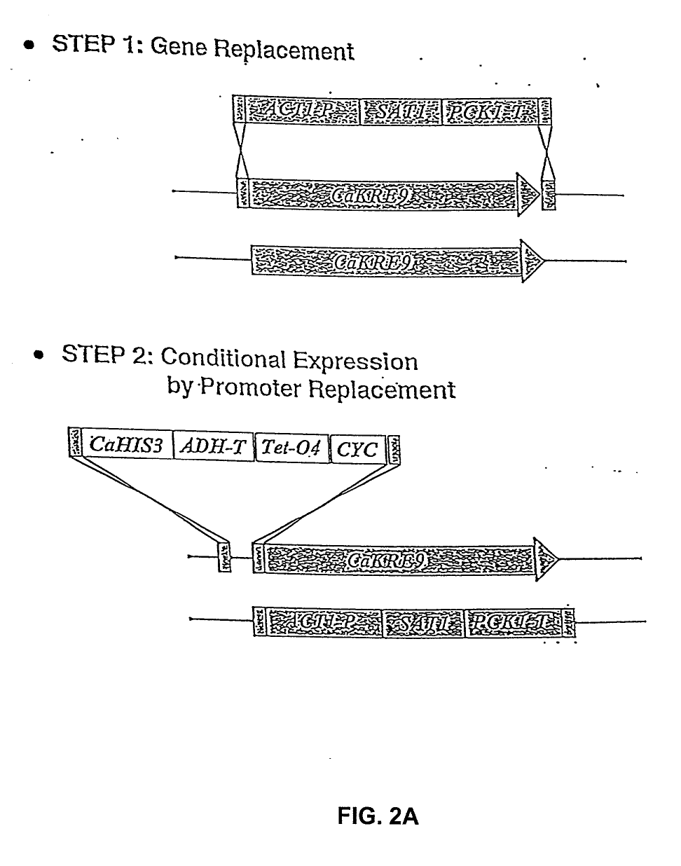

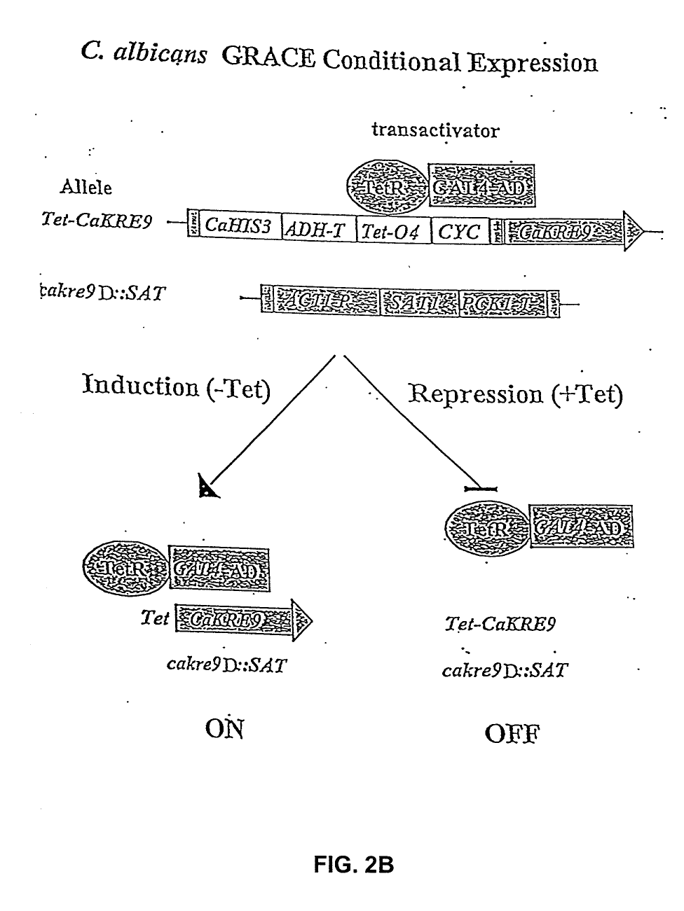

The GRACE method of the invention, as applied to a diploid cell involves two steps: (i) gene replacement resulting in disruption of the coding and/or non-coding region(s) of one wild type allele by insertion, truncation, and/or deletion, and (ii) conditional expression of the remaining wild type allele via promoter replacement or conditional protein instability (FIG. 2). Detailed descriptions of the method is provided in later sections.

Isolated mutant organisms resulting from the application of the GRACE method are referred to herein as GRACE strains of the organism. Such mutant strains of an organism are encompassed by the invention. In a particular embodiment, a collection of GRACE strains which are generated by subjecting substantially all the different genes in the genome of the organism to modification by the GRACE method is provided. In this collection, each strain comprises the modified alleles of a different gene, and substantially all the genes of the organism are represented in the collection. It is intended that a GRACE strain is generated for every gene in an organism of interest. Alternatively, a smaller collection of GRACE strains of an organism can be generated wherein a desired subset of the genes in the organism are modified by the GRACE method.

A gene is generally considered essential when viability and/or normal growth of the organism is substantially coupled to or dependent on the expression of the gene. An essential function for a cell depends in part on the genotype of the cell and in part the cell's environment. Multiple genes are required for some essential function, for example, energy metabolism, biosynthesis of cell structure, replication and repair of genetic material, etc. Thus, the expression of many genes in an organism are essential for its growth and/or survival. Accordingly, when the viability or normal growth of a GRACE strain under a defined set of conditions is coupled to or dependent on the conditional expression of the remaining functional allele of a modified allelic gene pair, the gene which has been modified in this strain by the GRACE method is referred to as an “essential gene” of the organism.

A gene is generally considered to contribute to the virulence/pathogenicity of an organism when pathogenicity of the organism is associated at least in part to the expression of the gene. Many genes in an organism are expected to contribute to the virulence and/or pathogenicity of the organism. Accordingly, when the virulence and/or pathogenicity of a GRACE strain to a defined host or to defined set of cells from a host is associated with the conditional expression of the remaining functional allele of a modified allelic gene pair, the gene which has been modified in this strain by the GRACE method is referred to as a “virulence gene” of the organism.

The present invention provides a convenient and efficient method to identify essential genes of a pathogenic organism, and to validate their usefulness in drug discovery programs. The method of the invention can similarly be used to identify virulence genes of a pathogenic organism. The identities of these essential genes and virulence genes of an organism as identified by the GRACE method are encompassed in the present invention. Substantially all of the essential genes and virulence genes of an organism can be identified and validated by the GRACE method of the invention.

Each of the essential genes and virulence genes so identified represent a potential drug target for the organism, and can be used individually or as a collection in various methods of drug screening. Depending on the objective of the drug screening program and the target disease, the essential genes and virulence genes of the invention can be classified and divided into subsets based on the structural features, functional properties, and expression profile of the gene products. The gene products encoded by the essential genes and virulence genes within each subset may share similar biological activity, similar intracellular localization, structural homology, and/or sequence homology. Subsets may also be created based on the homology or similarity in sequence to other organisms in a similar or distant taxonomic group, e.g. homology to Saccharomyces cerevisiae genes, or to human genes, or a complete lack of sequence similarity or homology to genes of other organisms, such as S. cerevisiae or human. Subsets may also be created based on the display of cidal terminal phenotype or static terminal phenotype by the organism bearing the modified gene. Such subsets, referred to as essential gene sets or virulence gene sets, which can be conveniently investigated as a group in a drug screening program, are provided by the present invention. Accordingly, the present invention provides a plurality of mutant organisms, such as a collection of GRACE strains, each comprising the modified alleles of a different gene, wherein each gene is essential for the growth and/or survival of the cells. The collection can be used according to the various methods of the invention, wherein the cells of each strain in the collection are separately subjected to the same manipulation or treatment related to the use. Alternatively, the cells of each strain in the collection are pooled before the manipulation or treatment related to the use. The concept of a collection is also extended to data collection, processing and interpretation where data arising from different strains of fungal cells or a pool of different fungal strains in the collection are handled coordinately as a set.

In a specific embodiment, substantially all of the essential genes in the genome of a pathogenic fungus are identified by the GRACE method, and the GRACE strains containing the modified allelic pairs of essential genes are included in a collection of GRACE strains. In another specific embodiment, substantially all of the virulence genes in the genome of a pathogenic fungus are identified by the GRACE method, and the GRACE strains containing the modified allelic pairs of virulence genes are included in a collection of GRACE strains.

For Candida albicans, based on analysis of the C. albicans genome sequence a collection of GRACE strains for the entire genome may comprise approximately 7000 strains each with a modified allelic pairs of genes. The complete set of essential genes of C. albicans is estimated to comprise approximately 1000 genes. The present invention provides the identities of many of these genes in C. albicans, and the various uses of these genes and their products as drug targets. In addition, estimates as to the number of genes participating in the virulence of this pathogen range between 100 and 400 genes. Once the identity of an essential gene is known, various types of mutants containing one or more copies of the mutated essential gene created by other methods beside the GRACE method are contemplated and encompassed by the invention.

The invention also provides biological and computational methods, and reagents that allow the isolation and identification of genes that are homologous to the identified essential and virulence genes of C. albicans. Information obtained from the GRACE strains of diploid organisms can be used to identify homologous sequences in haploid organisms. The identities and uses of such homologous genes are also encompassed by the present invention.

For clarity of discussion, the invention is described in the subsections below by way of example for the pathogenic fungus, Candida albicans. However, the principles may be analogously applied to the essential and virulence genes of other pathogens and parasites, of plants and animals including humans. The GRACE method can be applied to any pathogenic organisms that has a diploid phase in their life cycles. Hence, the term diploid pathogenic organism is not limited to organism that exist exclusively in diploid form, but encompasses also organisms that have both haploid and diploid phases in their life cycle.

For example, the GRACE method for drug target identification and validation can be directly applied to other pathogenic fungi. Deuteromycetous fungi, i.e. those lacking a sexual cycle and classical genetics, (in which C. albicans is included), represent the majority of human fungal pathogens. Aspergillus fumigatus is another medically-significant member of this phylum, which, more strictly, includes members of the Ascomycota and the Basidiomycota. A. fumigatus, an Ascomycte is the predominant air borne infectious fungal agent causing respiratory infection, or invasive aspergillosis (IA), in immunocompromised patients. While relatively unknown 20 years ago, today the number of IA cases is estimated to be several thousand per year. Moreover, IA exhibits a mortality rate exceeding 50% and neither amphothericin B nor fluconazole are highly efficacious. Compounding these problems is that identification of novel drug targets is limited by the current state of target validation in this organism.

The GRACE method demonstrated for C. albicans is readily adapted for use with A. fumigatus, for the following reasons. Although, A. fumigatus possesses a haploid genome, the GRACE method could be simplified to one step-conditional promoter replacement of the wild type promoter. Since A. fumigatus, in contrast to Candida albicans, adheres to the universal genetic code, extensive site-directed mutagenesis, like that required to engineer the GRACE method for C. albicans, would not be required. Moreover, essential molecular biology techniques such as transformation and gene disruption via homologous recombination have been developed for A. fumigatus. Selectable markers are available for these techniques in A. fumigatus, and include genes conferring antibiotic resistance to bygromycin B and phleomycin, and the auxotrophic marker, ura3. Furthermore, both public and private A. fumigatus genome sequencing projects exist. Therefore, sequence information is available both for the identification of putative essential genes as well as for the experimental validation of these drug targets using the GRACE method. Additional pathogenic deuteromycetous fungi to which the GRACE method may be applied include Aspergillus flavus, Aspergillus niger, and Coccidioides immitis.

In another aspect of the present invention, the GRACE method for drug target identification and validation is applied to Basidiomycetous pathogenic fungi. One particular, medically-significant member of this phylum is Cryptococcus neoformans. This air borne pathogen represents the fourth (7-8%) most commonly recognized cause of life-threatening infections in AIDS patients. Transformation and gene disruption strategies exist for C. neoformans and a publically funded genome sequencing project for this organism is in place. C. neoformans possesses a sexual cycle, thus enabling the GRACE method to be employed with both haploid and diploid strains. Other medically-significant Basidiomycetes include Trichosporon beigelii and Schizophylum commune.

In the same way medically relevant fungal pathogens are suitable for a rational drug target discovery using the present invention, so too may plant fungal pathogens and animal pathogens be examined to identify novel drug targets for agricultural and veterinary purposes. The quality and yield of many agricultural crops including fruits, nuts, vegetables, rice, soybeans, oats, barley and wheat are significantly reduced by plant fungal pathogens. Examples include the wheat fungal pathogens causing leaf blotch (Septoria tritici, glume blotch (Septoria nodorum), various wheat rusts (Puccinia recondita, Puccinia graminis); powdery mildew (various species), and stem/stock rot (Fusarium spp.). Other particularly destructive examples of plant pathogens include, Phytophthora infestans, the causative agent of the Irish potato famine, the Dutch elm disease causing ascomycetous fungus, Ophiostoma ulmi, the corn smut causing pathogen, Ustilago maydis, the rice-blast-causing pathogen Magnapurtla grisea, Peronospora parasitica (Century et al., Proc Natl Acad Sci U S A 1995 July 3;92(14):6597-601); Cladosporium fulvum (leaf mould pathogen of tomato); Fusarium graminearum, Fusarium culmorum, and Fusarium avenaceum, (wheat, Abramson et al., J Food Prot 2001 August;64(8):1220-5); Alternaria brassicicola (broccoli; Mora et al., Appl Microbiol Biotechnol 2001 April;55(3):306-10); Alternaria tagetica (Gamboa-Angulo et al., J Agric Food Chem 2001 March;49(3):1228-32); the cereal pathogen Bipolaris sorokiniana (Apoga et al., FEMS Microbiol Lett 2001 April 13; 197(2):145-50); the rice seedling blast fungus Pyricularia grisea (Lee et al., Mol Plant Microbe Interact 2001 April;14(4):527-35); the anther smut fungus Microbotryum violaceum (Bucheli et al.,: Mol Ecol 2001 February;10(2):285-94); Verticillium longisporum comb. Nov (wilt of oilseed rape, Karapapa et al., Curr Microbiol 2001 March;42(3):217-24); Aspergillus flavus infection of cotton bolls (Shieh et al., Appl Environ Microbiol 1997 September;63(9):3548-52; the eyespot pathogen Tapesia yallundae (Wood et al., FEMS Microbiol Lett 2001 March 15;196(2):183-7); Phytophthora cactorum strain P381 (strawberry leaf necrosis, Orsomando et al., J Biol Chem 2001 June 15;276(24):21578-84); Sclerotinia sclerotiorum, an ubiquitous necrotrophic fungus (sunflowers, Poussereau et al., Microbiology 2001 March;147(Pt 3):717-26); pepper plant/cranberry, anthracnose fungus Colletotrichum gloeosporioides (Kim et al., Mol Plant Microbe Interact 2001 January;14(1):80-5); Nectria haematococca (pea plants, Han et al., Plant J 2001 February;25(3):305-14); Cochliobolus heterostrophus (Monke et al., Mol Gen Genet 1993 October;241(1-2):73-80), Glomerella cingulata (Rodriquez et al., Gene 1987;54(1):73-81) obligate pathogen Bremia lactucae (lettuce downy mildew; Judelson et al., Mol Plant Microbe Interact 1990 July-August;3(4):225-32) Rhynchosporium secalis (Rohe et al., Curr Genet 1996 May;29(6):587-90), Gibberella pulicaris (Fusarium sambucinum), Leptosphaeria maculans (Farman et al., Mol Gen Genet 1992 January;231 (2):243-7), Cryphonectria parasitica and Mycosphaerella fijiensis and Mycosphaerella musicola, the causal agents of black and yellow Sigatoka, respectively, and Mycosphaerella eumusae, which causes Septoria leaf spot of banana (banana & plantain, Balint-Kurti et al., FEMS Microbiol Lett 2001 February 5;195(1):9-15). The emerging appearance of fungicidal-resistant plant pathogens and increasing reliance on monoculture practices, clearly indicate a growing need for novel and improved fungicidal compounds. The present invention encompasses identification and validation of drug targets in pathogens and parasites of plants and livestock. Accordingly, the application of the GRACE method to identify and validate drug targets in pathogens and parasites of plants and livestock are encompassed. Table I lists exemplary groups of haploid and diploid fungi of medical, agricultural, or commercial value.

TABLE I
Exemplary Haploid and Diploid Fungi

| | | |
|---|---|---|
| | | General Commercial |
| Animal pathogens: | Plant Pathogens: | Significance |

Ascomycota

| | | |
|---|---|---|
| Aspergillus fumigatus | Alternaria solanii | Aspergillus niger |
| Alternaria spp | Gaeumannomyces graminis | Schizosaccharomyces pombe |
| Blastomyces dermatidis | Cercospora zeae-maydis | Pichia pastoris |
| Candida spp including | Botrytis cinerea | Hansenula polymorpha |
| Candida dublinensis | Claviceps purpurea | Ashbya gossipii |
| Candida glabrata | Corticum rolfsii | Aspergillus nidulans |
| Candida krusei | Endothia parasitica | Trichoderma reesei |
| Candida lustaniae | Sclerotinia sclerotiorum | Aureobasidium pullulans |
| Candida parapsilopsis | Erysiphe gramini | Yarrowia lipolytica |
| Candida tropicalis | Erysiphe triticii | Candida utilis |
| Coccidioides immitis | Fusarium spp. | Kluveromyces lactis |
| Exophalia dermatiditis | Magnaporthe grisea | |
| Fusarium oxysporum | Plasmopara viticola | |
| Histoplasma capsulatum | Penicillium digitatum | |
| Pneumocystis carinii | Ophiostoma ulmi | |
| | Rhizoctonia species including oryzae | |
| | Septoria species including | |
| | Septoria avenae | |
| | Septoria nodorum | |
| | Septoria passerinii | |
| | Septoria triticii | |
| | Venturia inequalis | |
| | Verticillium dahliae | |
| | Verticillium albo-atrum | |

Basidiomycota

| | | |
|---|---|---|
| Cryptococcus neoformans | Puccinia spp including | Agaricus campestris |
| Trichosporon beigelii | Puccinia coronata | Phanerochaete chrysosporium |
| | Puccinia graminis | Gloeophyllum trabeum |
| | Puccinia recondita | Trametes versicolor |
| | Puccinia striiformis | |
| | Tilletia spp including | |
| | Tilletia caries | |
| | Tilletia controversa | |
| | Tilletia indica | |
| | Tilletia tritici | |
| | Tilletia foetida | |
| | Ustilago maydis | |
| | Ustilago hordeii | |

Zygomycota

Absidia corymbifera
Mucor rouxii
Rhizomucor pusillus
Rhizopus arrhizus

All Candida species except Candida glabrata are obligate diploid species that lack a haploid phase in its life cycle, and are thus subject to the application of the GRACE methods.

5.2 Construction of GRACE Strains

According to the invention, in a GRACE strain of a diploid organism, only one allele of a gene is eliminated, while the second allele is placed under the control of the heterologous promoter, the activity of which is regulatable. Where the gene is essential, elimination of both alleles will be lethal or severely crippling for growth. Therefore, in the present invention, a heterologous promoter is used to provide a range of levels of expression of the second allele. Depending on the conditions, the second allele can be non-expressing, underexpressing, overexpressing, or expressing at a normal level relative to that when the allele is linked to its native promoter. A heterologous promoter is a promoter from a different gene from the same pathogenic organism, or it can be a promoter from a different species.

Precise replacement of a target gene is facilitated by using a gene disruption cassette comprising a selectable marker, preferably a dominant selectable marker, that is expressible in the strain of interest. The availability of two distinct dominant selectable markers allows the gene replacement process to be engineered at both alleles of the target gene, without the required counterselection step inherent in existing methods.

In particular, the present invention encompasses a method for constructing a strain of diploid pathogenic fungal cells, in which both alleles of a gene are modified, the method comprising the steps of (a) modifying a first allele of a gene in diploid pathogenic fungal cells by recombination using a gene disruption cassette comprising a nucleotide sequence encoding a selectable marker that is expressible in the cells, thereby providing heterozygous pathogenic fungal cells in which the first allele of the gene is inactivated; and (b) modifying the second allele of the gene in the heterozygous diploid pathogenic fungal cells by recombination with a promoter replacement fragment comprising a heterologous promoter, such that the expression of the second allele of the gene is regulated by the heterologous promoter.

The process can be repeated for a desired subset of the genes such that a collection of GRACE strains is generated wherein each strain comprises a modified allelic pair of a different gene. By repeating this process for every gene in a pathogenic fungus, a complete set of GRACE strains representing the entire genome of the pathogenic fungus can be obtained. Thus, the present invention provides a method of assembling a collection of diploid pathogenic fungal cells, each of which comprises the modified alleles of a different gene. The method comprises repeating the steps of modifying pairs of alleles a plurality of times, wherein a different pair of gene alleles is modified with each repetition, thereby providing the collection of diploid pathogenic fungal cells each comprising the modified alleles of a different gene.

A preferred embodiment for the construction of GRACE strains, uses the following two-step method. C. albicans is used as an example.

5.2.1 Heterozygote Construction by Gene Disruption

Several art-known methods are available to create a heterozygote mutant. In less preferred embodiments, auxotrophic markers, such as but not limited to CaURA43, CaHIS3, CaLEU2, or CaTRP1, could be used for gene disruption if desired. However, the preferred method of heterozygote construction in diploid fungi employs a genetically modified dominant selectable marker. C. albicans is sensitive to the nucleoside-like antibiotic streptothricin at a concentration of 200 micrograms per milliliter. The presence of the Escherichia coli SAT1 gene within C. albicans allows acetylation of the drug rendering it nontoxic and permitting the strain to grow in the presence of streptothricin at a concentration of 200 micrograms per milliliter. Expression of the SAT1 gene in C. albicans is made possible by engineering the gene so that its DNA sequence is altered to conform to the genetic code of this organism and by providing a CaACT1 promoter (Morschhauser et al. (1998) Mol. Gen. Genet. 257:412-420) and a CaPCK1 terminator sequence (Leuker et al. (1997) Gene 192: 235-40). This genetically modified marker is referred to as CaSAT1 which is the subject of a copending U.S. nonprovisional application, filed Feb. 16, 2001.

C. albicans is also sensitive to a second fungicidal compound, blasticidin, whose cognate resistance gene from Bacillus cereus, BSR, has similarly been genetically engineered for expression in C. albicans (CaBSR1), and has been shown to confer a dominant drug resistance phenotype. PCR amplification of either dominant selectable marker so as to include about 65 bp of flanking sequence identical to the sequence 5′ and 3′ of the C. albicans gene to be disrupted, allows construction of a gene disruption cassette for any given C. albicans gene.

By employing the method of Baudin et al. (1993, Nucleic Acids Research 21:3329-30), a gene disruption event can be obtained following transformation of a C. albicans strain with the PCR-amplified gene disruption cassette and selection for drug resistant transformants that have precisely replaced the wild type gene with the dominant selectable marker. Such mutant strains can be selected for growth in the presence of a drug, such as but not limited to streptothricin. The resulting gene disruptions are generally heterozygous in the diploid C. albicans, with one copy of the allelic pair on one homologous chromosome disrupted, and the other allele on the other homologous chromosome remaining as a wild type allele as found in the initial parental strain. The disrupted allele is non-functional, and expression from this allele of the gene is nil. By repeating this process for all the genes in the genome of an organism, a set of gene disruptions can be obtained for every gene in the organism. The method can also be applied to a desired subset of genes.

5.2.2 Conditional Expression by a Tetracycline-Regulatable Promoter

The conditional expression system used in this embodiment of the invention comprises a regulatable promoter and a means for regulating promoter activity. Conditional expression of the remaining wild type allele in a heterozygote constructed as set forth in Section 5.1.1 is achieved by replacing its promoter with a tetracycline-regulatable promoter system that is developed initially for S. cerevisiae but which is modified for use in C. albicans. See Gari et al., 1997, Yeast 13:837-848; and Nagahashi et al., 1997, Mol. Gen. Genet. 255:372-375.

Briefly, conditional expression is achieved by first constructing a transactivation fusion protein comprising the E. coli TetR tetracycline repressor domain or DNA binding domain (amino acids 1-207) fused to the transcription activation domain of S. cerevisiae GAL4 (amino acids 785-881) or HAP4 (amino acids 424-554). Multiple CTG codon corrections were introduced to comply with the C. albicans genetic code. The nucleotide sequences encoding the transactivation fusion proteins of E. coli TetR (amino acids 1-207) plus S. cerevisiae GAL4 (amino acids 785-881), and of E. coli TetR (amino acids 1-207) plus S. cerevisiae HAP4 (amino acids 424-554), both of which have been modified for proper expression in C. albicans are encompassed by the present invention. Accordingly, the invention provides haploid or diploid cells that can comprise a nucleotide sequence encoding a transactivation fusion protein expressible in the cells, wherein the transactivation fusion protein comprises a DNA binding domain and a transcription activation domain.

Constitutive expression of the transactivation fusion protein in C. albicans can be achieved by providing a CaACT1 promoter and CaACT1 terminator sequence. However, it will be appreciated that any regulatory regions, promoters and terminators, that are functional in C. albicans can be used to express the fusion protein. Thus, a nucleic acid molecule comprising a promoter functional in C. albicans, the coding region of a transactivation fusion protein, and a terminator functional in C. albicans, are encompassed by the present invention. Such a nucleic acid molecule can be a plasmid, a cosmid, a transposon, or a mobile genetic element. In a preferred embodiment, the TetR-Gal4 or TetR-Hap4 transactivators can be stably integrated into a C. albicans strain, by using either ura3 and his3 auxotrophic markers.

In this embodiment, the invention further provides that a promoter replacement fragment comprising a nucleotide sequence encoding heterologous promoter which comprises at least one copy of a nucleotide sequence which is recognized by the DNA binding domain of the transactivation fusion protein, and wherein binding of the transactivation fusion protein increases transcription of the heterologous promoter. The heterologous tetracycline promoter initially developed for S. cerevisiae gene expression, contains an ADH1 3′ terminator sequence, variable number of copies of the tetracycline operator sequence (2, 4, or 7 copies), and the CYC1 basal promoter. The tetracycline promoter has been subcloned adjacent to both CaHIS3 and CaSAT1 selectable markers in the orientation favoring tetracycline promoter-dependent regulation when placed immediately upstream the open reading frame of the gene of interest. PCR amplification of the CaHIS3-Tet promoter cassette incorporates 65 bp of flanking sequence homologous to the promoter sequence around nucleotide positions −200 and −1 (relative to the start codon) of the target gene, thereby producing a conditional promoter replacement fragment for transformation. When transformed into a C. albicans strain made heterozygous as described in Section 5.1.1 using the CaSAT1 disruption cassette, homologous recombination between the promoter replacement fragment and the promoter of the wild type allele generates a strain in which the remaining wild type gene is conditionally regulated gene by the tetracycline promoter. Transformants are selected as His prototrophs and verified by Southern blot and PCR analysis.

In this particular embodiment, the promoter is induced in the absence of tetracycline, and repressed by the presence of tetracycline. Analogs of tetracycline, including but not limited to chlortetracycline, demeclocycline, doxycycline, meclocycline, methocycline, minocycline hydrochloride, anhydrotetracycline, and oxytetracycline, can also be used to repress the expression of the modified gene allele in a GRACE strain.

The present invention also encompasses alternative variants of the tetracycline promoter system, based upon a mutated tetracycline repressor (tetR) molecule, designated tetR′, which is activated (i.e. binds to its cognate operator sequence) by binding of the antibiotic effector molecule to promote expression, and is repressed (i.e. does not bind to the operator sequence) in the absence of the antibiotic effectors, when the tetR′ is used instead of, or in addition to, the wild-type tetR. For example, the GRACE method could be performed using tetR′ instead of tetR in cases where repression is desired under conditions which lack the presence of tetracycline, such as shut off of a gene participating in drug transport (e.g. CaCDR1, CaPDR5, or CaMDR1). Also, the GRACE method could be adapted to incorporate both the tetR and tetR′ molecules in a dual activator/repressor system where tetR is fused to an activator domain and tetR′ is fused to a general repressor (e.g. CaSsr6 or CaTup1) to enhance or further repress expression in the presence of the antibiotic effector molecules (Belli et al., 1998, Nucl Acid Res 26:942-947 which is incorporated herein by reference). These methods of providing conditional expression are also contemplated.

In another embodiment of the invention, the method may also be applied to haploid pathogenic fungi by modifying the single allele of the gene via recombination of the allele with a promoter replacement fragment comprising a nucleotide sequence encoding a heterologous promoter, such that the expression of the gene is conditionally regulated by the heterologous promoter. By repeating this process for a preferred subset of genes in a haploid pathogenic organism, or its entire genome, a collection or a complete set of conditional mutant strains can be obtained. A preferred subset of genes comprises genes that share substantial nucleotide sequence homology with target genes of other organisms, e.g., C. albicans and S. cerevisiae. For example, this variation to the method of the invention may be applied to haploid fungal pathogens including, but not limited to, animal fugal pathogens such as Aspergillus fumigatus, Aspergillus niger, Aspergillus flavis, Candida glabrata, Cryptococcus neoformans, Coccidioides immitis, Exophalia dermatiditis, Fusarium oxysporum, Histoplasma capsulatum, Pneumocystis carinii, Trichosporon beigelii, Rhizopus arrhizus, Mucor rouxii, Rhizomucor pusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species.

The means to achieve conditional expression are not restricted to the tetracycline promoter system and can be performed using other conditional promoters. Such conditional promoter may, for example, be regulated by a repressor which repress transcription from the promoter under particular condition or by a transactivator which increases transcription from the promoter, such as, when in the presence of an inducer. For example, the C. albicans CaPCK1 promoter is not transcribed in the presence of glucose but has a high level of expression in cells grown on other carbon sources, such as succinate, and therefore could also be adopted for conditional expression of the modified allele in a GRACE strain. To this end, it has been shown that both CaHIS1 and CaSAT1 are essential for growth on glucose-containing medium using the CaPCK1 promoter as an alternative to the tetracycline promoter in the above description. In this instance, the CaPCK1 promoter is heterologous to the gene expressed and not to the organism, and such heterologous promoters are also encompassed in the invention. Alternative promoters that could functionally replace the tetracycline promoter include but are not limited to other antibiotic-based regulatable promoter systems (e.g., pristinamycin-induced promoter or PIP) as well as Candida albicans conditionally-regulated promoters such as MET25, AML2, PHO5, GAL1,10, STE2, or STE3.

In a preferred embodiment of the GRACE method, performing the gene disruption first enables heterozygous strains to be constructed and separately collected as a heterozygote strain collection during the process of drug target validation. Such a C. albicans heterozygote strain collection enables drug screening approaches based on haploinsufficiency for validated targets within the collection. As used herein, the term “haploinsufficiency” refers to the phenomenon whereby heterozygous strains for a given gene express approximately half the normal diploid level of a particular gene product. Consequently, these strains provide constructions having a diminished level of the encoded gene product, and they may be used directly in screens for antifungal compounds. Here differential sensitivity of a diploid parent, as compared with its heterozygous derivative, will indicate that a drug is active against the encoded gene product.

It is clear to those skilled in the art that the order of allele modification followed in this embodiment of the invention is not critical, and that it is feasible to perform these steps in a different order such that the conditional-expressing allele is constructed first and the disruption of the remaining wild type gene allele be performed subsequently. However, where the promoter replacement step is carried out first, care should be taken to delete sequences homologous to those employed in the gene disruption step.

A specific application of the GRACE method, as used to construct modified alleles of the target gene CaKRE9 is provided in Section 6.

5.2.3 Alternative Methods of Conditional Expression

In other embodiments of the invention, conditional expression could be achieved by means other than the reliance of conditional promoters. For example, conditional expression could be achieved by the replacement of the wild type allele in heterozygous strains with temperature sensitive alleles derived in vitro, and their phenotype would then be analyzed at the nonpermissive temperature. In a related approach, insertion of a ubiquitination signal into the remaining wild type allele to destabilize the gene product during activation conditions can be adopted to examine phenotypic effects resulting from gene inactivation. Collectively, these examples demonstrate the manner in which C. albicans genes can be disrupted and conditionally regulated using the GRACE method.

In an alternative embodiment of the present invention, a constitutive promoter regulated by an excisable transactivator can be used. The promoter is placed upstream to a target gene to repress expression to the basal level characteristic of the promoter. For example, in a fungal cell, a heterologous promoter containing lexA operator elements may be used in combination with a fusion protein composed of the lexA DNA binding domain and any transcriptional activator domain (e.g. GAL4, HAP4, VP16) to provide constitutive expression of a target gene. Counterselection mediated by 5-FOA can be used to select those cells which have excised the gene encoding the fusion protein. This procedure enables an examination of the phenotype associated with repression of the target gene to the basal level of expression provided by the lexA heterologous promoter in the absence of a functional transcription activator. The GRACE strains generated by this approach can be used for drug target validation as described in detail in the sections below. In this system, the low basal level expression associated with the heterologous promoter is critical. Thus, it is preferable that the basal level of expression of the promoter is low to make this alternative shut-off system more useful for target validation.

Alternatively, conditional expression of a target gene can be achieved without the use of a transactivator containing a DNA binding, transcriptional activator domain. A cassette could be assembled to contain a heterologous constitutive promoter downstream of, for example, the URA3 selectable marker, which is flanked with a direct repeat containing homologous sequences to the 5′ portion of the target gene. Additional homologous sequences upstream of the target, when added to this cassette would facilitate homologous recombination and replacement of the native promoter withe above-described heterologous promoter cassette immediately upstream of the start codon of the target gene or open reading frame. Conditional expression is achieved by selecting strains, by using 5-FOA containing media, which have excised the heterologous constitutive promoter and URA3 marker (and consequently lack those regulatory sequences upstream of the target gene required for expression of the gene) and examining the growth of the resulting strain versus a wild type strain grown under identical conditions.

5.2.4 GRACE strains of Filamentous Plant Pathogenic Fungi

In specific embodiments, the methods of identifying drug targets of the invention can be applied to filamentous plant pathogenic fungi. A wide variety of filamentous fungi cause plant diseases; these fungi include species in the genera Ustilago, Fusarium, Colletotrichum, Botrytis, Septoria, Rhizoctonia, Puccinia, Tilletia and Gaemannomyces. In particular, pathogenic fungi of the Fusarium group cause many economically significant diseases on crop plants and some species also cause human infections. For example, plant pathogenic species such as F. graminearum, which causes head scab of wheat, can have devastating economic effects, e.g., $2.6 billion in crop losses over the last 10 years in the U.S.

A majority of techniques and reagents applicable to genetic engineering in fungi in general are useful in the present invention. The transformation procedure for most filamentous plant pathogenic fungi is based on the protocol developed for Aspergillus nidulans by Yelton et al. (1984. Proc. Natl. Acad. Sci. 81: 1470-1474). The protocol involved creating protoplasts by Novozyme 234 digestion of the cell wall material from mycelium or newly germinated conidial spores. Protoplasts are separated from the cell wall debris by filtration, centrifugation and (in some species) gradient purification. DNA is introduced in the presence of CaCl₂ and polyethylene glycol, and protoplasts are regenerated on medium containing an osmotic stabilizer (such as sorbitol). A. nidulans metabolic genes such as TrpC, ArgB and the amdS gene (growth on acetamide) have commonly been used as selectable markers. Metabolic markers for other fungi include the PyrG gene and the gene for nitrate reductase. Dominant selectable markers generally include genes for resistance to hygromycin, benomyl, bialophos, phleomycin and, more recently, pyrithiamine. By far, resistance to hygromycin is the most common selection for obtaining transformants and most vectors are based on the marker developed by Punt et al. (pAN7-1; Gene. 56: 117-124, 1987). Promoters to drive transcription of marker genes include the A. nidulans trpC and gpd promoters although many more of the characterized promoters can be used. Well-studied regulated promoters are available from genes involved in nitrogen metabolism (e.g. see publications by the laboratories of Michael Hynes, George Marzluf and Herb Arst). In addition, regulated promoters have been identified for plant pathogens such as the promoter for the pg1 gene encoding polygalacturonase which is induced upon growth with pectin as the carbon source (Di Pietro and Roncero. 1998. MPMI 11: 91-98.). Generally, targeted integration of transforming DNA occurs at a lower frequency than in S. cerevisiae, but nonetheless sufficient for gene replacement and the GRACE promoter replacement method.

In preferred embodiments, the invention encompasses modified strains and essential genes of basidiomycetes which comprises, for example, the Ustilago species. In particular, Ustilago maydis (corn smut) is a dimorphic basidiomycete fungus related to many fungal plant pathogens such as the economically important bunts and rusts. Other Ustilago species, such as U. hordei, are common pathogens of small grain cereals such as barley, oats and wheat. In the Ustilago species, the budding form is haploid, unicellular and nonpathogenic; this cell type serves as a genetically tractable model system in which molecular biological methods can readily be applied to identify essential genes (Banuett, F. Annual Reviews in Genetics (1995) 29;179-208). Fusion of two haploid cells of opposite mating type produces a dikaryotic filamentous form which is pathogenic and which requires the host plant for growth. The GRACE method can be adapted to target validation within U. maydis and U. hordei for identifying novel plant pathogen essential targets suitable for agricultural purposes. A comparative analysis with U. maydis and U. hordei may provide a significant advantage because the analyses could help identify essential genes.

U. maydis and U. hordei are preferred plant pathogenic fungi for constructing GRACE strains for use in the methods of drug targets identification of the invention. In Ustilago species, gene replacement by homologous recombination is efficient. Targeted disruptions using 1 kb flanking sequence yields as high as 70-90% correct integration Protoplast-based transformation protocols typically yield 50-100 colonies/μg. For example, dominant selectable markers including nourseothricin (NSR), hygromycin B (hygB), phleomycin, benomyl, carboxin, and geneticin, as well as autonomously replicating and integration plasmids are available (Kojic M, and Holloman W K Can J Microbiol 2000 46:333-8, and Gold, S., G. Bakkeren, J. Davies and J. W. Kronstad. 1994. Gene 142: 225-230). Accordingly, standard gene disruption experiments may be performed by those skilled in the art using gene disruption cassettes containing dominant selectable markers suitable for selection in U. maydis (e.g. nourseothricin, hygromycin B, phleomycin, or carboxin dominant selectable markers may be used). These may be amplified by three-way PCR methodology (Wach, A. 1996. Yeast Vol. 12:259-265) to add flanking homologous sequence of suitable length and permit precise gene replacement. Alternatively, auxotrophic markers may be used to select for stable integration of the disruption cassette within any corresponding U. maydis and U. hordei auxotrophic mutant. Alternative recombinant DNA methods to construct suitable U. maydis and U. hordei gene disruption cassettes are also readily available to those skilled in the art.

Transformation of the resulting disruption cassettes may be performed as described by Wang, et al. 1988. Proc. Natl. Acad. Sci., 85: 865-869. Briefly, transformation in U. maydis involves removing the cell wall with lysing enzyme (e.g. Novozyme or Sigma L1412), adding DNA, treating the cells with PEG and plating on medium with 1M sorbitol and antibiotic selection. Transformants appear in 3 to 5 days. Alternatively, diploid U. maydis strains are also publicly available and have been used for the analysis of essential genes (e.g., Holden et al., 1989. EMBO J. 8: 1927-1934.). Specifically, one allele is disrupted in the diploid strain, as outlined above, and the heterozygous strain is injected into corn seedlings. Diploid spores are harvested 14 days later, the spores are germinated to obtain meiotic progeny. Random spore analysis of the resulting progeny is then performed whereby haploid strains are screened for the absence of any identifiable disrupted allele within the population. A statistical analysis may then be performed to determine the essentiality of the examined gene based on the absence of identifying any viable haploid strains maintaining the deletion allele.

PCR-based promoter replacement experiments using the GRACE regulatable promoter system in U. maydis may be performed by those skilled in the art by first constructing a functional transactivator protein which regulates the GRACE tetracycline promoter. The transactivator protein must be constitutively expressed at high levels. Possible U. maydis regulatory sequence includes the UmTEF1 and UmHSP70 promoters and their respective 3′UTR sequence. The resulting transactivator may be subcloned into a suitable U. maydis plasmid (e.g., pCM54; Tsukuda, et al., 1988. Mol. Cell. Biol. 8:3703-3709.) containing a dominant selectable marker (e.g. HygB) and transformed into any U. maydis homothalic wild-type strain (e.g. 518 (a2 b2) and 521 (a1 b1) (Banuett, F. Trends in Genetics (1992) 8:174-180. Alternatively, a number U. maydis and U. hordei strains containing stable auxotrophic mutations are publicly available and may be used in conjunction with cognate auxotrophic marker cassettes to introduce and stably express the transactivator protein.

As U. maydis and U. hordei are haploid fungal organisms, the GRACE methodology may then be applied as a single step involving precise promoter replacement using a tetracycline promoter replacement cassette. Preferably, this may be performed using 3-way PCR products comprising a NSR dominant selectable marker fused to the Tet promoter and flanked with appropriate homologous sequence and transforming the promoter replacement cassette into a U. maydis strain constitutively expressing the Tet transactivator protein. Alternative dominant selectable markers may also be employed. Precise replacement by homologous recombination between the wild type promoter and the dominant marker-fused Tet conditional promoter facilitates conditional mutant U. maydis strain construction in a single step. Correct integration of the promoter replacement cassette may be experimentally determined by PCR-mediated genotyping and/or Southern blot analysis.

Alternatively endogenous regulatable promoters may be applied to constructing conditional mutant strains of U. maydis. Preferable regulatable promoters which may be used include, but are not restricted to, the crg1 gene promoter which is regulated by carbon source (Bottin, A., Kamper, J. and Kahmann, R. Mol. Gen. Genet. 253: 342-352 (1996) and the nar1 gene promoter (nitrate reductase) has also been developed for regulating gene expression (Brachmann, A. et al. 2001. Mol. Microbiol. 42: 1047-1063).

U. hordei has a very similar life cycle when compared with U. maydis except that the fungus grows more slowly in culture and crosses require the complete growth cycle of the barley plant (2 months) to complete. U. hordei is closely related to a large group of Ustilago species that cause economically more important diseases on small grain cereals. These other species include U. tritici, U. nuda, U. avenae and U. kolleri. U. hordei which are amenable to the methods of the invention also shows remarkably similarities to the bunt pathogens that cause important cereal diseases. Haploid and stable diploid strains of U. hordei are available and formation of stable U. hordei diploids (Int. J. Plant Sci. 155: 15-22) offers the ability to evaluate gene essentiality by random spore analysis as described above for U. maydis.

Gene disruption in U. hordei is accomplished in an identical fashion to that of U. maydis and the common selectable markers (e.g., hygromycin resistance) function in both species (Bakkeren, G. and J. Kronstad. 1996. Genetics 143: 1601-1613.). Gene replacement has been demonstrated for several genes at the mating type locus (Lee, N., G. Bakkeren, K. Wong, J. E. Sherwood and J. W. Kronstad. 1999. Proc. Natl. Acad. Sci., USA. 96: 15026-15031.). One minor technical difference in the transformation of U. hordei, compared with U. maydis, is that electroporation enhances the uptake of DNA in U. hordei. Preferred target genes for use in construction of GRACE strains include pan1 which participates in pantothenic acid biosynthesis (Bakkeren, G., and J. W. Kronstad. 1993. The Plant Cell 5: 123-136) and fil1 encoding a Gα subunit (Lichter A, Mills D. 1997. Mol Gen Genet. 256: 426-435).

In various embodiments, the hph gene isolated from E. coli, encoding hygromycin resistance, can be used generally as a selectable marker and GUS can be used as a reporter gene. Non-limiting examples of useful recombinant regulatable gene expression systems include the following: F. oxysporum panC promoter induced by steroidal glycoalkaloid alpha-tomatine (Perez-Espinosa et al., : Mol Genet Genomics 2001 July;265(5):922-9); Ustilago maydis hsp70-like gene promoter in a high-copy number autonomously replicating expression vector (Keon et al., Antisense Nucleic Acid Drug Dev 1999 February;9(1):101-4); Cochliobolus heterostrophus transient and stable gene expression systems using P1 or GPD1 (glyceraldehyde 3 phosphate dehydrogenase) promoter of C. heterostrophus or GUS or hygromycin B phosphotransferase gene (hph) of E. coli (Monke et al., Mol Gen Genet 1993 October;241(1-2):73-80); Rhynchosporium secalis (barley leaf scald fungus) transformed to hygromycin-B and phleomycin resistance using the hph gene from E. coli and the ble gene from Streptoalloteichus hindustanus under the control of Aspergillus nidulans promoter and terminator sequences, plasmid DNA introduced into fungal protoplasts by PEG/CaCl₂ treatment (Rohe et al., Curr Genet 1996 May;29(6):587-90). Pathogens of banana and plantain (Musa spp.) Mycosphaerella fijiensis and Mycosphaerella musicola, and Mycosphaerella eumusae can be transformed as taught in Balint-Kurti et al., FEMS Microbiol Lett 2001 February 5;195(1):9-15. Gibberella pulicaris (Fusarium sambucinum) a trichothecene-producing plant pathogen can be transformed with three different vectors: cosHyg1, pUCH1, and pDH25, all of which carry hph (encoding hygromycin B phosphotransferase) as the selectable marker (Salch et al., Curr Genet 1993;23(4):343-50). Leptosphaeria maculans, a fungal pathogen of Brassica spp. can be transformed with the vector pAN8-1, encoding phleomycin resistance; protoplasts can be retransformed using the partially homologous vector, pAN7-1 which encodes hygromycin B resistance. Farman et al., Mol Gen Genet 1992 January;231(2):243-7. Cryphonectria parasitica; targeted disruption of enpg-1 of this chestnut blight fungus was accomplished by homologous recombination with a cloned copy of the hph gene of Escherichia coli inserted into exon 1, see Gao et al., Appl Environ Microbiol 1996 June;62(6):1984-90.

Another example, Glomerella cingulata f sp. phaseoli (Gcp) was transformed using either of two selectable markers: the amdS+ gene of Aspergillus nidulans, which encodes acetamidase and permits growth on acetamide as the sole nitrogen source and the hygBR gene of Escherichia coli which permits growth in the presence of the antibiotic Hy. The amdS+ gene functioned in Gcp under control of A. nidulans regulatory signals and hygBR was expressed after fusion to a promoter from Cochliobolus heterostrophus, another filamentous ascomycete. Protoplasts to be transformed were generated with the digestive enzyme complex Novozym 234 and then were exposed to plasmid DNA in the presence of 10 mM CaCl₂ and polyethylene glycol. Transformation occurred by integration of single or multiple copies of either the amdS+ or hygBR plasmid into the fungal genome. (Rodriquez et al., Gene 1987;54(1):73-81); integration vectors for homologous recombination; deletion studies demonstrated that 505 bp (the minimum length of homologous promoter DNA analysed which was still capable of promoter function) was sufficient to target integration events. Homologous integration of the vector resulted in duplication of the gdpA promoter region. (Rikkerink et al., Curr Genet 1994 March;25(3):202-8).

5.3 Identification of Essential Genes and Virulence Genes

5.3.1 Essential Genes

The present invention provides methods for determining whether the gene that has been modified in a GRACE strain is an essential gene or a virulence gene in a pathogenic organism of interest. To determine whether a gene is an essential gene in an organism, a GRACE strain containing the modified alleles of the gene is cultured under conditions wherein the second modified allele of the gene which is under conditional expression, is substantially underexpressed or not expressed. The viability and/or growth of the GRACE strain is compared with that of a wild type strain cultured under the same conditions. A loss or reduction of viability or growth indicates that the gene is essential to the survival of a pathogenic fungus. Accordingly, the present invention provides a method for identifying essential genes in a diploid pathogenic organism comprising the steps of culturing a plurality of GRACE strains under culture conditions wherein the second allele of each of the gene modified in the respective GRACE strain is substantially underexpressed or not expressed; determining viability and/or growth indicator(s) of the cells; and comparing that with the viability and/or growth indicator(s) of wild type cells. The level of expression of the second allele can be less than 50% of the non-modified allele, less than 30%, less than 20%, and preferably less than 10%. Depending on the heterologous promoter used, the level of expression can be controlled by, for example, antibiotics, metal ions, specific chemicals, nutrients, pH, temperature, etc.

Candida albicans is used herein as an example which has been analyzed by the GRACE methodology.

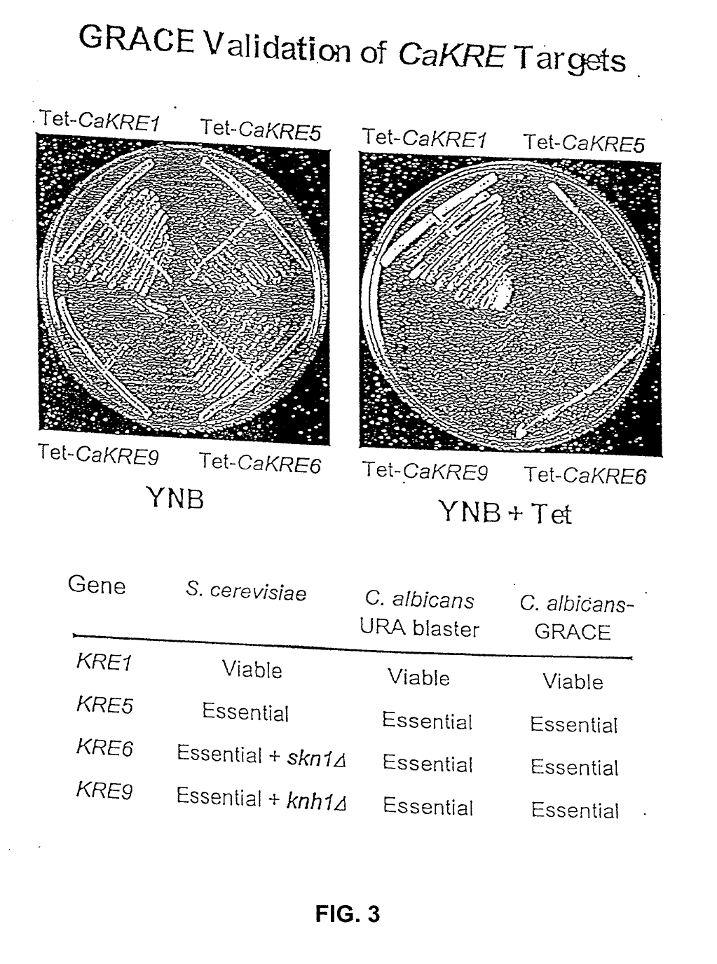

For example, C. albicans conditional gene expression using the GRACE method was performed using CaKRE1, CaKRE5, CaKRE6, and CaKRE9 (FIG. 3). CaKRE5, CaKRE6, and CaKRE9 are predicted to be essential or conditionally essential (CaKRE9 null strains are nonviable on glucose but viable on galactose), in C. albicans as demonstrated by gene disruption using the Ura blaster method. CaKRE1 has been demonstrated as a nonessential gene using the Ura blaster method in C. albicans. Strains heterozygous for the above genes were constructed by PCR-based gene disruption method using the CaSAT1 disruption cassette followed by tetracycline regulated promoter replacement of the native promoter of the wild type allele. Robust growth of each of these strains suggests expression proceeds normally in the absence of tetracycline. When tetracycline is added to the growth medium, expression of these tetracycline promoter-regulated genes is greatly reduced or abolished. In the presence of tetracycline, the GRACE strain cells containing each one of the three essential C. albicans genes cited above stop growing. As expected, only the CaKRE1 GRACE strain demonstrates robust growth despite repression of CaKRE1 expression.

To further examine the utility of the GRACE method in target validation, growth of four additional GRACE strains controlling expression of the known essential genes CaTUB1, CaALG7, CaAUR1, and CaFKS1, as well as the predicted essential gene CaSAT2, and CaKRE1 were compared under inducing versus repressing conditions (FIG. 4). As expected, GRACE strains of CaTUB1, CaALG7, CaAUR1 and CaFKS1 failed to grow under repressing conditions, unlike the non-essential CaKRE1 GRACE strain. Furthermore, as predicted, the CaSAT2 GRACE strain demonstrates essentiality of this gene in C. albicans. The CaSAT2 gene, which has been engineered as a dominant selectable marker for use in C. albicans, is a C. albicans gene that is homologous to a S. cerevisiae gene but is unrelated to the Sat1 gene of E. coli.

In all cases based on other disruption data that have been generated, this is the expected response if the tetracycline regulated gene is repressed to a level where it is nonfunctional in the presence of tetracycline. Furthermore, in applying the GRACE methodology of conditional gene disruption to two additional C. albicans genes (CaYPD1, and CaYNL194c) whose S. cerevisiae counterpart is known not to be essential, no inhibition of growth was observed when these strains were incubated in the presence of tetracycline. These results establish that the method of conditional gene expression using a GRACE strain is a reliable indicator of gene essentiality.

Furthermore, the utility of the present method, as a rapid and accurate means to identifying the complete set of essential genes in C. albicans, has been demonstrated by an analysis of the null phenotype of a large number of genes using the GRACE two-step method of gene disruption and conditional expression. Target genes were selected as being fungal specific and essential. Such genes are referred to as target essential genes in the screening assays described below.

A literature search identified reports of URA blaster-based gene disruption experiments on a total of 89 genes, of which 13 genes were presumed to be essential, based on the inability to construct homozygous deletion strains. The 13 genes are CaCCT8 Rademacher et al., Microbiology, UK 144, 2951-2960 (1998)); CaFKS1 (Mio et al., J. Bacteriol, 179, 4096-105 (1997); and Douglas, et al., Antimicrob Agents Chemother 41, 2471-9 (1997)); CaHSP90 (Swoboda et al., Infect Immun 63, 4506-14 (1995)); CaKRE6 (Mio et al., J. Bacteriol 179, 2363-72 (1997)); CaNMT1 (Weinberg et al., Mol Microbiol 16, 241-50 (1995)); CaPRS1 (Payne et al., J. Med. Vet. Mycol. 35, 305-12 (1997)); CaPSA1 (Care et al., Mol Microbiol 34, 792-798 (1999)); CaRAD6 (Care et al., Mol Microbiol 34, 792-798 (1999)); CaSEC4 (Mao et al., J. Bacteriol 181, 7235-7242 (1999)); CaSEC14 (Monteoliva et al., Yeast 12, 1097-105 (1996)); CaSNF1 (Petter et al., Infect Immun. 65, 4909-17 (1997)); CaTOP2 (Keller, et al., Biochem J., 329-39 (1997)); and CaEFT2 (Mendoza et al., Gene 229, 183-1991 (1999)). These 13 putatively essential genes and CaTUB1, CaALG1, and CaAUR1 of C. albicans are not initially identified by the GRACE method. However, GRACE strains containing modified alleles of any one of these 17 genes and their uses are encompassed by the invention, for example, the CaTUB1, CaALG1, and CaAUR1 GRACE strains in FIG. 4 and the CaKRE6 GRACE strain in FIG. 3. Any of these 17 genes may be included as a control for comparisons in the methods of the invention, or as a positive control for essentiality in the collections of essential genes of the invention. The nucleic acid molecules comprising a nucleotide sequence corresponding to any of these 17 genes may be used in the methods of drug discovery of the invention as drug targets, or they may be included individually or in subgroups as controls in a kit or in a nucleic acid microarray of the invention.

In contrast to the use of conventional method, application of the GRACE method has already identified significantly more C. albicans essential genes than previously determined by the collective efforts of the entire C. albicans research community. The data presented herewith establishes the speed inherent to the approach of the invention and, therefore, the feasibility of extending the GRACE method to the examination of all the genes of the C. albicans genome, the identification of the complete set of essential genes of this diploid fungal pathogen, and its application to other species.

An alternative method is available for assessing the essentiality of the modified gene in a GRACE strain. According to the invention, repression of expression of the modified gene allele within a GRACE strain may be achieved by homologous recombination-mediated excision of the gene encoding the transactivator protein. In a preferred embodiment, where conditional expression of a target gene is achieved using the tetracycline-regulated promoter, constitutive expression (under nonrepressing conditions) may be repressed by homologous recombination-mediated excision of the transactivator gene (TetR-GAL4AD). In this way, an absolute achievable repression level is produced independently of that produced by tetracycline-mediated inactivation of the transactivator protein. Excision of the transactivator gene is made possible by virtue of the selectable marker and integration strategy used in GRACE strain construction. Stable integration of the CaURA3-marked plasmid containing the TetR-GAL4AD transactivator gene into the CaLEU2 locus results in a tandem duplication of CaLEU2 flanking the integrated plasmid. Counterselection on 5-FOA-containing medium can then be performed to select for excision of the CaURA3-marked transactivator gene and to directly examine whether this alternative repression strategy reveals the target gene to be essential.

Three examples of genes defined as essential on 5-FOA containing medium but lacking any detectable growth impairment on tetracycline supplemented medium are the genes, CaYCL052c, CaYNL194c and CaYJR046c. Presumably, this is due to the target gene exhibiting a lower basal level of expression under conditions where the transactivator gene has been completely eliminated than its gene product incompletely inactivated by addition of tetracycline. Thus, the GRACE method offers two independent approaches for the determination of whether or not a given gene is essential for viability of the host strain.

5.3.2 Virulence/Pathogenicity Genes

The present invention also provides methods of using the GRACE strains of a diploid pathogenic organism to identify virulence/pathogenicity genes. In addition to uncovering essential genes of a pathogenic organism, the GRACE methodology enables the identification of other genes and gene products potentially relevant to the screening of drugs useful for the treatment of diseases caused by the pathogenic organism. Nonessential genes and their gene products of a pathogen which nevertheless display indispensable roles in the pathogenesis process, may therefore serve as potential drug targets for prophylactic drug development and could be used in combination with existing cidal therapeutics to improve treatment strategies. Thus, genes and their products implicated in virulence and/or pathogenicity represent another important class of potential drug targets. Moreover, some of the genes implicated in virulence and pathogenicity may be species-specific, and unique to a particular strain of pathogen. It has been estimated that approximately 6-7% of the genes identified through the C. albicans sequencing project are absent in S. cerevisiae. This represents as many as 420 Candida albicans-specific genes which potentially participate in the process of pathogenesis or virulence. Such a large scale functional evaluation of this gene set can only be achieved using the GRACE methodology of the invention.

Although essential genes provide preferred targets, value would also be placed on those nonessential C. albicans specific genes identified. The potential role of nonessential C. albicans-specific genes in pathogenesis may be evaluated and prioritized according to virulence assays (e.g. buccal epithelial cell adhesion assays and macrophage assays) and various C. albicans infection studies (e.g. oral, vaginal, systemic) using mouse or other animal models. In the same manner described above for essential genes, it is equally feasible to demonstrate whether nonessential genes comprising the GRACE strain collection are required for pathogenicity in a cellular assay or in a mouse model system. Accordingly, GRACE strains that fail to cause fungal infection in mice under conditions of gene inactivation by tetracycline (or alternative gene inactivation means) define the GRACE virulence/pathogenicity subset of genes. More defined subsets of virulence/pathogenicity genes, for example those genes required for particular steps in pathogenesis (e.g. adherence or invasion) can be determined by applying the GRACE pathogenicity subset of strains to in vitro assays which measure the corresponding process. For example, examining GRACE pathogenicity strains in a buccal adhesion or macrophage assay by conditional expression of individual genes would identify those pathogenicity factors required for adherence or cell invasion respectively. Moreover, essential genes that display substantially reduced virulence and growth rate when only partially inactivated represent “multifactorial” drug targets for which even minimally inhibitory high specificity compounds would display therapeutic value.

Accordingly, to determine whether a gene contributes toward the virulence/pathogenicity of a pathogenic organism in a host, a GRACE strain of the pathogen containing the modified alleles of the gene is allowed to infect host cells or animals under conditions wherein the second modified allele of the gene which is under conditional expression, is substantially underexpressed or not expressed. After the host cells and/or animals have been contacted with the GRACE strain for an appropriate period of time, the condition of the cells and/or animals is compared with cells and/or animals infected by a wild type strain under the same conditions. Various aspects of the infected cell's morphology, physiology, and/or biochemistry can be measured by methods known in the art. When an animal model is used, the progression of the disease, severity of the symptoms, and/or survival of the host can be determined. Any loss or reduction of virulence or pathogenicity displayed by the GRACE strain indicates that the gene modified in the strain contributes to or is critical to the virulence and/or pathogenicity of the virus. Such genes are referred to as target virulence genes in the screening assays described below.

In another aspect of the present invention, GRACE methodology can be used for the identification and delineation of genetic pathways known to be essential to the development of pathogenicity. For example, extensive work in S. cerevisiae has uncovered a number of processes including cell adhesion, signal transduction, cytoskeletal assembly, that play roles in the dimorphic transition between yeast and hyphal morphologies. Deletion of orthologous genes participating in functionally homologous cellular pathways in pathogenic fungi such as C. albicans, A. fumigatus, and C. neoformans, has clearly demonstrated a concomitant loss of virulence. Therefore, the use of GRACE strains of orthologous genes found in C. albicans and other pathogenic fungi could rapidly validate potential antifungal drug target genes whose inactivation impairs hyphal development and pathogenicity.

5.3.3 Validation of Genes Encoding Drug Targets

Target gene validation refers to the process by which a gene product is identified as suitable for use in screening methods or assays in order to find modulators of the function or structure of that gene product. Criteria used for validation of a gene product as a target for drug screening, however, may be varied depending on the desired mode of action that the compounds sought will have, as well as the host to be protected.

In one aspect of the present invention, a set of GRACE strains identified and grouped as having only modified alleles of essential genes can be used directly for drug screening.

In another aspect, the initial set of essential genes is further characterized using, for example, nucleotide sequence comparisons, to identify a subset of essential genes which include only those genes specific to fungi—that is, a subset of genes encoding essential genes products which do not have homologs in a host of the pathogen, such as humans. Modulators, and preferably inhibitors, of such a subset of genes in a fungal pathogen of humans would be predicted to be much less likely to have toxic side effects when used to treat humans.

Similarly, other subsets of the larger essential gene set could be defined to include only those GRACE strains carrying modified allele pairs that do not have a homologous sequence in one or more host (e.g., mammalian) species to allow the detection of compounds expected to be used in veterinary applications. In addition, using other homology criteria, a subset of GRACE strains could be identified that would be used for the detection of anti-fungal compounds active against agricultural pathogens, inhibiting targets that do not have homologs in the crop to be protected.

Current C. albicans gene disruption strategies identify nonessential genes and permit the inference that other genes are essential, based on a failure to generate a homozygous null mutant. The null phenotype of a drug target predicts the absolute efficaciousness of the “perfect” drug acting on this target. For example, the difference between a cidal (cell death) versus static (inhibitory growth) null terminal phenotype for a particular drug target. Gene disruption of CaERG11, the drug target of fluconazole, is presumed to be essential based on the failure to construct a homozygous CaERG11 deletion strain using the URA blaster method. However, direct evaluation of its null phenotype being cidal or static could not be performed in the pathogen, and only after the discovery of fluconazole was it possible to biochemically determine both the drug, and presumably the drug target to be static rather than as cidal. Despite the success fluconazole enjoys in the marketplace, its fungistatic mode of action contributes to its primary limitation, i.e., drug resistance after prolonged treatment. Therefore, for the first time, the ability to identify and evaluate cidal null phenotypes for validated drug targets within the pathogen as provided by the invention, now enables directed strategies to identifying antifungal drugs that specifically display a fungicidal mode of action.

Using a single GRACE strain or a desired collection of GRACE strains comprising essential genes, one or more target genes can be directly evaluated as displaying either a cidal or static null phenotype. This is determined by first incubating GRACE strains under repressing conditions for the conditional expression of the second allele for varying lengths of time in liquid culture, and measuring the percentage of viable cells following plating a defined number of cells onto growth conditions which relieve repression. The percentage of viable cells that remain after return to non-repressing conditions reflects either a cidal (low percent survival) or static (high percent survival) phenotype. Alternatively, vital dyes such as methylene blue or propidium iodide could be used to quantify percent viability of cells for a particular strain under repressing versus inducing conditions. As known fungicidal drug targets are included in the GRACE strain collection (e.g. CaAUR1), direct comparisons can be made between this standard fungicidal drug target and novel targets comprising the drug target set. In this way each member of the target set can be immediately ranked and prioritized against an industry standard cidal drug target to select appropriate drug targets and screening assays for the identification of the most rapid-acting cidal compounds.

5.4 Essential Genes and Virulence Genes

5.4.1 Nucleic Acids Encoding Targets, Vectors, and Host Cells

By practice of the methods of the invention, the essentiality and the contribution to virulence of substantially all the genes in the genome of an organism can be determined. The identities of essential genes and virulence genes of a diploid pathogenic organism, such as Candida albicans, once revealed by the methods of the invention, allow the inventors to study their functions and evaluate their usefulness as drug targets. Information regarding the structure and function of the gene product of the individual essential gene or virulence gene allows one to design reagents and assays to find compounds that interfere with its expression or function in the pathogenic organism. Accordingly, the present invention provides information on whether a gene or its product(s) is essential to growth, survival, or proliferation of the pathogenic organism, or that a gene or its product(s) contributes to virulence or pathogenicity of the organism with respect to a host. Based on this information, the invention further provides, in various embodiments, novel uses of the nucleotide and/or amino acid sequences of genes that are essential and/or that contributes to virulence or pathogenicity of a pathogenic organism, for purpose of discovering drugs that act against the pathogenic organism. Moreover, the present invention provides specifically the use of this information to identify orthologs of these essential genes in a non-pathogenic yeast, such as Saccharomyces cerevisiae, and the use of these orthologs in drug screening methods. Although the nucleotide sequence of the orthologs of these essential genes in S. cerevisiae may be known, it was not appreciated that these S. cerevisiae genes can be useful for discovering drugs against pathogenic fungi.

As used herein, the terms “gene” and “recombinant gene” refer to nucleic acid molecules comprising a nucleotide sequence encoding a polypeptide or a biologically active ribonucleic acid (RNA). The term can further include nucleic acid molecules comprising upstream, downstream, and/or intron nucleotide sequences. The term “open reading frame (ORF),” means a series of nucleotide triplets coding for amino acids without any termination codons and the triplet sequence is translatable into protein using the codon usage information appropriate for a particular organism.

As used herein, the term “target gene” refers to either an essential gene or a virulence gene useful in the invention, especially in the context of drug screening. The terms “target essential gene” and “target virulence gene” will be used where it is appropriate to refer to the two groups of genes separately. However, it is expected that some genes will contribute to virulence and be essential to the survival of the organism. The target genes of the invention may be partially characterized, fully characterized, or validated as a drug target, by methods known in the art and/or methods taught hereinbelow. As used herein, the term “target organism” refers to a pathogenic organism, the essential and/or virulence genes of which are useful in the invention.

The term “nucleotide sequence” refers to a heteropolymer of nucleotides, including but not limited to ribonucleotides and deoxyribonucleotides, or the sequence of these nucleotides. The terms “nucleic acid” and “polynucleotide” are also used interchangeably herein to refer to a heteropolymer of nucleotides, which may be unmodified or modified DNA or RNA. For example, polynucleotides can be single-stranded or double-stranded DNA, DNA that is a mixture of single-stranded and double-stranded regions, hybrid molecules comprising DNA and RNA with a mixture of single-stranded and double-stranded regions. In addition, the polynucleotide can be composed of triple-stranded regions comprising DNA, RNA, or both. A polynucleotide can also contain one or modified bases, or DNA or RNA backbones modified for nuclease resistance or other reasons. Generally, nucleic acid segments provided by this invention can be assembled from fragments of the genome and short oligonucleotides, or from a series of oligonucleotides, or from individual nucleotides, to provide a synthetic nucleic acid.

The term “recombinant,” when used herein to refer to a polypeptide or protein, means that a polypeptide or protein is derived from recombinant (e.g., microbial or mammalian) expression systems. “Microbial” refers to recombinant polypeptides or proteins made in bacterial or fungal (e.g., yeast) expression systems. As a product, “recombinant microbial” defines a polypeptide or protein essentially unaccompanied by associated native glycosylation. Polypeptides or proteins expressed in most bacterial cultures, e.g., E. coli, will be free of glycosylation modifications; polypeptides or proteins expressed in yeast will be glycosylated.

The term “expression vehicle or vector” refers to a plasmid or phage or virus, for expressing a polypeptide from a nucleotide sequence. An expression vehicle can comprise a transcriptional unit, also referred to as an expression construct, comprising an assembly of (1) a genetic element or elements having a regulatory role in gene expression, for example, promoters or enhancers, (2) a structural or coding sequence which is transcribed into mRNA and translated into protein, and which is operably linked to the elements of (1); and (3) appropriate transcription initiation and termination sequences. “Operably linked” refers to a link in which the regulatory regions and the DNA sequence to be expressed are joined and positioned in such a way as to permit transcription, and ultimately, translation. In the case of C. albicans, due to its unusual codon usage, modification of a coding sequence derived from other organisms may be necessary to ensure a polypeptide having the expected amino acid sequence is produced in this organism. Structural units intended for use in yeast or eukaryotic expression systems preferably include a leader sequence enabling extracellular secretion of translated protein by a host cell. Alternatively, where a recombinant protein is expressed without a leader or transport sequence, it may include an N-terminal methionine residue. This residue may or may not be subsequently cleaved from the expressed recombinant protein to provide a final product.

The term “recombinant host cells” means cultured cells which have stably integrated a recombinant transcriptional unit into chromosomal DNA or carry stably the recombinant transcriptional unit extrachromosomally. Recombinant host cells as defined herein will express heterologous polypeptides or proteins, and RNA encoded by the DNA segment or synthetic gene in the recombinant transcriptional unit. This term also means host cells which have stably integrated a recombinant genetic element or elements having a regulatory role in gene expression, for example, promoters or enhancers. Recombinant expression systems as defined herein will express RNA, polypeptides or proteins endogenous to the cell upon induction of the regulatory elements linked to the endogenous DNA segment or gene to be expressed. The cells can be prokaryotic or eukaryotic.

The term “polypeptide” refers to the molecule form by joining amino acids to each other by peptide bonds, and may contain amino acids other than the twenty commonly used gene-encoded amino acids. The term “active polypeptide” refers to those forms of the polypeptide which retain the biologic and/or immunologic activities of any naturally occurring polypeptide. The term “naturally occurring polypeptide” refers to polypeptides produced by cells that have not been genetically engineered and specifically contemplates various polypeptides arising from post-translational modifications of the polypeptide including, but not limited to, proteolytic processing, acetylation, carboxylation, glycosylation, phosphorylation, lipidation and acylation.

The term “isolated” as used herein refers to a nucleic acid or polypeptide separated from at least one macromolecular component (e.g., nucleic acid or polypeptide) present with the nucleic acid or polypeptide in its natural source. In one embodiment, the polynucleotide or polypeptide is purified such that it constitutes at least 95% by weight, more preferably at least 99.8% by weight, of the indicated biological macromolecules present (but water, buffers, and other small molecules, especially molecules having a molecular weight of less than 1000 daltons, can be present).

Table II lists a set of fungal specific genes that are demonstrated to be essential in C. albicans when conditionally expressed under the tetracycline repression system in the respective GRACE strains or when the gene encoding the transactivator protein is excised in the respective GRACE strain in a 5-FOA assay.

TABLE II
Sequence identifiers of essential genes and related oligonucleotides

| | | | | | | | DNA | Protein |
|---|---|---|---|---|---|---|---|---|
| Gene Name | KO-Up | KO-Down | Tet-Up | Tet-Down | Primer A | Primer B | Sequence | Sequence |
| CaYDL105W | 1 | 1001 | 2001 | 3001 | 4001 | 5001 | 6001 | 7001 |
| CaYJL090C | 2 | 1002 | 2002 | 3002 | 4002 | 5002 | 6002 | 7002 |
| CaYLR127C | 3 | 1003 | 2003 | 3003 | 4003 | 5003 | 6003 | 7003 |
| CaYNL151C | 4 | 1004 | 2004 | 3004 | 4004 | 5004 | 6004 | 7004 |
| CaYPL083C | 5 | 1005 | 2005 | 3005 | 4005 | 5005 | 6005 | 7005 |
| CaYHR036W | 6 | 1006 | 2006 | 3006 | 4006 | 5006 | 6006 | 7006 |
| CaYNL256W | 7 | 1007 | 2007 | 3007 | 4007 | 5007 | 6007 | 7007 |
| CaYOL149W | 8 | 1008 | 2008 | 3008 | 4008 | 5008 | 6008 | 7008 |
| CaYDR361C | 9 | 1009 | 2009 | 3009 | 4009 | 5009 | 6009 | 7009 |
| CaYDR407C | 10 | 1010 | 2010 | 3010 | 4010 | 5010 | 6010 | 7010 |
| CaYBR070C | 11 | 1011 | 2011 | 3011 | 4011 | 5011 | 6011 | 7011 |
| CaYOR148C | 12 | 1012 | 2012 | 3012 | 4012 | 5012 | 6012 | 7012 |
| CaYJR041C | 13 | 1013 | 2013 | 3013 | 4013 | 5013 | 6013 | 7013 |
| CaYGR090W | 14 | 1014 | 2014 | 3014 | 4014 | 5014 | 6014 | 7014 |
| CaYBR123C | 15 | 1015 | 2015 | 3015 | 4015 | 5015 | 6015 | 7015 |
| CaYHR118C | 16 | 1016 | 2016 | 3016 | 4016 | 5016 | 6016 | 7016 |
| CaYKR063C | 17 | 1017 | 2017 | 3017 | 4017 | 5017 | 6017 | 7017 |
| CaYOR004W | 18 | 1018 | 2018 | 3018 | 4018 | 5018 | 6018 | 7018 |
| CaYML025C | 19 | 1019 | 2019 | 3019 | 4019 | 5019 | 6019 | 7019 |
| CaYKL033W | 20 | 1020 | 2020 | 3020 | 4020 | 5020 | 6020 | 7020 |
| CaYDR498C | 21 | 1021 | 2021 | 3021 | 4021 | 5021 | 6021 | 7021 |
| CaYIR011C | 22 | 1022 | 2022 | 3022 | 4022 | 5022 | 6022 | 7022 |
| CaYMR220W | 23 | 1023 | 2023 | 3023 | 4023 | 5023 | 6023 | 7023 |
| CaYPR105C | 24 | 1024 | 2024 | 3024 | 4024 | 5024 | 6024 | 7024 |
| CaYDL153C | 25 | 1025 | 2025 | 3025 | 4025 | 5025 | 6025 | 7025 |
| CaYPL128C | 26 | 1026 | 2026 | 3026 | 4026 | 5026 | 6026 | 7026 |
| CaYER026C | 27 | 1027 | 2027 | 3027 | 4027 | 5027 | 6027 | 7027 |
| CaYKL004W | 28 | 1028 | 2028 | 3028 | 4028 | 5028 | 6028 | 7028 |
| CaYMR200W | 29 | 1029 | 2029 | 3029 | 4029 | 5029 | 6029 | 7029 |
| CaYPR165W | 30 | 1030 | 2030 | 3030 | 4030 | 5030 | 6030 | 7030 |
| CaYHR007C | 31 | 1031 | 2031 | 3031 | 4031 | 5031 | 6031 | 7031 |
| CaYJL087C | 32 | 1032 | 2032 | 3032 | 4032 | 5032 | 6032 | 7032 |
| CaYLR229C | 33 | 1033 | 2033 | 3033 | 4033 | 5033 | 6033 | 7033 |
| CaYER118C | 34 | 1034 | 2034 | 3034 | 4034 | 5034 | 6034 | 7034 |
| CaYPL228W | 35 | 1035 | 2035 | 3035 | 4035 | 5035 | 6035 | 7035 |
| CaYPL160W | 36 | 1036 | 2036 | 3036 | 4036 | 5036 | 6036 | 7036 |
| CaYHR101C | 37 | 1037 | 2037 | 3037 | 4037 | 5037 | 6037 | 7037 |
| CaYML085C | 38 | 1038 | 2038 | 3038 | 4038 | 5038 | 6038 | 7038 |
| CaYBR243C | 39 | 1039 | 2039 | 3039 | 4039 | 5039 | 6039 | 7039 |
| CaYLR342W | 40 | 1040 | 2040 | 3040 | 4040 | 5040 | 6040 | 7040 |
| CaYOL026C | 41 | 1041 | 2041 | 3041 | 4041 | 5041 | 6041 | 7041 |
| CaYGR251W | 42 | 1042 | 2042 | 3042 | 4042 | 5042 | 6042 | 7042 |
| CaYDR118W | 43 | 1043 | 2043 | 3043 | 4043 | 5043 | 6043 | 7043 |
| CaYJL085W | 44 | 1044 | 2044 | 3044 | 4044 | 5044 | 6044 | 7044 |
| CaYDR052C | 45 | 1045 | 2045 | 3045 | 4045 | 5045 | 6045 | 7045 |
| CaYGR002C | 46 | 1046 | 2046 | 3046 | 4046 | 5046 | 6046 | 7046 |
| CaYLL004W | 47 | 1047 | 2047 | 3047 | 4047 | 5047 | 6047 | 7047 |
| CaYOR075W | 48 | 1048 | 2048 | 3048 | 4048 | 5048 | 6048 | 7048 |
| CaYMR005W | 49 | 1049 | 2049 | 3049 | 4049 | 5049 | 6049 | 7049 |
| CaYHR172W | 50 | 1050 | 2050 | 3050 | 4050 | 5050 | 6050 | 7050 |
| CaYGL122C | 51 | 1051 | 2051 | 3051 | 4051 | 5051 | 6051 | 7051 |
| CaYOR287C | 52 | 1052 | 2052 | 3052 | 4052 | 5052 | 6052 | 7052 |
| CaYMR149W | 53 | 1053 | 2053 | 3053 | 4053 | 5053 | 6053 | 7053 |
| CaYKR071C | 54 | 1054 | 2054 | 3054 | 4054 | 5054 | 6054 | 7054 |
| CaYDR412W | 55 | 1055 | 2055 | 3055 | 4055 | 5055 | 6055 | 7055 |
| CaYKR025W | 56 | 1056 | 2056 | 3056 | 4056 | 5056 | 6056 | 7056 |
| CaYJR112W | 57 | 1057 | 2057 | 3057 | 4057 | 5057 | 6057 | 7057 |
| CaYMR277W | 58 | 1058 | 2058 | 3058 | 4058 | 5058 | 6058 | 7058 |
| CaYKR083C | 59 | 1059 | 2059 | 3059 | 4059 | 5059 | 6059 | 7059 |
| CaYNL245C | 60 | 1060 | 2060 | 3060 | 4060 | 5060 | 6060 | 7060 |
| CaYNL181W | 61 | 1061 | 2061 | 3061 | 4061 | 5061 | 6061 | 7061 |
| CaYNL260C | 62 | 1062 | 2062 | 3062 | 4062 | 5062 | 6062 | 7062 |
| CaYDR365C | 63 | 1063 | 2063 | 3063 | 4063 | 5063 | 6063 | 7063 |
| CaYNL149C | 64 | 1064 | 2064 | 3064 | 4064 | 5064 | 6064 | 7064 |
| CaYGL029W | 65 | 1065 | 2065 | 3065 | 4065 | 5065 | 6065 | 7065 |
| CaYOR057W | 66 | 1066 | 2066 | 3066 | 4066 | 5066 | 6066 | 7066 |
| CaYIL022W | 67 | 1067 | 2067 | 3067 | 4067 | 5067 | 6067 | 7067 |
| CaYMR203W | 68 | 1068 | 2068 | 3068 | 4068 | 5068 | 6068 | 7068 |
| CaYOR206W | 69 | 1069 | 2069 | 3069 | 4069 | 5069 | 6069 | 7069 |
| CaYBR167C | 70 | 1070 | 2070 | 3070 | 4070 | 5070 | 6070 | 7070 |
| CaYDR016C | 71 | 1071 | 2071 | 3071 | 4071 | 5071 | 6071 | 7071 |
| CaYNL306W | 72 | 1072 | 2072 | 3072 | 4072 | 5072 | 6072 | 7072 |
| CaYJR067C | 73 | 1073 | 2073 | 3073 | 4073 | 5073 | 6073 | 7073 |
| CaYDR362C | 74 | 1074 | 2074 | 3074 | 4074 | 5074 | 6074 | 7074 |
| CaYLR355C | 75 | 1075 | 2075 | 3075 | 4075 | 5075 | 6075 | 7075 |
| CaYLR105C | 76 | 1076 | 2076 | 3076 | 4076 | 5076 | 6076 | 7076 |
| CaYML127W | 77 | 1077 | 2077 | 3077 | 4077 | 5077 | 6077 | 7077 |
| CaYPL011C | 78 | 1078 | 2078 | 3078 | 4078 | 5078 | 6078 | 7078 |
| CaYKL108W | 79 | 1079 | 2079 | 3079 | 4079 | 5079 | 6079 | 7079 |
| CaYCR035C | 80 | 1080 | 2080 | 3080 | 4080 | 5080 | 6080 | 7080 |
| CaYML114C | 81 | 1081 | 2081 | 3081 | 4081 | 5081 | 6081 | 7081 |
| CaYNL118C | 82 | 1082 | 2082 | 3082 | 4082 | 5082 | 6082 | 7082 |
| CaYDR527W | 83 | 1083 | 2083 | 3083 | 4083 | 5083 | 6083 | 7083 |
| CaYBR256C | 84 | 1084 | 2084 | 3084 | 4084 | 5084 | 6084 | 7084 |
| CaYGL233W | 85 | 1085 | 2085 | 3085 | 4085 | 5085 | 6085 | 7085 |
| CaYLR103C | 86 | 1086 | 2086 | 3086 | 4086 | 5086 | 6086 | 7086 |
| CaYOR340C | 87 | 1087 | 2087 | 3087 | 4087 | 5087 | 6087 | 7087 |
| CaYPR175W | 88 | 1088 | 2088 | 3088 | 4088 | 5088 | 6088 | 7088 |
| CaYJR093C | 89 | 1089 | 2089 | 3089 | 4089 | 5089 | 6089 | 7089 |
| CaYCL031C | 90 | 1090 | 2090 | 3090 | 4090 | 5090 | 6090 | 7090 |
| CaYML130C | 91 | 1091 | 2091 | 3091 | 4091 | 5091 | 6091 | 7091 |
| CaYAL033W | 92 | 1092 | 2092 | 3092 | 4092 | 5092 | 6092 | 7092 |
| CaYNL062C | 93 | 1093 | 2093 | 3093 | 4093 | 5093 | 6093 | 7093 |
| CaYNL132W | 94 | 1094 | 2094 | 3094 | 4094 | 5094 | 6094 | 7094 |
| CaYDL193W | 95 | 1095 | 2095 | 3095 | 4095 | 5095 | 6095 | 7095 |
| CaYDR489W | 96 | 1096 | 2096 | 3096 | 4096 | 5096 | 6096 | 7096 |
| CaYJL069C | 97 | 1097 | 2097 | 3097 | 4097 | 5097 | 6097 | 7097 |
| CaYPL063W | 98 | 1098 | 2098 | 3098 | 4098 | 5098 | 6098 | 7098 |
| CaYNL232W | 99 | 1099 | 2099 | 3099 | 4099 | 5099 | 6099 | 7099 |
| CaYNR054C | 100 | 1100 | 2100 | 3100 | 4100 | 5100 | 6100 | 7100 |
| CaYGR245C | 101 | 1101 | 2101 | 3101 | 4101 | 5101 | 6101 | 7101 |
| CaYPR162C | 102 | 1102 | 2102 | 3102 | 4102 | 5102 | 6102 | 7102 |
| CaYHR058C | 103 | 1103 | 2103 | 3103 | 4103 | 5103 | 6103 | 7103 |
| CaYKR081C | 104 | 1104 | 2104 | 3104 | 4104 | 5104 | 6104 | 7104 |
| CaYNL240C | 105 | 1105 | 2105 | 3105 | 4105 | 5105 | 6105 | 7105 |
| CaYPR168W | 106 | 1106 | 2106 | 3106 | 4106 | 5106 | 6106 | 7106 |
| CaYKL099C | 107 | 1107 | 2107 | 3107 | 4107 | 5107 | 6107 | 7107 |
| CaYLR008C | 108 | 1108 | 2108 | 3108 | 4108 | 5108 | 6108 | 7108 |
| CaYOL142W | 109 | 1109 | 2109 | 3109 | 4109 | 5109 | 6109 | 7109 |
| CaYDL015C | 110 | 1110 | 2110 | 3110 | 4110 | 5110 | 6110 | 7110 |
| CaYDR472W | 111 | 1111 | 2111 | 3111 | 4111 | 5111 | 6111 | 7111 |
| CaYNR046W | 112 | 1112 | 2112 | 3112 | 4112 | 5112 | 6112 | 7112 |
| CaYDR473C | 113 | 1113 | 2113 | 3113 | 4113 | 5113 | 6113 | 7113 |
| CaYGL207W | 114 | 1114 | 2114 | 3114 | 4114 | 5114 | 6114 | 7114 |
| CaYHR088W | 115 | 1115 | 2115 | 3115 | 4115 | 5115 | 6115 | 7115 |
| CaYIR015W | 116 | 1116 | 2116 | 3116 | 4116 | 5116 | 6116 | 7116 |
| CaYHR197W | 117 | 1117 | 2117 | 3117 | 4117 | 5117 | 6117 | 7117 |
| CaYMR218C | 118 | 1118 | 2118 | 3118 | 4118 | 5118 | 6118 | 7118 |
| CaYKL182W | 119 | 1119 | 2119 | 3119 | 4119 | 5119 | 6119 | 7119 |
| CaYDR325W | 120 | 1120 | 2120 | 3120 | 4120 | 5120 | 6120 | 7120 |
| CaYLL003W | 121 | 1121 | 2121 | 3121 | 4121 | 5121 | 6121 | 7121 |
| CaYNR026C | 122 | 1122 | 2122 | 3122 | 4122 | 5122 | 6122 | 7122 |
| CaYNL251C | 123 | 1123 | 2123 | 3123 | 4123 | 5123 | 6123 | 7123 |
| CaYPL126W | 124 | 1124 | 2124 | 3124 | 4124 | 5124 | 6124 | 7124 |
| CaYLR002C | 125 | 1125 | 2125 | 3125 | 4125 | 5125 | 6125 | 7125 |
| CaYJL061W | 126 | 1126 | 2126 | 3126 | 4126 | 5126 | 6126 | 7126 |
| CaYLR071C | 127 | 1127 | 2127 | 3127 | 4127 | 5127 | 6127 | 7127 |
| CaYML031W | 128 | 1128 | 2128 | 3128 | 4128 | 5128 | 6128 | 7128 |
| CaYIL147C | 129 | 1129 | 2129 | 3129 | 4129 | 5129 | 6129 | 7129 |
| CaYJL025W | 130 | 1130 | 2130 | 3130 | 4130 | 5130 | 6130 | 7130 |
| CaYOR353C | 131 | 1131 | 2131 | 3131 | 4131 | 5131 | 6131 | 7131 |
| CaYKR008W | 132 | 1132 | 2132 | 3132 | 4132 | 5132 | 6132 | 7132 |
| CaYMR033W | 133 | 1133 | 2133 | 3133 | 4133 | 5133 | 6133 | 7133 |
| CaYNL313C | 134 | 1134 | 2134 | 3134 | 4134 | 5134 | 6134 | 7134 |
| CaYGL225W | 135 | 1135 | 2135 | 3135 | 4135 | 5135 | 6135 | 7135 |
| CaYNL308C | 136 | 1136 | 2136 | 3136 | 4136 | 5136 | 6136 | 7136 |
| CaYDR353W | 137 | 1137 | 2137 | 3137 | 4137 | 5137 | 6137 | 7137 |
| CaYIL068C | 138 | 1138 | 2138 | 3138 | 4138 | 5138 | 6138 | 7138 |
| CaYPR190C | 139 | 1139 | 2139 | 3139 | 4139 | 5139 | 6139 | 7139 |
| CaYOR174W | 140 | 1140 | 2140 | 3140 | 4140 | 5140 | 6140 | 7140 |
| CaYDL150W | 141 | 1141 | 2141 | 3141 | 4141 | 5141 | 6141 | 7141 |
| CaYAL041W | 142 | 1142 | 2142 | 3142 | 4142 | 5142 | 6142 | 7142 |
| CaYMR227C | 143 | 1143 | 2143 | 3143 | 4143 | 5143 | 6143 | 7143 |
| CaYPL043W | 144 | 1144 | 2144 | 3144 | 4144 | 5144 | 6144 | 7144 |
| CaYDR324C | 145 | 1145 | 2145 | 3145 | 4145 | 5145 | 6145 | 7145 |
| CaYOL022C | 146 | 1146 | 2146 | 3146 | 4146 | 5146 | 6146 | 7146 |
| CaYOL069W | 147 | 1147 | 2147 | 3147 | 4147 | 5147 | 6147 | 7147 |
| CaYGR156W | 148 | 1148 | 2148 | 3148 | 4148 | 5148 | 6148 | 7148 |
| CaYDL003W | 149 | 1149 | 2149 | 3149 | 4149 | 5149 | 6149 | 7149 |
| CaYDR228C | 150 | 1150 | 2150 | 3150 | 4150 | 5150 | 6150 | 7150 |
| CaYKR062W | 151 | 1151 | 2151 | 3151 | 4151 | 5151 | 6151 | 7151 |
| CaYDR398W | 152 | 1152 | 2152 | 3152 | 4152 | 5152 | 6152 | 7152 |
| CaYNL126W | 153 | 1153 | 2153 | 3153 | 4153 | 5153 | 6153 | 7153 |
| CaYKL089W | 154 | 1154 | 2154 | 3154 | 4154 | 5154 | 6154 | 7154 |
| CaYMR028W | 155 | 1155 | 2155 | 3155 | 4155 | 5155 | 6155 | 7155 |
| CaYDR299W | 156 | 1156 | 2156 | 3156 | 4156 | 5156 | 6156 | 7156 |
| CaYOL034W | 157 | 1157 | 2157 | 3157 | 4157 | 5157 | 6157 | 7157 |
| CaYGR119C | 158 | 1158 | 2158 | 3158 | 4158 | 5158 | 6158 | 7158 |
| CaYDL111C | 159 | 1159 | 2159 | 3159 | 4159 | 5159 | 6159 | 7159 |
| CaYHR052W | 160 | 1160 | 2160 | 3160 | 4160 | 5160 | 6160 | 7160 |
| CaYKL021C | 161 | 1161 | 2161 | 3161 | 4161 | 5161 | 6161 | 7161 |
| CaYLL031C | 162 | 1162 | 2162 | 3162 | 4162 | 5162 | 6162 | 7162 |
| CaYHR040W | 163 | 1163 | 2163 | 3163 | 4163 | 5163 | 6163 | 7163 |
| CaYML015C | 164 | 1164 | 2164 | 3164 | 4164 | 5164 | 6164 | 7164 |
| CaYIL004C | 165 | 1165 | 2165 | 3165 | 4165 | 5165 | 6165 | 7165 |
| CaYDR302W | 166 | 1166 | 2166 | 3166 | 4166 | 5166 | 6166 | 7166 |
| CaYPR133C | 167 | 1167 | 2167 | 3167 | 4167 | 5167 | 6167 | 7167 |
| CaYDL195W | 168 | 1168 | 2168 | 3168 | 4168 | 5168 | 6168 | 7168 |
| CaYCR052W | 169 | 1169 | 2169 | 3169 | 4169 | 5169 | 6169 | 7169 |
| CaYFR042W | 170 | 1170 | 2170 | 3170 | 4170 | 5170 | 6170 | 7170 |
| CaYNR017W | 171 | 1171 | 2171 | 3171 | 4171 | 5171 | 6171 | 7171 |
| CaYOR254C | 172 | 1172 | 2172 | 3172 | 4172 | 5172 | 6172 | 7172 |
| CaYFL029C | 173 | 1173 | 2173 | 3173 | 4173 | 5173 | 6173 | 7173 |
| CaYBR265W | 174 | 1174 | 2174 | 3174 | 4174 | 5174 | 6174 | 7174 |
| CaYNL312W | 175 | 1175 | 2175 | 3175 | 4175 | 5175 | 6175 | 7175 |
| CaYBR155W | 176 | 1176 | 2176 | 3176 | 4176 | 5176 | 6176 | 7176 |
| CaYGR280C | 177 | 1177 | 2177 | 3177 | 4177 | 5177 | 6177 | 7177 |
| CaYJL203W | 178 | 1178 | 2178 | 3178 | 4178 | 5178 | 6178 | 7178 |
| CaYIR012W | 179 | 1179 | 2179 | 3179 | 4179 | 5179 | 6179 | 7179 |
| CaYMR093W | 180 | 1180 | 2180 | 3180 | 4180 | 5180 | 6180 | 7180 |
| CaYPR137W | 181 | 1181 | 2181 | 3181 | 4181 | 5181 | 6181 | 7181 |
| CaYLR298C | 182 | 1182 | 2182 | 3182 | 4182 | 5182 | 6182 | 7182 |
| CaYBR192W | 183 | 1183 | 2183 | 3183 | 4183 | 5183 | 6183 | 7183 |
| CaYPR112C | 184 | 1184 | 2184 | 3184 | 4184 | 5184 | 6184 | 7184 |
| CaYLL011W | 185 | 1185 | 2185 | 3185 | 4185 | 5185 | 6185 | 7185 |
| CaYER082C | 186 | 1186 | 2186 | 3186 | 4186 | 5186 | 6186 | 7186 |
| CaYDL217C | 187 | 1187 | 2187 | 3187 | 4187 | 5187 | 6187 | 7187 |
| CaYFL035C | 188 | 1188 | 2188 | 3188 | 4188 | 5188 | 6188 | 7188 |
| CaYOR262W | 189 | 1189 | 2189 | 3189 | 4189 | 5189 | 6189 | 7189 |
| CaYLR323C | 190 | 1190 | 2190 | 3190 | 4190 | 5190 | 6190 | 7190 |
| CaYAR007C | 191 | 1191 | 2191 | 3191 | 4191 | 5191 | 6191 | 7191 |
| CaYBL023C | 192 | 1192 | 2192 | 3192 | 4192 | 5192 | 6192 | 7192 |
| CaYBL026W | 193 | 1193 | 2193 | 3193 | 4193 | 5193 | 6193 | 7193 |
| CaYBL030C | 194 | 1194 | 2194 | 3194 | 4194 | 5194 | 6194 | 7194 |
| CaYBL035C | 195 | 1195 | 2195 | 3195 | 4195 | 5195 | 6195 | 7195 |
| CaYBL040C | 196 | 1196 | 2196 | 3196 | 4196 | 5196 | 6196 | 7196 |
| CaYBL050W | 197 | 1197 | 2197 | 3197 | 4197 | 5197 | 6197 | 7197 |
| CaYBL076C | 198 | 1198 | 2198 | 3198 | 4198 | 5198 | 6198 | 7198 |
| CaYBR002C | 199 | 1199 | 2199 | 3199 | 4199 | 5199 | 6199 | 7199 |
| CaYBR029C | 200 | 1200 | 2200 | 3200 | 4200 | 5200 | 6200 | 7200 |
| CaYBR080C | 201 | 1201 | 2201 | 3201 | 4201 | 5201 | 6201 | 7201 |
| CaYBR091C | 202 | 1202 | 2202 | 3202 | 4202 | 5202 | 6202 | 7202 |
| CaYBR135W | 203 | 1203 | 2203 | 3203 | 4203 | 5203 | 6203 | 7203 |
| CaYBR142W | 204 | 1204 | 2204 | 3204 | 4204 | 5204 | 6204 | 7204 |
| CaYBR143C | 205 | 1205 | 2205 | 3205 | 4205 | 5205 | 6205 | 7205 |
| CaYBR160W | 206 | 1206 | 2206 | 3206 | 4206 | 5206 | 6206 | 7206 |
| CaYBR196C | 207 | 1207 | 2207 | 3207 | 4207 | 5207 | 6207 | 7207 |
| CaYBR198C | 208 | 1208 | 2208 | 3208 | 4208 | 5208 | 6208 | 7208 |
| CaYBR202W | 209 | 1209 | 2209 | 3209 | 4209 | 5209 | 6209 | 7209 |
| CaYBR234C | 210 | 1210 | 2210 | 3210 | 4210 | 5210 | 6210 | 7210 |
| CaYBR236C | 211 | 1211 | 2211 | 3211 | 4211 | 5211 | 6211 | 7211 |
| CaYBR237W | 212 | 1212 | 2212 | 3212 | 4212 | 5212 | 6212 | 7212 |
| CaYBR253W | 213 | 1213 | 2213 | 3213 | 4213 | 5213 | 6213 | 7213 |
| CaYBR254C | 214 | 1214 | 2214 | 3214 | 4214 | 5214 | 6214 | 7214 |
| CaYCL003W | 215 | 1215 | 2215 | 3215 | 4215 | 5215 | 6215 | 7215 |
| CaYCL017C | 216 | 1216 | 2216 | 3216 | 4216 | 5216 | 6216 | 7216 |
| CaYCL054W | 217 | 1217 | 2217 | 3217 | 4217 | 5217 | 6217 | 7217 |
| CaYCR012W | 218 | 1218 | 2218 | 3218 | 4218 | 5218 | 6218 | 7218 |
| CaYCR057C | 219 | 1219 | 2219 | 3219 | 4219 | 5219 | 6219 | 7219 |
| CaYCR072C | 220 | 1220 | 2220 | 3220 | 4220 | 5220 | 6220 | 7220 |
| CaYDL030W | 221 | 1221 | 2221 | 3221 | 4221 | 5221 | 6221 | 7221 |
| CaYDL043C | 222 | 1222 | 2222 | 3222 | 4222 | 5222 | 6222 | 7222 |
| CaYDL055C | 223 | 1223 | 2223 | 3223 | 4223 | 5223 | 6223 | 7223 |
| CaYDL060W | 224 | 1224 | 2224 | 3224 | 4224 | 5224 | 6224 | 7224 |
| CaYDL084W | 225 | 1225 | 2225 | 3225 | 4225 | 5225 | 6225 | 7225 |
| CaYDL087C | 226 | 1226 | 2226 | 3226 | 4226 | 5226 | 6226 | 7226 |
| CaYDL126C | 227 | 1227 | 2227 | 3227 | 4227 | 5227 | 6227 | 7227 |
| CaYDL132W | 228 | 1228 | 2228 | 3228 | 4228 | 5228 | 6228 | 7228 |
| CaYDL141W | 229 | 1229 | 2229 | 3229 | 4229 | 5229 | 6229 | 7229 |
| CaYKL059C | 230 | 1230 | 2230 | 3230 | 4230 | 5230 | 6230 | 7230 |
| CaYDL108W | 231 | 1231 | 2231 | 3231 | 4231 | 5231 | 6231 | 7231 |
| CaYKL060C | 232 | 1232 | 2232 | 3232 | 4232 | 5232 | 6232 | 7232 |
| CaYHR070W | 233 | 1233 | 2233 | 3233 | 4233 | 5233 | 6233 | 7233 |
| CaYGR195W | 234 | 1234 | 2234 | 3234 | 4234 | 5234 | 6234 | 7234 |
| CaYOL102C | 235 | 1235 | 2235 | 3235 | 4235 | 5235 | 6235 | 7235 |
| CaYOR074C | 236 | 1236 | 2236 | 3236 | 4236 | 5236 | 6236 | 7236 |
| CaYGL155W | 237 | 1237 | 2237 | 3237 | 4237 | 5237 | 6237 | 7237 |
| CaYLR305C | 238 | 1238 | 2238 | 3238 | 4238 | 5238 | 6238 | 7238 |
| CaYNL222W | 239 | 1239 | 2239 | 3239 | 4239 | 5239 | 6239 | 7239 |
| CaYDR236C | 240 | 1240 | 2240 | 3240 | 4240 | 5240 | 6240 | 7240 |
| CaYBL020W | 241 | 1241 | 2241 | 3241 | 4241 | 5241 | 6241 | 7241 |
| CaYNL261W | 242 | 1242 | 2242 | 3242 | 4242 | 5242 | 6242 | 7242 |
| CaYDR246W | 243 | 1243 | 2243 | 3243 | 4243 | 5243 | 6243 | 7243 |
| CaYNL075W | 244 | 1244 | 2244 | 3244 | 4244 | 5244 | 6244 | 7244 |
| CaYOR145C | 245 | 1245 | 2245 | 3245 | 4245 | 5245 | 6245 | 7245 |
| CaYOL077C | 246 | 1246 | 2246 | 3246 | 4246 | 5246 | 6246 | 7246 |
| CaYBR257W | 247 | 1247 | 2247 | 3247 | 4247 | 5247 | 6247 | 7247 |
| CaYHR170W | 248 | 1248 | 2248 | 3248 | 4248 | 5248 | 6248 | 7248 |
| CaYNL263C | 249 | 1249 | 2249 | 3249 | 4249 | 5249 | 6249 | 7249 |
| CaYKR068C | 250 | 1250 | 2250 | 3250 | 4250 | 5250 | 6250 | 7250 |
| CaYPR016C | 251 | 1251 | 2251 | 3251 | 4251 | 5251 | 6251 | 7251 |
| CaYGR172C | 252 | 1252 | 2252 | 3252 | 4252 | 5252 | 6252 | 7252 |
| CaYHR089C | 253 | 1253 | 2253 | 3253 | 4253 | 5253 | 6253 | 7253 |
| CaYMR197C | 254 | 1254 | 2254 | 3254 | 4254 | 5254 | 6254 | 7254 |
| CaYHR188C | 255 | 1255 | 2255 | 3255 | 4255 | 5255 | 6255 | 7255 |
| CaYPL266W | 256 | 1256 | 2256 | 3256 | 4256 | 5256 | 6256 | 7256 |
| CaYBR011C | 257 | 1257 | 2257 | 3257 | 4257 | 5257 | 6257 | 7257 |
| CaYCL059C | 258 | 1258 | 2258 | 3258 | 4258 | 5258 | 6258 | 7258 |
| CaYDL008W | 259 | 1259 | 2259 | 3259 | 4259 | 5259 | 6259 | 7259 |
| CaYDL097C | 260 | 1260 | 2260 | 3260 | 4260 | 5260 | 6260 | 7260 |
| CaYDL143W | 261 | 1261 | 2261 | 3261 | 4261 | 5261 | 6261 | 7261 |
| CaYDL205C | 262 | 1262 | 2262 | 3262 | 4262 | 5262 | 6262 | 7262 |
| CaYDL208W | 263 | 1263 | 2263 | 3263 | 4263 | 5263 | 6263 | 7263 |
| CaYDR002W | 264 | 1264 | 2264 | 3264 | 4264 | 5264 | 6264 | 7264 |
| CaYDR013W | 265 | 1265 | 2265 | 3265 | 4265 | 5265 | 6265 | 7265 |
| CaYDR023W | 266 | 1266 | 2266 | 3266 | 4266 | 5266 | 6266 | 7266 |
| CaYDR037W | 267 | 1267 | 2267 | 3267 | 4267 | 5267 | 6267 | 7267 |
| CaYDR045C | 268 | 1268 | 2268 | 3268 | 4268 | 5268 | 6268 | 7268 |
| CaYDR054C | 269 | 1269 | 2269 | 3269 | 4269 | 5269 | 6269 | 7269 |
| CaYDR086C | 270 | 1270 | 2270 | 3270 | 4270 | 5270 | 6270 | 7270 |
| CaYDR087C | 271 | 1271 | 2271 | 3271 | 4271 | 5271 | 6271 | 7271 |
| CaYDR091C | 272 | 1272 | 2272 | 3272 | 4272 | 5272 | 6272 | 7272 |
| CaYDR167W | 273 | 1273 | 2273 | 3273 | 4273 | 5273 | 6273 | 7273 |
| CaYDR172W | 274 | 1274 | 2274 | 3274 | 4274 | 5274 | 6274 | 7274 |
| CaYDR189W | 275 | 1275 | 2275 | 3275 | 4275 | 5275 | 6275 | 7275 |
| CaYDR196C | 276 | 1276 | 2276 | 3276 | 4276 | 5276 | 6276 | 7276 |
| CaYDR212W | 277 | 1277 | 2277 | 3277 | 4277 | 5277 | 6277 | 7277 |
| CaYDR238C | 278 | 1278 | 2278 | 3278 | 4278 | 5278 | 6278 | 7278 |
| CaYDR280W | 279 | 1279 | 2279 | 3279 | 4279 | 5279 | 6279 | 7279 |
| CaYDR331W | 280 | 1280 | 2280 | 3280 | 4280 | 5280 | 6280 | 7280 |
| CaYDR373W | 281 | 1281 | 2281 | 3281 | 4281 | 5281 | 6281 | 7281 |
| CaYDR376W | 282 | 1282 | 2282 | 3282 | 4282 | 5282 | 6282 | 7282 |
| CaYDR390C | 283 | 1283 | 2283 | 3283 | 4283 | 5283 | 6283 | 7283 |
| CaYDR394W | 284 | 1284 | 2284 | 3284 | 4284 | 5284 | 6284 | 7284 |
| CaYDR404C | 285 | 1285 | 2285 | 3285 | 4285 | 5285 | 6285 | 7285 |
| CaYDR429C | 286 | 1286 | 2286 | 3286 | 4286 | 5286 | 6286 | 7286 |
| CaYDR454C | 287 | 1287 | 2287 | 3287 | 4287 | 5287 | 6287 | 7287 |
| CaYEL020W-A | 288 | 1288 | 2288 | 3288 | 4288 | 5288 | 6288 | 7288 |
| CaYEL026W | 289 | 1289 | 2289 | 3289 | 4289 | 5289 | 6289 | 7289 |
| CaYER003C | 290 | 1290 | 2290 | 3290 | 4290 | 5290 | 6290 | 7290 |
| CaYER006W | 291 | 1291 | 2291 | 3291 | 4291 | 5291 | 6291 | 7291 |
| CaYER012W | 292 | 1292 | 2292 | 3292 | 4292 | 5292 | 6292 | 7292 |
| CaYER021W | 293 | 1293 | 2293 | 3293 | 4293 | 5293 | 6293 | 7293 |
| CaYER036C | 294 | 1294 | 2294 | 3294 | 4294 | 5294 | 6294 | 7294 |
| CaYER094C | 295 | 1295 | 2295 | 3295 | 4295 | 5295 | 6295 | 7295 |
| CaYER125W | 296 | 1296 | 2296 | 3296 | 4296 | 5296 | 6296 | 7296 |
| CaYER148W | 297 | 1297 | 2297 | 3297 | 4297 | 5297 | 6297 | 7297 |
| CaYER159C | 298 | 1298 | 2298 | 3298 | 4298 | 5298 | 6298 | 7298 |
| CaYFL002C | 299 | 1299 | 2299 | 3299 | 4299 | 5299 | 6299 | 7299 |
| CaYFL005W | 300 | 1300 | 2300 | 3300 | 4300 | 5300 | 6300 | 7300 |
| CaYFL017C | 301 | 1301 | 2301 | 3301 | 4301 | 5301 | 6301 | 7301 |
| CaYFL022C | 302 | 1302 | 2302 | 3302 | 4302 | 5302 | 6302 | 7302 |
| CaYFL038C | 303 | 1303 | 2303 | 3303 | 4303 | 5303 | 6303 | 7303 |
| CaYFL045C | 304 | 1304 | 2304 | 3304 | 4304 | 5304 | 6304 | 7304 |
| CaYFR004W | 305 | 1305 | 2305 | 3305 | 4305 | 5305 | 6305 | 7305 |
| CaYFR037C | 306 | 1306 | 2306 | 3306 | 4306 | 5306 | 6306 | 7306 |
| CaYFR050C | 307 | 1307 | 2307 | 3307 | 4307 | 5307 | 6307 | 7307 |
| CaYFR052W | 308 | 1308 | 2308 | 3308 | 4308 | 5308 | 6308 | 7308 |
| CaYDL029W | 309 | 1309 | 2309 | 3309 | 4309 | 5309 | 6309 | 7309 |
| CaYDL147W | 310 | 1310 | 2310 | 3310 | 4310 | 5310 | 6310 | 7310 |
| CaYDL148C | 311 | 1311 | 2311 | 3311 | 4311 | 5311 | 6311 | 7311 |
| CaYDR060W | 312 | 1312 | 2312 | 3312 | 4312 | 5312 | 6312 | 7312 |
| CaYDR062W | 313 | 1313 | 2313 | 3313 | 4313 | 5313 | 6313 | 7313 |
| CaYDR211W | 314 | 1314 | 2314 | 3314 | 4314 | 5314 | 6314 | 7314 |
| CaYDR328C | 315 | 1315 | 2315 | 3315 | 4315 | 5315 | 6315 | 7315 |
| CaYER025W | 316 | 1316 | 2316 | 3316 | 4316 | 5316 | 6316 | 7316 |
| CaYER136W | 317 | 1317 | 2317 | 3317 | 4317 | 5317 | 6317 | 7317 |
| CaYER171W | 318 | 1318 | 2318 | 3318 | 4318 | 5318 | 6318 | 7318 |
| CaYFL008W | 319 | 1319 | 2319 | 3319 | 4319 | 5319 | 6319 | 7319 |
| CaYGL001C | 320 | 1320 | 2320 | 3320 | 4320 | 5320 | 6320 | 7320 |
| CaYGL008C | 321 | 1321 | 2321 | 3321 | 4321 | 5321 | 6321 | 7321 |
| CaYGL011C | 322 | 1322 | 2322 | 3322 | 4322 | 5322 | 6322 | 7322 |
| CaYGL022W | 323 | 1323 | 2323 | 3323 | 4323 | 5323 | 6323 | 7323 |
| CaYGL044C | 324 | 1324 | 2324 | 3324 | 4324 | 5324 | 6324 | 7324 |
| CaYGL048C | 325 | 1325 | 2325 | 3325 | 4325 | 5325 | 6325 | 7325 |
| CaYGL068W | 326 | 1326 | 2326 | 3326 | 4326 | 5326 | 6326 | 7326 |
| CaYGL097W | 327 | 1327 | 2327 | 3327 | 4327 | 5327 | 6327 | 7327 |
| CaYGL112C | 328 | 1328 | 2328 | 3328 | 4328 | 5328 | 6328 | 7328 |
| CaYGL120C | 329 | 1329 | 2329 | 3329 | 4329 | 5329 | 6329 | 7329 |
| CaYGL130W | 330 | 1330 | 2330 | 3330 | 4330 | 5330 | 6330 | 7330 |
| CaYGR029W | 331 | 1331 | 2331 | 3331 | 4331 | 5331 | 6331 | 7331 |
| CaYGR060W | 332 | 1332 | 2332 | 3332 | 4332 | 5332 | 6332 | 7332 |
| CaYGR094W | 333 | 1333 | 2333 | 3333 | 4333 | 5333 | 6333 | 7333 |
| CaYGR103W | 334 | 1334 | 2334 | 3334 | 4334 | 5334 | 6334 | 7334 |
| CaYGR185C | 335 | 1335 | 2335 | 3335 | 4335 | 5335 | 6335 | 7335 |
| CaYGR211W | 336 | 1336 | 2336 | 3336 | 4336 | 5336 | 6336 | 7336 |
| CaYGR218W | 337 | 1337 | 2337 | 3337 | 4337 | 5337 | 6337 | 7337 |
| CaYGR246C | 338 | 1338 | 2338 | 3338 | 4338 | 5338 | 6338 | 7338 |
| CaYGR253C | 339 | 1339 | 2339 | 3339 | 4339 | 5339 | 6339 | 7339 |
| CaYHL015W | 340 | 1340 | 2340 | 3340 | 4340 | 5340 | 6340 | 7340 |
| CaYHR005C-A | 341 | 1341 | 2341 | 3341 | 4341 | 5341 | 6341 | 7341 |
| CaYHR019C | 342 | 1342 | 2342 | 3342 | 4342 | 5342 | 6342 | 7342 |
| CaYHR020W | 343 | 1343 | 2343 | 3343 | 4343 | 5343 | 6343 | 7343 |
| CaYHR024C | 344 | 1344 | 2344 | 3344 | 4344 | 5344 | 6344 | 7344 |
| CaYHR062C | 345 | 1345 | 2345 | 3345 | 4345 | 5345 | 6345 | 7345 |
| CaYHR072W | 346 | 1346 | 2346 | 3346 | 4346 | 5346 | 6346 | 7346 |
| CaYHR072W-A | 347 | 1347 | 2347 | 3347 | 4347 | 5347 | 6347 | 7347 |
| CaYHR090C | 348 | 1348 | 2348 | 3348 | 4348 | 5348 | 6348 | 7348 |
| CaYHR122W | 349 | 1349 | 2349 | 3349 | 4349 | 5349 | 6349 | 7349 |
| CaYHR143W-A | 350 | 1350 | 2350 | 3350 | 4350 | 5350 | 6350 | 7350 |
| CaYHR148W | 351 | 1351 | 2351 | 3351 | 4351 | 5351 | 6351 | 7351 |
| CaYHR165C | 352 | 1352 | 2352 | 3352 | 4352 | 5352 | 6352 | 7352 |
| CaYHR166C | 353 | 1353 | 2353 | 3353 | 4353 | 5353 | 6353 | 7353 |
| CaYHR169W | 354 | 1354 | 2354 | 3354 | 4354 | 5354 | 6354 | 7354 |
| CaYHR190W | 355 | 1355 | 2355 | 3355 | 4355 | 5355 | 6355 | 7355 |
| CaYIL003W | 356 | 1356 | 2356 | 3356 | 4356 | 5356 | 6356 | 7356 |
| CaYIL021W | 357 | 1357 | 2357 | 3357 | 4357 | 5357 | 6357 | 7357 |
| CaYIL075C | 358 | 1358 | 2358 | 3358 | 4358 | 5358 | 6358 | 7358 |
| CaYIL078W | 359 | 1359 | 2359 | 3359 | 4359 | 5359 | 6359 | 7359 |
| CaYIL142W | 360 | 1360 | 2360 | 3360 | 4360 | 5360 | 6360 | 7360 |
| CaYIR008C | 361 | 1361 | 2361 | 3361 | 4361 | 5361 | 6361 | 7361 |
| CaYIR022W | 362 | 1362 | 2362 | 3362 | 4362 | 5362 | 6362 | 7362 |
| CaYJL001W | 363 | 1363 | 2363 | 3363 | 4363 | 5363 | 6363 | 7363 |
| CaYJL014W | 364 | 1364 | 2364 | 3364 | 4364 | 5364 | 6364 | 7364 |
| CaYJL050W | 365 | 1365 | 2365 | 3365 | 4365 | 5365 | 6365 | 7365 |
| CaYJL074C | 366 | 1366 | 2366 | 3366 | 4366 | 5366 | 6366 | 7366 |
| CaYJL081C | 367 | 1367 | 2367 | 3367 | 4367 | 5367 | 6367 | 7367 |
| CaYJL104W | 368 | 1368 | 2368 | 3368 | 4368 | 5368 | 6368 | 7368 |
| CaYJL111W | 369 | 1369 | 2369 | 3369 | 4369 | 5369 | 6369 | 7369 |
| CaYJL143W | 370 | 1370 | 2370 | 3370 | 4370 | 5370 | 6370 | 7370 |
| CaYJL167W | 371 | 1371 | 2371 | 3371 | 4371 | 5371 | 6371 | 7371 |
| CaYJL194W | 372 | 1372 | 2372 | 3372 | 4372 | 5372 | 6372 | 7372 |
| CaYJR006W | 373 | 1373 | 2373 | 3373 | 4373 | 5373 | 6373 | 7373 |
| CaYJR017C | 374 | 1374 | 2374 | 3374 | 4374 | 5374 | 6374 | 7374 |
| CaYJR064W | 375 | 1375 | 2375 | 3375 | 4375 | 5375 | 6375 | 7375 |
| CaYJR065C | 376 | 1376 | 2376 | 3376 | 4376 | 5376 | 6376 | 7376 |
| CaYJR072C | 377 | 1377 | 2377 | 3377 | 4377 | 5377 | 6377 | 7377 |
| CaYJR123W | 378 | 1378 | 2378 | 3378 | 4378 | 5378 | 6378 | 7378 |
| CaYKL012W | 379 | 1379 | 2379 | 3379 | 4379 | 5379 | 6379 | 7379 |
| CaYKL019W | 380 | 1380 | 2380 | 3380 | 4380 | 5380 | 6380 | 7380 |
| CaYKL028W | 381 | 1381 | 2381 | 3381 | 4381 | 5381 | 6381 | 7381 |
| CaYKL058W | 382 | 1382 | 2382 | 3382 | 4382 | 5382 | 6382 | 7382 |
| CaYKL104C | 383 | 1383 | 2383 | 3383 | 4383 | 5383 | 6383 | 7383 |
| CaYKL144C | 384 | 1384 | 2384 | 3384 | 4384 | 5384 | 6384 | 7384 |
| CaYKL145W | 385 | 1385 | 2385 | 3385 | 4385 | 5385 | 6385 | 7385 |
| CaYKL172W | 386 | 1386 | 2386 | 3386 | 4386 | 5386 | 6386 | 7386 |
| CaYKL210W | 387 | 1387 | 2387 | 3387 | 4387 | 5387 | 6387 | 7387 |
| CaYKR079C | 388 | 1388 | 2388 | 3388 | 4388 | 5388 | 6388 | 7388 |
| CaYKR086W | 389 | 1389 | 2389 | 3389 | 4389 | 5389 | 6389 | 7389 |
| CaYLL018C | 390 | 1390 | 2390 | 3390 | 4390 | 5390 | 6390 | 7390 |
| CaYLR005W | 391 | 1391 | 2391 | 3391 | 4391 | 5391 | 6391 | 7391 |
| CaYLR009W | 392 | 1392 | 2392 | 3392 | 4392 | 5392 | 6392 | 7392 |
| CaYLR022C | 393 | 1393 | 2393 | 3393 | 4393 | 5393 | 6393 | 7393 |
| CaYLR026C | 394 | 1394 | 2394 | 3394 | 4394 | 5394 | 6394 | 7394 |
| CaYLR051C | 395 | 1395 | 2395 | 3395 | 4395 | 5395 | 6395 | 7395 |
| CaYLR060W | 396 | 1396 | 2396 | 3396 | 4396 | 5396 | 6396 | 7396 |
| CaYLR078C | 397 | 1397 | 2397 | 3397 | 4397 | 5397 | 6397 | 7397 |
| CaYLR100W | 398 | 1398 | 2398 | 3398 | 4398 | 5398 | 6398 | 7398 |
| CaYLR116W | 399 | 1399 | 2399 | 3399 | 4399 | 5399 | 6399 | 7399 |
| CaYLR117C | 400 | 1400 | 2400 | 3400 | 4400 | 5400 | 6400 | 7400 |
| CaYLR129W | 401 | 1401 | 2401 | 3401 | 4401 | 5401 | 6401 | 7401 |
| CaYLR147C | 402 | 1402 | 2402 | 3402 | 4402 | 5402 | 6402 | 7402 |
| CaYLR153C | 403 | 1403 | 2403 | 3403 | 4403 | 5403 | 6403 | 7403 |
| CaYLR163C | 404 | 1404 | 2404 | 3404 | 4404 | 5404 | 6404 | 7404 |
| CaYLR175W | 405 | 1405 | 2405 | 3405 | 4405 | 5405 | 6405 | 7405 |
| CaYLR186W | 406 | 1406 | 2406 | 3406 | 4406 | 5406 | 6406 | 7406 |
| CaYLR197W | 407 | 1407 | 2407 | 3407 | 4407 | 5407 | 6407 | 7407 |
| CaYLR208W | 408 | 1408 | 2408 | 3408 | 4408 | 5408 | 6408 | 7408 |
| CaYLR222C | 409 | 1409 | 2409 | 3409 | 4409 | 5409 | 6409 | 7409 |
| CaYLR259C | 410 | 1410 | 2410 | 3410 | 4410 | 5410 | 6410 | 7410 |
| CaYLR276C | 411 | 1411 | 2411 | 3411 | 4411 | 5411 | 6411 | 7411 |
| CaYLR277C | 412 | 1412 | 2412 | 3412 | 4412 | 5412 | 6412 | 7412 |
| CaYLR291C | 413 | 1413 | 2413 | 3413 | 4413 | 5413 | 6413 | 7413 |
| CaYLR293C | 414 | 1414 | 2414 | 3414 | 4414 | 5414 | 6414 | 7414 |
| CaYLR347C | 415 | 1415 | 2415 | 3415 | 4415 | 5415 | 6415 | 7415 |
| CaYLR378C | 416 | 1416 | 2416 | 3416 | 4416 | 5416 | 6416 | 7416 |
| CaYLR397C | 417 | 1417 | 2417 | 3417 | 4417 | 5417 | 6417 | 7417 |
| CaYML064C | 418 | 1418 | 2418 | 3418 | 4418 | 5418 | 6418 | 7418 |
| CaYML069W | 419 | 1419 | 2419 | 3419 | 4419 | 5419 | 6419 | 7419 |
| CaYML092C | 420 | 1420 | 2420 | 3420 | 4420 | 5420 | 6420 | 7420 |
| CaYML093W | 421 | 1421 | 2421 | 3421 | 4421 | 5421 | 6421 | 7421 |
| CaYML125C | 422 | 1422 | 2422 | 3422 | 4422 | 5422 | 6422 | 7422 |
| CaYML126C | 423 | 1423 | 2423 | 3423 | 4423 | 5423 | 6423 | 7423 |
| CaYMR113W | 424 | 1424 | 2424 | 3424 | 4424 | 5424 | 6424 | 7424 |
| CaYMR131C | 425 | 1425 | 2425 | 3425 | 4425 | 5425 | 6425 | 7425 |
| CaYMR146C | 426 | 1426 | 2426 | 3426 | 4426 | 5426 | 6426 | 7426 |
| CaYMR208W | 427 | 1427 | 2427 | 3427 | 4427 | 5427 | 6427 | 7427 |
| CaYMR213W | 428 | 1428 | 2428 | 3428 | 4428 | 5428 | 6428 | 7428 |
| CaYMR240C | 429 | 1429 | 2429 | 3429 | 4429 | 5429 | 6429 | 7429 |
| CaYMR260C | 430 | 1430 | 2430 | 3430 | 4430 | 5430 | 6430 | 7430 |
| CaYMR308C | 431 | 1431 | 2431 | 3431 | 4431 | 5431 | 6431 | 7431 |
| CaYMR314W | 432 | 1432 | 2432 | 3432 | 4432 | 5432 | 6432 | 7432 |
| CaYNL002C | 433 | 1433 | 2433 | 3433 | 4433 | 5433 | 6433 | 7433 |
| CaYNL006W | 434 | 1434 | 2434 | 3434 | 4434 | 5434 | 6434 | 7434 |
| CaYNL061W | 435 | 1435 | 2435 | 3435 | 4435 | 5435 | 6435 | 7435 |
| CaYNL102W | 436 | 1436 | 2436 | 3436 | 4436 | 5436 | 6436 | 7436 |
| CaYNL113W | 437 | 1437 | 2437 | 3437 | 4437 | 5437 | 6437 | 7437 |
| CaYNL178W | 438 | 1438 | 2438 | 3438 | 4438 | 5438 | 6438 | 7438 |
| CaYNL189W | 439 | 1439 | 2439 | 3439 | 4439 | 5439 | 6439 | 7439 |
| CaYNL244C | 440 | 1440 | 2440 | 3440 | 4440 | 5440 | 6440 | 7440 |
| CaYNL247W | 441 | 1441 | 2441 | 3441 | 4441 | 5441 | 6441 | 7441 |
| CaYNL287W | 442 | 1442 | 2442 | 3442 | 4442 | 5442 | 6442 | 7442 |
| CaYNR043W | 443 | 1443 | 2443 | 3443 | 4443 | 5443 | 6443 | 7443 |
| CaYOL005C | 444 | 1444 | 2444 | 3444 | 4444 | 5444 | 6444 | 7444 |
| CaYOL010W | 445 | 1445 | 2445 | 3445 | 4445 | 5445 | 6445 | 7445 |
| CaYOL094C | 446 | 1446 | 2446 | 3446 | 4446 | 5446 | 6446 | 7446 |
| CaYOL139C | 447 | 1447 | 2447 | 3447 | 4447 | 5447 | 6447 | 7447 |
| CaYOR048C | 448 | 1448 | 2448 | 3448 | 4448 | 5448 | 6448 | 7448 |
| CaYOR056C | 449 | 1449 | 2449 | 3449 | 4449 | 5449 | 6449 | 7449 |
| CaYOR063W | 450 | 1450 | 2450 | 3450 | 4450 | 5450 | 6450 | 7450 |
| CaYOR103C | 451 | 1451 | 2451 | 3451 | 4451 | 5451 | 6451 | 7451 |
| CaYOR116C | 452 | 1452 | 2452 | 3452 | 4452 | 5452 | 6452 | 7452 |
| CaYOR117W | 453 | 1453 | 2453 | 3453 | 4453 | 5453 | 6453 | 7453 |
| CaYOR151C | 454 | 1454 | 2454 | 3454 | 4454 | 5454 | 6454 | 7454 |
| CaYOR157C | 455 | 1455 | 2455 | 3455 | 4455 | 5455 | 6455 | 7455 |
| CaYOR159C | 456 | 1456 | 2456 | 3456 | 4456 | 5456 | 6456 | 7456 |
| CaYOR168W | 457 | 1457 | 2457 | 3457 | 4457 | 5457 | 6457 | 7457 |
| CaYOR194C | 458 | 1458 | 2458 | 3458 | 4458 | 5458 | 6458 | 7458 |
| CaYOR207C | 459 | 1459 | 2459 | 3459 | 4459 | 5459 | 6459 | 7459 |
| CaYOR210W | 460 | 1460 | 2460 | 3460 | 4460 | 5460 | 6460 | 7460 |
| CaYOR217W | 461 | 1461 | 2461 | 3461 | 4461 | 5461 | 6461 | 7461 |
| CaYOR224C | 462 | 1462 | 2462 | 3462 | 4462 | 5462 | 6462 | 7462 |
| CaYOR232W | 463 | 1463 | 2463 | 3463 | 4463 | 5463 | 6463 | 7463 |
| CaYOR259C | 464 | 1464 | 2464 | 3464 | 4464 | 5464 | 6464 | 7464 |
| CaYOR261C | 465 | 1465 | 2465 | 3465 | 4465 | 5465 | 6465 | 7465 |
| CaYOR272W | 466 | 1466 | 2466 | 3466 | 4466 | 5466 | 6466 | 7466 |
| CaYOR294W | 467 | 1467 | 2467 | 3467 | 4467 | 5467 | 6467 | 7467 |
| CaYOR310C | 468 | 1468 | 2468 | 3468 | 4468 | 5468 | 6468 | 7468 |
| CaYOR335C | 469 | 1469 | 2469 | 3469 | 4469 | 5469 | 6469 | 7469 |
| CaYOR341W | 470 | 1470 | 2470 | 3470 | 4470 | 5470 | 6470 | 7470 |
| CaYPL010W | 471 | 1471 | 2471 | 3471 | 4471 | 5471 | 6471 | 7471 |
| CaYPL076W | 472 | 1472 | 2472 | 3472 | 4472 | 5472 | 6472 | 7472 |
| CaYPL094C | 473 | 1473 | 2473 | 3473 | 4473 | 5473 | 6473 | 7473 |
| CaYPL117C | 474 | 1474 | 2474 | 3474 | 4474 | 5474 | 6474 | 7474 |
| CaYPL122C | 475 | 1475 | 2475 | 3475 | 4475 | 5475 | 6475 | 7475 |
| CaYPL131W | 476 | 1476 | 2476 | 3476 | 4476 | 5476 | 6476 | 7476 |
| CaYPL211W | 477 | 1477 | 2477 | 3477 | 4477 | 5477 | 6477 | 7477 |
| CaYPL235W | 478 | 1478 | 2478 | 3478 | 4478 | 5478 | 6478 | 7478 |
| CaYPL252C | 479 | 1479 | 2479 | 3479 | 4479 | 5479 | 6479 | 7479 |
| CaYPR019W | 480 | 1480 | 2480 | 3480 | 4480 | 5480 | 6480 | 7480 |
| CaYPR025C | 481 | 1481 | 2481 | 3481 | 4481 | 5481 | 6481 | 7481 |
| CaYPR034W | 482 | 1482 | 2482 | 3482 | 4482 | 5482 | 6482 | 7482 |
| CaYPR055W | 483 | 1483 | 2483 | 3483 | 4483 | 5483 | 6483 | 7483 |
| CaYPR056W | 484 | 1484 | 2484 | 3484 | 4484 | 5484 | 6484 | 7484 |
| CaYPR082C | 485 | 1485 | 2485 | 3485 | 4485 | 5485 | 6485 | 7485 |
| CaYPR103W | 486 | 1486 | 2486 | 3486 | 4486 | 5486 | 6486 | 7486 |
| CaYPR107C | 487 | 1487 | 2487 | 3487 | 4487 | 5487 | 6487 | 7487 |
| CaYPR108W | 488 | 1488 | 2488 | 3488 | 4488 | 5488 | 6488 | 7488 |
| CaYPR110C | 489 | 1489 | 2489 | 3489 | 4489 | 5489 | 6489 | 7489 |
| CaYPR113W | 490 | 1490 | 2490 | 3490 | 4490 | 5490 | 6490 | 7490 |
| CaYPR176C | 491 | 1491 | 2491 | 3491 | 4491 | 5491 | 6491 | 7491 |
| CaYPR183W | 492 | 1492 | 2492 | 3492 | 4492 | 5492 | 6492 | 7492 |
| CaYPR186C | 493 | 1493 | 2493 | 3493 | 4493 | 5493 | 6493 | 7493 |
| CaYPR187W | 494 | 1494 | 2494 | 3494 | 4494 | 5494 | 6494 | 7494 |
| CaYGL123W | 495 | 1495 | 2495 | 3495 | 4495 | 5495 | 6495 | 7495 |
| CaYHR042W | 496 | 1496 | 2496 | 3496 | 4496 | 5496 | 6496 | 7496 |
| CaYIL062C | 497 | 1497 | 2497 | 3497 | 4497 | 5497 | 6497 | 7497 |
| CaYJR042W | 498 | 1498 | 2498 | 3498 | 4498 | 5498 | 6498 | 7498 |
| CaYJR063W | 499 | 1499 | 2499 | 3499 | 4499 | 5499 | 6499 | 7499 |
| CaYJR076C | 500 | 1500 | 2500 | 3500 | 4500 | 5500 | 6500 | 7500 |
| CaYKL013C | 501 | 1501 | 2501 | 3501 | 4501 | 5501 | 6501 | 7501 |
| CaYLR196W | 502 | 1502 | 2502 | 3502 | 4502 | 5502 | 6502 | 7502 |
| CaYLR272C | 503 | 1503 | 2503 | 3503 | 4503 | 5503 | 6503 | 7503 |
| CaYNR035C | 504 | 1504 | 2504 | 3504 | 4504 | 5504 | 6504 | 7504 |
| CaYPR088C | 505 | 1505 | 2505 | 3505 | 4505 | 5505 | 6505 | 7505 |
| CaYDR397C | 506 | 1506 | 2506 | 3506 | 4506 | 5506 | 6506 | 7506 |
| CaYAL032C | 507 | 1507 | 2507 | 3507 | 4507 | 5507 | 6507 | 7507 |
| CaYBR060C | 508 | 1508 | 2508 | 3508 | 4508 | 5508 | 6508 | 7508 |
| CaYBR154C | 509 | 1509 | 2509 | 3509 | 4509 | 5509 | 6509 | 7509 |
| CaYDL028C | 510 | 1510 | 2510 | 3510 | 4510 | 5510 | 6510 | 7510 |
| CaYDR088C | 511 | 1511 | 2511 | 3511 | 4511 | 5511 | 6511 | 7511 |
| CaYDR235W | 512 | 1512 | 2512 | 3512 | 4512 | 5512 | 6512 | 7512 |
| CaYDR267C | 513 | 1513 | 2513 | 3513 | 4513 | 5513 | 6513 | 7513 |
| CaYDR460W | 514 | 1514 | 2514 | 3514 | 4514 | 5514 | 6514 | 7514 |
| CaYEL032W | 515 | 1515 | 2515 | 3515 | 4515 | 5515 | 6515 | 7515 |
| CaYER013W | 516 | 1516 | 2516 | 3516 | 4516 | 5516 | 6516 | 7516 |
| CaYER048W-A | 517 | 1517 | 2517 | 3517 | 4517 | 5517 | 6517 | 7517 |
| CaYER172C | 518 | 1518 | 2518 | 3518 | 4518 | 5518 | 6518 | 7518 |
| CaYFR031C | 519 | 1519 | 2519 | 3519 | 4519 | 5519 | 6519 | 7519 |
| CaYGL065C | 520 | 1520 | 2520 | 3520 | 4520 | 5520 | 6520 | 7520 |
| CaYGL073W | 521 | 1521 | 2521 | 3521 | 4521 | 5521 | 6521 | 7521 |
| CaYGL091C | 522 | 1522 | 2522 | 3522 | 4522 | 5522 | 6522 | 7522 |
| CaYGL103W | 523 | 1523 | 2523 | 3523 | 4523 | 5523 | 6523 | 7523 |
| CaYGL116W | 524 | 1524 | 2524 | 3524 | 4524 | 5524 | 6524 | 7524 |
| CaYGL201C | 525 | 1525 | 2525 | 3525 | 4525 | 5525 | 6525 | 7525 |
| CaYGL245W | 526 | 1526 | 2526 | 3526 | 4526 | 5526 | 6526 | 7526 |
| CaYGL247W | 527 | 1527 | 2527 | 3527 | 4527 | 5527 | 6527 | 7527 |
| CaYGR047C | 528 | 1528 | 2528 | 3528 | 4528 | 5528 | 6528 | 7528 |
| CaYGR074W | 529 | 1529 | 2529 | 3529 | 4529 | 5529 | 6529 | 7529 |
| CaYGR083C | 530 | 1530 | 2530 | 3530 | 4530 | 5530 | 6530 | 7530 |
| CaYGR128C | 531 | 1531 | 2531 | 3531 | 4531 | 5531 | 6531 | 7531 |
| CaYHR074W | 532 | 1532 | 2532 | 3532 | 4532 | 5532 | 6532 | 7532 |
| CaYHR107C | 533 | 1533 | 2533 | 3533 | 4533 | 5533 | 6533 | 7533 |
| CaYIL126W | 534 | 1534 | 2534 | 3534 | 4534 | 5534 | 6534 | 7534 |
| CaYJL010C | 535 | 1535 | 2535 | 3535 | 4535 | 5535 | 6535 | 7535 |
| CaYJL011C | 536 | 1536 | 2536 | 3536 | 4536 | 5536 | 6536 | 7536 |
| CaYJL026W | 537 | 1537 | 2537 | 3537 | 4537 | 5537 | 6537 | 7537 |
| CaYJL039C | 538 | 1538 | 2538 | 3538 | 4538 | 5538 | 6538 | 7538 |
| CaYJL041W | 539 | 1539 | 2539 | 3539 | 4539 | 5539 | 6539 | 7539 |
| CaYJR045C | 540 | 1540 | 2540 | 3540 | 4540 | 5540 | 6540 | 7540 |
| CaYKL049C | 541 | 1541 | 2541 | 3541 | 4541 | 5541 | 6541 | 7541 |
| CaYKL152C | 542 | 1542 | 2542 | 3542 | 4542 | 5542 | 6542 | 7542 |
| CaYKL181W | 543 | 1543 | 2543 | 3543 | 4543 | 5543 | 6543 | 7543 |
| CaYLR086W | 544 | 1544 | 2544 | 3544 | 4544 | 5544 | 6544 | 7544 |
| CaYLR115W | 545 | 1545 | 2545 | 3545 | 4545 | 5545 | 6545 | 7545 |
| CaYLR223C | 546 | 1546 | 2546 | 3546 | 4546 | 5546 | 6546 | 7546 |
| CaYLR274W | 547 | 1547 | 2547 | 3547 | 4547 | 5547 | 6547 | 7547 |
| CaYLR336C | 548 | 1548 | 2548 | 3548 | 4548 | 5548 | 6548 | 7548 |
| CaYML065W | 549 | 1549 | 2549 | 3549 | 4549 | 5549 | 6549 | 7549 |
| CaYML098W | 550 | 1550 | 2550 | 3550 | 4550 | 5550 | 6550 | 7550 |
| CaYMR043W | 551 | 1551 | 2551 | 3551 | 4551 | 5551 | 6551 | 7551 |
| CaYMR112C | 552 | 1552 | 2552 | 3552 | 4552 | 5552 | 6552 | 7552 |
| CaYMR281W | 553 | 1553 | 2553 | 3553 | 4553 | 5553 | 6553 | 7553 |
| CaYMR288W | 554 | 1554 | 2554 | 3554 | 4554 | 5554 | 6554 | 7554 |
| CaYMR290C | 555 | 1555 | 2555 | 3555 | 4555 | 5555 | 6555 | 7555 |
| CaYMR309C | 556 | 1556 | 2556 | 3556 | 4556 | 5556 | 6556 | 7556 |
| CaYNL039W | 557 | 1557 | 2557 | 3557 | 4557 | 5557 | 6557 | 7557 |
| CaYNL110C | 558 | 1558 | 2558 | 3558 | 4558 | 5558 | 6558 | 7558 |
| CaYNL221C | 559 | 1559 | 2559 | 3559 | 4559 | 5559 | 6559 | 7559 |
| CaYNL317W | 560 | 1560 | 2560 | 3560 | 4560 | 5560 | 6560 | 7560 |
| CaYNR053C | 561 | 1561 | 2561 | 3561 | 4561 | 5561 | 6561 | 7561 |
| CaYOL038W | 562 | 1562 | 2562 | 3562 | 4562 | 5562 | 6562 | 7562 |
| CaYOR095C | 563 | 1563 | 2563 | 3563 | 4563 | 5563 | 6563 | 7563 |
| CaYOR204W | 564 | 1564 | 2564 | 3564 | 4564 | 5564 | 6564 | 7564 |
| CaYOR249C | 565 | 1565 | 2565 | 3565 | 4565 | 5565 | 6565 | 7565 |
| CaYOR250C | 566 | 1566 | 2566 | 3566 | 4566 | 5566 | 6566 | 7566 |
| CaYOR257W | 567 | 1567 | 2567 | 3567 | 4567 | 5567 | 6567 | 7567 |
| CaYOR370C | 568 | 1568 | 2568 | 3568 | 4568 | 5568 | 6568 | 7568 |
| CaYPL151C | 569 | 1569 | 2569 | 3569 | 4569 | 5569 | 6569 | 7569 |
| CaYPL204W | 570 | 1570 | 2570 | 3570 | 4570 | 5570 | 6570 | 7570 |
| CaYPL209C | 571 | 1571 | 2571 | 3571 | 4571 | 5571 | 6571 | 7571 |
| CaYPL242C | 572 | 1572 | 2572 | 3572 | 4572 | 5572 | 6572 | 7572 |
| CaYPR048W | 573 | 1573 | 2573 | 3573 | 4573 | 5573 | 6573 | 7573 |
| CaYPR086W | 574 | 1574 | 2574 | 3574 | 4574 | 5574 | 6574 | 7574 |
| CaYPR178W | 575 | 1575 | 2575 | 3575 | 4575 | 5575 | 6575 | 7575 |
| CaYIL109C | 576 | 1576 | 2576 | 3576 | 4576 | 5576 | 6576 | 7576 |
| CaYKL045W | 577 | 1577 | 2577 | 3577 | 4577 | 5577 | 6577 | 7577 |
| CaYLR316C | 578 | 1578 | 2578 | 3578 | 4578 | 5578 | 6578 | 7578 |
| CaYBR087W | 579 | 1579 | 2579 | 3579 | 4579 | 5579 | 6579 | 7579 |
| CaYGR048W | 580 | 1580 | 2580 | 3580 | 4580 | 5580 | 6580 | 7580 |
| CaYPL169C | 581 | 1581 | 2581 | 3581 | 4581 | 5581 | 6581 | 7581 |
| CaYGR186W | 582 | 1582 | 2582 | 3582 | 4582 | 5582 | 6582 | 7582 |
| CaYNL131W | 583 | 1583 | 2583 | 3583 | 4583 | 5583 | 6583 | 7583 |
| CaYLR088W | 584 | 1584 | 2584 | 3584 | 4584 | 5584 | 6584 | 7584 |
| CaYKL193C | 585 | 1585 | 2585 | 3585 | 4585 | 5585 | 6585 | 7585 |
| CaYJR007W | 586 | 1586 | 2586 | 3586 | 4586 | 5586 | 6586 | 7586 |
| CaYJL034W | 587 | 1587 | 2587 | 3587 | 4587 | 5587 | 6587 | 7587 |
| CaYDL207W | 588 | 1588 | 2588 | 3588 | 4588 | 5588 | 6588 | 7588 |
| CaYDL017W | 589 | 1589 | 2589 | 3589 | 4589 | 5589 | 6589 | 7589 |
| CaYAL035W | 590 | 1590 | 2590 | 3590 | 4590 | 5590 | 6590 | 7590 |
| CaYBR038W | 591 | 1591 | 2591 | 3591 | 4591 | 5591 | 6591 | 7591 |
| CaYBR159W | 592 | 1592 | 2592 | 3592 | 4592 | 5592 | 6592 | 7592 |
| CaYDR120C | 593 | 1593 | 2593 | 3593 | 4593 | 5593 | 6593 | 7593 |
| CaYER070W | 594 | 1594 | 2594 | 3594 | 4594 | 5594 | 6594 | 7594 |
| CaYGL003C | 595 | 1595 | 2595 | 3595 | 4595 | 5595 | 6595 | 7595 |
| CaYGL206C | 596 | 1596 | 2596 | 3596 | 4596 | 5596 | 6596 | 7596 |
| CaYAL043C | 597 | 1597 | 2597 | 3597 | 4597 | 5597 | 6597 | 7597 |
| CaYBL097W | 598 | 1598 | 2598 | 3598 | 4598 | 5598 | 6598 | 7598 |
| CaYBL105C | 599 | 1599 | 2599 | 3599 | 4599 | 5599 | 6599 | 7599 |
| CaYBR079C | 600 | 1600 | 2600 | 3600 | 4600 | 5600 | 6600 | 7600 |
| CaYBR088C | 601 | 1601 | 2601 | 3601 | 4601 | 5601 | 6601 | 7601 |
| CaYDL145C | 602 | 1602 | 2602 | 3602 | 4602 | 5602 | 6602 | 7602 |
| CaYDL166C | 603 | 1603 | 2603 | 3603 | 4603 | 5603 | 6603 | 7603 |
| CaYDR145W | 604 | 1604 | 2604 | 3604 | 4604 | 5604 | 6604 | 7604 |
| CaYDR170C | 605 | 1605 | 2605 | 3605 | 4605 | 5605 | 6605 | 7605 |
| CaYDR301W | 606 | 1606 | 2606 | 3606 | 4606 | 5606 | 6606 | 7606 |
| CaYDR531W | 607 | 1607 | 2607 | 3607 | 4607 | 5607 | 6607 | 7607 |
| CaYFL024C | 608 | 1608 | 2608 | 3608 | 4608 | 5608 | 6608 | 7608 |
| CaYFR002W | 609 | 1609 | 2609 | 3609 | 4609 | 5609 | 6609 | 7609 |
| CaYGR264C | 610 | 1610 | 2610 | 3610 | 4610 | 5610 | 6610 | 7610 |
| CaYHR023W | 611 | 1611 | 2611 | 3611 | 4611 | 5611 | 6611 | 7611 |
| CaYHR027C | 612 | 1612 | 2612 | 3612 | 4612 | 5612 | 6612 | 7612 |
| CaYJL008C | 613 | 1613 | 2613 | 3613 | 4613 | 5613 | 6613 | 7613 |
| CaYJL033W | 614 | 1614 | 2614 | 3614 | 4614 | 5614 | 6614 | 7614 |
| CaYJL054W | 615 | 1615 | 2615 | 3615 | 4615 | 5615 | 6615 | 7615 |
| CaYJL109C | 616 | 1616 | 2616 | 3616 | 4616 | 5616 | 6616 | 7616 |
| CaYJL125C | 617 | 1617 | 2617 | 3617 | 4617 | 5617 | 6617 | 7617 |
| CaYJL156C | 618 | 1618 | 2618 | 3618 | 4618 | 5618 | 6618 | 7618 |
| CaYJR002W | 619 | 1619 | 2619 | 3619 | 4619 | 5619 | 6619 | 7619 |
| CaYKL192C | 620 | 1620 | 2620 | 3620 | 4620 | 5620 | 6620 | 7620 |
| CaYLL034C | 621 | 1621 | 2621 | 3621 | 4621 | 5621 | 6621 | 7621 |
| CaYLR029C | 622 | 1622 | 2622 | 3622 | 4622 | 5622 | 6622 | 7622 |
| CaYLR167W | 623 | 1623 | 2623 | 3623 | 4623 | 5623 | 6623 | 7623 |
| CaYLR243W | 624 | 1624 | 2624 | 3624 | 4624 | 5624 | 6624 | 7624 |
| CaYLR249W | 625 | 1625 | 2625 | 3625 | 4625 | 5625 | 6625 | 7625 |
| CaYLR321C | 626 | 1626 | 2626 | 3626 | 4626 | 5626 | 6626 | 7626 |
| CaYLR383W | 627 | 1627 | 2627 | 3627 | 4627 | 5627 | 6627 | 7627 |
| CaYMR239C | 628 | 1628 | 2628 | 3628 | 4628 | 5628 | 6628 | 7628 |
| CaYNL088W | 629 | 1629 | 2629 | 3629 | 4629 | 5629 | 6629 | 7629 |
| CaYNL163C | 630 | 1630 | 2630 | 3630 | 4630 | 5630 | 6630 | 7630 |
| CaYNR038W | 631 | 1631 | 2631 | 3631 | 4631 | 5631 | 6631 | 7631 |
| CaYOL097C | 632 | 1632 | 2632 | 3632 | 4632 | 5632 | 6632 | 7632 |
| CaYOR260W | 633 | 1633 | 2633 | 3633 | 4633 | 5633 | 6633 | 7633 |
| CaYPL028W | 634 | 1634 | 2634 | 3634 | 4634 | 5634 | 6634 | 7634 |
| CaYPL153C | 635 | 1635 | 2635 | 3635 | 4635 | 5635 | 6635 | 7635 |
| CaYPL210C | 636 | 1636 | 2636 | 3636 | 4636 | 5636 | 6636 | 7636 |
| CaYPL217C | 637 | 1637 | 2637 | 3637 | 4637 | 5637 | 6637 | 7637 |
| CaYPR010C | 638 | 1638 | 2638 | 3638 | 4638 | 5638 | 6638 | 7638 |
| CaYPR144C | 639 | 1639 | 2639 | 3639 | 4639 | 5639 | 6639 | 7639 |
| CaYPR169W | 640 | 1640 | 2640 | 3640 | 4640 | 5640 | 6640 | 7640 |
| CaYDL140C | 641 | 1641 | 2641 | 3641 | 4641 | 5641 | 6641 | 7641 |
| CaYDL031W | 642 | 1642 | 2642 | 3642 | 4642 | 5642 | 6642 | 7642 |
| CaYHR186C | 643 | 1643 | 2643 | 3643 | 4643 | 5643 | 6643 | 7643 |
| CaYPL093W | 644 | 1644 | 2644 | 3644 | 4644 | 5644 | 6644 | 7644 |
| CaYKL035W | 645 | 1645 | 2645 | 3645 | 4645 | 5645 | 6645 | 7645 |
| CaYDL058W | 646 | 1646 | 2646 | 3646 | 4646 | 5646 | 6646 | 7646 |
| CaYDR341C | 647 | 1647 | 2647 | 3647 | 4647 | 5647 | 6647 | 7647 |
| CaYGL238W | 648 | 1648 | 2648 | 3648 | 4648 | 5648 | 6648 | 7648 |
| CaYFR028C | 649 | 1649 | 2649 | 3649 | 4649 | 5649 | 6649 | 7649 |
| CaYNL172W | 650 | 1650 | 2650 | 3650 | 4650 | 5650 | 6650 | 7650 |
| CaYDR190C | 651 | 1651 | 2651 | 3651 | 4651 | 5651 | 6651 | 7651 |
| CaYEL055C | 652 | 1652 | 2652 | 3652 | 4652 | 5652 | 6652 | 7652 |
| CaYPR041W | 653 | 1653 | 2653 | 3653 | 4653 | 5653 | 6653 | 7653 |
| CaYGR255C | 654 | 1654 | 2654 | 3654 | 4654 | 5654 | 6654 | 7654 |
| CaYBR055C | 655 | 1655 | 2655 | 3655 | 4655 | 5655 | 6655 | 7655 |
| CaYER022W | 656 | 1656 | 2656 | 3656 | 4656 | 5656 | 6656 | 7656 |
| CaYKL014C | 657 | 1657 | 2657 | 3657 | 4657 | 5657 | 6657 | 7657 |
| CaYIL046W | 658 | 1658 | 2658 | 3658 | 4658 | 5658 | 6658 | 7658 |
| CaYMR015C | 659 | 1659 | 2659 | 3659 | 4659 | 5659 | 6659 | 7659 |
| CaYNL280C | 660 | 1660 | 2660 | 3660 | 4660 | 5660 | 6660 | 7660 |
| CaYML075C | 661 | 1661 | 2661 | 3661 | 4661 | 5661 | 6661 | 7661 |
| CaYCR042C | 662 | 1662 | 2662 | 3662 | 4662 | 5662 | 6662 | 7662 |
| CaYMR235C | 663 | 1663 | 2663 | 3663 | 4663 | 5663 | 6663 | 7663 |
| CaYIL026C | 664 | 1664 | 2664 | 3664 | 4664 | 5664 | 6664 | 7664 |
| CaYPL085W | 665 | 1665 | 2665 | 3665 | 4665 | 5665 | 6665 | 7665 |
| CaYGR005C | 666 | 1666 | 2666 | 3666 | 4666 | 5666 | 6666 | 7666 |
| CaYOL144W | 667 | 1667 | 2667 | 3667 | 4667 | 5667 | 6667 | 7667 |
| CaYHR005C | 668 | 1668 | 2668 | 3668 | 4668 | 5668 | 6668 | 7668 |
| CaYGR013W | 669 | 1669 | 2669 | 3669 | 4669 | 5669 | 6669 | 7669 |
| CaYIL115C | 670 | 1670 | 2670 | 3670 | 4670 | 5670 | 6670 | 7670 |
| CaYGR147C | 671 | 1671 | 2671 | 3671 | 4671 | 5671 | 6671 | 7671 |
| CaYOR336W | 672 | 1672 | 2672 | 3672 | 4672 | 5672 | 6672 | 7672 |
| CaYPR159W | 673 | 1673 | 2673 | 3673 | 4673 | 5673 | 6673 | 7673 |
| CaYJL174W | 674 | 1674 | 2674 | 3674 | 4674 | 5674 | 6674 | 7674 |
| CaYOL130W | 675 | 1675 | 2675 | 3675 | 4675 | 5675 | 6675 | 7675 |
| CaYNL048W | 676 | 1676 | 2676 | 3676 | 4676 | 5676 | 6676 | 7676 |
| CaYER007W | 677 | 1677 | 2677 | 3677 | 4677 | 5677 | 6677 | 7677 |
| CaYGL106W | 678 | 1678 | 2678 | 3678 | 4678 | 5678 | 6678 | 7678 |
| CaYDL102W | 679 | 1679 | 2679 | 3679 | 4679 | 5679 | 6679 | 7679 |
| CaYDL007W | 680 | 1680 | 2680 | 3680 | 4680 | 5680 | 6680 | 7680 |
| CaYER031C | 681 | 1681 | 2681 | 3681 | 4681 | 5681 | 6681 | 7681 |
| CaYDR226W | 682 | 1682 | 2682 | 3682 | 4682 | 5682 | 6682 | 7682 |
| CaYOR349W | 683 | 1683 | 2683 | 3683 | 4683 | 5683 | 6683 | 7683 |
| CaYNL148C | 684 | 1684 | 2684 | 3684 | 4684 | 5684 | 6684 | 7684 |
| CaYPR119W | 685 | 1685 | 2685 | 3685 | 4685 | 5685 | 6685 | 7685 |
| CaYMR055C | 686 | 1686 | 2686 | 3686 | 4686 | 5686 | 6686 | 7686 |
| CaYFL018C | 687 | 1687 | 2687 | 3687 | 4687 | 5687 | 6687 | 7687 |
| CaYNL238W | 688 | 1688 | 2688 | 3688 | 4688 | 5688 | 6688 | 7688 |
| CaYPL231W | 689 | 1689 | 2689 | 3689 | 4689 | 5689 | 6689 | 7689 |
| CaYNL025C | 690 | 1690 | 2690 | 3690 | 4690 | 5690 | 6690 | 7690 |
| CaYJL141C | 691 | 1691 | 2691 | 3691 | 4691 | 5691 | 6691 | 7691 |
| CaYLR306W | 692 | 1692 | 2692 | 3692 | 4692 | 5692 | 6692 | 7692 |
| CaYLR300W | 693 | 1693 | 2693 | 3693 | 4693 | 5693 | 6693 | 7693 |
| CaYKL046C | 694 | 1694 | 2694 | 3694 | 4694 | 5694 | 6694 | 7694 |
| CaYDR311W | 695 | 1695 | 2695 | 3695 | 4695 | 5695 | 6695 | 7695 |
| CaYDR449C | 696 | 1696 | 2696 | 3696 | 4696 | 5696 | 6696 | 7696 |
| CaYER023W | 697 | 1697 | 2697 | 3697 | 4697 | 5697 | 6697 | 7697 |
| CaYGL040C | 698 | 1698 | 2698 | 3698 | 4698 | 5698 | 6698 | 7698 |
| CaYGR009C | 699 | 1699 | 2699 | 3699 | 4699 | 5699 | 6699 | 7699 |
| CaYNR003C | 700 | 1700 | 2700 | 3700 | 4700 | 5700 | 6700 | 7700 |
| CaYOL066C | 701 | 1701 | 2701 | 3701 | 4701 | 5701 | 6701 | 7701 |
| CaYOR119C | 702 | 1702 | 2702 | 3702 | 4702 | 5702 | 6702 | 7702 |
| CaYMR049C | 703 | 1703 | 2703 | 3703 | 4703 | 5703 | 6703 | 7703 |
| CaYNR050C | 704 | 1704 | 2704 | 3704 | 4704 | 5704 | 6704 | 7704 |
| CaYPL203W | 705 | 1705 | 2705 | 3705 | 4705 | 5705 | 6705 | 7705 |
| CaYER113C | 706 | 1706 | 2706 | 3706 | 4706 | 5706 | 6706 | 7706 |
| CaYOR280C | 707 | 1707 | 2707 | 3707 | 4707 | 5707 | 6707 | 7707 |
| CaYGR006W | 708 | 1708 | 2708 | 3708 | 4708 | 5708 | 6708 | 7708 |
| CaYJL122W | 709 | 1709 | 2709 | 3709 | 4709 | 5709 | 6709 | 7709 |
| CaORF6_3320 | 710 | 1710 | 2710 | 3710 | 4710 | 5710 | 6710 | 7710 |
| CaORF6_7574 | 711 | 1711 | 2711 | 3711 | 4711 | 5711 | 6711 | 7711 |
| CaORF6_6275 | 712 | 1712 | 2712 | 3712 | 4712 | 5712 | 6712 | 7712 |
| CaORF6_1979 | 713 | 1713 | 2713 | 3713 | 4713 | 5713 | 6713 | 7713 |
| CaORF6_8942 | 714 | 1714 | 2714 | 3714 | 4714 | 5714 | 6714 | 7714 |
| CaYJL153C | 715 | 1715 | 2715 | 3715 | 4715 | 5715 | 6715 | 7715 |
| CaYNL277W | 716 | 1716 | 2716 | 3716 | 4716 | 5716 | 6716 | 7716 |
| CaYIL104C | 717 | 1717 | 2717 | 3717 | 4717 | 5717 | 6717 | 7717 |
| CaYOL027C | 718 | 1718 | 2718 | 3718 | 4718 | 5718 | 6718 | 7718 |
| CaYJL134W | 719 | 1719 | 2719 | 3719 | 4719 | 5719 | 6719 | 7719 |
| CaYLL012W | 720 | 1720 | 2720 | 3720 | 4720 | 5720 | 6720 | 7720 |
| CaORF6_7779 | 721 | 1721 | 2721 | 3721 | 4721 | 5721 | 6721 | 7721 |
| CaORF6_3262 | 722 | 1722 | 2722 | 3722 | 4722 | 5722 | 6722 | 7722 |
| CaORF6_7304 | 723 | 1723 | 2723 | 3723 | 4723 | 5723 | 6723 | 7723 |
| CaORF6_2028 | 724 | 1724 | 2724 | 3724 | 4724 | 5724 | 6724 | 7724 |
| CaORF6_1717 | 725 | 1725 | 2725 | 3725 | 4725 | 5725 | 6725 | 7725 |
| CaORF6_1780 | 726 | 1726 | 2726 | 3726 | 4726 | 5726 | 6726 | 7726 |
| CaORF6_1932 | 727 | 1727 | 2727 | 3727 | 4727 | 5727 | 6727 | 7727 |
| CaORF6_1934 | 728 | 1728 | 2728 | 3728 | 4728 | 5728 | 6728 | 7728 |
| CaORF6_2193 | 729 | 1729 | 2729 | 3729 | 4729 | 5729 | 6729 | 7729 |
| CaORF6_2398 | 730 | 1730 | 2730 | 3730 | 4730 | 5730 | 6730 | 7730 |
| orf6.3168 | 731 | 1731 | 2731 | 3731 | 4731 | 5731 | 6731 | 7731 |
| orf6.3295 | 732 | 1732 | 2732 | 3732 | 4732 | 5732 | 6732 | 7732 |
| orf6.3939 | 733 | 1733 | 2733 | 3733 | 4733 | 5733 | 6733 | 7733 |
| orf6.4497 | 734 | 1734 | 2734 | 3734 | 4734 | 5734 | 6734 | 7734 |
| orf6.4499 | 735 | 1735 | 2735 | 3735 | 4735 | 5735 | 6735 | 7735 |
| orf6.4537 | 736 | 1736 | 2736 | 3736 | 4736 | 5736 | 6736 | 7736 |
| orf6.4747 | 737 | 1737 | 2737 | 3737 | 4737 | 5737 | 6737 | 7737 |
| orf6.4899 | 738 | 1738 | 2738 | 3738 | 4738 | 5738 | 6738 | 7738 |
| orf6.4974 | 739 | 1739 | 2739 | 3739 | 4739 | 5739 | 6739 | 7739 |
| orf6.5147 | 740 | 1740 | 2740 | 3740 | 4740 | 5740 | 6740 | 7740 |
| orf6.5199 | 741 | 1741 | 2741 | 3741 | 4741 | 5741 | 6741 | 7741 |
| orf6.5210 | 742 | 1742 | 2742 | 3742 | 4742 | 5742 | 6742 | 7742 |
| orf6.5520 | 743 | 1743 | 2743 | 3743 | 4743 | 5743 | 6743 | 7743 |
| orf6.569 | 744 | 1744 | 2744 | 3744 | 4744 | 5744 | 6744 | 7744 |
| orf6.5739 | 745 | 1745 | 2745 | 3745 | 4745 | 5745 | 6745 | 7745 |
| orf6.6011 | 746 | 1746 | 2746 | 3746 | 4746 | 5746 | 6746 | 7746 |
| orf6.7375 | 747 | 1747 | 2747 | 3747 | 4747 | 5747 | 6747 | 7747 |
| orf6.7629 | 748 | 1748 | 2748 | 3748 | 4748 | 5748 | 6748 | 7748 |
| orf6.8025 | 749 | 1749 | 2749 | 3749 | 4749 | 5749 | 6749 | 7749 |
| orf6.804 | 750 | 1750 | 2750 | 3750 | 4750 | 5750 | 6750 | 7750 |
| orf6.8362 | 751 | 1751 | 2751 | 3751 | 4751 | 5751 | 6751 | 7751 |
| orf6.8377 | 752 | 1752 | 2752 | 3752 | 4752 | 5752 | 6752 | 7752 |
| orf6.8395 | 753 | 1753 | 2753 | 3753 | 4753 | 5753 | 6753 | 7753 |
| orf6.8482 | 754 | 1754 | 2754 | 3754 | 4754 | 5754 | 6754 | 7754 |
| orf6.8837 | 755 | 1755 | 2755 | 3755 | 4755 | 5755 | 6755 | 7755 |
| orf6.889 | 756 | 1756 | 2756 | 3756 | 4756 | 5756 | 6756 | 7756 |
| orf6.8938 | 757 | 1757 | 2757 | 3757 | 4757 | 5757 | 6757 | 7757 |
| orf6.9113 | 758 | 1758 | 2758 | 3758 | 4758 | 5758 | 6758 | 7758 |
| CaLYS4 | 759 | 1759 | 2759 | 3759 | 4759 | 5759 | 6759 | 7759 |
| CaTRP5 | 760 | 1760 | 2760 | 3760 | 4760 | 5760 | 6760 | 7760 |
| CaPRO1 | 761 | 1761 | 2761 | 3761 | 4761 | 5761 | 6761 | 7761 |
| CaPBS2 | 762 | 1762 | 2762 | 3762 | 4762 | 5762 | 6762 | 7762 |
| CaYBL041W | 763 | 1763 | 2763 | 3763 | 4763 | 5763 | 6763 | 7763 |
| CaYBR170C | 764 | 1764 | 2764 | 3764 | 4764 | 5764 | 6764 | 7764 |
| CaYDR188W | 765 | 1765 | 2765 | 3765 | 4765 | 5765 | 6765 | 7765 |
| CaYGR098C | 766 | 1766 | 2766 | 3766 | 4766 | 5766 | 6766 | 7766 |
| CaYGR267C | 767 | 1767 | 2767 | 3767 | 4767 | 5767 | 6767 | 7767 |
| CaYGR274C | 768 | 1768 | 2768 | 3768 | 4768 | 5768 | 6768 | 7768 |
| CaYJL002C | 769 | 1769 | 2769 | 3769 | 4769 | 5769 | 6769 | 7769 |
| CaYKL125W | 770 | 1770 | 2770 | 3770 | 4770 | 5770 | 6770 | 7770 |
| CaYLL035W | 771 | 1771 | 2771 | 3771 | 4771 | 5771 | 6771 | 7771 |
| CaYPL016W | 772 | 1772 | 2772 | 3772 | 4772 | 5772 | 6772 | 7772 |
| CaYPL218W | 773 | 1773 | 2773 | 3773 | 4773 | 5773 | 6773 | 7773 |
| CaYKL141W | 774 | 1774 | 2774 | 3774 | 4774 | 5774 | 6774 | 7774 |
| CaYHR174W | 775 | 1775 | 2775 | 3775 | 4775 | 5775 | 6775 | 7775 |
| CaYDR356W | 776 | 1776 | 2776 | 3776 | 4776 | 5776 | 6776 | 7776 |
| CaYNL124W | 777 | 1777 | 2777 | 3777 | 4777 | 5777 | 6777 | 7777 |
| CaYAL015C | 778 | 1778 | 2778 | 3778 | 4778 | 5778 | 6778 | 7778 |
| CaYBR001C | 779 | 1779 | 2779 | 3779 | 4779 | 5779 | 6779 | 7779 |
| CaYCL035C | 780 | 1780 | 2780 | 3780 | 4780 | 5780 | 6780 | 7780 |
| CaYCR048W | 781 | 1781 | 2781 | 3781 | 4781 | 5781 | 6781 | 7781 |
| CaYDR379W | 782 | 1782 | 2782 | 3782 | 4782 | 5782 | 6782 | 7782 |
| CaYER059W | 783 | 1783 | 2783 | 3783 | 4783 | 5783 | 6783 | 7783 |
| CaYGR070W | 784 | 1784 | 2784 | 3784 | 4784 | 5784 | 6784 | 7784 |
| CaYGR209C | 785 | 1785 | 2785 | 3785 | 4785 | 5785 | 6785 | 7785 |
| orf6.1498 | 786 | 1786 | 2786 | 3786 | 4786 | 5786 | 6786 | 7786 |
| orf6.2086 | 787 | 1787 | 2787 | 3787 | 4787 | 5787 | 6787 | 7787 |
| orf6.3026 | 788 | 1788 | 2788 | 3788 | 4788 | 5788 | 6788 | 7788 |
| orf6.3261 | 789 | 1789 | 2789 | 3789 | 4789 | 5789 | 6789 | 7789 |
| orf6.3819 | 790 | 1790 | 2790 | 3790 | 4790 | 5790 | 6790 | 7790 |
| orf6.3864 | 791 | 1791 | 2791 | 3791 | 4791 | 5791 | 6791 | 7791 |
| orf6.3972 | 792 | 1792 | 2792 | 3792 | 4792 | 5792 | 6792 | 7792 |
| orf6.4005 | 793 | 1793 | 2793 | 3793 | 4793 | 5793 | 6793 | 7793 |
| orf6.4010 | 794 | 1794 | 2794 | 3794 | 4794 | 5794 | 6794 | 7794 |
| orf6.4114 | 795 | 1795 | 2795 | 3795 | 4795 | 5795 | 6795 | 7795 |
| orf6.4153 | 796 | 1796 | 2796 | 3796 | 4796 | 5796 | 6796 | 7796 |
| orf6.4206 | 797 | 1797 | 2797 | 3797 | 4797 | 5797 | 6797 | 7797 |
| orf6.4293 | 798 | 1798 | 2798 | 3798 | 4798 | 5798 | 6798 | 7798 |
| orf6.4463 | 799 | 1799 | 2799 | 3799 | 4799 | 5799 | 6799 | 7799 |
| orf6.4555 | 800 | 1800 | 2800 | 3800 | 4800 | 5800 | 6800 | 7800 |
| orf6.4628 | 801 | 1801 | 2801 | 3801 | 4801 | 5801 | 6801 | 7801 |
| orf6.4837 | 802 | 1802 | 2802 | 3802 | 4802 | 5802 | 6802 | 7802 |
| orf6.4854 | 803 | 1803 | 2803 | 3803 | 4803 | 5803 | 6803 | 7803 |
| orf6.4923 | 804 | 1804 | 2804 | 3804 | 4804 | 5804 | 6804 | 7804 |
| orf6.4927 | 805 | 1805 | 2805 | 3805 | 4805 | 5805 | 6805 | 7805 |
| orf6.5092 | 806 | 1806 | 2806 | 3806 | 4806 | 5806 | 6806 | 7806 |
| orf6.5279 | 807 | 1807 | 2807 | 3807 | 4807 | 5807 | 6807 | 7807 |
| orf6.5786 | 808 | 1808 | 2808 | 3808 | 4808 | 5808 | 6808 | 7808 |
| orf6.5919 | 809 | 1809 | 2809 | 3809 | 4809 | 5809 | 6809 | 7809 |
| orf6.5920 | 810 | 1810 | 2810 | 3810 | 4810 | 5810 | 6810 | 7810 |
| orf6.6022 | 811 | 1811 | 2811 | 3811 | 4811 | 5811 | 6811 | 7811 |
| orf6.6026 | 812 | 1812 | 2812 | 3812 | 4812 | 5812 | 6812 | 7812 |
| orf6.6030 | 813 | 1813 | 2813 | 3813 | 4813 | 5813 | 6813 | 7813 |
| orf6.6069 | 814 | 1814 | 2814 | 3814 | 4814 | 5814 | 6814 | 7814 |
| orf6.6140 | 815 | 1815 | 2815 | 3815 | 4815 | 5815 | 6815 | 7815 |
| orf6.6218 | 816 | 1816 | 2816 | 3816 | 4816 | 5816 | 6816 | 7816 |
| orf6.6390 | 817 | 1817 | 2817 | 3817 | 4817 | 5817 | 6817 | 7817 |
| orf6.6550 | 818 | 1818 | 2818 | 3818 | 4818 | 5818 | 6818 | 7818 |
| orf6.6562 | 819 | 1819 | 2819 | 3819 | 4819 | 5819 | 6819 | 7819 |
| orf6.6660 | 820 | 1820 | 2820 | 3820 | 4820 | 5820 | 6820 | 7820 |
| orf6.6664 | 821 | 1821 | 2821 | 3821 | 4821 | 5821 | 6821 | 7821 |
| orf6.6670 | 822 | 1822 | 2822 | 3822 | 4822 | 5822 | 6822 | 7822 |
| orf6.6700 | 823 | 1823 | 2823 | 3823 | 4823 | 5823 | 6823 | 7823 |
| orf6.6933 | 824 | 1824 | 2824 | 3824 | 4824 | 5824 | 6824 | 7824 |
| orf6.6939 | 825 | 1825 | 2825 | 3825 | 4825 | 5825 | 6825 | 7825 |
| orf6.7203 | 826 | 1826 | 2826 | 3826 | 4826 | 5826 | 6826 | 7826 |
| orf6.7214 | 827 | 1827 | 2827 | 3827 | 4827 | 5827 | 6827 | 7827 |
| orf6.7847 | 828 | 1828 | 2828 | 3828 | 4828 | 5828 | 6828 | 7828 |
| orf6.7893 | 829 | 1829 | 2829 | 3829 | 4829 | 5829 | 6829 | 7829 |
| orf6.8239 | 830 | 1830 | 2830 | 3830 | 4830 | 5830 | 6830 | 7830 |
| orf6.8461 | 831 | 1831 | 2831 | 3831 | 4831 | 5831 | 6831 | 7831 |
| orf6.8607 | 832 | 1832 | 2832 | 3832 | 4832 | 5832 | 6832 | 7832 |
| orf6.8654 | 833 | 1833 | 2833 | 3833 | 4833 | 5833 | 6833 | 7833 |
| orf6.8716 | 834 | 1834 | 2834 | 3834 | 4834 | 5834 | 6834 | 7834 |
| CaYPR198W | 835 | 1835 | 2835 | 3835 | 4835 | 5835 | 6835 | 7835 |
| orf6.1031 | 836 | 1836 | 2836 | 3836 | 4836 | 5836 | 6836 | 7836 |
| orf6.1035 | 837 | 1837 | 2837 | 3837 | 4837 | 5837 | 6837 | 7837 |
| orf6.1302 | 838 | 1838 | 2838 | 3838 | 4838 | 5838 | 6838 | 7838 |
| orf6.1752 | 839 | 1839 | 2839 | 3839 | 4839 | 5839 | 6839 | 7839 |
| orf6.2119 | 840 | 1840 | 2840 | 3840 | 4840 | 5840 | 6840 | 7840 |
| orf6.2277 | 841 | 1841 | 2841 | 3841 | 4841 | 5841 | 6841 | 7841 |
| orf6.2355 | 842 | 1842 | 2842 | 3842 | 4842 | 5842 | 6842 | 7842 |
| orf6.2417 | 843 | 1843 | 2843 | 3843 | 4843 | 5843 | 6843 | 7843 |
| orf6.2463 | 844 | 1844 | 2844 | 3844 | 4844 | 5844 | 6844 | 7844 |
| orf6.2715 | 845 | 1845 | 2845 | 3845 | 4845 | 5845 | 6845 | 7845 |
| orf6.2865 | 846 | 1846 | 2846 | 3846 | 4846 | 5846 | 6846 | 7846 |
| orf6.3078 | 847 | 1847 | 2847 | 3847 | 4847 | 5847 | 6847 | 7847 |
| orf6.3170 | 848 | 1848 | 2848 | 3848 | 4848 | 5848 | 6848 | 7848 |
| orf6.3203 | 849 | 1849 | 2849 | 3849 | 4849 | 5849 | 6849 | 7849 |
| orf6.3517 | 850 | 1850 | 2850 | 3850 | 4850 | 5850 | 6850 | 7850 |
| orf6.3623 | 851 | 1851 | 2851 | 3851 | 4851 | 5851 | 6851 | 7851 |
| orf6.3676 | 852 | 1852 | 2852 | 3852 | 4852 | 5852 | 6852 | 7852 |
| orf6.3804 | 853 | 1853 | 2853 | 3853 | 4853 | 5853 | 6853 | 7853 |
| orf6.4003 | 854 | 1854 | 2854 | 3854 | 4854 | 5854 | 6854 | 7854 |
| orf6.4037 | 855 | 1855 | 2855 | 3855 | 4855 | 5855 | 6855 | 7855 |
| orf6.4386 | 856 | 1856 | 2856 | 3856 | 4856 | 5856 | 6856 | 7856 |
| orf6.4898 | 857 | 1857 | 2857 | 3857 | 4857 | 5857 | 6857 | 7857 |
| orf6.5286 | 858 | 1858 | 2858 | 3858 | 4858 | 5858 | 6858 | 7858 |
| orf6.5335 | 859 | 1859 | 2859 | 3859 | 4859 | 5859 | 6859 | 7859 |
| orf6.5517 | 860 | 1860 | 2860 | 3860 | 4860 | 5860 | 6860 | 7860 |
| orf6.5752 | 861 | 1861 | 2861 | 3861 | 4861 | 5861 | 6861 | 7861 |
| orf6.7453 | 862 | 1862 | 2862 | 3862 | 4862 | 5862 | 6862 | 7862 |
| orf6.7500 | 863 | 1863 | 2863 | 3863 | 4863 | 5863 | 6863 | 7863 |
| orf6.7621 | 864 | 1864 | 2864 | 3864 | 4864 | 5864 | 6864 | 7864 |
| orf6.7699 | 865 | 1865 | 2865 | 3865 | 4865 | 5865 | 6865 | 7865 |
| orf6.7983 | 866 | 1866 | 2866 | 3866 | 4866 | 5866 | 6866 | 7866 |
| orf6.8076 | 867 | 1867 | 2867 | 3867 | 4867 | 5867 | 6867 | 7867 |
| orf6.8143 | 868 | 1868 | 2868 | 3868 | 4868 | 5868 | 6868 | 7868 |
| orf6.8690 | 869 | 1869 | 2869 | 3869 | 4869 | 5869 | 6869 | 7869 |
| orf6.9105 | 870 | 1870 | 2870 | 3870 | 4870 | 5870 | 6870 | 7870 |
| orf6.1740 | 871 | 1871 | 2871 | 3871 | 4871 | 5871 | 6871 | 7871 |
| orf6.2265 | 872 | 1872 | 2872 | 3872 | 4872 | 5872 | 6872 | 7872 |
| orf6.2307 | 873 | 1873 | 2873 | 3873 | 4873 | 5873 | 6873 | 7873 |
| orf6.2584 | 874 | 1874 | 2874 | 3874 | 4874 | 5874 | 6874 | 7874 |
| orf6.2709 | 875 | 1875 | 2875 | 3875 | 4875 | 5875 | 6875 | 7875 |
| orf6.2898 | 876 | 1876 | 2876 | 3876 | 4876 | 5876 | 6876 | 7876 |
| orf6.3102 | 877 | 1877 | 2877 | 3877 | 4877 | 5877 | 6877 | 7877 |
| orf6.3507 | 878 | 1878 | 2878 | 3878 | 4878 | 5878 | 6878 | 7878 |
| orf6.3754 | 879 | 1879 | 2879 | 3879 | 4879 | 5879 | 6879 | 7879 |
| orf6.3955 | 880 | 1880 | 2880 | 3880 | 4880 | 5880 | 6880 | 7880 |
| orf6.4050 | 881 | 1881 | 2881 | 3881 | 4881 | 5881 | 6881 | 7881 |
| orf6.4735 | 882 | 1882 | 2882 | 3882 | 4882 | 5882 | 6882 | 7882 |
| orf6.4748 | 883 | 1883 | 2883 | 3883 | 4883 | 5883 | 6883 | 7883 |
| orf6.4766 | 884 | 1884 | 2884 | 3884 | 4884 | 5884 | 6884 | 7884 |
| orf6.5263 | 885 | 1885 | 2885 | 3885 | 4885 | 5885 | 6885 | 7885 |
| orf6.5701 | 886 | 1886 | 2886 | 3886 | 4886 | 5886 | 6886 | 7886 |
| orf6.5918 | 887 | 1887 | 2887 | 3887 | 4887 | 5887 | 6887 | 7887 |
| orf6.6139 | 888 | 1888 | 2888 | 3888 | 4888 | 5888 | 6888 | 7888 |
| orf6.6214 | 889 | 1889 | 2889 | 3889 | 4889 | 5889 | 6889 | 7889 |
| orf6.6250 | 890 | 1890 | 2890 | 3890 | 4890 | 5890 | 6890 | 7890 |
| orf6.6255 | 891 | 1891 | 2891 | 3891 | 4891 | 5891 | 6891 | 7891 |
| orf6.6561 | 892 | 1892 | 2892 | 3892 | 4892 | 5892 | 6892 | 7892 |
| orf6.6665 | 893 | 1893 | 2893 | 3893 | 4893 | 5893 | 6893 | 7893 |
| orf6.6753 | 894 | 1894 | 2894 | 3894 | 4894 | 5894 | 6894 | 7894 |
| orf6.6768 | 895 | 1895 | 2895 | 3895 | 4895 | 5895 | 6895 | 7895 |
| orf6.6808 | 896 | 1896 | 2896 | 3896 | 4896 | 5896 | 6896 | 7896 |
| orf6.6921 | 897 | 1897 | 2897 | 3897 | 4897 | 5897 | 6897 | 7897 |
| orf6.6972 | 898 | 1898 | 2898 | 3898 | 4898 | 5898 | 6898 | 7898 |
| orf6.6993 | 899 | 1899 | 2899 | 3899 | 4899 | 5899 | 6899 | 7899 |
| orf6.6997 | 900 | 1900 | 2900 | 3900 | 4900 | 5900 | 6900 | 7900 |
| orf6.7164 | 901 | 1901 | 2901 | 3901 | 4901 | 5901 | 6901 | 7901 |
| orf6.7180 | 902 | 1902 | 2902 | 3902 | 4902 | 5902 | 6902 | 7902 |
| orf6.7314 | 903 | 1903 | 2903 | 3903 | 4903 | 5903 | 6903 | 7903 |
| orf6.7478 | 904 | 1904 | 2904 | 3904 | 4904 | 5904 | 6904 | 7904 |
| orf6.7883 | 905 | 1905 | 2905 | 3905 | 4905 | 5905 | 6905 | 7905 |
| orf6.7923 | 906 | 1906 | 2906 | 3906 | 4906 | 5906 | 6906 | 7906 |
| orf6.8232 | 907 | 1907 | 2907 | 3907 | 4907 | 5907 | 6907 | 7907 |
| orf6.8441 | 908 | 1908 | 2908 | 3908 | 4908 | 5908 | 6908 | 7908 |
| orf6.8465 | 909 | 1909 | 2909 | 3909 | 4909 | 5909 | 6909 | 7909 |
| orf6.8543 | 910 | 1910 | 2910 | 3910 | 4910 | 5910 | 6910 | 7910 |
| orf6.8747 | 911 | 1911 | 2911 | 3911 | 4911 | 5911 | 6911 | 7911 |
| orf6.8766 | 912 | 1912 | 2912 | 3912 | 4912 | 5912 | 6912 | 7912 |
| orf6.8967 | 913 | 1913 | 2913 | 3913 | 4913 | 5913 | 6913 | 7913 |
| orf6.848 | 914 | 1914 | 2914 | 3914 | 4914 | 5914 | 6914 | 7914 |
| orf6.893 | 915 | 1915 | 2915 | 3915 | 4915 | 5915 | 6915 | 7915 |
| orf6.1000 | 916 | 1916 | 2916 | 3916 | 4916 | 5916 | 6916 | 7916 |
| orf6.1364 | 917 | 1917 | 2917 | 3917 | 4917 | 5917 | 6917 | 7917 |
| orf6.2113 | 918 | 1918 | 2918 | 3918 | 4918 | 5918 | 6918 | 7918 |
| orf6.2481 | 919 | 1919 | 2919 | 3919 | 4919 | 5919 | 6919 | 7919 |
| orf6.2565 | 920 | 1920 | 2920 | 3920 | 4920 | 5920 | 6920 | 7920 |
| orf6.3020 | 921 | 1921 | 2921 | 3921 | 4921 | 5921 | 6921 | 7921 |
| orf6.3597 | 922 | 1922 | 2922 | 3922 | 4922 | 5922 | 6922 | 7922 |
| orf6.6093 | 923 | 1923 | 2923 | 3923 | 4923 | 5923 | 6923 | 7923 |
| orf6.6260 | 924 | 1924 | 2924 | 3924 | 4924 | 5924 | 6924 | 7924 |
| orf6.6934 | 925 | 1925 | 2925 | 3925 | 4925 | 5925 | 6925 | 7925 |
| orf6.6946 | 926 | 1926 | 2926 | 3926 | 4926 | 5926 | 6926 | 7926 |
| orf6.2546 | 927 | 1927 | 2927 | 3927 | 4927 | 5927 | 6927 | 7927 |
| orf6.4262 | 928 | 1928 | 2928 | 3928 | 4928 | 5928 | 6928 | 7928 |
| orf6.4676 | 929 | 1929 | 2929 | 3929 | 4929 | 5929 | 6929 | 7929 |
| orf6.5777 | 930 | 1930 | 2930 | 3930 | 4930 | 5930 | 6930 | 7930 |
| orf6.7959 | 931 | 1931 | 2931 | 3931 | 4931 | 5931 | 6931 | 7931 |
| orf6.8887 | 932 | 1932 | 2932 | 3932 | 4932 | 5932 | 6932 | 7932 |

In one embodiment, the present invention provides the identities of 932 essential genes. Although the nucleotide sequence and the reading frame of a number of these genes are known, the fact that these genes are essential to the growth and/or survival of Candida albicans was not known until the inventors' discovery. Thus, the uses of these genes and their gene products are encompassed by the present invention. Also provided in Table II are SEQ ID NOs: that are used herein to identify the open reading frame, the deduced amino acid sequence and related oligonucleotide sequences for each identified essential gene. To facilitate correlation of the nucleotide sequence of each essential gene with its corresponding amino acid sequence and related oligonucleotide sequences, the sequence identifiers have been organized into eight blocks of each with one thousand SEQ ID numbers. Each block of SEQ ID numbers, which corresponds to a type of sequence, has 932 sequences with SEQ ID NOs., and 166 SEQ ID NOs. with no sequence which serve as place holders. Accordingly, the SEQ ID NO. for each of the eight related sequences of an essential gene are separated by 1000. For example, SEQ ID NOs: 1, 1001, 2001, 3001, 4001, 5001, 6001, and 7001 are directed to, respectively, the upstream and downstream knockout (KO) primers, upstream and downstream tet promoter primers, identification primers A and B, the nucleotide sequence of the coding region and the amino acid sequence of one essential gene, and in this example, the essential gene is CaYDL105W.

In accordance with the above numbering scheme, SEQ ID NOs: 6001 through to SEQ ID NO: 6932 each identifies a nucleotide sequence of the opening reading frame (ORF) of an identified essential gene. The nucleotide sequences labeled as SEQ ID NOs: 6001-6932 were obtained from a Candida albicans genomic sequence database version 6 assembled by the Candida albicans Sequencing Project and is accessible by internet at the web sites of Stanford University and University of Minnesota (See http://www-sequence.stanford.edu:8080/and http://alces.med.umn.edu/Candida.html).

The predicted amino acid sequence of the identified essential genes are set forth in SEQ ID NO: 7001 through to SEQ ID NO: 7932 which are obtained by conceptual translation of the nucleotide sequences of SEQ ID NOs: 6001 through to 6932 once the reading frame is determined. As it is well known in the art, the codon CTG is translated to a serine residue in C. albicans, instead of the usual leucine in other organisms. Accordingly, the conceptual translation of the ORF is performed using the codon usage of C. albicans.

The DNA sequences were generated by sequencing reactions and may contain minor errors which may exist as misidentified nucleotides, insertions, and/or deletions. However, such minor errors, if present, in the sequence database should not disturb the identification of an ORF as that of an essential gene of the invention. Since sequences of the ORFs are provided herein and can be used individually to uniquely identify the corresponding gene in the C. albicans genome, a clone of the gene corresponding to the ORFs can readily be isolated by any of several art-known methods. The sequencing can then be repeated to confirm the sequence or correct the error(s). The disclosure of the ORFs or a portion thereof essentially provides the complete gene by uniquely identifying the coding sequence in question, and providing sufficient guidance to obtain the complete cDNA or genomic sequence. The uses of an essential gene that corresponds to an ORF identified by the methods of the invention are not affected by the minor errors in the ORF.

For example, minor sequence errors and variation in splicing do not affect the construction of conditional-expression C. albicans mutant strains or GRACE strains based on the sequences provided herein, and the uses of those strains, since these methods do not require absolute sequence identity between the chromosomal DNA sequences and the sequences of the gene in the primers or recombinant DNA. In some instances, the correct reading frame of the C. albicans gene can be identified by comparing its overall amino acid sequence with known Saccharomyces cerevisiae sequences. Accordingly, the present invention encompasses C. albicans genes which correspond to the ORFs identified in the invention, polypeptides encoded by C. albicans genes which correspond to the ORFs identified in the invention, and the various uses of the polynucleotides and polypeptides of the genes which correspond to the ORFs of the invention. As used herein in referring to the relationship between a specified nucleotide sequence and a gene, the term “corresponds” or “corresponding” indicates that the specified sequence effectively identifies the gene. In general, correspondence is substantial sequence identity barring minor errors in sequencing, allelic variations and/or variations in splicing. Correspondence can be a transcriptional relationship between the gene sequence and the mRNA or a portion thereof which is transcribed from that gene. This correspondence is present also between portions of an mRNA which is not translated into polypeptide and DNA sequence of the gene.

SEQ ID NOs: 1-5932 identify oligonucleotide primers and probes that were designed for and used in the construction of the GRACE strain for the corresponding identified essential gene. (i.e., SEQ ID NOs: 1-932 are knockout upstream primers (KO-UP); SEQ ID NOs: 1001-1932 are knockout downstream primers (KO-Down); SEQ ID NOs: 2001-2932 are tetracycline promoter upstream primers (Tet-Up); SEQ ID NOs: 3001-3932 are tetracycline promoter downstream primers (Tet-Down); and SEQ ID NOs: 4001-4932, and 5001-5932 are primers for identification of the respective GRACE strains (primers A and B respectively). Therefore, each set of oligonucleotides can be used to identify a unique essential gene and a unique GRACE strain, e.g. by hybridization, or PCR.

The essential genes listed in Table II can be obtained using cloning methods well known to those of skill in the art, and include but are not limited to the use of appropriate probes to detect the genes within an appropriate cDNA or gDNA (genomic DNA) library. (See, for example, Sambrook et al., 1989, Molecular Cloning: A Laboratory Manual, Cold Spring Harbor Laboratories, which is incorporated herein by reference in its entirety.) Probes for the sequences identified herein can be synthesized based on the DNA sequences disclosed herein in SEQ ID NOs: 6001-6932.

As used herein, “target gene” (i.e., essential and/or virulence gene) refers to (a) a gene containing at least one of the DNA sequences and/or fragments thereof that are set forth in SEQ ID NO: 6001 through to SEQ ID NO: 6932; (b) any DNA sequence or fragment thereof that encodes the amino acid sequence that are set forth in SEQ ID NO: 7001 through to SEQ ID NO: 7932 using the universal genetic code or the codon usage of C. albicans; (c) any DNA sequence that hybridizes to the complement of the nucleotide sequences set forth in SEQ ID NO: 6001 through to SEQ ID NO: 6932 under stringent conditions, e.g., hybridization to filter-bound DNA in 6× sodium chloride/sodium citrate (SSC) at about 45° C. followed by one or more washes in 0.2×SSC/0.1% SDS at about 50-65° C., or under highly stringent conditions, e.g., hybridization to filter-bound nucleic acid in 6×SSC at about 45° C. followed by one or more washes in 0.1×SSC/0.2% SDS at about 68° C., or under other hybridization conditions which are apparent to those of skill in the art (see, for example, Ausubel, F. M. et al., eds., 1989, Current Protocols in Molecular Biology, Vol. I, Green Publishing Associates, Inc. and John Wiley & Sons, Inc., New York, at pp. 6.3.1-6.3.6 and 2.10.3). Preferably, the polynucleotides that hybridize to the complements of the DNA sequences disclosed herein encode gene products, e.g., gene products that are functionally equivalent to a gene product encoded by a target gene.

As described above, target gene sequences include not only degenerate nucleotide sequences that encode a polypeptide comprising or consisting essentially of one of the amino acid sequences of SEQ ID NO: 7001 through to SEQ ID NO: 7932 in C. albicans, but also degenerate nucleotide sequences that when translated in organisms other than C. albicans, would yield a polypeptide comprising or consisting essentially of one of the amino acid sequences of SEQ ID NO: 7001 through to SEQ ID NO: 7932, or a fragment thereof. One of skill in the art would know how to select the appropriate codons or modify the nucleotide sequences of SEQ ID NO: 6001 through to SEQ ID NO: 6932 when using the target gene sequences in C. albicans or in other organisms. Moreover, the term “target gene”, in certain embodiments, encompasses genes that are naturally occurring in Saccharomyces cerevisiae or variants thereof, that share extensive nucleotide sequence homology with C. albicans genes having one of the DNA sequences that are set forth in SEQ ID NO: 6001 through to SEQ ID NO: 6932, i.e., the orthologs in S. cerevisiae. It is contemplated that methods for drug screening that can be applied to C. albicans genes can also be applied to orthologs of the same genes in the non-pathogenic S. cerevisiae. However, in certain embodiments, target genes excluding genes of Saccharomyces cerevisiae are used.

In another embodiment, the invention also encompasses the following polynucleotides, host cells expressing such polynucleotides and the expression products of such nucleotides: (a) polynucleotides that encode portions of target gene product that corresponds to its functional domains, and the polypeptide products encoded by such nucleotide sequences, and in which, in the case of receptor-type gene products, such domains include, but are not limited to signal sequences, extracellular domains (ECD), transmembrane domains (TM) and cytoplasmic domains (CD); (b) polynucleotides that encode mutants of a target gene product, in which all or part of one of its domains is deleted or altered, and which, in the case of receptor-type gene products, such mutants include, but are not limited to, mature proteins in which the signal sequence is cleaved, soluble receptors in which all or a portion of the TM is deleted, and nonfunctional receptors in which all or a portion of CD is deleted; and (d) polynucleotides that encode fusion proteins containing a target gene product or one of its domains fused to another polypeptide.

The invention also includes polynucleotides, preferably DNA molecules, that hybridize to, and are therefore the complements of, the DNA sequences of the target gene sequences. Also included are polynucleotides that hybridize to the complement of the DNA sequences of the target genes. Such hybridization conditions can be highly stringent or less highly stringent, as described above and known in the art. The nucleic acid molecules of the invention that hybridize to the above described DNA sequences include oligodeoxynucleotides (“oligos”) which hybridize to the target gene under highly stringent or stringent conditions. In general, for oligos between 14 and 70 nucleotides in length the melting temperature (Tm) is calculated using the formula:

Tm(° C.)=81.5+16.6(log[monovalent cations (molar)]+0.41(% G+C)−(500/N)

where N is the length of the probe. If the hybridization is carried out in a solution containing formamide, the melting temperature may be calculated using the equation:

Tm(° C.)=81.5+16.6(log[monovalent cations (molar)])+0.41(% G+C)−(0.61)(% formamide)−(500/N).

where N is the length of the probe. In general, hybridization is carried out at about 20-25 degrees below Tm (for DNA-DNA hybrids) or about 10-15 degrees below Tm (for RNA-DNA hybrids). Other exemplary highly stringent conditions may refer, e.g., to washing in 6×SSC/0.05% sodium pyrophosphate at 37° C. (for 14-base oligos), 48° C. (for 17-base oligos), 55° C. (for 20-base oligos), and 60° C. (for 23-base oligos). Examples of such oligos are set forth in SEQ ID NOs: 4001 to 4932 and 5001 to 5932.

These nucleic acid molecules can encode or act as target gene antisense molecules, useful, for example, in target gene regulation and/or as antisense primers in amplification reactions of target gene nucleotide sequences. Further, such sequences can be used as part of ribozyme and/or triple helix sequences, also useful for target gene regulation. Still further, such molecules can be used as components of diagnostic methods whereby the presence of the pathogen can be detected. The uses of these nucleic acid molecules are discussed in detail below.

Fragments of the target genes of the invention can be at least 10 nucleotides in length. In alternative embodiments, the fragments can be about 20, 30, 40, 50, 60, 70, 80, 90, 100, 200, 300, 400, 500, 1000, 1500, 2000, 2500, 3000, 3500, 4000, 4500, 5000 or more contiguous nucleotides in length. Alternatively, the fragments can comprise nucleotide sequences that encode at least 10, 20, 30, 40, 50, 100, 150, 200, 250, 300, 350, 400, 450 or more contiguous amino acid residues of the target gene products. Fragments of the target genes of the invention can also refer to exons or introns of the above described nucleic acid molecules, as well as portions of the coding regions of such nucleic acid molecules that encode functional domains such as signal sequences, extracellular domains (ECD), transmembrane domains (TM) and cytoplasmic domains (CD).

To identify and characterize the essential genes of the invention, computer a algorithms are employed to perform searches in computer databases and comparative analysis, and the results of such analyses are stored in or displayed on a computer. Such computerized tools for analyzing sequence information are very useful in determining the relatedness of structure of genes and gene products with respect to other genes and gene products in the same species or a different species, and may provide putative functions to novel genes and their products. Biological information such as nucleotide and amino acid sequences are coded and represented as streams of data in a computer. As used here, the term “computer” includes but is not limited to personal computers, data terminals, computer workstations, networks, computerized storage and retrieval systems, and graphical displays for presentation of sequence information, and results of analyses. Typically, a computer comprises a data entry means, a display means, a programmable processing unit, and a data storage means. A “computer readable medium” can be used to store information such as sequence data, lists, and databases, and includes but is not limited to computer memory, magnetic storage devices, such as floppy discs and magnetic tapes, optical-magnetic storage devices, and optical storage devices, such as compact discs. Accordingly, the present invention also encompass a computer or a computer readable medium that comprises at least one nucleotide sequence selected from the group consisting of SEQ ID NOs: 1-932, 1001-1932, 2001-2932, 3001-3932, 4001-4932, 5001-5932, and 6001-6932, or at least one amino acid sequence selected from the group consisting of SEQ ID NOs: 7001-7932. In preferred embodiments, the sequences are curated and stored in a form with links to other annotations and biological information associated with the sequences. It is also an object of the invention to provide one or more computers programmed with instructions to perform sequence homology searching, sequence alignment, structure prediction and model construction, using the nucleotide sequences of the invention, preferably one or more nucleotide sequences selected from the group consisting of SEQ ID NO: SEQ ID NOs: 1-932, 1001-1932, 2001-2932, 3001-3932, 4001-4932, 5001-5932, and 6001-6932, and/or one or more amino acid sequence selected from the group consisting of SEQ ID NO: SEQ ID NOs: 7001-7932. Computers that comprise, and that can transmit and distribute the nucleotide and/or amino acid sequences of the invention are also contemplated. Also encompassed by the present invention are the uses of one or more nucleotide sequences selected from the group consisting of SEQ ID NOs: 1-932, 1001-1932, 2001-2932, 3001-3932, 4001-4932, 5001-5932, and 6001-6932, and/or one or more amino acid sequence selected from the group consisting of SEQ ID NOs: 7001-7932 in computer-assisted methods for identifying homologous sequences in public and private sequence databases, in computer-assisted methods for providing putative functional characteristics of a gene product based on structural homology with other gene products with known function(s), in computer-assisted methods of constructing a model of the gene product. In one specific embodiment, the invention encompasses a method assisted by a computer for identifying a putatively essential gene of a fungus, comprising detecting sequence homology between a fungal nucleotide sequence or fungal amino acid sequence with at least one nucleotide sequence selected from the group consisting of SEQ ID NOs: 1-932, 1001-1932, 2001-2932, 3001-3932, 4001-4932, 5001-5932, and 6001-6932, or at least one amino acid sequence selected from the group consisting of SEQ ID NOs: 7001-7932.

5.4.2 Homologous Target Genes

In addition to the nucleotide sequences of Candida albicans described above, homologs or orthologs of these target gene sequences, as can be present in other species, can be identified and isolated by molecular biological techniques well known in the art, and without undue experimentation, used in the methods of the invention. For example, homologous target genes in Aspergillus fumigatus, Aspergillus flavus, Aspergillus niger, Coccidioides immitis, Cryptococcus neoformans, Histoplasma capsulatum, Phytophthora infestans, Puccinia seconditii, Pneumocystis carinii, or any species falling within the genera of any of the above species. Other yeasts in the genera of Candida, Saccharomyces, Schizosaccharomyces, Sporobolomyces, Torulopsis, Trichosporon, Tricophyton, Dermatophytes, Microsproum, Wickerhamia, Ashbya, Blastomyces, Candida, Citeromyces, Crebrothecium, Cryptococcus, Debaryomyces, Endomycopsis, Geotrichum, Hansenula, Kloeckera, Kluveromyces, Lipomyces, Pichia, Rhodosporidium, Rhodotorula, and Yarrowia are also contemplated. Also included are homologs of these target gene sequences can be identified in and isolated from animal fugal pathogens such as Aspergillus fumigatus, Aspergillus niger, Aspergillus flavis, Candida tropicalis, Candida parapsilopsis, Candida krusei, Cryptococcus neoformans, Coccidioides immitis, Exophalia dermatiditis, Fusarium oxysporum, Histoplasma capsulatum, Phneumocystis carinii, Trichosporon beigelii, Rhizopus arrhizus, Mucor rouxii, Rhizomucor pusillus, or Absidia corymbigera, or the plant fungal pathogens, such as Alternaria solanii, Botrytis cinerea, Erysiphe graminis, Magnaporthe grisea, Puccinia recodita, Sclerotinia sclerotiorum, Septoria triticii, Tilletia controversa, Ustilago maydis, Venturia inequalis, Verticullium dahliae or any species falling within the genera of any of the above species.

Accordingly, the present invention provides polynucleotides that comprise nucleotide sequences allowing them to hybridize to the polynucleotides of the target genes. In one embodiment, the present invention encompasses an isolated nucleic acid comprising a nucleotide sequence that is at least 50% identical to a nucleotide sequence selected from the group consisting of SEQ ID NO: 6001 through to SEQ ID NO: 6932, and that is of a species other than Saccharomyces cerevisiae and/or Candida albicans. In another embodiment, the present invention encompasses an isolated nucleic acid comprising a nucleotide sequence that hybridizes under medium stringency conditions to a second nucleic acid that consists of a nucleotide sequence selected from the group consisting of SEQ ID NO: 6001 through to SEQ ID NO: 6932, and that is of a species other than Saccharomyces cerevisiae and/or Candida albicans. In a specific embodiment, the nucleotide sequence that is at least 50% identical or hybridizes under medium stringency conditions to any one of the sequences SEQ ID NO: 6001 through to SEQ ID NO: 6932 is from Aspergillus fumigatus or Cryptococcus neoformans. In another specific embodiment, the nucleotide sequence that is at least 50% identical or hybridizes under medium stringency conditions to any one of the sequences SEQ ID NO: 6001 through to SEQ ID NO: 6932 is of a species other than Aspergillus fumigatus and/or Cryptococcus neoformans.

In yet another embodiment, the present invention includes an isolated nucleic acid comprising a nucleotide sequence that encodes a polypeptide the amino acid sequence of which is at least 50% identical to an amino acid sequence selected from the group consisting of SEQ ID NO. 7001 through to 7932, wherein the polypeptide is that of a species other than Saccharomyces cerevisiae and/or Candida albicans. In a specific embodiment, the amino acid sequence that is at least 50% identical to any one of the sequences SEQ ID NO: 7001 through to SEQ ID NO: 7932 is from Aspergillus fumigatus or Cryptococcus neoformans. In another specific embodiment, the amino acid sequence that is at least 50% identical to any one of the sequences SEQ ID NO: 7001 through to SEQ ID NO: 7932 is of a species other than Aspergillus fumigatus and/or Cryptococcus neoformans.

Although the nucleotide sequences and amino acid sequences of homologs or orthologs of the essential/virulence genes in S. cerevisiae are mostly published, uses of such homologs or orthologs in S. cerevisiae in drug screening are mostly not known and are thus specifically provided by the invention. To use such nucleotide and/or amino acid sequences of S. cerevisiae, public databases, such as Stanford Genomic Resources (www-genome.stanford.edu), Munich Information Centre for Protein Sequences (www.mips.biochem.mpg.de) or Proteome (www.proteome.com) may be used to identify and retrieve the sequences. Orthologs of S. cerevisiae can also be identified by hybridization assays using nucleic acid probes consisting of any one of the nucleotide sequences of SEQ ID NOs: 6001 to 6932.

The nucleotide sequences of the invention still further include nucleotide sequences that have at least 40%, 45%, 55%, 60%, 65%, 70%, 75%, 80%, 85%, 90%, 95%, 98% or more nucleotide sequence identity to the nucleotide sequences set forth in SEQ ID NO: 6001 through to SEQ ID NO: 6932. The nucleotide sequences of the invention also include nucleotide sequences that encode polypeptides having at least 25%, 30%, 40%, 50%, 55%, 60%, 65%, 70%, 75%, 80%, 85%, 90%, 95%, 98% or higher amino acid sequence identity or similarity to the amino acid sequences set forth in SEQ ID NOs: 7001 15 through to 7932.

To determine the percent identity of two amino acid sequences or of two nucleotide sequences, the sequences are aligned for optimal comparison purposes (e.g., gaps can be introduced in the sequence of a first amino acid or nucleotide sequence for optimal alignment with a second amino acid or nucleotide sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity number of identical overlapping positions/total number of positions×100%). In one embodiment, the two sequences are the same length.

The determination of percent identity between two sequences can also be accomplished using a mathematical algorithm and computer-assisted methods. A preferred, non-limiting example of a mathematical algorithm utilized for the comparison of two sequences is the algorithm of Karlin and Altschul (1990) Proc. Natl. Acad. Sci. U.S.A. 87:2264-2268, modified as in Karlin and Altschul (1993) Proc. Natl. Acad. Sci. U.S.A. 90:5873-5877. Such an algorithm is incorporated into the NBLAST and XBLAST programs of Altschul et al., 1990, J. Mol. Biol. 215:403-0. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., http://www.ncbi.nlm.nih.gov). Another preferred, non-limiting example of a mathematical algorithm utilized for the comparison of sequences is the algorithm of Myers and Miller, (1988) CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Any of these algorithms can be coded as a set of instructions for use in a computer that comprises the sequences of the invention.

To isolate homologous target genes, the C. albicans target gene sequence described above can be labeled and used to screen a cDNA library constructed from mRNA obtained from the organism of interest. Hybridization conditions should be of a lower stringency when the cDNA library was derived from an organism different from the type of organism from which the labeled sequence was derived. cDNA screening can also identify clones derived from alternatively spliced transcripts in the same or different species. Alternatively, the labeled fragment can be used to screen a genomic library derived from the organism of interest, again, using appropriately stringent conditions. Low stringency conditions will be well known to those of skill in the art, and will vary predictably depending on the specific organisms from which the library and the labeled sequences are derived. For guidance regarding such conditions see, for example, Sambrook et al., 1989, Molecular Cloning, A Laboratory Manual, Cold Spring Harbor Press, N.Y.; and Ausubel et al., 1989, Current Protocols in Molecular Biology, (Green Publishing Associates and Wiley Interscience, N.Y.).

Further, a homologous target gene sequence can be isolated by performing a polymerase chain reaction (PCR) using two degenerate oligonucleotide primer pools designed on the basis of amino acid sequences within the target gene of interest. The template for the reaction can be cDNA obtained by reverse transcription of mRNA prepared from the organism of interest. The PCR product can be subcloned and sequenced to ensure that the amplified sequences represent the sequences of a homologous target gene sequence.

The PCR fragment can then be used to isolate a full length cDNA clone by a variety of methods well known to those of ordinary skill in the art. Alternatively, the labeled fragment can be used to screen a genomic library.

PCR technology can also be utilized to isolate full length cDNA sequences. For example, RNA can be isolated, following standard procedures, from an organism of interest. A reverse transcription reaction can be performed on the RNA using an oligonucleotide primer specific for the most 5′ end of the amplified fragment for the priming of first strand synthesis. The resulting RNA/DNA hybrid can then be “tailed” with guanines using a standard terminal transferase reaction, the hybrid can be digested with RNAase H, and second strand synthesis can then be primed with a poly-C primer. Thus, cDNA sequences upstream of the amplified fragment can easily be isolated. For a review of cloning strategies which can be used, see e.g., Sambrook et al., 1989, Molecular Cloning, A Laboratory Manual, Cold Springs Harbor Press, N.Y.; and Ausubel et al., 1989, Current Protocols in Molecular Biology, (Green Publishing Associates and Wiley Interscience, N.Y.).

Additionally, an expression library can be constructed utilizing DNA isolated from or cDNA synthesized from the organism of interest. In this manner, gene products made by the homologous target gene can be expressed and screened using standard antibody screening techniques in conjunction with antibodies raised against the C. albicans gene product, as described, below. (For screening techniques, see, for example, Harlow, E. and Lane, eds., 1988, “Antibodies: A Laboratory Manual,” Cold Spring Harbor Press, Cold Spring Harbor). Library clones detected via their reaction with such labeled antibodies can be purified and subjected to sequence analysis by well known methods.

Alternatively, homologous target genes or polypeptides may be identified by searching a database to identify sequences having a desired level of homology to a target gene or polypeptide involved in proliferation, virulence or pathogenicity. A variety of such databases are available to those skilled in the art, including GenBank and GenSeq. In various embodiments, the databases are screened to identify nucleic acids with at least 97%, at least 95%, at least 90%, at least 85%, at least 80%, at least 70%, at least 60%, at least 50%, or at least 40% identity to a target nucleotide sequence, or a portion thereof. In other embodiments, the databases are screened to identify polypeptides having at least 99%, at least 95%, at least 90%, at least 85%, at least 80%, at least 70%, at least 60%, at least 50%, at least 40% or at least 25% identity or similarity to a polypeptide involved in proliferation, virulence or pathogenicity or a portion thereof.

Alternatively, functionally homologous target sequences or polypeptides may be identified by creating mutations that have phenotypes by removing or altering the function of a gene. This can be done for one or all genes in a given fungal species including, for example: Saccharomyces cerevisiae, Candida albicans, and Aspergillus fumigatus. Having mutants in the genes of one fungal species offers a method to identify functionally similar genes (orthologs) or related genes (paralogs) in another species, by use of a functional complementation test.

A library of gene or cDNA copies of messenger RNA of genes can be made from a given species, e.g. Candida albicans, and the library cloned into a vector permitting expression (for example, with the Candida albicans promoters or a Saccharomyces cerevisiae promoter) of the genes in a second species, e.g. Saccharomyces cerevisiae. Such a library is referred to as a “heterologous library.” Transformation of the Candida albicans heterologous library into a defined mutant of Saccharomyces cerevisiae that is functionally deficient with respect to the identified gene, and screening or selecting for a gene in the heterologous library that restores phenotypic function in whole or in part of the mutational defect is said to be “heterologous functional complementation” and in this example, permits identification of gene in Candida albicans that are functionally related to the mutated gene in Saccharomyces cerevisiae. Inherent in this functional-complementation method, is the ability to restore gene function without the requirement for sequence similarity of nucleic acids or polypeptides; that is, this method permits interspecific identification of genes with conserved biological function, even where sequence similarity comparisons fail to reveal or suggest such conservation.

In those instances in which the gene to be tested is an essential gene, a number of possibilities exist regarding performing heterologous functional complementation tests. The mutation in the essential gene can be a conditional allele, including but not limited to, a temperature-sensitive allele, an allele conditionally expressed from a regulatable promoter, or an allele that has been rendered the mRNA transcript or the encoded gene product conditionally unstable. Alternatively, the strain carrying a mutation in an essential gene can be propagated using a copy of the native gene (a wild type copy of the gene mutated from the same species) on a vector comprising a marker that can be selected against, permitting selection for those strains carrying few or no copies of the vector and the included wild type allele. A strain constructed in this manner is transformed with the heterologous library, and those clones in which a heterologous gene can functionally complement the essential gene mutation, are selected on medium non-permissive for maintenance of the plasmid carrying the wild type gene.

In the following example, the identification, by functional complementation, of a Candida albicans homolog of a Saccharomyces cerevisiae gene, KRE 9, is described. (Lussier et al. 1998, “The Candida albicans KRE 9 gene is required for cell wall β-1,6-glucan synthesis and is essential for growth on glucose,” Proc. Natl. Acad. Sci. USA 95: 9825-30). The host strain was a Saccharomyces cerevisiae haploid null mutant in KRE 9, kre 9::HIS3, which has a severe growth defect phenotype. The host strain carried a wild type copy of the native Saccharomyces cerevisiae KRE 9 gene on a LYS-2 based pRS317 shuttle vector and was transformed with a Candida albicans genomic library. This heterologous library was constructed using, as a vector, the multicopy plasmid YEp352, which carries the URA3 gene as a selectable marker. To screen for plasmids supporting growth of the kre 9::HIS 3 mutant host, approximately 20,000 colonies capable of growth in the absence of histidine, lysine, and uracil, were replica-plated onto minimal medium containing α-amino adipate as a nitrogen source to allow selection for cells that have lost the LYS2 plasmid-based copy of KRE 9 and that possess a copy of a functionally-complementing Candida albicans ortholog, CaKRE 9. These cells were tested further for loss of the pRS317-KRE 9 plasmid by their inability to grow in the absence of lysine, and YEp352-based Candida albicans genomic DNA was recovered from them. On retransformation of the Saccharomyces cerevisiae kre 9::HIS3 mutant, a specific genomic insert of 8 kb of Candida albicans was recovered that was able to restore growth partially. Following further subcloning using functional complementation for selection, a 1.6 kb DNA fragment was obtained that contained the functional Candida albicans KRE 9 gene.

A heterologous functional complementation test is not restricted to the exchange of genetic information between Candida albicans and Saccharomyces cerevisiae; functional complementation tests can be performed, as described above, using any pair of fungal species. For example, the CRE1 gene of the fungus Sclerotininia sclerotiorum can functionally complement the creAD30 mutant of the CREA gene of Aspergillus nidulans (see Vautard et al. 1999, “The glucose repressor gene CRE1 from Sclerotininia sclerotiorum is functionally related to CREA from Aspergillus nidulans but not to the Mig proteins from Saccharomyces cerevisiae,” FEBS Lett. 453: 54-58).

In yet another embodiment, where the source of nucleic acid deposited on a gene expression array and the source of the nucleic acid probe being hybridized to the array are from two different species of organisms, the results allow rapid identification of homologous genes in the two species.

In yet another embodiment, the invention also encompasses (a) DNA vectors that contain a nucleotide sequence comprising any of the foregoing coding sequences of the target gene and/or their complements (including antisense); (b) DNA expression vectors that contain a nucleotide sequence comprising any of the foregoing coding sequences operably linked with a regulatory element that directs the expression of the coding sequences; and (c) genetically engineered host cells that contain any of the foregoing coding sequences of the target gene operably linked with a regulatory element that directs the expression of the coding sequences in the host cell. Vectors, expression constructs, expression vectors, and genetically engineered host cells containing the coding sequences of homologous target genes of other species (excluding S. cerevisiae) are also contemplated. Also contemplated are genetically engineered host cells containing mutant alleles in homologous target genes of the other species. As used herein, regulatory elements include but are not limited to inducible and non-inducible promoters, enhancers, operators and other elements known to those skilled in the art that drive and regulate expression. Such regulatory elements include but are not limited to the lac system, the trp system, the tet system and other antibiotic-based repression systems (e.g. PIP), the TAC system, the TRC system, the major operator and promoter regions of phage A, the control regions of fd coat protein, and the fungal promoters for 3-phosphoglycerate kinase, acid phosphatase, the yeast mating pheromone responsive promoters (e.g. STE2 and STE3), and promoters isolated from genes involved in carbohydrate metabolism (e.g. GAL promoters), phosphate-responsive promoters (e.g. PHO5), or amino acid metabolism (e.g. MET genes). The invention includes fragments of any of the DNA vector sequences disclosed herein.

A variety of techniques can be utilized to further characterize the identified essential genes and virulence genes. First, the nucleotide sequence of the identified genes can be used to reveal homologies to one or more known sequence motifs which can yield information regarding the biological function of the identified gene product. Computer programs well known in the art can be employed to identify such relationships. Second, the sequences of the identified genes can be used, utilizing standard techniques such as in situ hybridization, to place the genes onto chromosome maps and genetic maps which can be correlated with similar maps constructed for another organism, e.g., Saccharomyces cerevisiae. The information obtained through such characterizations can suggest relevant methods for using the polynucleotides and polypeptides for discovery of drugs against Candida albicans and other pathogens.

Methods for performing the uses listed above are well known to those skilled in the art. References disclosing such methods include without limitation “Molecular Cloning: A Laboratory Manual,” 2d ed., Cold Spring Harbor Laboratory Press, Sambrook, J., E. F. Fritsch and T. Maniatis eds., 1989, and “Methods in Enzymology: Guide to Molecular Cloning Techniques,” Academic Press, Berger, S. L. and A. R. Kimmel eds., 1987. Many of the uses of the polynucleotides and polypeptides of the identified essential genes are discussed in details hereinbelow.

5.4.3 Target Gene Products

The target gene products used and encompassed in the methods and compositions of the present invention include those gene products (e.g, RNA or proteins) that are encoded by the target essential gene sequences as described above, such as, the target gene sequences set forth in SEQ ID NOs: 6001 through to 6932. In Table II, the amino acid sequences of SEQ ID NOs: 7001 through to 7932 are deduced using the codon usage of C. albicans from the respective nucleotide sequences of SEQ ID NOs: 6001 through to 6932. However, when expressed in an organism other than C. albicans, protein products of the target genes comprising the amino acid sequences of SEQ ID NOs: 7001 through to 7932 may be encoded by nucleotide sequences that are translated using the universal genetic code. One of skill in the art would know the modifications that are necessary to accommodate for such a difference in codon usage.

In addition, however, the methods and compositions of the invention also use and encompass proteins and polypeptides that represent functionally equivalent gene products. Such functionally equivalent gene products include, but are not limited to, natural variants of the polypeptides comprising or consisting essentially of an amino acid sequence set forth in SEQ ID NOs: 7001 through to 7932.

Such equivalent target gene products can contain, e.g., deletions, additions or substitutions of amino acid residues within the amino acid sequences encoded by the target gene sequences described above, but which result in a silent change, thus producing a functionally equivalent target gene product. Conservative amino acid substitutions can be made on the basis of similarity in polarity, charge, solubility, hydrophobicity, hydrophilicity and/or the amphipathic nature of the residues involved. For example, nonpolar (i.e., hydrophobic) amino acid residues can include alanine (Ala or A), leucine (Leu or L), isoleucine (Ile or D, valine (Val or V), proline (Pro or P), phenylalanine (Phe or F), tryptophan (Trp or W) and methionine (Met or M); polar neutral amino acid residues can include glycine (Gly or G), serine (Ser or S), threonine (Thr or T), cysteine (Cys or C), tyrosine (Tyr or Y), asparagine (Asn or N) and glutamine (Gln or Q); positively charged (i.e., basic) amino acid residues can include arginine (Arg or R), lysine (Lys or K) and histidine (His or H); and negatively charged (i.e., acidic) amino acid residues can include aspartic acid (Asp or D) and glutamic acid (Glu or E).

In one particular embodiment, a composition comprising a mixture of natural variants of the polypeptides having one of SEQ ID NOs: 7001 through to 7932 is provided. Since it is known in the art that, in C. albicans, 99% of the tRNA molecules that recognize the codon CTG is charged with a serine residue, and 1% are charged with a leucine residue, there is a possibility that during biosynthesis, a leucine is incorporated into a growing polypeptide chain. Accordingly, when a nucleotide sequence comprising the codon CTG is translated in C. albicans, a small percentage of the resulting polypeptides may have a leucine residue in positions where a serine residue encoded by CTG (conforming to the codon usage of C. albicans) is expected. The product of translation of such a nucleotide sequence may comprise a mixture of polypeptides with minor leucine/serine variations at positions that correspond to a CTG codon in the nucleotide sequence.

“Functionally equivalent,” as the term is utilized herein, refers to a polypeptide capable of exhibiting a substantially similar in vivo activity as the Candida albicans target gene product encoded by one or more of the target gene sequences described in Table II. Alternatively, when utilized as part of assays described hereinbelow, the term “functionally equivalent” can refer to peptides or polypeptides that are capable of interacting with other cellular or extracellular molecules in a manner substantially similar to the way in which the corresponding portion of the target gene product would interact with such other molecules. Preferably, the functionally equivalent target gene products of the invention are also the same size or about the same size as a target gene product encoded by one or more of the target gene sequences described in Table II.

The biological function of the target gene products encoded by the C. albicans essential genes of the invention can be predicted by the function of their corresponding homologs in Saccharomyces cerevisiae. Accordingly, the C. albicans gene products of the invention may have one or more of the following biological functions:

Metabolism: amino-acid metabolism, amino-acid biosynthesis, assimilatory reduction of sulfur and biosynthesis of the serine family, regulation of amino-acid metabolism, amino-acid transport, amino-acid degradation (catabolism), other amino-acid metabolism activities, nitrogen and sulphur metabolism, nitrogen and sulphur utilization, regulation of nitrogen and sulphur utilization, nitrogen and sulphur transport, nucleotide metabolism, purine-ribonucleotide metabolism, pyrimidine-ribonucleotide metabolism, deoxyribonucleotide metabolism, metabolism of cyclic and unusual nucleotides, regulation of nucleotide metabolism, polynucleotide degradation, nucleotide transport, other nucleotide-metabolism activities, phosphate metabolism, phosphate utilization, regulation of phosphate utilization, phosphate transport, other phosphate metabolism activities, C-compound and carbohydrate metabolism, C-compound and carbohydrate utilization, regulation of C-compound and carbohydrate utilization, C-compound, carbohydrate transport, other carbohydrate metabolism activities, lipid, fatty-acid and isoprenoid metabolism, lipid, fatty-acid and isoprenoid biosynthesis, phospholipid biosynthesis, glycolipid biosynthesis, breakdown of lipids, fatty acids and isoprenoids, lipid, fatty-acid and isoprenoid utilization, regulation of lipid, fatty-acid and isoprenoid biosynthesis, lipid and fatty-acid transport, lipid and fatty-acid binding, other lipid, fatty-acid and isoprenoid metabolism activities, metabolism of vitamins, cofactors, and prosthetic groups, biosynthesis of vitamins, cofactors, and prosthetic groups, utilization of vitamins, cofactors, and prosthetic groups, regulation of vitamins, cofactors, and prosthetic groups, transport of vitamins, cofactors, and prosthetic groups, other vitamin, cofactor, and prosthetic group activities, secondary metabolism, metabolism of primary metabolic sugars derivatives, biosynthesis of glycosides, biosynthesis of secondary products derived from primary amino acids, biosynthesis of amines.

Energy: glycolysis and gluconeogenesis, pentose-phosphate pathway, tricarboxylic-acid pathway, electron transport and membrane-associated energy conservation, accessory proteins of electron transport and membrane-associated energy conservation, other electron transport and membrane-associated energy conservation proteins, respiration, fermentation, metabolism of energy reserves (glycogen, trehalose), glyoxylate cycle, oxidation of fatty acids, other energy generation activities.

Cell Growth, Cell Division and DNA Synthesis: cell growth, budding, cell polarity and filament formation, pheromone response, mating-type determination, sex-specific proteins, sporulation and germination, meiosis, DNA synthesis and replication, recombination and DNA repair, cell cycle control and mitosis, cell cycle check point proteins, cytokinesis, other cell growth, cell division and DNA synthesis activities.

Transcription: rRNA transcription, rRNA synthesis, rRNA processing, other rRNA-transcription activities, tRNA transcription, tRNA synthesis, tRNA processing, tRNA modification, other tRNA-transcription activities, mRNA transcription, mRNA synthesis, general transcription activities, transcriptional control, chromatin modification, mRNA processing (splicing), mRNA processing (5′-, 3′-end processing, mRNA degradation), 3′-end processing, mRNA degradation, other mRNA-transcription activities, RNA transport, other transcription activities.

Protein Synthesis: ribosomal proteins, translation, translational control, tRNA-synthetases, other protein-synthesis activities.

Protein Destination: protein folding and stabilization, protein targeting, sorting and translocation, protein modification, modification with fatty acids (e.g. myristylation, pabnitylation, farnesylation), modification by phosphorylation, dephosphorylation, modification by acetylation, other protein modifications, assembly of protein complexes, proteolysis, cytoplasmic and nuclear degradation, lysosomal and vacuolar degradation, other proteolytic degradation, other proteolytic proteins, other protein-destination activities.

Transport Facilitation: channels/pores, ion channels, ion transporters, metal ion transporters (Cu, Fe, etc.), other cation transporters (Na, K, Ca, NH₄, etc.), anion transporters (Cl, SO₄, PO₄, etc.), C-compound and carbohydrate transporters, other C-compound transporters, amino-acid transporters, peptide-transporters, lipid transporters, purine and pyrimidine transporters, allantoin and allantoate transporters, transport ATPases, ABC transporters, drug transporters, other transport facilitators.

Cellular Transport and Transport Mechanisms: nuclear transport, mitochondrial transport, vesicular transport (Golgi network, etc.), peroxisomal transport, vacuolar transport, extracellular transport (secretion), cellular import, cytoskeleton-dependent transport, transport mechanism, other transport mechanisms, other intracellular-transport activities.

Cellular Biogenesis: biogenesis of cell wall (cell envelope), biogenesis of plasma membrane, biogenesis of cytoskeleton, biogenesis of endoplasmatic reticulum, biogenesis of Golgi, biogenesis of intracellular transport vesicles, nuclear biogenesis, biogenesis of chromosome structure, mitochondrial biogenesis, peroxisomal biogenesis, endosomal biogenesis, vacuolar and lysosomal biogenesis, other cellular biogenesis activities.

Cellular Communication/signal Transduction: intracellular communication, unspecified signal transduction, second messenger formation, regulation of G-protein activity, key kinases, other unspecified signal transduction activities, morphogenesis, G-proteins, regulation of G-protein activity, key kinases, other morphogenetic activities, osmosensing, receptor proteins, mediator proteins, key kinases, key phosphatases, other osmosensing activities, nutritional response pathway, receptor proteins, second messenger formation, G-proteins, regulation of G-protein activity, key kinases, key phosphatases, other nutritional-response activities, pheromone response generation, receptor proteins, G-proteins, regulation of G-protein activity, key kinases, key phosphatases, other pheromone response activities, other signal-transduction activities.

Cell Rescue, Defense, Cell Death and Ageing: stress response, DNA repair, other DNA repair, detoxification, detoxification involving cytochrome P450, other detoxification, cell death, ageing, degradation of exogenous polynucleotides, other cell rescue activities.

Ionic Homeostasis: homeostasis of cations, homeostasis of metal ions, homeostasis of protons, homeostasis of other cations, homeostasis of anions, homeostasis of sulfates, homeostasis of phosphate, homeostasis of chloride, homeostasis of other anions.

Cellular Organization (proteins are localized to the corresponding organelle): organization of cell wall, organization of plasma membrane, organization of cytoplasm, organization of cytoskeleton, organization of centrosome, organization of endoplasmatic reticulum, organization of Golgi, organization of intracellular transport vesicles, nuclear organization, organization of chromosome structure, mitochondrial organization, peroxisomal organization, endosomal organization, vacuolar and lysosomal organization, inner membrane organization, extracellular/secretion proteins.

In another embodiment of the invention, the use of target gene products that are RNA or proteins of Saccharomyces cerevisiae are provided.

Peptides and polypeptides corresponding to one or more domains of the target gene products (e.g., signal sequence, TM, ECD, CD, or ligand-binding domains), truncated or deleted target gene products (e.g, polypeptides in which one or more domains of a target gene product are deleted) and fusion target gene proteins (e.g, proteins in which a full length or truncated or deleted target gene product, or a peptide or polypeptide corresponding to one or more domains of a target gene product is fused to an unrelated protein) are also within the scope of the present invention. Such peptides and polypeptides (also referred to as chimeric protein or polypeptides) can be readily designed by those skilled in the art on the basis of the target gene nucleotide and amino acid sequences listed in Table II. Exemplary fusion proteins can include, but are not limited to, epitope tag-fusion proteins which facilitates isolation of the target gene product by affinity chromatography using reagents that binds the epitope. Other exemplary fusion proteins include fusions to any amino acid sequence that allows, e.g., the fusion protein to be anchored to a cell membrane, thereby allowing target gene polypeptides to be exhibited on a cell surface; or fusions to an enzyme (e.g., β-galactosidase encoded by the LAC4 gene of Kluyveronmyces lactis (Leuker et al., 1994, Mol. Gen. Genet., 245:212-217)), to a fluorescent protein (e.g., from Renilla reniformis (Srikantha et al., 1996, J. Bacteriol. 178:121-129), or to a luminescent protein which can provide a marker function. Accordingly, the invention provides a fusion protein comprising a fragment of a first polypeptide fused to a second polypeptide, said fragment of the first polypeptide consisting of at least 6 consecutive residues of an amino acid sequence selected from one of SEQ ID NOs: 7001 through to 7932.

Other modifications of the target gene product coding sequences described above can be made to generate polypeptides that are better suited, e.g., for expression, for scale up, etc. in a chosen host cell. For example, cysteine residues can be deleted or substituted with another amino acid in order to eliminate disulfide bridges.

The target gene products of the invention preferably comprise at least as many contiguous amino acid residues as are necessary to represent an epitope fragment (that is, for the gene products to be recognized by an antibody directed to the target gene product). For example, such protein fragments or peptides can comprise at least about 8 contiguous amino acid residues from a full length differentially expressed or pathway gene product. In alternative embodiments, the protein fragments and peptides of the invention can comprise about 6, 10, 20, 30, 40, 50, 60, 70, 80, 90, 100, 150, 200, 250, 300, 350, 400, 450 or more contiguous amino acid residues of a target gene product.

The target gene products used and encompassed in the methods and compositions of the present invention also encompass amino acid sequences encoded by one or more of the above-described target gene sequences of the invention wherein domains often encoded by one or more exons of those sequences, or fragments thereof, have been deleted. The target gene products of the invention can still further comprise post translational modifications, including, but not limited to, glycosylations, acetylations and myristylations.

The target gene products of the invention can be readily produced, e.g., by synthetic techniques or by methods of recombinant DNA technology using techniques that are well known in the art. Thus, methods for preparing the target gene products of the invention are discussed herein. First, the polypeptides and peptides of the invention can be synthesized or prepared by techniques well known in the art. See, for example, Creighton, 1983, Proteins: Structures and Molecular Principles, W. H. Freeman and Co., N.Y., which is incorporated herein by reference in its entirety. Peptides can, for example, be synthesized on a solid support or in solution.

Alternatively, recombinant DNA methods which are well known to those skilled in the art can be used to construct expression vectors containing target gene protein coding sequences such as those set forth in SEQ ID NOs: 6001 through to 6932, and appropriate transcriptional/translational control signals. These methods include, for example, in vitro recombinant DNA techniques, synthetic techniques and in vivo recombination/genetic recombination. See, for example, the techniques described in Sambrook et al., 1989, Molecular Cloning: A Laboratory Manual, Cold Spring Harbor Press, Cold Spring Harbor, N.Y., Pla et al., Yeast 12:1677-1702 (1996), which are incorporated by reference herein in their entireties, and Ausubel, 1989, supra. Alternatively, RNA capable of encoding target gene protein sequences can be chemically synthesized using, for example, synthesizers. See, for example, the techniques described in Oligonucleotide Synthesis, 1984, Gait, M. J. ed., IRL Press, Oxford, which is incorporated by reference herein in its entirety.

A variety of host-expression vector systems can be utilized to express the target gene coding sequences of the invention. Such host-expression systems represent vehicles by which the coding sequences of interest can be produced and subsequently purified, but also represent cells which can, when transformed or transfected with the appropriate nucleotide coding sequences, exhibit the target gene protein of the invention in situ. These include but are not limited to microorganisms such as bacteria (e.g., E. coli, B. subtilis) transformed with recombinant bacteriophage DNA, plasmid DNA or cosmid DNA expression vectors containing target gene protein coding sequences; yeast (e.g., Saccharomyces, Schizosaccharomyces, Neurospora, Aspergillus, Candida, Pichia) transformed with recombinant yeast expression vectors containing the target gene protein coding sequences; insect cell systems infected with recombinant virus expression vectors (e.g, baculovirus) containing the target gene protein coding sequences; plant cell systems infected with recombinant virus expression vectors (e.g, cauliflower mosaic virus, CaMV; tobacco mosaic virus, TMV) or transformed with recombinant plasmid expression vectors (e.g, Ti plasmid) containing target gene protein coding sequences; or mammalian cell systems (e.g. COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter). If necessary, the nucleotide sequences of coding regions may be modified according to the codon usage of the host such that the translated product has the correct amino acid sequence.

In bacterial systems, a number of expression vectors can be advantageously selected depending upon the use intended for the target gene protein being expressed. For example, when a large quantity of such a protein is to be produced, for the generation of antibodies or to screen peptide libraries, for example, vectors which direct the expression of high levels of fusion protein products that are readily purified can be desirable. Such vectors include, but are not limited, to the E. coli expression vector pUR278 (Ruther et al., 1983, EMBO J. 2:1791), in which the target gene protein coding sequence can be ligated individually into the vector in frame with the lacZ coding region so that a fusion protein is produced; pIN vectors (Inouye & Inouye, 1985, Nucleic Acids Res. 13:3101-3109; Van Heeke & Schuster, 1989, J. Biol. Chem. 264:5503-5509); and the like. pGEX vectors can also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene protein can be released from the GST moiety.

When a target gene is to be expressed in mammalian host cells, a number of viral-based expression systems can be utilized. In cases where an adenovirus is used as an expression vector, the target gene coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing target gene protein in infected hosts, (e.g., See Logan & Shenk, 1984, Proc. Natl. Acad Sci. USA 81:3655-3659). Specific initiation signals can also be required for efficient translation of inserted target gene coding sequences. These signals include the ATG initiation codon and adjacent sequences. In cases where an entire target gene, including its own initiation codon and adjacent sequences, is inserted into the appropriate expression vector, no additional translational control signals can be needed. However, in cases where only a portion of the target gene coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see Bittner et al., 1987, Methods in Enzymol. 153:516-544).

In addition, a host cell strain can be chosen which modulates the expression of the inserted sequences, or modifies and processes the gene product in the specific fashion desired. Such modifications (e.g, glycosylation) and processing (e.g., cleavage) of protein products can be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used.

For long-term, high-yield production of recombinant proteins, stable expression is preferred. For example, cell lines which stably express the target gene protein can be engineered. Host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express the target gene protein. Such engineered cell lines can be particularly useful in screening and evaluation of compounds that affect the endogenous activity of the target gene protein.

A number of selection systems can be used, including but not limited to the herpes simplex virus thymidine kinase (Wigler et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy et al., 1980, Cell 22:817) genes can be employed in tk⁻, hgprt⁻ or aprt⁻ cells, respectively. Also, antimetabolite resistance can be used as the basis of selection for dhfr, which confers resistance to methotrexate (Wigler et al., 1980, Proc. Natl. Acad. Sci USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci USA 78:2072); neo, which confers resistance to the aminoglycoside G-418 (Colberre-Garapin et al., 1981, J. Mol. Biol. 150:1); and hygro, which confers resistance to hygromycin (Santerre et al., 1984, Gene 30:147) genes.

Alternatively, any fusion protein may be readily purified by utilizing an antibody specific for the fusion protein being expressed. For example, a system described by Janknecht et al. allows for the ready purification of non-denatured fusion proteins expressed in human cells lines (Janknecht et al., 1991, Proc. Natl. Acad. Sci. USA 88: 8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers. Fusions at the carboxy terminal of the target gene product are also contemplated.

When used as a component in assay systems such as those described herein, the target gene protein can be labeled, either directly or indirectly, to facilitate detection of a complex formed between the target gene protein and a test substance. Any of a variety of suitable labeling systems can be used including but not limited to radioisotopes such as ¹²⁵I; enzyme labeling systems that generate a detectable colorimetric signal or light when exposed to substrate; and fluorescent labels.

Indirect labeling involves the use of a protein, such as a labeled antibody, which specifically binds to either a target gene product. Such antibodies include but are not limited to polyclonal antibodies, monoclonal antibodies (mAbs), human, humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

Following expression of the target gene protein encoded by the identified target nucleotide sequence, the protein is purified. Protein purification techniques are well known in the art. Proteins encoded and expressed from identified exogenous nucleotide sequences can be partially purified using precipitation techniques, such as precipitation with polyethylene glycol. Alternatively, epitope tagging of the protein can be used to allow simple one step purification of the protein. In addition, chromatographic methods such as ion-exchange chromatography, gel filtration, use of hydroxyapaptite columns, immobilized reactive dyes, chromatofocusing, and use of high-performance liquid chromatography, may also be used to purify the protein. Electrophoretic methods such as one-dimensional gel electrophoresis, high-resolution two-dimensional polyacrylamide electrophoresis, isoelectric focusing, and others are contemplated as purification methods. Also, affinity chromatographic methods, comprising solid phase bound-antibody, ligand presenting columns and other affinity chromatographic matrices are contemplated as purification methods in the present invention.

In addition, the purified target gene products, fragments thereof, or derivatives thereof may be administered to an individual in a pharmaceutically acceptable carrier to induce an immune response against the protein or polypeptide. Preferably, the immune response is a protective immune response which protects the individual. Methods for determining appropriate dosages of the protein (including use of adjuvants) and pharmaceutically acceptable carriers are familiar to those skilled in the art.

5.4.4 Antibodies Specific for Target Gene Products

Described herein are methods for the production of antibodies capable of specifically recognizing epitopes of one or more of the target gene products described above. Such antibodies can include, but are not limited to, polyclonal antibodies, monoclonal antibodies (mAbs), human, humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

For the production of antibodies to a target gene or gene product, various host animals can be immunized by injection with a target gene protein, or a portion thereof. Such host animals can include but are not limited to rabbits, mice, and rats, to name but a few. Various adjuvants can be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Accordingly, the invention provides a method of eliciting an immune response in an animal, comprising introducing into the animal an immunogenic composition comprising an isolated polypeptide, the amino acid sequence of which comprises at least 6 or at least 8 consecutive residues of one of SEQ ID NOs: 7001 through to 7932.

Polyclonal antibodies are heterogeneous populations of antibody molecules derived from the sera of animals immunized with an antigen, such as target gene product, or an antigenic functional derivative thereof. For the production of polyclonal antibodies, host animals such as those described above, can be immunized by injection with differentially expressed or pathway gene product supplemented with adjuvants as also described above. The antibody titer in the immunized animal can be monitored over time by standard techniques, such as with an enzyme linked immunosorbent assay (ELISA) using immobilized polypeptide. If desired, the antibody molecules can be isolated from the animal (e.g, from the blood) and further purified by well-known techniques, such as protein A chromatography to obtain the IgG fraction.

Monoclonal antibodies, which are homogeneous populations of antibodies to a particular antigen, can be obtained by any technique which provides for the production of antibody molecules by continuous cell lines in culture. These include, but are not limited to the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies can be of any immunoglobulin class including IgG, IgM, IgE, IgA, IgD and any subclass thereof. The hybridoma producing the mAb of this invention can be cultivated in vitro or in vivo. Production of high titers of mAbs in vivo makes this the presently preferred method of production.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine mAb and a human immunoglobulin constant region. (See, e.g, Cabilly et al., U.S. Pat. No. 4,816,567; and Boss et al., U.S. Pat. No. 4,816397, which are incorporated herein by reference in their entirety.) Humanized antibodies are antibody molecules from non-human species having one or more complimentarily determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. Nos. 5,625,126; 5,633,425; 5,569,825; 5,661,016; and 5,545,806.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al. (1994) Bio/technology 12:899-903).

Antibody fragments which recognize specific epitopes can be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries can be constructed (Huse et al., 1989, Science-246:1275-1281) to allow rapid and easy identification of monoclonal Fab fragments with the desired specificity.

Antibodies of the present invention may also be described or specified in terms of their binding affinity to a target gene product. Preferred binding affinities include those with a dissociation constant or Kd less than 5×10⁻⁶M, 10⁻⁶M, 5×10⁻⁷M, 10⁻⁷M, 5×10⁻⁸M, 10⁻⁸M, 5×10⁻⁹M, 10⁻⁹M, 5×10⁻¹⁰M, 10⁻¹⁰M, 5×10⁻¹¹M, 10⁻¹¹M, 5×10⁻¹²M, 10⁻¹²M, 5×10⁻¹³M, 10⁻¹³M, 5×10⁻¹⁴M, 10⁻¹⁴M, 5×10⁻¹⁵M, or 10⁻¹⁵M.

Antibodies directed against a target gene product or fragment thereof can be used to detect the a target gene product in order to evaluate the abundance and pattern of expression of the polypeptide under various environmental conditions, in different morphological forms (mycelium, yeast, spores) and stages of an organism's life cycle. Antibodies directed against a target gene product or fragment thereof can be used diagnostically to monitor levels of a target gene product in the tissue of an infected host as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidinibiotin; examples of suitable fluorescent materials include umbelliferone, fluorescein, fluorescein isothiocyanate, rhodamine, dichlorotriazinylamine fluorescein, dansyl chloride or phycoery tirin; an example of a luminescent material includes luminol; examples of bioluminescent materials include luciferase, luciferin, and aequorin, and examples of suitable radioactive material include ¹²⁵I, 131I, ³⁵S or ³H.

Further, antibodies directed against a target gene product or fragment thereof can be used therapeutically to treat an infectious disease by preventing infection, and/or inhibiting growth of the pathogen. Antibodies can also be used to modify a biological activity of a target gene product. Antibodies to gene products related to virulence or pathogenicity can also be used to prevent infection and alleviate one or more symptoms associated with infection by the organism. To facilitate or enhance its therapeutic effect, an antibody (or fragment thereof) may be conjugated to a therapeutic moiety such as a toxin or fungicidal agent. Techniques for conjugating a therapeutic moiety to antibodies are well known, see, e.g., Thorpe et al., “The Preparation And Cytotoxic Properties Of Antibody-Toxin Conjugates”, Immunol. Rev., 62:119-58 (1982).

An antibody with or without a therapeutic moiety conjugated to it can be used as a therapeutic that is administered alone or in combination with chemotherapeutic agents.

5.4.5 Antisense Molecules

The use of antisense molecules as inhibitors of gene expression may be a specific, genetically based therapeutic approach (for a review, see Stein, in Ch. 69, Section 5 “Cancer: Principle and Practice of Oncology”, 4th ed., ed. by DeVita et al., J. B. Lippincott, Philadelphia 1993). The present invention provides the therapeutic or prophylactic use of nucleic acids of at least six nucleotides that are antisense to a target essential or virulence gene or a portion thereof. An “antisense” target nucleic acid as used herein refers to a nucleic acid capable of hybridizing to a portion of a target gene RNA (preferably mRNA) by virtue of some sequence complementarity. The invention further provides pharmaceutical compositions comprising an effective amount of the antisense nucleic acids of the invention in a pharmaceutically acceptable carrier, as described infra.

In another embodiment, the invention is directed to methods for inhibiting the expression of a target gene in an organism of interest, such as C. albicans in vitro or in vivo comprising providing the cell with an effective amount of a composition comprising an antisense nucleic acid of the invention. Multiple antisense polynucleotides hybridizable to different target genes may be used in combinations, sequentially or simultaneously.

In another embodiment, the present invention is directed toward methods for modulating expression of an essential gene which has been identified by the methods described supra, in which an antisense RNA molecule, which inhibits translation of mRNA transcribed from an essential gene, is expressed from a regulatable promoter. In one aspect of this embodiment, the antisense RNA molecule is expressed in a GRACE strain of Candida albicans or another GRACE strain constructed from another diploid pathogenic organism. In other aspects of this embodiment, the antisense RNA molecule is expressed in a wild-type or other non-GRACE strain of Candida albicans or another diploid pathogenic organism, including animal fugal pathogens such as Aspergillus fumigatus, Aspergillus niger, Aspergillus flavis, Candida tropicalis, Candida parapsilopsis, Candida krusei, Cryptococcus neoformans, Coccidioides immitis, Exophalia dermatiditis, Fusarium oxysporum, Histoplasma capsulatum, Phneumocystis carinii, Trichosporon beigelii, Rhizopus arrhizus, Mucor rouxii, Rhizomucor pusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species.

The nucleic acid molecule comprising an antisense nucleotide sequence of the invention may be complementary to a coding and/or noncoding region of a target gene mRNA. The antisense molecules will bind to the complementary target gene mRNA transcripts and reduce or prevent translation. Absolute complementarity, although preferred, is not required. A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. One skilled in the art can ascertain a tolerable degree of mismatch by use of standard procedures to determine the melting point of the hybridized complex.

Nucleic acid molecules that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. See generally, Wagner, R., 1994, Nature 372:333-335.

Nucleic acid molecules comprising nucleotide sequences complementary to the 5′ untranslated region of the mRNA can include the complement of the AUG start codon. Antisense nucleic acid molecules complementary to mRNA coding regions are less efficient inhibitors of translation but could be used in accordance with the invention. Whether designed to hybridize to the 5′-, 3′- or coding region of target gene mRNA, antisense nucleic acids should be at least six nucleotides in length, and are preferably oligonucleotides ranging from 6 to about 50 nucleotides in length. In specific aspects, the oligonucleotide is at least 10 nucleotides, at least 17 nucleotides, at least 25 nucleotides, at least 50 nucleotides, or at least 200 nucleotides.

Regardless of the choice of target gene sequence, it is preferred that in vitro studies are first performed to quantitate the ability of the antisense molecule to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The antisense molecule can be DNA or RNA or chimeric mixtures or derivatives or modified versions thereof, single-stranded or double-stranded. The antisense molecule can be modified at the base moiety, sugar moiety, or phosphate backbone, for example, to improve stability of the molecule, hybridization, etc. The antisense molecule may include other appended groups such as peptides (e.g., for targeting cell receptors in vivo), hybridization-triggered cleavage agents. (See, e.g., Krol et al., 1988, BioTechniques 6:958-976) or intercalating agents. (See, e.g., Zon, 1988, Pharm. Res. 5:539-549). To this end, the antisense molecule may be conjugated to another molecule, e.g., a peptide, hybridization triggered cross-linking agent, transport agent, hybridization-triggered cleavage agent, etc.

The antisense molecule may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxymethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense molecule may also comprise at least one modified sugar moiety selected from the group including but not limited to arabinose, 2-fluoroarabinose, xylulose, and hexose.

In yet another embodiment, the antisense molecule comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense molecule is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641). The oligonucleotide is a 2′-0-methylribonucleotide (Inouye et al., 1987, Nucl. Acids Res. 15:6131-6148), or a chimeric RNA-DNA analogue (Inouye et al., 1987, FEBS Lett. 215:327-330).

Antisense molecules of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

While antisense nucleotides complementary to the coding region of a target gene could be used, those complementary to the transcribed untranslated region are also preferred.

Pharmaceutical compositions of the invention comprising an effective amount of an antisense nucleic acid in a pharmaceutically acceptable carrier, can be administered to a subject infected with the pathogen of interest.

The amount of antisense nucleic acid which will be effective in the treatment of a particular disease caused by the pathogen will depend on the site of the infection or condition, and can be determined by standard techniques. Where possible, it is desirable to determine the antisense cytotoxicity of the pathogen to be treated in vitro, and then in useful animal model systems prior to testing and use in humans.

A number of methods have been developed for delivering antisense DNA or RNA to cells; e.g., antisense molecules can be injected directly into the tissue site in which the pathogens are residing, or modified antisense molecules, designed to target the desired cells (e.g., antisense molecule linked to peptides or antibodies that specifically bind receptors or antigens expressed on the pathogen's cell surface) can be administered systemically. Antisense molecules can be delivered to the desired cell population via a delivery complex. In a specific embodiment, pharmaceutical compositions comprising antisense nucleic acids of the target genes are administered via biopolymers (e.g., poly-β-1→4-N-acetylglucosamine polysaccharide), liposomes, microparticles, or microcapsules. In various embodiments of the invention, it may be useful to use such compositions to achieve sustained release of the antisense nucleic acids. In a specific embodiment, it may be desirable to utilize liposomes targeted via antibodies to specific identifiable pathogen antigens (Leonetti et al., 1990, Proc. Natl. Acad. Sci. U.S.A. 87:2448-2451; Renneisen et al., 1990, J. Biol. Chem. 265:16337-16342).

5.4.6 Ribozyme Molecules

Ribozymes are enzymatic RNA molecules capable of catalyzing the specific cleavage of RNA (For a review see, for example Rossi, J., 1994, Current Biology 4:469-471). The mechanism of ribozyme action involves sequence specific hybridization of the ribozyme molecule to complementary target RNA, followed by a endonucleolytic cleavage. The composition of ribozyme molecules must include one or more sequences complementary to the target gene mRNA, and must include the well known catalytic sequence responsible for mRNA cleavage. For this sequence, see U.S. Pat. No. 5,093,246, which is incorporated by reference herein in its entirety. As such, within the scope of the invention are engineered hammerhead motif ribozyme molecules that specifically and efficiently catalyze endonucleolytic cleavage of RNA sequences encoding target gene proteins.

Ribozyme molecules designed to catalytically cleave specific target gene mRNA transcripts can also be used to prevent translation of target gene mRNA and expression of target genes. While ribozymes that cleave mRNA at site specific recognition sequences can be used to destroy target gene mRNAs, the use of hammerhead ribozymes is preferred. Hammerhead ribozymes cleave mRNAs at locations dictated by flanking regions that form complementary base pairs with the target gene mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591. Preferably the ribozyme is engineered so that the cleavage recognition site is located near the 5′ end of the target gene mRNA; i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO 88/04300 by University Patents Inc.; Been and Cech, 1986, Cell, 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in a target gene.

As in the antisense approach, the ribozymes can be composed of modified oligonucleotides (e.g. for improved stability, targeting, etc.) and should be delivered to cells which express the target gene in vivo. Because ribozymes unlike antisense molecules, are catalytic, a lower intracellular concentration is required for efficiency. Multiple ribozyme molecules directed against different target genes can also be used in combinations, sequentially or simultaneously.

Anti-sense RNA and DNA, ribozyme, and triple helix molecules of the invention can be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules can be generated by in vitro and in vivo transcription of DNA sequences encoding the antisense RNA molecule. Such DNA sequences can be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. These nucleic acid constructs can be administered selectively to the desired cell population via a delivery complex.

Various well-known modifications to the DNA molecules can be introduced as a means of increasing intracellular stability and half-life. Possible modifications include, but are not limited to, the addition of flanking sequences of ribo- or deoxy- nucleotides to the 5′ and/or 3′ ends of the molecule or the use of phosphorothioate or 2′ O-methyl rather than phospho-diesterase linkages within the oligodeoxyribonucleotide backbone.

5.5 Screening Assays

The following assays are designed to identify compounds that bind to target gene products, bind to other cellular proteins that interact with the target gene product, and to compounds that interfere with the interaction of the target gene product with other cellular proteins. Compounds identified via such methods can include compounds which modulate the activity of a polypeptide encoded by a target gene of the invention (that is, increase or decrease its activity, relative to activity observed in the absence of the compound). Alternatively, compounds identified via such methods can include compounds which modulate the expression of the polynucleotide (that is, increase or decrease expression relative to expression levels observed in the absence of the compound), or increase or decrease the stability of the expressed product encoded by that polynucleotide. Compounds, such as compounds identified via the methods of the invention, can be tested using standard assays well known to those of skill in the art for their ability to modulate activity/expression.

Accordingly, the present invention provides a method for identifying an antimycotic compound comprising screening a plurality of compounds to identify a compound that modulates the activity or level of a gene product, said gene product being encoded by a nucleotide sequence selected from the group consisting of SEQ ID NOs: 6001 through to 6932, or a nucleotide sequence that is naturally occurring in Saccharomyces cerevisiae and that is the ortholog of a gene having a nucleotide sequence selected from the group consisting of SEQ ID NOs: 6001 through to 6932.

5.5.1 In Vitro Screening Assays

In vitro systems are designed to identify compounds capable of binding the target gene products of the invention. Compounds identified in this manner are useful, for example, in modulating the activity of wild type and/or mutant target gene products, are useful in elucidating the biological function of target gene products, are utilized in screens for identifying other compounds that disrupt normal target gene product interactions, or are useful themselves for the disruption of such interactions.

The principle of the assays used to identify compounds that bind to the target gene product involves preparing a reaction mixture comprising the target gene product and the test compound under conditions and for a time sufficient to allow the two components to interact and bind, thus forming a complex which is removed and/or detected within the reaction mixture. These assays are conducted in a variety of ways. For example, one method involves anchoring target gene product or the test substance onto a solid phase and detecting target gene product/test compound complexes anchored, via the intermolecular binding reaction, to the solid phase at the end of the reaction. In one embodiment of such a method, the target gene product is anchored onto a solid surface, and the test compound, which is not anchored, is labeled, either directly or indirectly.

In practice, microtiter plates are conveniently utilized as the solid phase. The anchored component is immobilized by non-covalent or covalent attachments. Non-covalent attachment can be accomplished by simply coating the solid surface with a solution of the protein and drying the coated surface. Alternatively, an immobilized antibody, preferably a monoclonal antibody, specific for the protein to be immobilized is used to anchor the protein to the solid surface. The surfaces are prepared in advance and stored.

In order to conduct the assay, the nonimmobilized component is added to the coated surface containing the anchored component. After the reaction is complete, unreacted components are removed (e. g, by washing) under conditions such that any complexes formed will remain immobilized on the solid surface. The detection of complexes anchored on the solid surface is accomplished in a number of ways. Where the previously nonimmobilized component is pre-labeled, the detection of label immobilized on the surface indicates that complexes were formed. Where the previously nonimmobilized component is not pre-labeled, an indirect label is used to detect complexes anchored on the surface; e., using a labeled antibody specific for the previously nonimmobilized component (the antibody, in turn, is directly labeled or indirectly labeled with a labeled anti-Ig antibody).

Alternatively, a reaction is conducted in a liquid phase, the reaction products are separated from unreacted components, and complexes are detected; e.g, using an immobilized antibody specific for the target gene product or for the test compound, to anchor complexes formed in solution, and a second labeled antibody, specific for the other component of the complex to allow detection of anchored complexes.

5.5.1.1 Assays for Proteins that Interact with a Target Gene Product

Any method suitable for detecting protein-protein interactions can be employed for identifying novel target protein-cellular or extracellular protein interactions.

The target gene products of the invention interact, in vivo, with one or more cellular or extracellular macromolecules, such as proteins. Such macromolecules include, but are not limited to, nucleic acid molecules and proteins identified via methods such as those described above. For purposes of this discussion, such cellular and extracellular macromolecules are referred to herein as “binding partners.” Compounds that disrupt such interactions can be useful in regulating the activity of the target gene protein, especially mutant target gene proteins. Such compounds include, but are not limited to molecules such as antibodies, peptides, and the like, as described.

The basic principle of the assay systems used to identify compounds that interfere with the interaction between the target gene product and its cellular or extracellular binding partner or partners involves preparing a reaction mixture containing the target gene product and the binding partner under conditions and for a time sufficient to allow the two to interact and bind, thus forming a complex. In order to test a compound for inhibitory activity, the reaction mixture is prepared in the presence and absence of the test compound. The test compound is initially included in the reaction mixture, or added at a time subsequent to the addition of target gene product and its cellular or extracellular binding partner. Control reaction mixtures are incubated without the test compound. The formation of complexes between the target gene protein and the cellular or extracellular binding partner is then detected. The formation of a complex in the control reaction, but not in the reaction mixture containing the test compound, indicates that the compound interferes with the interaction of the target gene protein and the interactive binding partner. Additionally, complex formation within reaction mixtures containing the test compound and normal target gene protein can also be compared to complex formation within reaction mixtures containing the test compound and a mutant target gene protein. This comparison can be important in those cases wherein it is desirable to identify compounds that disrupt intermolecular interactions involving mutant but not normal target gene proteins.

The assay for compounds that interfere with the interaction of the target gene products and binding partners is conducted in either a heterogeneous or a homogeneous format. Heterogeneous assays involve anchoring either the target gene product or the binding partner onto a solid phase and detecting complexes anchored on the solid phase at the end of the reaction. In homogeneous assays, the entire reaction is carried out in a liquid phase. In either approach, the order of addition of reactants is varied to obtain different information about the compounds being tested. For example, test compounds that interfere with the interaction between the target gene products and the binding partners, e.g, by competition, are identified by conducting the reaction in the presence of the test substance; i.e., by adding the test substance to the reaction mixture prior to or simultaneously with the target gene protein and an interacting cellular or extracellular binding partner. Alternatively, test compounds that disrupt preformed complexes, e.g. compounds with higher binding constants that displace one of the components from the complex, are tested by adding the test compound to the reaction mixture after complexes have been formed. The various formats are described briefly below.

In a heterogeneous assay system, either the target gene protein or the interactive cellular or extracellular binding partner, is anchored onto a solid surface, while the non-anchored species is labeled, either directly or indirectly. In practice, microtiter plates are conveniently utilized. The anchored species is immobilized either by non-covalent or covalent attachment. Non-covalent attachment is accomplished simply by coating the solid surface with a solution of the target gene product or binding partner and drying the coated surface. Alternatively, an immobilized antibody specific for the species to be anchored is used to anchor the species to the solid surface. The surfaces can be prepared in advance and stored.

In order to conduct the assay, the partner of the immobilized species is exposed to the coated surface with or without the test compound. After the reaction is complete, unreacted components are removed (e.g, by washing) and any complexes formed will remain immobilized on the solid surface. The detection of complexes anchored on the solid surface is accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g, using a labeled antibody specific for the initially non-immobilized species (the antibody, in turn, is directly labeled or indirectly labeled with a labeled anti-Ig antibody). Depending upon the order of addition of reaction components, test compounds which inhibit complex formation or which disrupt preformed complexes are detected.

Alternatively, the reaction is conducted in a liquid phase in the presence or absence of the test compound, the reaction products separated from unreacted components, and complexes detected; e.g., using an immobilized antibody specific for one of the binding components to anchor any complexes formed in solution, and a second, labeled antibody specific for the other partner to detect anchored complexes. Again, depending upon the order of addition of reactants to the liquid phase, test compounds which inhibit complex or which disrupt preformed complexes are identified.

In an alternate embodiment of the invention, a homogeneous assay can be used. In this approach, a preformed complex of the target gene protein and the interacting cellular or extracellular binding partner is prepared in which either the target gene product or its binding partner is labeled, but the signal generated by the label is quenched due to complex formation (see, e.g, U.S. Pat. No. 4,109,496 by Rubenstein which utilizes this approach for immunoassays). The addition of a test substance that competes with and displaces one of the species from the preformed complex results in the generation of a signal above background. In this way, test substances which disrupt target gene protein/cellular or extracellular binding partner interaction are identified.

In a particular embodiment, the target gene product is prepared for immobilization using recombinant DNA techniques described above. For example, the target gene coding region is fused to a glutathione-S-transferase (GST) gene using a fusion vector, such as pGEX-5X-1, in such a manner that its binding activity is maintained in the resulting fusion protein. The interactive cellular or extracellular binding partner is purified and used to raise a monoclonal antibody, using methods routinely practiced in the art and as described above. This antibody is labeled with the radioactive isotope ¹²⁵I, for example, by methods routinely practiced in the art. In a heterogeneous assay, e.g., the GST-target gene fusion protein is anchored to glutathione-agarose beads. The interactive cellular or extracellular binding partner is then added in the presence or absence of the test compound in a manner that allows interaction and binding to occur. At the end of the reaction period, unbound material can be washed away, and the labeled monoclonal antibody is added to the system and allowed to bind to the complexed components. The interaction between the target gene protein and the interactive cellular or extracellular binding partner is detected by measuring the amount of radioactivity that remains associated with the glutathione-agarose beads. A successful inhibition of the interaction by the test compound results in a decrease in measured radioactivity.

Alternatively, the GST-target gene fusion protein and the interactive cellular or extracellular binding partner are mixed together in liquid in the absence of the solid glutathione-agarose beads. The test compound is added either during or after the species are allowed to interact. This mixture is added to the glutathione-agarose beads and unbound material is washed away. Again the extent of inhibition of the target gene product/binding partner interaction is detected by adding the labeled antibody and measuring the radioactivity associated with the beads.

In another embodiment of the invention, these same techniques are employed using peptide fragments that correspond to the binding domains of the target gene product and/or the interactive cellular or extracellular binding partner (in cases where the binding partner is a protein), in place of one or both of the full length proteins. Any number of methods routinely practiced in the art are used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex are then selected. Sequence analysis of the genes encoding the respective proteins reveals the mutations that correspond to the region of the protein involved in interactive binding. Alternatively, one protein is anchored to a solid surface using methods described above, and allowed to interact with and bind to its labeled binding partner, which has been treated with a proteolytic enzyme, such as trypsin. After washing, a short, labeled peptide comprising the binding domain remains associated with the solid material, and can be isolated and identified by amino acid sequencing. Also, once the gene coding for the cellular or extracellular binding partner is obtained, short gene segments are engineered to express peptide fragments of the protein, which are tested for binding activity and purified or synthesized.

For example, and not by way of limitation, a target gene product is anchored to a solid material as described, above, by making a GST-target gene fusion protein and allowing it to bind to glutathione agarose beads. The interactive cellular or extracellular binding partner is labeled with a radioactive isotope, such as ³⁵S, and cleaved with a proteolytic enzyme such as trypsin. Cleavage products are added to the anchored GST-target gene fusion protein and allowed to bind. After washing away unbound peptides, labeled bound material, representing the cellular or extracellular binding partner binding domain, is eluted, purified, and analyzed for amino acid sequence by well known methods. Peptides so identified are produced synthetically or fused to appropriate facilitative proteins using well known recombinant DNA technology.

5.5.1.2 Screening a Combinatorial Chemical Library

In one embodiment of the present invention, the proteins encoded by the fungal genes identified using the methods of the present invention are isolated and expressed. These recombinant proteins are then used as targets in assays to screen libraries of compounds for potential drug candidates. The generation of chemical libraries is well known in the art. For example, combinatorial chemistry is used to generate a library of compounds to be screened in the assays described herein. A combinatorial chemical library is a collection of diverse chemical compounds generated by either chemical synthesis or biological synthesis by combining a number of chemical “building block” reagents. For example, a linear combinatorial chemical library such as a polypeptide library is formed by combining amino acids in every possible combination to yield peptides of a given length. Millions of chemical compounds theoretically can be synthesized through such combinatorial mixings of chemical building blocks. For example, one commentator observed that the systematic, combinatorial mixing of 100 interchangeable chemical building blocks results in the theoretical synthesis of 100 million tetrameric compounds or 10 billion pentameric compounds. (Gallop et al., “Applications of Combinatorial Technologies to Drug Discovery, Background and Peptide Combinatorial Libraries,” Journal of Medicinal Chemistry, Vol. 37, No. 9, 1233-1250 (1994). Other chemical libraries known to those in the art may also be used, including natural product libraries.

Once generated, combinatorial libraries are screened for compounds that possess desirable biological properties. For example, compounds which may be useful as drugs or to develop drugs would likely have the ability to bind to the target protein identified, expressed and purified as discussed above. Further, if the identified target protein is an enzyme, candidate compounds would likely interfere with the enzymatic properties of the target protein. For example, the enzymatic function of a target protein may be to serve as a protease, nuclease, phosphatase, dehydrogenase, transporter protein, transcriptional enzyme, replication component, and any other type of enzyme known or unknown. Thus, the present invention contemplates using the protein products described above to screen combinatorial chemical libraries.

In some embodiments of the present invention, the biochemical activity of the protein, as well as the chemical structure of a substrate on which the protein acts is known. In other embodiments of the present invention, the biochemical activity of the target protein is unknown and the target protein has no known substrates.

In some embodiments of the present invention, libraries of compounds are screened to identify compounds that function as inhibitors of the target gene product. First, a library of small molecules is generated using methods of combinatorial library formation well known in the art. U.S. Pat. Nos. 5,463,564 and 5,574, 656, to Agrafiotis, et al., entitled “System and Method of Automatically Generating Chemical Compounds with Desired Properties,” the disclosures of which are incorporated herein by reference in their entireties, are two such teachings. Then the library compounds are screened to identify those compounds that possess desired structural and functional properties. U.S. Pat. No. 5,684,711, the disclosure of which is incorporated herein by reference in its entirety, also discusses a method for screening libraries.

To illustrate the screening process, the target gene product, an enzyme, and chemical compounds of the library are combined and permitted to interact with one another. A labeled substrate is added to the incubation. The label on the substrate is such that a detectable signal is emitted from metabolized substrate molecules. The emission of this signal permits one to measure the effect of the combinatorial library compounds on the enzymatic activity of target enzymes by comparing it to the signal emitted in the absence of combinatorial library compounds. The characteristics of each library compound are encoded so that compounds demonstrating activity against the enzyme can be analyzed and features common to the various compounds identified can be isolated and combined into future iterations of libraries.

Once a library of compounds is screened, subsequent libraries are generated using those chemical building blocks that possess the features shown in the first round of screen to have activity against the target enzyme. Using this method, subsequent iterations of candidate compounds will possess more and more of those structural and functional features required to inhibit the function of the target enzyme, until a group of enzyme inhibitors with high specificity for the enzyme can be found. These compounds can then be further tested for their safety and efficacy as antibiotics for use in mammals.

It will be readily appreciated that this particular screening methodology is exemplary only. Other methods are well known to those skilled in the art. For example, a wide variety of screening techniques are known for a large number of naturally-occurring targets when the biochemical function of the target protein is known. For example, some techniques involve the generation and use of small peptides to probe and analyze target proteins both biochemically and genetically in order to identify and develop drug leads. Such techniques include the methods described in PCT publications No. WO9935494, WO9819162, WO9954728, the disclosures of which are incorporated herein by reference in their entireties.

Similar methods may be used to identify compounds which inhibit the activity of proteins from organisms other than Candida albicans which are homologous to the Candida albicans target proteins described herein. For example, the proteins may be from animal fugal pathogens such as Aspergillus fumigatus, Aspergillus niger, Aspergillus flavis, Candida tropicalis, Candida parapsilopsis, Candida krusei, Cryptococcus neoformans, Coccidioides immitis, Exophalia dermatiditis, Fusarium oxysporum, Histoplasma capsulatum, Phneumocystis carinii, Trichosporon beigelii, Rhizopus arrhizus, Mucor rouxii, Rhizomucor pusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species. In some embodiments, the proteins are from an organism other than Saccharomyces cerevisiae.

5.5.1.3 In vitro Enzyme Assays

In one embodiment, GRACE methods and strains can be used to develop in vitro assays for biochemical activities that are shown to be essential to cell viability. A number of essential genes identified by the GRACE conditional expression methodologies display statistically significant similarity to biochemically characterized gene products from other organisms. For example, based on amino acid sequence similarity, a number of essential and fungal specific genes listed in Table II are predicted to possess the following biochemical activities:

| | |
|---|---|
| CaRHO1 | GTPase involved in (1,3)-β-glucan synthesis |
| | and polarity |
| CaYHR118c (ORC6) | Origin of replication complex subunit |
| CaYPL128c (TBP1) | Telomere binding protein |
| CaYNL256w | Dihydropteroate synthase |
| CaYKL004w (AUR1) | Phosphatidylinositol: ceramide phosphoinositol |
| | transferase |
| CaYJL090c (DPB11) | DNA polB subunit |
| CaYOL149w (DCP1) | mRNA decapping enzyme |
| CaYNL151c (RPC31) | RNA polIII subunit |
| CaYOR148c (SPP2) | RNA splicing |
| CaYER026c (CHO1) | Phosphatidylserine synthase |

Therefore, a number of well characterized standard in vitro biochemical assays (e.g, DNA binding, RNA processing, GTP binding and hydrolysis, and phosphorylation) are readily adapted for these validated drug targets. For example the validated target, CaRHO1, is used within a in vitro-based drug screen by adapting standard GTPase assays developed for a wide range of such proteins. Alternatively, novel assays are developed using biochemical information pertaining to validated drug targets within our GRACE strain collection. Any assays known in the art for enzymes with similar biochemical activities (e.g., mechanism of action, class of substrate) are adapted for screening for inhibitors of the enzymes encoded by these essential C. albicans genes.

For example, a number of features make the C. albicans gene, CaTBF1, a candidate for in vitro assay development. CaTBF1 shares significant homology to its S. cerevisiae counterpart, TBF1, a telomere binding factor. In addition, the DNA sequence CaTBF1p recognizes is known and is relatively short (Koering et al., Nucleic Acid Res. 28:2519-2526, which is incorporated herein by reference in its entirety), enabling inexpensive synthesis of oligonucleotides corresponding to this element. Moreover since this assay only requires the target protein and a DNA fragment containing the nucleotide sequence it recognizes, only purification of CaTBF1p protein is necessary in order to develop an in vitro binding assay. One preferred embodiment of this in vitro assay involves crosslinking the DNA element to the bottom of a well, incubation of radiolabeled CaTBF1p to facilitate protein-DNA binding, a series of washes to remove unbound material, and determination of the percentage of bound radiolabeled CaTBF1p. Alternatively, purified CaTBF1p is attached to the well and radiolabeled oligonucleotides added. Drug screening, including the use of high throughput screening technique, is performed by searching for compounds that inhibit the protein-DNA binding measured in this assay.

Similarly, a second validated drug target, CaORC6, is used in this type of assay since its S. cerevisiae homolog, ORC6, directly binds a DNA element within the origin of replication of yeast chromosomes (Mizushima et al., 2000, Genes & Development 14:1631-1641, which is incorporated herein by reference in its entirety). Biochemical purification of any of these targets could be achieved, for example, by PCR-based construction of C. albicans heterozygous strains in which the gene encoding the CaORC6 protein has been modified to include a carboxy-terminal hexahistidine tag enabling purification of the chimeric protein using standard Ni⁺² affinity column chromatography techniques.

For other targets like CaDPB11, a homolog of which in S. cerevisiae encode proteins that physically associate with Sld2p (Kamimura et al., 1998, Cell Biol. 18:6102-6109, which is incorporated herein by reference in its entirety), in vitro assays similar to those described above are developed. In addition, two-hybrid assays based on known physical interactions are developed for any validated targets within the GRACE strain collection.

The present invention also provides cell extracts useful in establishing in vitro assays for suitable biochemical targets. For example, in an embodiment of the present invention, GRACE-derived C. albicans strains are grown either under constitutive expression conditions or transcription repression conditions to either overproduce or deplete a particular gene product. Cellular extracts resulting from strains incubated under these two conditions are compared with extracts prepared from identically-grown wild type strains. These extracts are then used for the rapid evaluation of targets using existing in vitro assays or new assays directed toward novel gene products, without having to purify the gene product. Such a whole cell extract approach to in vitro assay development is typically necessary for targets involved in cell wall biosynthetic pathways (e.g. (1,3)-β-glucan synthesis or chitin synthesis) which involve multiple gene products that transit the secretory pathway before receiving essential post-translational modifications required for their functional activity. GRACE-derived strains for conditional expression of target genes involved in these, or other cell wall pathways (e.g. (1,6)-β-glucan synthesis) enable in vitro assays to be performed directly in C. albicans.

5.5.2 Cell-Based Screening Assays

In various embodiments, the essential genes identified by the methods of the invention can be used in cell-based screening assays. Generally, the target essential gene in a cell can be engineered to be overexpressed or underexpressed constitutively or inducible. Given that the identity of an essential gene is known, the construction of such cells can be accomplished by methods well known in the art. The GRACE strains of the invention is a non-limiting example of the type of genetically engineered cells that can be used in the cell-based screening assays of the invention.

Current cell-based assays used to identify or to characterize compounds for drug discovery and development frequently depend on detecting the ability of a test compound to modulate the activity of a target molecule located within a cell or located on the surface of a cell. Most often such target molecules are proteins such as enzymes, receptors and the like. However, target molecules also include other molecules such as DNAs, lipids, carbohydrates and RNAs including messenger RNAs, ribosomal RNAs, tRNAs and the like. A number of highly sensitive cell-based assay methods are available to those of skill in the art to detect binding and interaction of test compounds with specific target molecules. However, these methods are generally not highly effective when the test compound binds to or otherwise interacts with its target molecule with moderate or low affinity. In addition, the target molecule may not be readily accessible to a test compound in solution, such as when the target molecule is located inside the cell or within a cellular compartment such as the periplasm of a bacterial cell. Thus, current cell-based assay methods are limited in that they are not effective in identifying or characterizing compounds that interact with their targets with moderate to low affinity or compounds that interact with targets that are not readily accessible.

The cell-based assay methods of the present invention have substantial advantages over current cell-based assays. These advantages derive from the use of sensitized cells in which the level or activity of at least one gene product required for fungal proliferation, virulence, or pathogenicity (the target molecule) has been reduced, and preferably specifically reduced to the point where the presence or absence of its function becomes a rate-determining step for fungal growth, survival, proliferation, virulence, or pathogenicity. Such sensitized cells become much more sensitive to compounds that are active against the affected target molecule. For example, sensitized cells are obtained by growing a GRACE strain in the presence of a concentration of inducer or repressor which provides a level of a gene product required for fungal growth, survival, proliferation, virulence, or pathogenicity such that the presence or absence of its function becomes a rate-determining step for fungal growth, survival, proliferation, virulence, or pathogenicity. Thus, cell-based assays of the present invention are capable of detecting compounds exhibiting low or moderate potency against the target molecule of interest because such compounds are substantially more potent on sensitized cells than on non-sensitized cells. The effect may be such that a test compound may be two to several times more potent, at least 10 times more potent, at least 20 times more potent, at least 50 times more potent, at least 100 times more potent, at least 1000 times more potent, or even more than 1000 times more potent when tested on the sensitized cells as compared to the non-sensitized cells.

Due in part to the increased appearance of antibiotic resistance in pathogenic microorganisms and to the significant side-effects associated with some currently used antibiotics, novel antibiotics acting at new targets are highly sought after in the art. Yet, another limitation in the current art related to cell-based assays is the problem of repeatedly identifying hits against the same kinds of target molecules in the same limited set of biological pathways. This may occur when compounds acting at such new targets are discarded, ignored or fail to be detected because compounds acting at the “old” targets are encountered more frequently and are more potent than compounds acting at the new targets. As a result, the majority of antibiotics in use currently interact with a relatively small number of target molecules within an even more limited set of biological pathways.

The use of sensitized cells of the current invention provides a solution to the above problems in two ways. First, desired compounds acting at a target of interest, whether a new target or a previously known but poorly exploited target, can now be detected above the “noise” of compounds acting at the “old” targets due to the specific and substantial increase in potency of such desired compounds when tested on the sensitized cells of the current invention. Second, the methods used to sensitize cells to compounds acting at a target of interest may also sensitize these cells to compounds acting at other target molecules within the same biological pathway. For example, expression of a gene encoding a ribosomal protein at a level such that the function of the ribosomal protein becomes rate limiting for fungal growth, survival, proliferation, virulence, or pathogenicity is expected to sensitize the cell to compounds acting at that ribosomal protein to compounds acting at any of the ribosomal components (proteins or rRNA) or even to compounds acting at any target which is part of the protein synthesis pathway. Thus an important advantage of the present invention is the ability to reveal new targets and pathways that were previously not readily accessible to drug discovery methods.

Sensitized cells of the present invention are prepared by reducing the activity or level of a target molecule. The target molecule may be a gene product, such as an RNA or polypeptide produced from the nucleic acids required for fungal growth, survival, proliferation, virulence, or pathogenicity described herein. In addition, the target may be an RNA or polypeptide in the same biological pathway as the nucleic acids required for fungal growth, survival, proliferation, virulence, or pathogenicity as described herein. Such biological pathways include, but are not limited to, enzymatic, biochemical and metabolic pathways as well as pathways involved in the production of cellular structures such as the cell membrane.

Current methods employed in the arts of medicinal and combinatorial chemistries are able to make use of structure-activity relationship information derived from testing compounds in various biological assays including direct binding assays and cell-based assays. Occasionally compounds are directly identified in such assays that are sufficiently potent to be developed as drugs. More often, initial hit compounds exhibit moderate or low potency. Once a hit compound is identified with low or moderate potency, directed libraries of compounds are synthesized and tested in order to identify more potent leads. Generally these directed libraries are combinatorial chemical libraries consisting of compounds with structures related to the hit compound but containing systematic variations including additions, subtractions and substitutions of various structural features. When tested for activity against the target molecule, structural features are identified that either alone or in combination with other features enhance or reduce activity. This information is used to design subsequent directed libraries containing compounds with enhanced activity against the target molecule. After one or several iterations of this process, compounds with substantially increased activity against the target molecule are identified and may be further developed as drugs. This process is facilitated by use of the sensitized cells of the present invention since compounds acting at the selected targets exhibit increased potency in such cell-based assays, thus; more compounds can now be characterized providing more useful information than would be obtained otherwise.

Thus, it is now possible using cell-based assays of the present invention to identify or characterize compounds that previously would not have been readily identified or characterized including compounds that act at targets that previously were not readily exploited using cell-based assays. The process of evolving potent drug leads from initial hit compounds is also substantially improved by the cell-based assays of the present invention because, for the same number of test compounds, more structure-function relationship information is likely to be revealed.

The method of sensitizing a cell entails selecting a suitable gene. A suitable gene is one whose expression is required for the growth, survival, proliferation, virulence, or pathogenicity of the cell to be sensitized. The next step is to obtain a cell in which the level or activity of the target can be reduced to a level where it is rate limiting for growth, survival, proliferation, virulence or pathogenicity. For example, the cell may be a GRACE strain in which the selected gene is under the control of a regulatable promoter. The amount of RNA transcribed from the selected gene is limited by varying the concentration of an inducer or repressor which acts on the regulatable promoter, thereby varying the activity of the promoter driving transcription of the RNA. Thus, cells are sensitized by exposing them to an inducer or repressor concentration that results in an RNA level such that the function of the selected gene product becomes rate limiting for fungal growth, survival, proliferation, virulence, or pathogenicity.

In one embodiment of the cell-based assays, GRACE strains, in which the sequences required for fungal growth, survival, proliferation, virulence, or pathogenicity of Candida albicans described herein are under the control of a regulatable promoter, are grown in the presence of a concentration of inducer or repressor which causes the function of the gene products encoded by these sequences to be rate limiting for fungal growth, survival, proliferation, virulence, or pathogenicity. To achieve that goal, a growth inhibition dose curve of inducer or repressor is calculated by plotting various doses of inducer or repressor against the corresponding growth inhibition caused by the limited levels of the gene product required for fungal proliferation. From this dose-response curve, conditions providing various growth rates, from 1 to 100% as compared to inducer or repressor-free growth, can be determined. For example, if the regulatable promoter is repressed by tetracycline, the GRACE strain may be grown in the presence of varying levels of tetracycline. Similarly, inducible promoters may be used. In this case, the GRACE strains are grown in the presence of varying concentrations of inducer. For example, the highest concentration of the inducer or repressor that does not reduce the growth rate significantly can be estimated from the dose-response curve. Cellular proliferation can be monitored by growth medium turbidity via OD measurements. In another example, the concentration of inducer or repressor that reduces growth by 25% can be predicted from the dose-response curve. In still another example, a concentration of inducer or repressor that reduces growth by 50% can be calculated from the dose-response curve. Additional parameters such as colony forming units (cfu) are also used to measure cellular growth, survival and/or viability.

In another embodiment of the present invention, an individual haploid strain may similarly be used as the basis for detection of an antifungal or therapeutic agent. In this embodiment, the test organism (e.g. Aspergillus fumigatus, Cryptococcus neoformans, Magnaporthe grisea or any other haploid organisms represented in Table I) is a strain constructed by modifying the single allele of the target gene in one step by recombination with a promoter replacement fragment comprising a heterologous regulatable promoter, such that the expression of the gene is conditionally regulated by the heterologous promoter. Like individual diploid GRACE strains, sensitized haploid cells may similarly be used in whole cell-based assay methods to identify compounds displaying a preferential activity against the affected target.

In various embodiments, the modified strain is grown under a first set of conditions where the heterologous promoter is expressed at a relatively low level (i.e. partially repressed) and the extent of growth determined. This experiment is repeated in the presence of a test compound and a second measurement of growth obtained. The extent of growth in the presence and in the absence of the test compound are then compared to provide a first indicator value. Two further experiments are performed, using non-repressing growth conditions where the target gene is expressed at substantially higher levels than in the first set of conditions. The extent of growth is determined in the presence and absence of the test compound under the second set of conditions to obtain a second indicator value. The first and second indicator values are then compared. If the indicator values are essentially the same, the data suggest that the test compound does not inhibit the test target. However, if the two indicator values are substantially different, the data indicates that the level of expression of the target gene product may determine the degree of inhibition by the test compound and, therefore, it is likely that the gene product is the target of that test compound. Whole-cell assays comprising collections or subsets of multiple sensitized strains may also be screened, for example, in a series of 96-well, 384-well, or even 1586-well microtiter plates, with each well containing individual strains sensitized to identify compounds displaying a preferential activity against each affected target comprising a target set or subset selected from, but not limited to the group consisting of fungal-specific, pathogen-specific, desired biochemical-function, human-homolog, cellular localization, and signal transduction cascade target sets.

Cells to be assayed are exposed to the above-determined concentrations of inducer or repressor. The presence of the inducer or repressor at this sub-lethal concentration reduces the amount of the proliferation-required gene product to the lowest amount in the cell that will support growth. Cells grown in the presence of this concentration of inducer or repressor are therefore specifically more sensitive to inhibitors of the proliferation-required protein or RNA of interest as well as to inhibitors of proteins or RNAs in the same biological pathway as the proliferation-required protein or RNA of interest but not specifically more sensitive to inhibitors of unrelated proteins or RNAs.

Cells pretreated with sub-inhibitory concentrations of inducer or repressor, which therefore contain a reduced amount of proliferation-required target gene product, are used to screen for compounds that reduce cell growth. The sub-lethal concentration of inducer or repressor may be any concentration consistent with the intended use of the assay to identify candidate compounds to which the cells are more sensitive than are control cells in which this gene product is not rate-limiting. For example, the sub-lethal concentration of the inducer or repressor may be such that growth inhibition is at least about 5%, at least about 8%, at least about 10%, at least about 20%, at least about 30%, at least about 40%, at least about 50%, at least about 60% at least about 75%, at least 80%, at least 90%, at least 95% or more than 95%. Cells which are pre-sensitized using the preceding method are more sensitive to inhibitors of the target protein because these cells contain less target protein to inhibit than wild-type cells.

It will be appreciated that similar methods may be used to identify compounds which inhibit virulence or pathogenicity. In such methods, the virulence or pathogenicity of cells exposed to the candidate compound which express rate limiting levels of a gene product involved in virulence or pathogenicity is compared to the virulence or pathogenicity of cells exposed to the candidate compound in which the levels of the gene product are not rate limiting. Virulence or pathogenicity may be measured using the techniques described herein.

In another embodiment of the cell-based assays of the present invention, the level or activity of a gene product required for fungal growth, survival, proliferation, virulence, or pathogenicity is reduced using a mutation, such as a temperature sensitive mutation, in the sequence required for fungal growth, survival, proliferation, virulence, or pathogenicity and an inducer or repressor level which, in conjunction with the temperature sensitive mutation, provides levels of the gene product required for fungal growth, survival, proliferation, virulence, or pathogenicity which are rate limiting for proliferation. Growing the cells at an intermediate temperature between the permissive and restrictive temperatures of the temperature sensitive mutant where the mutation is in a gene required for fungal growth, survival, proliferation, virulence, or pathogenicity produces cells with reduced activity of the gene product required for growth, survival, proliferation, virulence, or pathogenicity. The concentration of inducer or repressor is chosen so as to further reduces the activity of the gene product required for fungal growth, survival, proliferation, virulence, or pathogenicity. Drugs that may not have been found using either the temperature sensitive mutation or the inducer or repressor alone may be identified by determining whether cells in which expression of the nucleic acid encoding the proliferation-required gene product has been reduced and which are grown at a temperature between the permissive temperature and the restrictive temperature are substantially more sensitive to a test compound than cells in which expression of the gene product required for fungal growth, survival, proliferation, virulence, or pathogenicity has not been reduced and which are grown at a permissive temperature. Also drugs found previously from either the use of the inducer or repressor alone or the temperature sensitive mutation alone may have a different sensitivity profile when used in cells combining the two approaches, and that sensitivity profile may indicate a more specific action of the drug in inhibiting one or more activities of the gene product.

Temperature sensitive mutations may be located at different sites within a gene and may lie within different domains of the protein. For example, the dnaB gene of Escherichia coli encodes the replication fork DNA helicase. DnaB has several domains, including domains for oligomerization, ATP hydrolysis, DNA binding, interaction with primase, interaction with DnaC, and interaction with DnaA. Temperature sensitive mutations in different domains of DnaB confer different phenotypes at the restrictive temperature, which include either an abrupt stop or a slow stop in DNA replication either with or without DNA breakdown (Wechsler, J. A. and Gross, J. D. 1971 Escherichia coli mutants temperature-sensitive for DNA synthesis. Mol. Gen. Genetics 113:273-284) and termination of growth or cell death. Thus, temperature sensitive mutations in different domains of the protein may be used in conjunction with GRACE strains in which expression of the protein is under the control of a regulatable promoter.

It will be appreciated that the above method may be performed with any mutation which reduces but does not eliminate the activity or level of the gene product which is required for fungal growth, survival, proliferation, virulence, or pathogenicity.

When screening for antimicrobial agents against a gene product required for fungal growth, survival, proliferation, virulence, or pathogenicity, growth inhibition, virulence or pathogenicity of cells containing a limiting amount of that gene product can be assayed. Growth inhibition can be measured by directly comparing the amount of growth, measured by the optical density of the culture relative to uninoculated growth medium, between an experimental sample and a control sample. Alternative methods for assaying cell proliferation include measuring green fluorescent protein (GFP) reporter construct emissions, various enzymatic activity assays, and other methods well known in the art. Virulence and pathogenicity may be measured using the techniques described herein.

It will be appreciated that the above method may be performed in solid phase, liquid phase, a combination of the two preceding media, or in vivo. For example, cells grown on nutrient agar containing the inducer or repressor which acts on the regulatable promoter used to express the proliferation required gene product may be exposed to compounds spotted onto the agar surface. A compound's effect may be judged from the diameter of the resulting killing zone, the area around the compound application point in which cells do not grow. Multiple compounds may be transferred to agar plates and simultaneously tested using automated and semi-automated equipment including but not restricted to multi-channel pipettes (for example the Beckman Multimek) and multi-channel spotters (for example the Genomic Solutions Flexys). In this way multiple plates and thousands to millions of compounds may be tested per day.

The compounds are also tested entirely in liquid phase using microtiter plates as described below. Liquid phase screening may be performed in microtiter plates containing 96, 384, 1536 or more wells per microtiter plate to screen multiple plates and thousands to millions of compounds per day. Automated and semi-automated equipment are used for addition of reagents (for example cells and compounds) and for determination of cell density.

The compounds are also tested in vivo using the methods described herein.

It will be appreciated that each of the above cell-based assays may be used to identify compounds which inhibit the activity of gene products from organisms other than Candida albicans which are homologous to the Candida albicans gene products described herein. For example, the target gene products may be from animal fugal pathogens such as Aspergillus fumigatus, Aspergillus niger, Aspergillus flavis, Candida tropicalis, Candida parapsilopsis, Candida krusei, Cryptococcus neoformans, Coccidioides immitis, Exophalia dermatiditis, Fusarium oxysporum, Histoplasma capsulatum, Phneumocystis carinii, Trichosporon beigelii, Rhizopus arrhizus, Mucor rouxii, Rhizomucor pusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species. In some embodiments, the gene products are from an organism other than Saccharomyces cerevisiae.

5.5.2.1 Cell-Based Assays Using GRACE Strains

GRACE strains in which one allele of a gene required for fungal growth, survival, proliferation, virulence, or pathogenicity is inactivated while the other allele is under the control of a regulatable promoter are constructed using the methods described herein. For the purposes of the present example, the regulatable promoter may be the tetracycline regulated promoter described herein, but it will be appreciated that any regulatable promoter may be used.

In one embodiment of the present invention, an individual GRACE strain is used as the basis for detection of a therapeutic agent active against a diploid pathogenic fungal cell. In this embodiment, the test organism is a GRACE strain having a modified allelic gene pair, where the first allele of the gene has been inactivated by the insertion of, or replacement by, a nucleotide sequence encoding an expressible, dominant selectable marker and the second allele has been modified, by recombination, to place the second allele under the controlled expression of a heterologous promoter. This test GRACE strain is then grown under a first set of conditions where the heterologous promoter is expressed at a relatively low level (“repressing”) and the extent of growth determined. This measurement may be carried out using any appropriate standard known to those skilled in the art including optical density, wet weight of pelleted cells, total cell count, viable count, DNA content, and the like. This experiment is repeated in the presence of a test compound and a second measurement of growth obtained. The extent of growth in the presence and in the absence of the test compound, which can conveniently be expressed in terms of indicator values, are then compared. A dissimilarity in the extent of growth or indicator values provides an indication that the test compound may interact with the target essential gene product.

To gain more information, two further experiments are performed, using a second set of “non-repressing” growth conditions where the second allele, under the control of the heterologous promoter, is expressed at a level substantially higher than in the first set of conditions described above. The extent of growth or indicator values is determined in the presence and absence of the test compound under this second set of conditions. The extent of growth or indicator values in the presence and in the absence of the test compound are then compared. A dissimilarity in the extent of growth or indicator values provides an indication that may interact with the target essential gene product.

Furthermore, the extent of growth in the first and in the second set of growth conditions can also be compared. If the extent of growth is essentially the same, the data suggest that the test compound does not inhibit the gene product encoded by the modified allelic gene pair carried by the GRACE strain tested. However, if the extent of growth are substantially different, the data indicate that the level of expression of the subject gene product may determine the degree of inhibition by the test compound and, therefore, it is likely that the subject gene product is the target of that test compound.

Although each GRACE strain can be tested individually, it will be more efficient to screen entire sets or subsets of a GRACE strain collection at one time. Therefore in one aspect of this invention, arrays may be established, for example in a series of 96-well microtiter plates, with each well containing a single GRACE strain. In one representative, but not limiting approach, four microtiter plates are used, comprising two pairs where the growth medium in one pair supports greater expression of the heterologous promoter controlling the remaining active allele in each strain, than the medium in the other pair of plates. One member of each pair is supplemented with a compound to be tested and measurements of growth of each GRACE strain is determined using standard procedures to provide indicator values for each isolate tested. The collection of diploid pathogenic GRACE strains used in such a method for screening for therapeutic agents may comprise, for example, a substantially complete set of all the modified allelic gene pairs of the organism, the substantially complete set of all the modified allelic essential gene pairs of the organism or the collection may be selected from a subset of GRACE strains selected from, but not limited to the group consisting of fungal-specific, pathogen-specific, desired biochemical-function, human-homolog, cellular localization, and signal transduction cascade target sets.

The GRACE strains are grown in medium comprising a range of tetracycline concentrations to obtain the growth inhibitory dose-response curve for each strain. First, seed cultures of the GRACE strains are grown in the appropriate medium. Subsequently, aliquots of the seed cultures are diluted into medium containing varying concentrations of tetracycline. For example, the GRACE strains may be grown in duplicate cultures containing two-fold serial dilutions of tetracycline. Additionally, control cells are grown in duplicate without tetracycline. The control cultures are started from equal amounts of cells derived from the same initial seed culture of a GRACE strain of interest. The cells are grown for an appropriate period of time and the extent of growth is determined using any appropriate technique. For example, the extent of growth may be determined by measuring the optical density of the cultures. When the control culture reaches mid-log phase the percent growth (relative to the control culture) for each of the tetracycline containing cultures is plotted against the log concentrations of tetracycline to produce a growth inhibitory dose response curve for tetracycline. The concentration of tetracycline that inhibits cell growth to 50% (IC₅₀) as compared to the 0 mM tetracyline control (0% growth inhibition) is then calculated from the curve. Alternative methods of measuring growth are also contemplated. Examples of these methods include measurements of proteins, the expression of which is engineered into the cells being tested and can readily be measured. Examples of such proteins include green fluorescent protein (GFP) and various enzymes.

Cells are pretreated with the selected concentration of tetracycline and then used to test the sensitivity of cell populations to candidate compounds. For example, the cells may be pretreated with a concentration of tetracycline which inhibits growth by at least about 5%, at least about 8%, at least about 10%, at least about 20%, at least about 30%, at least about 40%, at least about 50%, at least about 60% at least about 75%, at least 80%, at least 90%, at least 95% or more than 95%. The cells are then contacted with the candidate compound and growth of the cells in tetracycline containing medium is compared to growth of the control cells in medium which lacks tetracycline to determine whether the candidate compound inhibits growth of the sensitized cells (i.e. the cells grown in the presence of tetracycline). For example, the growth of the cells in tetracycline containing medium may be compared to the growth of the cells in medium lacking tetracycline to determine whether the candidate compound inhibits the growth of the sensitized cells (i.e. the cells grown in the presence of tetracyline) to a greater extent than the candidate compound inhibits the growth of cells grown in the absence of tetracycline. For example, if a significant difference in growth is observed between the sensitized cells (i.e. the cells grown in the presence of tetracycline) and the non-sensitized cells (i.e. the cells grown in the absence of tetracycline), the candidate compound may be used to inhibit the proliferation of the organism or may be further optimized to identify compounds which have an even greater ability to inhibit the growth, survival, or proliferation of the organism.

Similarly, the virulence or pathogenicity of cells exposed to a candidate compound which express a rate limiting amount of a gene product required for virulence or pathogenicity may be compared to the virulence or pathogenicity of cells exposed to the candidate compound in which the level of expression of the gene product required for virulence or pathogenicity is not rate limiting. In such methods, test animals are challenged with the GRACE strain and fed a diet containing the desired amount of tetracycline and the candidate compound. Thus, the GRACE strain infecting the test animals expresses a rate limiting amount of a gene product required for virulence or pathogenicity (i.e. the GRACE cells in the test animals are sensitized). Control animals are challenged with the GRACE strain and are fed a diet containing the candidate compound but lacking tetracycline. The virulence or pathogenicity of the GRACE strain in the test animals is compared to that in the control animals. For example, the virulence or pathogenicity of the GRACE strain in the test animals may be compared to that in the control animals to determine whether the candidate compound inhibits the virulence or pathogenicity of the sensitized GRACE cells (i.e. the cells in the animals whose diet included tetracycline) to a greater extent than the candidate compound inhibits the growth of the GRACE cells in animals whose diet lacked tetracycline. For example, if a significant difference in growth is observed between the sensitized GRACE cells (i.e. the cells in animals whose diet included tetracycline) and the non-sensitized cells (i.e. the GRACE cells in animals whose diet did not include tetracycline), the candidate compound may be used to inhibit the virulence or pathogenicity of the organism or may be further optimized to identify compounds which have an even greater ability to inhibit the virulence or pathogenicity of the organism. Virulence or pathogenicity may be measured using the techniques described therein.

It will be appreciated that the above cell-based assays may be used to identify compounds which inhibit the activity of gene products from organisms other than Candida albicans which are homologous to the Candida albicans gene products described herein. For example, the gene products may be from animal fugal pathogens such as Aspergillus fumigatus, Aspergillus niger, Aspergillus flavis, Candida tropicalis, Candida parapsilopsis, Candida krusei, Cryptococcus neoformans, Coccidioides immitis, Exophalia dermatiditis, Fusarium oxysporum, Histoplasma capsulatum, Phneumocystis carinii, Trichosporon beigelii, Rhizopus arrhizus, Mucor rouxii, Rhizomucor pusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species. In some embodiments, the gene products are from an organism other than Saccharomyces cerevisiae.

The cell-based assay described above may also be used to identify the biological pathway in which a nucleic acid required for fungal proliferation, virulence or pathogenicity or the gene product of such a nucleic acid lies. In such methods, cells expressing a rate limiting level of a target nucleic acid required for fungal proliferation, virulence or pathogenicity and control cells in which expression of the target nucleic acid is not rate limiting are contacted with a panel of antibiotics known to act in various pathways. If the antibiotic acts in the pathway in which the target nucleic acid or its gene product lies, cells in which expression of target nucleic acid is rate limiting will be more sensitive to the antibiotic than cells in which expression of the target nucleic acid is not rate limiting.

As a control, the results of the assay may be confirmed by contacting a panel of cells in which the levels of many different genes required for proliferation, virulence or pathogenicity, including the target gene, is rate limiting. If the antibiotic is acting specifically, heightened sensitivity to the antibiotic will be observed only in the cells in which the target gene is rate limiting (or cells in which genes in the same pathway as the target gene is rate limiting) but will not be observed generally in which a gene product required for proliferation, virulence or pathogenicity is rate limiting.

It will be appreciated that the above method for identifying the biological pathway in which a nucleic acid required for proliferation, virulence or pathogenicity lies may be applied to nucleic acids from organisms other than Candida albicans which are homologous to the Candida albicans nucleic acids described herein. For example, the nucleic acids may be from animal fugal pathogens such as Aspergillus fumigatus, Aspergillus niger, Aspergillus flavis, Candida tropicalis, Candida parapsilopsis, Candida krusei, Cryptococcus neoformans, Coccidioides immitis, Exophalia dermatiditis, Fusarium oxysporum, Histoplasma capsulatum, Phneumocystis carinii, Trichosporon beigelii, Rhizopus arrhizus, Mucor rouxii, Rhizomucor pusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species. In some embodiments, the nucleic acids are from an organism other than Saccharomyces cerevisiae.

Similarly, the above method may be used to determine the pathway on which a test compound, such as a test antibiotic acts. A panel of cells, each of which expresses a rate limiting amount of a gene product required for fungal proliferation, virulence or pathogenicity where the gene product lies in a known pathway, is contacted with a compound for which it is desired to determine the pathway on which it acts. The sensitivity of the panel of cells to the test compound is determined in cells in which expression of the nucleic acid encoding the gene product required for proliferation, virulence or pathogenicity is at a rate limiting level and in control cells in which expression of the gene product required for proliferation, virulence or pathogenicity is not at a rate limiting level. If the test compound acts on the pathway in which a particular gene product required for proliferation, virulence, or pathogenicity lies, cells in which expression of that particular gene product is at a rate limiting level will be more sensitive to the compound than the cells in which gene products in other pathways are at a rate limiting level. In addition, control cells in which expression of the particular gene required for fungal proliferation, virulence or pathogenicity is not rate limiting will not exhibit heightened sensitivity to the compound. In this way, the pathway on which the test compound acts may be determined.

It will be appreciated that the above method for determining the pathway on which a test compound acts may be applied to organisms other than Candida albicans by using panels of cells in which the activity or level of gene products which are homologous to the Candida albicans gene products described herein is rate limiting. For example, the gene products may be from animal fugal pathogens such as Aspergillus fumigatus, Aspergillus niger, Aspergillus flavis, Candida tropicalis, Candida parapsilopsis, Candida krusei, Cryptococcus neoformans, Coccidioides immitis, Exophalia dermatiditis, Fusarium oxysporum, Histoplasma capsulatum, Pneumocystis carinii, Trichosporon beigelii, Rhizopus arrhizus, Mucor rouxii, Rhizomucor pusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species. In some embodiments, the gene products are from an organism other than Saccharomyces cerevisiae. Example 6.4, infra, provided below describes one method for performing such assays.

One skilled in the art will appreciate that further optimization of the assay conditions, such as the concentration of inducer or repressor used to produce rate limiting levels of a gene product required for fungal proliferation, virulence or pathogenicity and/or the growth conditions used for the assay (for example incubation temperature and medium components) may further increase the selectivity and/or magnitude of the antibiotic sensitization exhibited.

It will be appreciated that the above methods for identifying the pathway in which a gene required for growth, survival, proliferation, virulence or pathogenicity lies or the pathway on which an antibiotic acts may be performed using organisms other than Candida albicans in which gene products homologous to the Candida albicans gene products described herein are rate limiting. For example, the gene products may be from animal fugal pathogens such as Aspergillus fumigatus, Aspergillus niger, Aspergillus flavis, Candida tropicalis, Candida parapsilopsis, Candida krusei, Cryptococcus neoformans, Coccidioides immitis, Exophalia dermatiditis, Fusarium oxysporum, Histoplasma capsulatum, Pneumocystis carinii, Trichosporon beigelii, Rhizopus arrhizus, Mucor rouxii, Rhizomucor pusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species. In some embodiments, the gene products are from an organism other than Saccharomyces cerevisiae.

Furthermore, as discussed above, panels of GRACE strains may be used to characterize the point of intervention of any compound affecting an essential biological pathway including antibiotics with no known mechanism of action.

Another embodiment of the present invention is a method for determining the pathway against which a test antibiotic compound is active, in which the activity of proteins or nucleic acids involved in pathways required for fungal growth, survival, proliferation, virulence or pathogenicity is reduced by contacting cells with a sub-lethal concentration of a known antibiotic which acts against the protein or nucleic acid. The method is similar to those described above for determining which pathway a test antibiotic acts against, except that rather than reducing the activity or level of a gene product required for fungal proliferation, virulence or pathogenicity by expressing the gene product at a rate limiting amount in a GRACE strain, the activity or level of the gene product is reduced using a sub-lethal level of a known antibiotic which acts against the gene product.

Growth inhibition resulting from the presence of sub-lethal concentration of the known antibiotic may be at least about 5%, at least about 8%, at least about 10%, at least about 20%, at least about 30%, at least about 40%, at least about 50%, at least about 60%, or at least about 75%, at least 80%, at least 90%, at least 95% or more than 95%.

Alternatively, the sub-lethal concentration of the known antibiotic may be determined by measuring the activity of the target proliferation-required gene product rather than by measuring growth inhibition.

Cells are contacted with a combination of each member of a panel of known antibiotics at a sub-lethal level and varying concentrations of the test antibiotic. As a control, the cells are contacted with varying concentrations of the test antibiotic alone. The IC₅₀ of the test antibiotic in the presence and absence of the known antibiotic is determined. If the IC₅₀s in the presence and absence of the known drug are substantially similar, then the test drug and the known drug act on different pathways. If the IC₅₀s are substantially different, then the test drug and the known drug act on the same pathway.

Similar methods may be performed using known antibiotics which act on a gene product homologous to the Candida albicans sequences described herein. The homolgous gene product may be from animal fugal pathogens such as Aspergillus fumigatus, Aspergillus niger, Aspergillus flavis, Candida tropicalis, Candida parapsilopsis, Candida krusei, Cryptococcus neoformans, Coccidioides immitis, Exophalia dermatiditis, Fusarium oxysporum, Histoplasma capsulatum, Pneumocystis carinii, Trichosporon beigelii, Rhizopus arrhizus, Mucor rouxii, Rhizomucor pusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species. In some embodiments, the gene products are from an organism other than Saccharomyces cerevisiae.

Another embodiment of the present invention is a method for identifying a candidate compound for use as an antibiotic in which the activity of target proteins or nucleic acids involved in pathways required for fungal proliferation, virulence or pathogenicity is reduced by contacting cells with a sub-lethal concentration of a known antibiotic which acts against the target protein or nucleic acid. The method is similar to those described above for identifying candidate compounds for use as antibiotics except that rather than reducing the activity or level of a gene product required for proliferation, virulence or pathogenicity using GRACE strains which express a rate limiting level of the gene product, the activity or level of the gene product is reduced using a sub-lethal level of a known antibiotic which acts against the proliferation required gene product.

The growth inhibition from the sub-lethal concentration of the known antibiotic may be at least about 5%, at least about 8%, at least about 10%, at least about 20%, at least about 30%, at least about 40%, at least about 50%, at least about 60%, or at least about 75%, or more.

Alternatively, the sub-lethal concentration of the known antibiotic may be determined by measuring the activity of the target proliferation-required gene product rather than by measuring growth inhibition.

In order to characterize test compounds of interest, cells are contacted with a panel of known antibiotics at a sub-lethal level and one or more concentrations of the test compound. As a control, the cells are contacted with the same concentrations of the test compound alone. The IC₅₀ of the test compound in the presence and absence of the known antibiotic is determined. If the IC₅₀ of the test compound is substantially different in the presence and absence of the known drug then the test compound is a good candidate for use as an antibiotic. As discussed above, once a candidate compound is identified using the above methods its structure may be optimized using standard techniques such as combinatorial chemistry.

Similar methods may be performed using known antibiotics which act on a gene product homologous to the Candida albicans sequences described herein. The homolgous gene product may be from animal fugal pathogens such as Aspergillus fumigatus, Aspergillus niger, Aspergillus flavis, Candida tropicalis, Candida parapsilopsis, Candida krusei, Cryptococcus neoformans, Coccidioides immitis, Exophalia dermatiditis, Fusarium oxysporum, Histoplasma capsulatum, Pneumocystis carinii, Trichosporon beigelii, Rhizopus arrhizus, Mucor rouxii, Rhizomucor pusillus, or Absidia corymbigera, or the plant fungal pathogens, such as Botrytis cinerea, Erysiphe graminis, Magnaporthe grisea, Puccinia recodita, Septoria triticii, Tilletia controversa, Ustilago maydis, or any species falling within the genera of any of the above species. In some embodiments, the gene products are from an organism other than Saccharomyces cerevisiae.

An exemplary target gene product is encoded by CaTBF1. A number of features make this C. albicans gene product a valuable drug target. First, the protein encoded by CaTBF1 is compatible with in vitro high throughput screening of compounds that inhibit its activity. Modulated expression of this gene product in whole cell assays could be performed in parallel with in vitro assays to broaden the spectrum of possible inhibitory compounds identified. In addition, demonstration of the predicted physical interaction between CaTbf1p and chromosomal telomerases could be used to develop two-hybrid assays for drug screening purposes. Finally, because CaTBF1 is a fungal specific gene, its nucleotide sequence could serve in designing PCR-based diagnostic tools for fungal infection.

Other validated drug targets included in the GRACE-derived strain collection that represent preferred drug targets include the products encoded by the following C. albicans genes: CaRHO1, CaERG8, CaAUR1, and CaCHO1, as well as those encoded by SEQ ID NOs.: 6001-6932. The ability to manipulate these genes using GRACE methods of the present invention will improve drug screening practices now in use that are designed to identify inhibitors of these critical gene products.

In another embodiment of the present invention, all potential drug targets of a pathogen could be screened simultaneously against a library of compounds using, for example a 96 well microtiter plate format, where growth, measured by optical density or pellet size after centrifugation, may be determined for each well. A genomic approach to drug screening eliminates reliance upon potentially arbitrary and artificial criteria used in evaluating which target to screen and instead allows all potential targets to be screened. This approach not only offers the possibility of identifying specific compounds which inhibit a preferred process (e.g. cell wall biosynthetic gene products) but also the possibility of identifying all fungicidal compounds within that library and linking them to their cognate cellular targets.

In still another embodiment of the present invention, GRACE strains could be screened to identify synthetic lethal mutations, and thereby uncover a potentially novel class of drug targets of significant therapeutic value. For example two separate genes may encode homologous proteins that participate in a common and essential cellular function, where the essential nature of this function will only become apparent upon inactivation of both family members. Accordingly, examination of the null phenotype of each gene separately would not reveal the essential nature of the combined gene products, and consequently, this potential drug target would not be identified. Provided the gene products are highly homologous to one another, compounds found to inhibit one family member are likely to inhibit the other and are therefore predicted to approximate the synthetic growth inhibition demonstrated genetically. In other cases however, synthetic lethality may uncover seemingly unrelated (and often nonessential) processes, which when combined produce a synergistic growth impairment (cell death). For example, although disruption of the S. cerevisiae gene RVS161 does not present any discernable vegetative growth phenotype in yeast carrying this single mutation, at least 9 other genes are known to display a synthetic lethal effect when combined with inactivation of RVS161. These genes participate in processes ranging from cytoskeletal assembly and endocytosis, to signal transduction and lipid metabolism and identifies multiple avenues to pursuing a combination drug target strategy. A directed approach to uncovering synthetic lethal interactions with essential and nonessential drug targets is now performed where a GRACE strain or heterozygote strain is identified as displaying an enhanced sensitivity to the tested compound, not because it expresses a reduced level of activity for the drug target, but because its mutation is synthetically lethal in combination with inhibition of a second drug target. Discerning whether the compound specifically inhibits the drug target in the sensitized GRACE strain or heterozygote strain or a second target may be achieved by screening the entire GRACE or heterozygote strain sets for additional mutant strains displaying equal or greater sensitivity to the compound, followed by genetic characterization of a double mutant strain demonstrating synthetic lethality between the two mutations.

5.5.2.2 Screening for Non-Antifungal Therapeutic Agents with GRACE Strains

The biochemical similarity existing between pathogenic fungi and the mammalian hosts they infect limits the range of clinically useful antimycotic compounds. However, this similarity can be exploited using a GRACE strain collection to facilitate the discovery of therapeutics that are not used as antimycotics, but are useful for treatment a wide-range of diseases, such as cancer, inflammation, etc.

In this embodiment of the invention, fungal genes that are homologous to disease-causing genes in an animal or plant, are selected and GRACE strains of this set of genes are used for identification of compounds that display potent and specific bioactivity towards the products of these genes, and therefore have potential medicinal value for the treatment of diseases. Essential and non-essential genes and the corresponding GRACE strains carrying modified allelic pairs of such genes are useful in this embodiment of the invention. It has been predicted that as many as 40% of the genes found within the C. albicans genome share human functional homologs. It has also been predicted that as many as 1% of human genes are involved in human diseases and therefore may serve as potential drug targets. Accordingly, many genes within the GRACE strain collection are homologs to disease-causing human genes and compounds that specifically inactivate individual members of this gene set may in fact have alternative therapeutic value. The invention provides a pluralities of GRACE strains in which the modified alleles are fungal genes that share sequence, structural and/or functional similarities to genes that are associated with one or more diseases of the animal or plant.

For example, much of the signal transduction machinery that promotes cell cycle progression and is often perturbed in a variety of cancers is conserved in fungi. Many of these genes encode for cyclins, cyclin-dependent kinases (CDK), CDK inhibitors, phosphatases, and transcription factors that are both structurally and functionally related. As a result, compounds found to display specificity towards any of these functional classes of proteins could be evaluated by secondary screens to test for potential anticancer activity. However, cytotoxic compounds identified in this way need not act on cancer causing targets to display therapeutic potential. For example the taxol family of anti-cancer compounds, which hold promise as therapeutics for breast and ovarian cancers, bind tubulin and promote microtubule assembly, thereby disrupting normal microtubule dynamics. Yeast tubulin displays similar sensitivity to taxol, suggesting that additional compounds affecting other fundamental cellular processes shared between yeast and man could similarly be identified and assessed for antitumor activity.

The phenomenon of pathogenesis extends far beyond the taxonomic borders of microbes and ultimately reflects the underlying physiology. In many ways, the phenomenon of cancer is analogous to the process of pathogenesis by an opportunistic pathogen such as C. albicans. Both are non-infectious diseases caused by either the body's own cells, or microbes from its natural fauna. These cells grow in a manner unchecked by the immune system and in both cases disease manifests itself by colonization of vital organs and eventual tissue damage resulting in death. Effective drug-based treatment is also elusive for both diseases primarily because the causative agent in both cases is highly related to the host.

In fact, a number of successful therapeutic drugs affecting processes unrelated to cancer have also been discovered through anti-fungal drug screening programs. One clinically-important class of compounds includes the immunosuppressant molecules rapamycin, cyclosporin A, and FK506, which inhibit conserved signal transduction components. Cyclosporin A and FK506, form distinct drug-prolyl isomerase complexes (CyPA- Cyclosporin A and FKBP12-FK506 respectively) which bind and inactivate the regulatory subunit of the calcium and calmodulin-dependent phosphatase, calcineurin. Rapamycin also complexes with FKBP12, but this drug-protein complex also binds to the TOR family of phosphatidylinositol kinases to inhibit translation and cell cycle progression. In each case, both the mechanism of drug action, and the drug targets themselves are highly conserved from yeast to humans.

The identification of C. albicans drug targets, and grouping the targets into essential-gene, fungal-specific, and pathogen-specific target sets provide the basis for the development of whole-cell screens for compounds that interact with and inhibit individual members of any of these targets. Therefore, similar analyses can be used to identify other sets of GRACE strains having modified allelic pairs of genes encoding drug targets with other specific common functions or attributes. For example, GRACE strain subsets can be established which comprise gene targets that are highly homologous to human genes, or gene targets that display a common biochemical function, enzymatic activity, or that are involved in carbon compound catabolism, biosynthesis, transport of molecules (transporter activity), cellular localization, signal transduction cascades, cell cycle control, cell adhesion, transcription, translation, DNA replication, etc. An exemplary list of biochemical functions is provided in Section 5.4.3.

5.5.2.3 Target Gene Dosage-Based Whole Cell Assays

Experiments involving modulating the expression levels of the encoding gene to reveal phenotypes from which gene function may be inferred can be carried out in a pathogenic diploid fungus, such as Candida albicans, using the strains and methods of the present intention. The principle of drug-target-level variation in drug screening involves modulating the expression level of a drug target to identify specific drug resistance or drug sensitivity phenotypes, thereby linking a drug target to a particular compound. Often, these phenotypes are indicative of the target gene encoding the bona fide drug target of this compound. In examples where this is not the case, the candidate target gene may nonetheless provide important insight into the true target gene that is functioning either in a pathway or process related to that inhibited by the compound (e.g. producing synthetic phenotype), or instead functioning as a drug resistance mechanism associated with the identified compound.

Variation of the expression levels of the target protein is also incorporated within both drug screening and drug target identification procedures. The total, cellular expression level of a gene product in a diploid organism is modified by disrupting one allele of the gene encoding that product, thereby reducing its functional activity in half, creating a “haploinsufficient” phenotype. A heterozygous S. cerevisiae strain collection has been used in such a haploinsufficiency screen to link drug-based resistance and hypersensitive phenotypes to heterozygous drug targets. Nonessential genes are screened directly using a haploid deletion strain collection against a compound library for specific phenotypes or “chemotypes.” However, this procedure cannot be used in a haploid organism where the target gene is an essential one.

The expression level of a given gene product is also elevated by cloning the gene into a plasmid vector that is maintained at multiple copies in the cell. Overexpression of the encoding gene is also achieved by fusing the corresponding open reading frame of the gene product to a more powerful promoter carried on a multicopy plasmid. Using these strategies, a number of overexpression screens have been successfully employed in S. cerevisiae to discover novel compounds that interact with characterized drug targets as well as to identify the protein targets bound by existing therapeutic compounds.

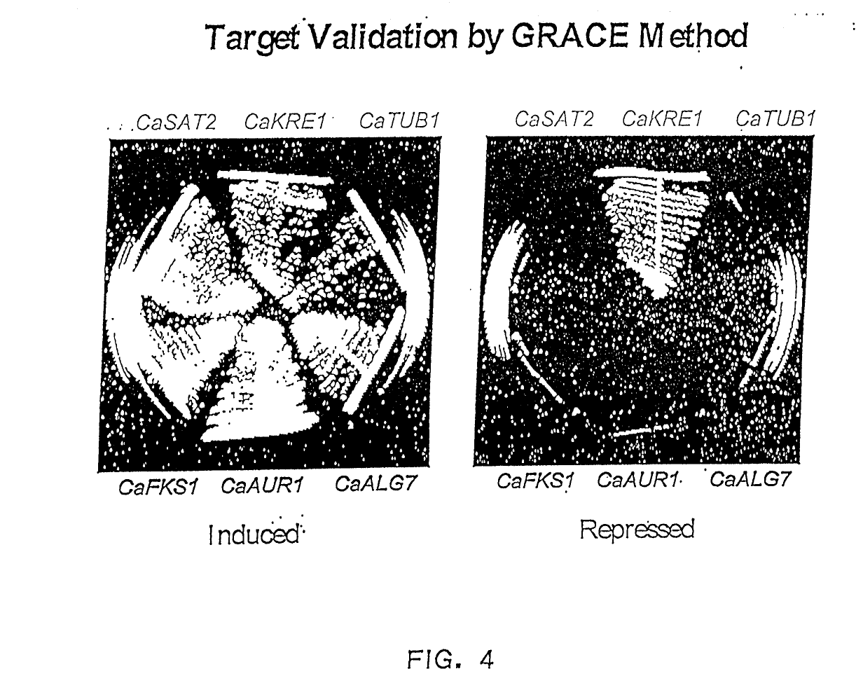

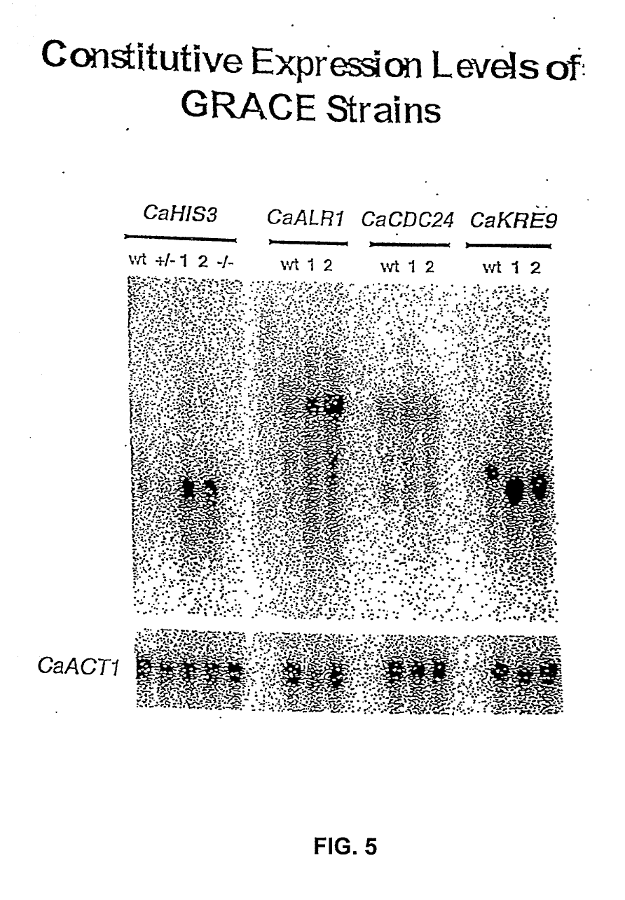

The GRACE strain collection replaces the surrogate use of S. cerevisiae in whole cell drug screening by providing a dramatic range in gene expression levels for drug targets directly within the pathogen (FIG. 5). In one embodiment of the invention, this is achieved using the C. albicans-adapted tetracycline promoter system to construct GRACE strains. Northern Blot analysis of 30 different GRACE strains grown under nonrepressing conditions (i.e. no tetracycline) reveals that 83% of conditionally expressed genes tested maintain an overexpression level greater than or equal to 3 fold of wild type, and 60% of all genes examined express greater than or equal to 5 times that of the wild type C. albicans strain used for GRACE strain construction. As each GRACE strain is in fact heterozygous, this expression range is presumably doubled if compared against their respective heterozygote strain. For most GRACE strains then, this represents an elevated expression level rivaling that typically achieved in S. cerevisiae using standard 2 μ-based multicopy plasmids, and an absolute level of constitutive expression comparable to that provided by the CaACT1 promoter. Therefore, the GRACE strain collections of the invention are not only useful in target validation under repressing conditions, but are also useful as a collection of strains overexpressing these same validated drug targets under nonrepressing conditions for whole cell assay development and drug screening.

Variation in the level of expression of a target gene product in a GRACE strain is also used to explore resistance to antimycotic compounds. Resistance to existing antifungal therapeutic agents reflects both the limited number of antifungal drugs available and the alarming dependence and reliance clinicians have in prescribing them. For example, dependence on azole-based compounds such as fluconazole for the treatment of fungal infections, has dramatically undermined the clinical therapeutic value for this compound. The GRACE strain collection is used to combat fluconazole resistance by identifying gene products that interact with the cellular target of fluconazole. Such products are used to identify drug targets which, when inactivated in concert with fluconazole, provide a synergistic effect and thereby overcome resistance to fluconazole seen when this compound is used alone. This is accomplished, for example, by using the GRACE strain collection to overexpress genes that enhance drug resistance. Such genes include novel or known plasma membrane exporters including ATP-binding cassette (ABC) transporters and multidrug resistance (MDR) efflux pumps, pleiotropic drug resistance (PDR) transcription factors, and protein kinases and phosphatases. Alternatively, genes specifically displaying a differential drug sensitivity are identified by screening GRACE strains expressing reduced levels (either by haploinsufficiency or threshold expression via the tetracycline promoter) individual members of the target set. Identifying such genes provides important clues to drug resistance mechanisms that could be targeted for drug-based inactivation to enhance the efficacy of existing antifungal therapeutics.

In another aspect of the present invention, overexpression of the target gene for whole cell assay purposes is supported with promoters other than the tetracycline promoter system. (see Section 5.3.1) For example, the CaPGK1 promoter is used to overexpress C. albicans drug targets genes. In S. cerevisiae, the PGK1 promoter is known to provide strong constitutive expression in the presence of glucose. See, Guthrie, C., and G. R. Fink. 1991. Guide to yeast genetics and molecular biology. Methods Enzymol. 194:373-398. A preliminary analysis of five C. albicans genes placed under the control of the CaPGK1 promoter (CaKRE9, CaERG11, CaALG7, CaTUB1 and CaAUR1) revealed dramatic overexpression versus wild type as judged by Northern blot analysis. The level of overexpression achieved for all genes exceeds that obtained by the tetracycline promoter by 3-4 fold. Moreover, CaAUR1, which was not overexpressed significantly when constitutively expressed using the tetracycline promoter, was overexpressed 5-fold relative to wild type CaAUR1 expression levels, suggesting that the CaPGK1 promoter is useful in overexpressing genes normally not overexpressed by the tetracycline promoter.

In another aspect of the present invention, intermediate expression levels of individual drug targets within the GRACE strain collection may are engineered to provide strains tailored for the development of unique whole cell assays. In this embodiment of the invention, GRACE strains are grown in a medium containing a tetracycline concentration determined to provide only a partial repression of transcription. Under these conditions, it is possible to maintain an expression level between that of the constitutively expressed overproducing strain and that of wild type strain, as well as levels of expression lower than that of the wild-type strain. That is, it is possible to titrate the level of expression to the minimum required for cell viability. By repressing gene expression to this critical state, novel phenotypes, resembling those produced by a partial loss of function mutation (i.e. phenocopies of hypomorphic mutants) may be produced and offer additional target expression levels applicable for whole cell assay development and drug screening. Repressing expression of the remaining allele of an essential gene to the threshold level required for viability, therefore will provide a strain with enhanced sensitivity toward compounds active against this essential gene product.

In order to demonstrate the utility of target level expression in whole cell assays for drug screening, both a CaHIS3 heterozygote strain and a tetracycline promoter-regulated CaHIS3 GRACE strain were compared against a wild type (diploid) CaHIS3 strain for sensitivity towards the 3-aminotriazole (3-AT) (Example 6.3). The data derived from these experiments clearly indicate that distinct levels of target gene products synthesized within the pathogen could be directly applied in whole cell assay based drug screens to identify novel antifungal compounds active against novel drug targets validated using the GRACE method.

5.5.2.4 Uses of Tagged Strains

In still another aspect of the present invention, unique oligonucleotide sequence tags or “bar codes” are incorporated into individual mutant strains included within a heterozygous strain collection of validated targets. The presence of these sequence tags enables an alternative whole cell assay approach to drug screening. Multiple target strains may be screened simultaneously in a mixed population (rather than separately) to identify phenotypes between a particular drug target and its inhibitory agent.

Large-scale parallel analyses are performed using mixed populations of the entire bar coded heterozygous essential strain collection target set and comparing the relative representation of individual strains within a mixed population prior to and after growth in the presence of a compound. Drug-dependent depletion or overrepresentation of a unique bar-coded strain is determined by PCR-amplifying and fluorescently labeling all bar codes within the mixed population and hybridizing the resulting PCR products to an array of complementary oligonucleotides. Differential representation between bar coded strains indicates gene-specific hypersensitivity or resistance and suggests the corresponding gene product may represent the molecular target of the compound tested.

In one specific embodiment, the mutant strains are GRACE strains, and each of the GRACE strains of the set comprises at least one, and preferably two unique molecular tags, which, generally, are incorporated within the cassette used to replace the first allele of the gene pair to be modified. Each molecular tag is flanked by primer sequences which are common to all members of the set being tested. Growth is carried out in repressive and non-repressive media, in the presence and absence of the compound to be tested. The relative growth of each strain is assessed by carrying out simultaneous PCR amplification of the entire collection of embedded sequence tags.

In one non-limiting aspect of the present invention, the PCR amplification is performed in an asymmetric manner with fluorescent primers and the resulting single stranded nucleic acid product hybridized to an oligonucleotide array fixed to a surface and comprises the entire corresponding set of complementary sequences. Analysis of the level of each fluorescent molecular tag sequence is then determined to estimate the relative amount of growth of GRACE strain of the set, in those media, in the presence and absence of the compound tested.

Therefore, for each GRACE strain of the set tested, there could be, in one non-limiting example of this method, four values for the level of the corresponding molecular tag found within the surviving population. They would correspond to cell growth under repressing and non-repressing conditions, both in the presence and absence of the compound being tested. Comparison of growth in the presence and absence of the test compound provides a value or “indicator” for each set of growth media; that is, an indicator derived under repressing and non-repressing conditions. Again, comparison of the two indicator values will reveal if the test compound is active against the gene product expressed by the modified allelic gene pair carried by that specific member of the GRACE set tested.

In still another aspect of the present invention, each potential drug target gene in a heterozygous tagged or bar-coded collection, may be overexpressed. For example, in the heterozygous tagged or bar-coded collection described above, each of the potential target gene can be overexpressed by introducing either the Tet promoter or another strong, constitutively expressed promoter (e.g. CaACT1, CaADH1 and CaPGK1) upstream of the remaining non-disrupted allele. These constructions allow a further increase in the dosage of the encoded target gene product of individual essential genes to be used in mixed-population drug susceptibility studies. Although overexpression may itself disrupt the normal growth rate of numerous members of the population, reliable comparisons could still be made between mock and drug-treated mixed cultures to identify compound-specific growth differences.

In S. cerevisiae, the molecular drug targets of several well-characterized compounds including 3-amino-triazol, benomyl, tunicamycin and fluconazole were identified by a similar approach. In that study, bar-coded strains bearing heterozygous mutations in HIS3, TUB1, ALG7, and ERG11, (i.e. the respective drug targets to the compounds listed above) displayed significantly greater sensitivity when challenged with their respective compound than other heterozygote bar-coded strains when grown together in a mixed population.

In another aspect of the present invention, screens for antifungal compounds can be carried out using complex mixtures of compounds that comprise at least one compound active against the target strain. Tagging or bar-coding the GRACE strain collection facilitates a number of large scale analyses necessary to identify gene sets as well as evaluate and ultimately evaluate individual targets within particular gene sets. For example, mixed-population drug screening using a bar-coded GRACE strain collection effectively functions as a comprehensive whole cell assay. Minimal amounts of a complex compound library are sufficient to identify compounds that act on individual essential target genes within the collection. This is done without the need to array the collection. Also, strong predictions as to the ‘richness’ of any particular compound library could be made before committing to it in drug screening. It becomes possible then to assess whether, for example, a carbohydrate-based chemical library possesses greater fungicidal activity than a natural product or synthetic compound library. Particularly potent compounds within any complex library of molecules can be immediately identified and evaluated according to the priority of targets and assays available for drug screening. Alternatively, the invention provides applying this information to developing “tailored” screens, in which only those targets which were demonstrated to be inactivated in mixed population experiments by a particular compound library would be included in subsequent array-formatted screens.

Traditionally, drug discovery programs have relied on an individual or a limited set of validated drug targets. The preceding examples emphasize that such an approach is no longer necessary and that high throughput target evaluation and drug screening are now possible. However, a directed approach based on selecting individual targets may still be preferred depending on the expertise, interest, strategy, or budget of a drug discovery program.

5.5.3 Target Evaluation in an Animal Model System

Currently, validation of an essential drug target is demonstrated by examining the effect of gene inactivation under standard laboratory conditions. Putative drug target genes deemed nonessential under standard laboratory conditions may be examined within an animal model, for example, by testing the pathogenicity of a strain homozygous for a deletion in the target gene versus wild type. However, essential drug targets are precluded from animal model studies. Therefore, the most desirable drug targets are omitted from the most pertinent conditions to their target evaluation.

In one specific embodiment of the invention, conditional expression, provided by the GRACE essential strain collection, overcomes this longstanding limitation to target validation within a host environment. Animal studies can be performed using mice inoculated with GRACE essential strains and examining the effect of gene inactivation by conditional expression. In a preferred embodiment of the invention, the effect on mice injected with a lethal inoculum of a GRACE essential strain could be determined depending on whether the mice were provided with an appropriate concentration of tetracycline to inactivate expression of a drug target gene. The lack of expression of a gene demonstrated to be essential under laboratory conditions can thus be correlated with prevention of a terminal C. albicans infection. In this type of experiment, only mice “treated” with tetracycline-supplemented water, are predicted to survive infection because inactivation of the target gene has killed the GRACE strain pathogen within the host.

In yet another embodiment of the invention, conditional expression could be achieved using a temperature-responsive promoter to regulate expression of the target gene or a temperature sensitive allele of a particular drug target, such that the gene is functional at 30° C. but inactivated within the normal body temperature of the mouse.

In the same manner as described above for essential genes, it is equally feasible to demonstrate whether nonessential genes comprising the GRACE strain collection are required for pathogenicity in a mouse model system. Included in this set are multiple genes whose null phenotype results in a reduced growth rate and may attenuate the virulence of the pathogen. Many mutants demonstrating a slow growth phenotype may represent hypomorphic mutations in otherwise essential genes (as demonstrated by alternative methods) which are simply not completely inactivated by the conditional expression method used to construct the GRACE strain. One important use of such strains is to assess whether any given essential gene doubly functions in the process of virulence. Essential genes that display substantially reduced virulence and growth rate when only partially inactivated represent “multifactorial” drug targets for which even minimally inhibitory high specificity compounds would display therapeutic value. Collectively, all GRACE strains that fail to cause fungal infection in mice under conditions of gene inactivation by tetracycline (or alternative gene inactivation means) define a subset of genes that are required for pathogenicity, i.e., GRACE pathogenicity subset. More defined subsets of pathogenicity genes, for example those genes required for particular steps in pathogenesis (e.g. adherence or invasion) may be determined by applying the GRACE pathogenicity subset of strains to in vitro assays which measure the corresponding process. For example, examining GRACE pathogenicity strains in a buccal adhesion or macrophage assay by conditional expression of individual genes would identify those pathogenicity factors required for adherence or cell invasion respectively.

The GRACE strain collection or a desired subset thereof is also well suited for evaluating acquired resistance/suppression or distinguishing between fungicidal/fungistatic phenotypes for an inactivated drug target within an animal model system. In this embodiment of the invention, GRACE strains repressed for expression of different essential drug target genes would be inoculated into mice raised on tetracycline-supplemented water. Each of the GRACE strains would then be compared according to the frequency of death associated with the different mice populations they infected. It is expected that the majority of infected mice will remain healthy due to fungal cell death caused by tetracycline-dependent inactivation of the essential gene in the GRACE strain. However, a GRACE strain harboring a drug target more likely to develop extragenic suppressors because it is a fungistatic target rather than fungicidal one, or suppressed by an alternative physiological process active within a host environment, can be identified by the higher incidence of lethal infections detected in mice infected with this particular strain. By this method, it is possible to evaluate/rank the likelihood that individual drug target genes may develop resistance within the host environment.

Although a GRACE strain is highly suited for this purpose, it is also contemplated that a strain with a modified allele of an essential gene or a modified essential gene is used in an animal model for drug target evaluation.

5.5.4 Rational Design of Binding Compounds

Compounds identified via assays such as those described herein can be useful, for example, for inhibiting the growth of the infectious agent and/or ameliorating the symptoms of an infection. Compounds can include, but are not limited to, other cellular proteins. Binding compounds can also include, but are not limited to, peptides such as, for example, soluble peptides, comprising, for example, extracellular portions of target gene product transmembrane receptors, and members of random peptide libraries (see, e.g., Lam et al., 1991, Nature 354:82-84; Houghten et al., 1991, Nature 354:84-86) made of D-and/or L-configuration amino acids, rationally-designed antipeptide peptides, (see e.g., Hurby et al., Application of Synthetic Peptides: Antisense Peptides,” In Synthetic Peptides, A User's Guide, W. H. Freeman, NY (1992), pp. 289-307), antibodies (including, but not limited to polyclonal, monoclonal, human, humanized, anti-idiotypic, chimeric or single chain antibodies, and FAb, F(ab′)₂ and FAb expression library fragments, and epitope-binding fragments thereof), and small organic or inorganic molecules. In the case of receptor-type target molecules, such compounds can include organic molecules (e.g., peptidomimetics) that bind to the ECD and either mimic the activity triggered by the natural ligand (i.e., agonists); as well as peptides, antibodies or fragments thereof, and other organic compounds that mimic the ECD (or a portion thereof) and bind to a “neutralize” natural ligand.

Computer modeling and searching technologies permit identification of compounds, or the improvement of already identified compounds, that can modulate target gene expression or activity. Having identified such a compound or composition, the active sites or regions are preferably identified. In the case of compounds affecting receptor molecules, such active sites might typically be ligand binding sites, such as the interaction domains of ligand with receptor itself. The active site is identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods are used to find the active site by finding where on the factor the complexed ligand is found.

The three-dimensional geometric structure of the active site is then preferably determined. This is done by known methods, including X-ray crystallographic, which determines a complete molecular structure. Solid or liquid phase NMR is also used to determine certain intra-molecular distances within the active site and/or in the ligand binding complex. Other experimental methods of structure determination known to those of skill in the art, are also used to obtain partial or complete geometric structures. The geometric structures are measured with a complexed ligand, natural or artificial, which increases the accuracy of the active site structure determined. Methods of computer based numerical modeling are used to complete the structure (e.g., in embodiments wherein an incomplete or insufficiently accurate structure is determined) or to improve its accuracy.

Finally, having determined the structure of the active site, either experimentally, by modeling, or by a combination, candidate modulating compounds are identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential target or pathway gene product modulating compounds.

Alternatively, these methods are used to identify improved modulating compounds from an already known modulating compound or ligand. The composition of the known compound is modified and the structural effects of modification are determined using the experimental and computer modeling methods described above applied to the new composition. The altered structure is then compared to the active site structure of the compound to determine if an improved fit or interaction results. In this manner systematic variations in composition, such as by varying side groups, are quickly evaluated to obtain modified modulating compounds or ligands of improved specificity or activity.

Further experimental and computer modeling methods useful to identify modulating compounds based upon identification of the active sites of target or pathway gene or gene products and related transduction and transcription factors are apparent to those of skill in the art.

There are a number of articles that review the art of computer modeling of drugs that interact with specific proteins, including the following: Rotivinen et al., 1988, Acta Pharmaceutical Fennica 97:159-166; Ripka, (Jun. 16, 1988), New Scientist 54-57; McKinaly and Rossmann, 1989, Annu. Rev. Pharmacol. Toxicol. 29:111-122; Perry and Davies, OSAR: Quantitative Structure-Activity Relationships in Drug Design pp. 189-193 (Alan R. Liss, Inc. 1989); Lewis and Dean, 1989 Proc. R. Soc. Lond. 236:125-140 and 1-162; and, with respect to a model receptor for nucleic acid components, Askew et al., 1989, J. Am. Chem. Soc. 111:1082-1090.

Although generally described above with reference to design and generation of compounds which could alter binding, one could also screen libraries of known compounds, including natural products or synthetic chemicals, as well as other biologically active materials, including proteins, for compounds which are inhibitors or activators.

5.6 Transcriptional Profiling

5.6.1 Analysis of Gene Expression

Gene expression profiling techniques are important tools for the identification of suitable biochemical targets, as well as for the determination of the mode of action of known compounds. Completion of the C. albicans genome sequence and development of nucleic acid microarrays incorporating this information, will enable genome-wide gene expression analyses to be carried out with this diploid pathogenic fungus. Therefore, the present invention provides methods for obtaining the transcriptional response profiles for both essential and virulence/pathogenicity genes of Candida albicans. Conditional expression of essential genes serves to delineate, for example, regulatory interactions valuable for the design of drug screening programs focused upon C. albicans.

In an embodiment of the present invention, a strain or a strain collection wherein the expression of an essential gene identified by the method of the invention is modified can be used for the analysis of expression of essential genes within this pathogen. In one specific embodiment, a GRACE strain collection is used. One particularly powerful application of such a strain collection involves the construction of a comprehensive transcriptional profile database for the entire essential gene set or a desired subset of essential genes within a pathogen. Such a database is used to compare the response profile characteristic of lead antimycotic compounds with the profile obtained with new anti-fungal compounds to distinguish those with similar from those with distinct modes of action. Matching (or even partially overlapping) the transcriptional response profiles determined after treatment of the strain with the lead compound with that obtained with a particular essential target gene under repressing conditions, is used to identity the target and possible mode of action of the drug.

Gene expression analysis of essential genes also permits the biological function and regulation of those genes to be examined within the pathogen, and this information is incorporated within a drug screening program. For example, transcriptional profiling of essential drug targets in C. albicans permits the identification of novel drug targets which participate in the same cellular process or pathway uncovered for the existing drug target and which could not otherwise be identified without direct experimentation within the pathogen. These include genes not only unique to the pathogen but also broad-range gene classes possessing a distinct function or subject to different regulation in the pathogen. Furthermore, pathogen-specific pathways may be uncovered and exploited for the first time.

In another aspect of the present invention, the gene expression profile of mutant strains, such as GRACE-derived strains, under nonrepressing or induced conditions is established to evaluate the overexpression response profile for one or more drug targets. For example, overexpression of genes functioning in signal transduction pathways often display unregulated activation of the pathway under such conditions. Moreover, several signaling pathways have been demonstrated to function in the pathogenesis process. Transcriptional response profiles generated by overexpressing C. albicans GRACE strains provide information concerning the set of genes regulated by such pathways; any of which may potentially serve an essential role in pathogenesis and therefore representing promising drug targets. Furthermore, analysis of the expression profile may reveal one or more genes whose expression is critical to the subsequent expression of an entire regulatory cascade. Accordingly, these genes are particularly important targets for drug discovery and mutants carrying the corresponding modified allelic pair of genes form the basis of a mechanism-of-action based screening assays. Presently such an approach is not possible. Current drug discovery practices result in an exceedingly large number of “candidate” compounds and little understanding of their mode of action. A transcriptional response database comprising both gene shut-off and overexpression profiles generated using the GRACE strain collection offers a solution to this drug discovery bottleneck by 1) determining the transcriptional response or profile resulting from an antifungal's inhibition of a wild type strain, and 2) comparing this response to the transcriptional profiles resulting from inactivation or overexpression of drug targets comprising the GRACE strain collection.

Matching or significantly correlating transcriptional profiles resulting from both genetic alteration of a drug target and chemical/compound inhibition of wild type cells provides evidence linking the compound to its cellular drug target and suggests its mechanism of action.

Accordingly, the invention provides a method for evaluating a compound against a target gene product encoded by a nucleotide sequence comprising one of SEQ ID NOs: 6001 through to 6932, said method comprising the steps of (a) contacting wild type diploid fungal cells or control cells with the compound and generating a first transcription profile; (b) determining the transcription profile of mutant diploid fungal cells, such as a GRACE strain, which have been cultured under conditions wherein the second allele of the target gene is substantially underexpressed, not expressed or overexpressed and generating a second transcription profile for the cultured cells; and comparing the first transcription profile with the second transcription profile to identify similarities in the profiles. For comparisons, similarities of profiles can be expressed as an indicator value; and the higher the indicator value, the more desirable is the compound.

5.6.2 Identification of Secondary Targets

Methods are described herein for the identification of secondary targets. “Secondary target,” as used herein, refers to a gene whose gene product exhibits the ability to interact with target gene products involved in the growth and/or survival of an organism (i.e., target essential gene products), under a set of defined conditions, or in the pathogenic mechanism of the organism, (i.e., target virulence gene products) during infection of a host.

Any method suitable for detecting protein-protein interactions can be employed for identifying secondary target gene products by identifying interactions between gene products and target gene products. Such known gene products can be cellular or extracellular proteins. Those gene products which interact with such known gene products represent secondary target gene products and the genes which encode them represent secondary targets.

Among the traditional methods employed are co-immunoprecipitation, crosslinking and co-purification through gradients or chromatographic columns. Utilizing procedures such as these allows for the identification of secondary target gene products. Once identified, a secondary target gene product is used, in conjunction with standard techniques, to identify its corresponding secondary target. For example, at least a portion of the amino acid sequence of the secondary target gene product is ascertained using techniques well known to those of skill in the art, such as via the Edman degradation technique (see, e.g., Creighton, 1983, “Proteins: Structures and Molecular Principles,” W. H. Freeman & Co., N.Y., pp.34-49). The amino acid sequence obtained can be used as a guide for the generation of oligonucleotide mixtures that can be used to screen for secondary target gene sequences. Screening can be accomplished, for example, by standard hybridization or PCR techniques. Techniques for the generation of oligonucleotide mixtures and for screening are well-known. (See, e.g., Ausubel, supra., and PCR Protocols: A Guide to Methods and Applications, 1990, Innis, M. et al., eds. Academic Press, Inc., New York).

Additionally, methods are employed which result in the simultaneous identification of secondary targets which encode proteins interacting with a protein involved in the growth and/or survival of an organism under a set of defined conditions, or in the pathogenic mechanism of the organism during infection of a host. These methods include, for example, probing expression libraries with labeled primary target gene protein known or suggested to be involved in or critical to these mechanisms, using this protein in a manner similar to the well known technique of antibody probing of λgt11 phage libraries.

One method which detects protein interactions in vivo, the two-hybrid system, is described in detail for illustration purposes only and not by way of limitation. One version of this system has been described (Chien et al., 1991, Proc. Natl. Acad. Sci. USA, 88:9578-9582) and is commercially available from Clontech (Palo Alto, Calif.).

Briefly, utilizing such a system, plasmids are constructed that encode two hybrid proteins: one consists of the DNA-binding domain of a transcription activator protein fused to a known protein, in this case, a protein known to be involved in growth of the organism, or in pathogenicity, and the other consists of the activator protein's activation domain fused to an unknown protein that is encoded by a cDNA which has been recombined into this plasmid as part of a cDNA library. The plasmids are transformed into a strain of the yeast S. cerevisiae that contains a reporter gene (e.g., lacz) whose regulatory region contains the transcription activator's binding sites. Either hybrid protein alone cannot activate transcription of the reporter gene, the DNA-binding domain hybrid cannot because it does not provide activation function, and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology is used to screen activation domain libraries for proteins that interact with a known “bait” gene product. By way of example, and not by way of limitation, target essential gene products and target virulence gene products are used as the bait gene products. Total genomic or cDNA sequences encoding the target essential gene product, target virulence gene product, or portions thereof, are fused to the DNA encoding an activation domain. This library and a plasmid encoding a hybrid of the bait gene product fused to the DNA-binding domain are cotransformed into a yeast reporter strain, and the resulting transformants are screened for those that express the reporter gene. For example, and not by way of limitation, the bait gene is cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with bait gene product are to be detected is made using methods routinely practiced in the art. According to the particular system described herein, for example, the cDNA fragments are inserted into a vector such that they are translationally fused to the activation domain of GAL4. This library is co-transformed along with the bait gene-GAL4 fusion plasmid into a yeast strain which contains a lacZ gene driven by a promoter which contains GAL4 activation sequence. A cDNA encoded protein, fused to GAL4 activation domain, that interacts with bait gene product reconstitutes an active GAL4 protein and thereby drive expression of the lacZ gene. Colonies which express lacZ are detected by their blue color in the presence of X-gal. The cDNA can then be purified from these strains, and used to produce and isolate the bait gene-interacting protein using techniques routinely practiced in the art.

Once a secondary target has been identified and isolated, it is farther characterized and used in drug discovery by the methods of the invention.

5.6.3 Use of Gene Expression Arrays

To carry out profiling, gene expression arrays and microarrays can be employed. Gene expression arrays are high density arrays of DNA samples deposited at specific locations on a glass surface, silicon, nylon membrane, or the like. Such arrays are used by researchers to quantify relative gene expression under different conditions. An example of this technology is found in U.S. Pat. No. 5,807,522, which is hereby incorporated by reference.

It is possible to study the expression of substantially all of the genes in the genome of a particular microbial organism using a single array. For example, the arrays may consist of 12×24 cm nylon filters containing PCR products corresponding to ORFs from Candida albicans. An appropriate amount of each PCR product (e.g., 10 ng) are spotted every 1.5 mm on the filter. Single stranded labeled cDNAs are prepared for hybridization to the array and placed in contact with the filter. In an embodiment, no second strand synthesis or amplification step is done, and thus the labeled cDNAs are of “antisense” orientation. Quantitative analysis is done using a phosphorimager.

In one embodiment, PCR products of essential genes can be generated using pairs of oligonucleotide primers of the invention, i.e., SEQ ID NOs: 4001 to 4932, and SEQ ID NOs: 5001 to 5932. Ten ngs of each PCR product are spotted every 1.5 mm on the filter. Each PCR product comprises a nucleotide sequence selected from the group of nucleotide sequences consisting of SEQ ID NOs: 6001 to 6932.

Hybridization of cDNA made from a sample of total cell mRNA to such an array followed by detection of binding by one or more of various techniques known to those in the art provides a signal at each location on the array to which cDNA hybridized. The intensity of the hybridization signal obtained at each location in the array thus reflects the amount of mRNA for that specific gene that was present in the sample. Comparing the results obtained for mRNA isolated from cells grown under different conditions thus allows for a comparison of the relative amount of expression of each individual gene during growth under the different conditions.

Gene expression arrays are used to analyze the total mRNA expression pattern at various time points after reduction in the level or activity of a gene product required for fungal proliferation, virulence or pathogenicity. Reduction of the level or activity of the gene product is accomplished by growing a GRACE strain under conditions in which the product of the nucleic acid linked to the regulatable promoter is rate limiting for fungal growth, survival, proliferation, virulence or pathogenicity or by contacting the cells with an agent which reduces the level or activity of the target gene product. Analysis of the expression pattern indicated by hybridization to the array provides information on other genes whose expression is influenced by reduction in the level or activity of the gene product. For example, levels of other mRNAs may be observed to increase, decrease or stay the same following reduction in the level or activity of the gene product required for growth, survival, proliferation, virulence or pathogenicity. Thus, the mRNA expression pattern observed following reduction in the level or activity of a gene product required for growth, survival, proliferation, virulence or pathogenicity identifies other nucleic acids required for growth, survival, proliferation, virulence or pathogenicity. In addition, the mRNA expression patterns observed when the fungi are exposed to candidate drug compounds or known antibiotics are compared to those observed when the level or activity of a gene product required for fungal growth, survival, proliferation, virulence or pathogenicity is reduced. If the mRNA expression pattern observed with the candidate drug compound is similar to that observed when the level of the gene product is reduced, the drug compound is a promising therapeutic candidate. Thus, the assay is useful in assisting in the selection of promising candidate drug compounds for use in drug development.

In cases where the source of nucleic acid deposited on the array and the source of the nucleic acid being hybridized to the array are from two different microorganisms, gene expression identify homologous genes in the two microorganisms.

5.7 Proteomics Assays

In another embodiment of the present invention, and in much the same way that a mutant strain collection (e.g, GRACE strain collection) enables transcriptional profiling within a pathogen, a mutant strain collection provides an invaluable resource for the analysis of the expressed protein complement of a genome. By evaluating the overall protein expression by members of a mutant strain collection under repressing and non-repressing growth conditions, a correlation between the pattern of protein expression of a cell can be made with the non-expression or the level of expression of an essential gene. Accordingly, the invention provides a pattern of expression of a set of proteins in a mutant strain as determined by methods well known in the art for establishing a protein expression pattern, such as but not limited to two-dimensional gel electrophoresis. The set of proteins comprises proteins comprising an amino acid sequence selected from the group consisting of the amino acid sequences of SEQ ID NOs: 7000 to 7932. A plurality of protein expression patterns will be generated for a mutant strain when the strain is cultured under different conditions and different levels of expression of one of the modified allele. A preferred mutant strain collection is a GRACE strain collection.

In yet another embodiment, defined genetic mutations can be constructed to create strains exhibiting protein expression profiles comparable to those observed upon treatment of the strain with a previously uncharacterized compound. In this way, it is possible to distinguish between antimycotic compounds that act on multiple targets in a complicated manner from other potential lead compounds that act on unique fungal-specific targets and whose mode of action can be determined.

Evaluation of the full complement of proteins expressed within a cell depends upon definitive identification of all protein species detectable on two-dimensional polyacrylamide gels or by other separation techniques. However, a significant fraction of these proteins are of lower abundance and fall below the threshold level required for positive identification by peptide sequencing or mass spectrometry. Nevertheless, these “orphan” proteins are detectable using an analysis of protein expression by individual mutant strain (GRACE strains). Conditional expression of low abundance gene products facilitates their positive identification by comparing protein profiles of mutant strains (GRACE strains) under repressing versus nonrepressing or overexpression conditions. In some cases, a more complex protein profile results because of changes of steady state levels for multiple proteins, which is caused indirectly by manipulating the low abundance gene in question. Overexpression of individual targets within the GRACE strain collection can also directly aid orphan protein identification by providing sufficient material for peptide sequencing or mass spectrometry.

In various embodiments, the present invention provides a method of quantitative analysis of the expressed protein complement of a diploid pathogenic fungal cell: a first protein expression profile is developed for a control diploid pathogenic fungus, which has two, unmodified alleles for the target gene. Mutants of the control strain, in which one allele of the target gene is inactivated, for example, in a GRACE strain, by insertion by or replacement with a disruption cassette, is generated. The other allele is modified such that expression of that second allele is under the control of a heterologous regulated promoter. A second protein expression profile is developed for this mutant fungus, under conditions where the second allele is substantially overexpressed as compared to the expression of the two alleles of the gene in the control strain. Similarly, if desired, a third protein expression profile is developed, under conditions where the second allele is substantially underexpressed as compared to the expression of the two alleles of the gene in the control strain. The first protein expression profile is then compared with the second expression profile, and if applicable, a third protein expression profile to identify an expressed protein detected at a higher level in the second profile, and if applicable, at a lower level in the third profile, as compared to the level in first profile.

Accordingly, the invention provides a method for evaluating a compound against a target gene product encoded by a nucleotide sequence comprising one of SEQ ID NOs: 6001 through to 6932, said method comprising the steps of (a) contacting wild type diploid fungal cells or control cells with the compound and generating a first protein expression profile; (b) determining the protein expression profile of mutant diploid fungal cells, such as a GRACE strain, which have been cultured under conditions wherein the second allele of the target gene is substantially underexpressed, not expressed or overexpressed and generating a second protein expression profile for the cultured cells; and comparing the first protein expression profile with the second protein expression profile to identify similarities in the profiles. For comparisons, similarities of profiles can be expressed as an indicator value; and the higher the indicator value, the more desirable is the compound.

5.8 Pharmaceutical Compositions and Uses Thereof

Compounds including nucleic acid molecules that are identified by the methods of the invention as described herein can be administered to a subject at therapeutically effective doses to treat or prevent infections by a pathogenic organism, such as Candida albicans. Depending on the target, the compounds may also be useful for treatment of a non-infectious disease in a subject, such as but not limited to, cancer. A therapeutically effective dose refers to that amount of a compound (including nucleic acid molecules) sufficient to result in a healthful benefit in the treated subject. Typically, but not so limited, the compounds act by reducing the activity or level of a gene product encoded by a nucleic acid comprising a sequence selected from the group consisting of SEQ ID NOs: 6001 through to 6932. The subject to be treated can be a plant, a vertebrate, a mammal, an avian, or a human. These compounds can also be used for preventing or containing contamination of an object by Candida albicans, or used for preventing or inhibiting formation on a surface of a biofilm comprising Candida albicans. Biofilm comprising C. albicans are found on surfaces of medical devices, such as but not limited to surgical tools, implanted devices, catheters and stents.

5.8.1 Effective Dose

Toxicity and therapeutic efficacy of compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography. A useful dosage can range from 0.001 mg/kg body weight to 10 mg/kg body weight.

5.8.2 Formulations and Use

Pharmaceutical compositions for use in accordance with the present invention can be formulated in conventional manner using one or more physiologically acceptable carriers or excipients.

Thus, the compounds and their physiologically acceptable salts and solvents can be formulated for administration by inhalation or insufflation (either through the mouth or the nose) or oral, buccal, parenteral or rectal administration.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound.

For buccal administration the compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator can be formulated containing a powder mix of the compound and a suitable powder base such as lactose or starch.

The compounds can be formulated for parenteral administration (i.e., intravenous or intramuscular) by injection, via, for example, bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

### 6. EXAMPLES

6.1 Construction of a GRACE Strain Containing Modified Alleles of CaKRE9

Oligonucleotide primers for PCR amplification of the SAT selectable marker used in Step 1 (i.e. gene replacement) contain 25 nucleotides complementary to the SAT disruption cassette in pRC18-ASP, and 65 nucleotides homologous to regions flanking the CaKRE9 open reading frame. FIG. 2 illustrates the 2.2 kb cakre9Δ::SAT disruption fragment produced after PCR amplification and resulting gene replacement of the first wild type CaKRE9 allele via homologous recombination following transformation. PCR conditions were as follows: 5-50 ng pRC18-ASP, 100 pmol of each primer, 200 μM dNTPs, 10 mM Tris-pH 8.3, 1.5 mM MgCl2, 50 mM KCl, 1 unit Taq DNA polymerase (Gibco). PCR amplification times were: 5 min 94° C., 1 min 54° C., 2 min 72° C., for 1 cycle; 45 sec 94° C., 45 sec 54° C., 2 min 72° C., for 30 cycles. Transformation was performed using the lithium acetate method adapted for C. albicans, by Braun and Johnson, (Braun, B. R., and A. D. Johnson (1997), Control of filament formation in Candida albicans by the transcriptional repressor TUP1, Science 277:105-109), with minor modifications, including shorter incubation times at 30° C. and 42° C. (1 hr and 5 min respectively) and a greater amount of material transformed (50 μg of ethanol-precipitated cakre9Δ::SAT PCR product). Transformed cells were spread onto YPD plates and incubated overnight at 30° C., providing a preincubation period for expression of SAT prior to replica plating onto YPD medium containing streptothricin (400 μg/ml). Streptothricin-resistant colonies were detected after 36 hr and cakre9Δ::SAT/CaKRE9 heterozygotes identified by PCR analysis using suitable primers which amplify both CaKRE9 and cakre9Δ::SAT alleles.

Oligonucleotide primers for PCR amplification of the conditional promoter used in Step 2 (i.e. promoter replacement) contain 25 nucleotides complementary to the CaHIS3-marked tetracycline regulated promoter cassette in pBSK-HT4 and 65 nucleotides of homologous sequence corresponding to promoter regions −270 to −205, relative to the point of transcription initiation, and nucleotides 1-65 of the CaKRE9 open reading frame. The resulting 2.2 kb PCR product was transformed into the cakre9Δ:SAT/CaKRE9 heterozygous strain produced in step 1, and His⁺ transformants selected on YNB agar. Bonafide CaKRE9 GRACE strains containing both a cakre9Δ::SAT allele and CaHIS3-Tet-CaKRE9 allele were determined by PCR analysis. Typically, 2 independent GRACE strains are constructed and evaluated to provide a reliable determination of the terminal phenotype of any given drug target. Terminal phenotype is that phenotype caused by the absence of the gene product of an essential gene.

6.2 Phenotype Determination of the CaKRE9 Grace Strain

The terminal phenotype of the resulting GRACE strains was evaluated in three independent methods. In the first, rapid determination of the CaKRE9 GRACE strain terminal phenotype was achieved by streaking approximately 1.0×10⁶ cells onto both a YNB plate and YNB plate containing 100 μg/ml tetracycline and comparing growth rate after 48 hr at room temperature. For essential genes, such as CaKRE9, no significant growth is detected in the presence of tetracycline. In the second approach, the essential nature of a gene may be determined by streaking CaKRE9 GRACE cells onto a casamino acid plate containing 625 μg/ml 5-fluroorotic acid (5FOA) and 100 μg/ml uridine to select for ura-cells which have excised (via recombination between CaLEU2 sequence duplications created during targeted integration) the transactivator gene that is normally required for expression of the tetracycline promoter-regulated target gene. Again, whereas nonessential GRACE strains demonstrate robust growth under such conditions, essential GRACE strains fail to grow. Quantitative evaluation of the terminal phenotype associated with an essential GRACE strain is performed using 2×10³ cells/ml of overnight culture inoculated into 5.0 ml YNB either lacking or supplemented with 100 μg/ml tetracycline and measuring optical density (O.D.₆₀₀) after 24 and 48 hr incubation at 30° C. Typically, for essential GRACE strains, no significant increase in optical density is detected after 48 hrs. Discrimination between cell death (cidal) and growth inhibitory (static) terminal phenotypes for a demonstrated essential gene is achieved by determining the percentage of viable cells (as judged by the number of colony forming units (CFU) from an equivalent of 2×10³ washed cells at T=0) from the above tetracycline-treated cultures after 24 and 48 hours of incubation. Essential GRACE strains producing a cidal terminal phenotype are those which display a reduction in percent viable cells (i.e. <2×10³ CFU) following incubation under repressing conditions.

6.3 Target Level Variation in Whole Cell Assays

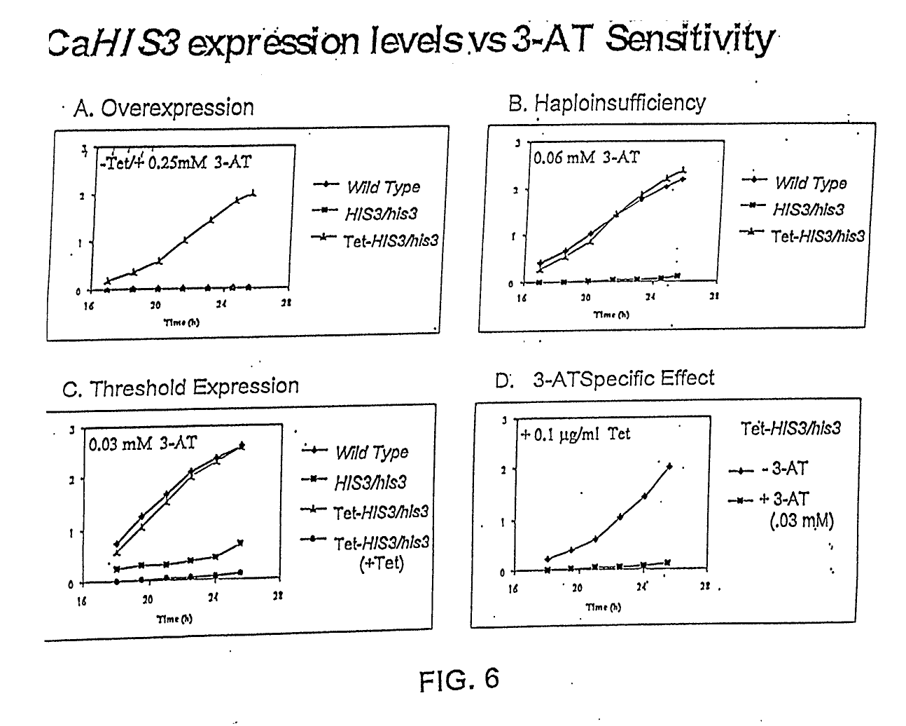

In order to demonstrate the utility of target level expression in whole cell assays for drug screening, both a CaHIS3 heterozygote strain and a tetracycline promoter-regulated CaHIS3 GRACE strain were compared against a wild type (diploid) CaHIS3 strain for sensitivity towards the 3-aminotriazole (3-AT) (FIG. 6). 3-AT is a competitive inhibitor of the enzyme encoded by CaHIS3, imidazoleglycerol phosphate dehydratase, and together serve as a model for a drug and drug target respectively. Overexpression, achieved by the constitutive expression level of CaHIS3 maintained by the tetracycline promoter, confers 3-AT resistance at concentrations sufficient to completely inhibit growth of both wild type and CaHIS3 heterozygote strains (FIG. 6A). The phenotype observed is consistent with that expected in light of the predicted 7.5 fold overexpression of CaHIS3 determined by Northern bolt analysis (see FIG. 5). A heterozygous CaHIS3 strain demonstrates enhanced sensitivity (i.e. haploinsufficient phenotype) to an intermediate 3-AT concentration unable to effect either wild type or tetracycline promoter-based overproducing CaHIS3 strains noticeably (FIG. 6B). A third CaHIS3 expression level evaluated for differential sensitivity to 3-AT was produced by partial repression of the GRACE CaHIS3 strain using a threshold concentration of tetracycline 0.1% that normally is used to achieve complete shut-off.

This level of CaHIS3 expression represents the minimum expression level required for viability and as predicted, demonstrates an enhanced drug sensitivity relative the heterozygous CaHIS3 strain at an intermediate 3-AT concentration (FIG. 6C). Similarly, GRACE strain-specific drug resistance and sensitivity phenotypes to fluconazole and tunicamycin have been demonstrated by increasing and decreasing the level of expression of their respective known drug targets, CaERG11 and CaALG7. Together these results demonstrate that three different levels of expression are achieved using the C. albicans GRACE strain collection, and that they exhibit the predicted drug sensitivity phenotypes between known drugs and their known drug target. Moreover, these experiments clearly indicate how distinct levels of target gene products synthesized within the pathogen could be directly applied in whole cell assay based drug screens to identify novel antifungal compounds against those novel drug targets validated using the GRACE method.

6.4 Identification of a Target Pathway

A target pathway is a genetic or biochemical pathway wherein one or more of the components of the pathway (e.g., enzymes, signaling molecules, etc) is a drug target as determined by the methods of the invention.

6.4.1. Preparation of Stocks of GRACE Strains for Assay

To provide a consistent source of cells to screen, frozen stocks of host GRACE strains are prepared using standard microbiological techniques. For example, a single clone of the microorganism can be isolated by streaking out a sample of the original stock onto an agar plate containing nutrients for cell growth and an antibiotic for which the GRACE strain contains a gene which confers resistance. After overnight growth an isolated colony is picked from the plate with a sterile needle and transferred to an appropriate liquid growth medium containing the antibiotic to which the GRACE strain is resistant. The cells are incubated under appropriate growth conditions to yield a culture in exponential growth. Cells are frozen using standard techniques.

6.4.2. Growth of GRACE Strains for Use in the Assay

Prior to performing an assay, a stock vial is removed from the freezer, rapidly thawed and a loop of culture is streaked out on an agar plate containing nutrients for cell growth and an antibiotic for which the GRACE strain contains a gene which confers resistance. After overnight growth, randomly chosen, isolated colonies are transferred from the plate (sterile inoculum loop) to a sterile tube containing medium containing the antibiotic to which the GRACE strain contains a gene which confers resistance. After vigorous mixing to form a homogeneous cell suspension, the optical density of the suspension is measured and if necessary an aliquot of the suspension is diluted into a second tube of medium plus antibiotic. The culture is then incubated until the cells reach an optical density suitable for use in the assay.

6.4.3. Selection of Medium to be Used in Assay

Two-fold dilution series of the inducer or repressor for the regulatable promoter which is linked to the gene required for the fungal proliferation, virulence or pathogenicity of the GRACE strain are generated in culture medium containing the appropriate antibiotic for which the GRACE strain contains a gene which confers resistance. Several medium are tested side by side and three to four wells are used to evaluate the effects of the inducer or repressor at each concentration in each media. Equal volumes of test media-inducer or repressor and GRACE cells are added to the wells of a 384 well microtiter plate and mixed. The cells are prepared as described above and diluted in the appropriate medium containing the test antibiotic immediately prior to addition to the microtiter plate wells. For a control, cells are also added to several wells of each medium that do not contain inducer or repressor. Cell growth is monitored continuously by incubation by monitoring the optical density of the wells. The percent inhibition of growth produced by each concentration of inducer or repressor is calculated by comparing the rates of logarithmic growth against that exhibited by cells growing in medium without inducer or repressor. The medium yielding greatest sensitivity to inducer or repressor is selected for use in the assays described below.

6.4.4. Measurement of Test Antibiotic Sensitivity in GRACE Strains in which the Level of the Target Gene Product is Not Rate Limiting

Two-fold dilution series of antibiotics of known mechanism of action are generated in the culture medium selected for further assay development that has been supplemented with the antibiotic used to maintain the GRACE strain. A panel of test antibiotics known to act on different pathways is tested side by side with three to four wells being used to evaluate the effect of a test antibiotic on cell growth at each concentration. Equal volumes of test antibiotic and cells are added to the wells of a 384 well microtiter plate and mixed. Cells are prepared as described above using the medium selected for assay development supplemented with the antibiotic required to maintain the GRACE strain and are diluted in identical medium immediately prior to addition to the microtiter plate wells. For a control, cells are also added to several wells that lack antibiotic, but contain the solvent used to dissolve the antibiotics. Cell growth is monitored continuously by incubation in a microtiter plate reader monitoring the optical density of the wells. The percent inhibition of growth produced by each concentration of antibiotic is calculated by comparing the rates of logarithmic growth against that exhibited by cells growing in medium without antibiotic. A plot of percent inhibition against log [antibiotic concentration] allows extrapolation of an IC₅₀ value for each antibiotic.

6.4.5. Measurement of Test Antibiotic Sensitivity in the GRACE Strains in which the Level of the Target Gene Product is Rate Limiting

The culture medium selected for use in the assay is supplemented with inducer or repressor at concentrations shown to inhibit cell growth by a desired amount as described above, as well as the antibiotic used to maintain the GRACE strain. Two fold dilution series of the panel of test antibiotics used above are generated in each of these media. Several antibiotics are tested side by side in each medium with three to four wells being used to evaluate the effects of an antibiotic on cell growth at each concentration. Equal volumes of test antibiotic and cells are added to the wells of a 384 well microtiter plate and mixed. Cells are prepared as described above using the medium selected for use in the assay supplemented with the antibiotic required to maintain the GRACE strain. The cells are diluted 1:100 into two aliquots of identical medium containing concentrations of inducer that have been shown to inhibit cell growth by the desired amount and incubated under appropriate growth conditions. Immediately prior to addition to the microtiter plate wells, the cultures are adjusted to an appropriate optical density by dilution into warm sterile medium supplemented with identical concentrations of the inducer and antibiotic used to maintain the GRACE strain. For a control, cells are also added to several wells that contain solvent used to dissolve test antibiotics but which contain no antibiotic. Cell growth is monitored continuously by incubation under suitable growth conditions in a microtiter plate reader monitoring the optical density of the wells. The percent inhibition of growth produced by each concentration of antibiotic is calculated by comparing the rates of logarithmic growth against that exhibited by cells growing in medium without antibiotic. A plot of percent inhibition against log [antibiotic concentration] allows extrapolation of an IC₅₀ value for each antibiotic.

6.4.6. Determining the Specificity of the Test Antibiotics

A comparison of the IC₅₀s generated by antibiotics of known mechanism of action under conditions in which the level of the gene product required for fungal proliferation, virulence or pathogenicity is rate limiting or is not rate limiting allows the pathway in which a gene product required for fungal proliferation, virulence or pathogenicity lies to be identified. If cells expressing a rate limiting level of a gene product required for fungal proliferation, virulence or pathogenicity are selectively sensitive to an antibiotic acting via a particular pathway, then the gene product encoded by the gene linked to the regulatable promoter in the GRACE strain is involved in the pathway on which the antibiotic acts.

6.4.7. Identification of Pathway in which a Test Antibiotic Acts

As discussed above, the cell-based assay may also be used to determine the pathway against which a test antibiotic acts. In such an analysis, the pathways against in which the gene under the control of the regulatable promoter in each member of a panel of GRACE strains lies is identified as described above. A panel of cells, each containing a regulatable promoter which directs transcription of a proliferation, virulence or pathogenicity-required nucleic acid which lies in a known biological pathway required for fungal proliferation, virulence or pathogenicity, is contacted with a test antibiotic for which it is desired to determine the pathway on which it acts under conditions in which the gene product of the nucleic acid is rate limiting or is not rate limiting. If heightened sensitivity is observed in cells in which the gene product is rate limiting for a gene product which lies in a particular pathway but not in cells expressing rate limiting levels of gene products which lie in other pathways, then the test antibiotic acts against the pathway for which heightened sensitivity was observed.

All references cited herein are incorporated herein by reference in their entirety and for all purposes to the same extent as if each individual publication or patent or patent application was specifically and individually indicated to be incorporated by reference in its entirety for all purposes.

The present invention is not to be limited in scope by the specific embodiments described herein. Indeed, various modifications of the invention in addition to those described herein will become apparent to those skilled in the art from the foregoing description and accompanying figures. Such modifications can be made without departing from its spirit and scope, and are intended to fall within the scope of the appended claims.

What is claimed is:

1. A method for constructing a strain of diploid fungal cells in which both alleles of a gene are modified, the method comprising the steps of:
(a) modifying a first allele of a gene in diploid fungal cells by recombination using a gene disruption cassette comprising a first nucleotide sequence encoding an expressible selectable marker, thereby providing heterozygous diploid fungal cells in which the first allele of the gene is inactivated; and
(b) modifying the second allele of the gene in the heterozygous diploid fungal cells by recombination using a promoter replacement fragment comprising a second nucleotide sequence encoding a heterologous promoter, such that expression of the second allele of the gene is regulated by the heterologous promoter; and
wherein the gene encodes a polypeptide consisting essentially of an amino acid sequence selected from the group consisting of SEQ ID NOs: 7001 to 7932.

2. A method of assembling a collection of diploid fungal cells each of which comprises modified alleles of a different gene, the method comprising the steps of:
(a) modifying a first allele of a first gene in diploid fungal cells by recombination using a gene disruption cassette comprising a first nucleotide sequence encoding an expressible selectable marker, thereby providing heterozygous diploid fungal cells in which the first allele of the gene is inactivated;
(b) modifying a second allele of the first gene in the heterozygous diploid fungal cells by recombination using a promoter replacement fragment comprising a second nucleotide sequence encoding a heterologous promoter, such that expression of the second allele of the gene is regulated by the heterologous promoter, thereby providing a first strain of diploid fungal cells comprising a modified allelic pair of the first gene; and
(c) repeating steps (a) and (b) a plurality of times, wherein a different gene is modified with each repetition, thereby providing the collection of diploid fungal cells each comprising the modified alleles of a different gene, and
wherein each different gene encodes a different polypeptide consisting essentially of an amino acid sequence selected from the group consisting of SEQ ID NOs: 7001 to 7932.

3. The method of claim 1 or 2, wherein the selectable marker in the gene disruption cassette is disposed between a first region and a second region, wherein the first region and the second region hybridize separately to non-contiguous regions of the first allele of the gene in the diploid fungal cells.

4. The method of claim 3, wherein the selectable marker is selected from the group consisting of CaSAT1, CaBSR1, CaURA3, CaHIS3, CaLEU2, CaTRP1, and combinations thereof.

5. The method of claim 1, wherein the diploid fungal cells are cells of fungal species selected from the group consisting of Aspergillus fumigatus, Aspergillus niger, Aspergillus flavis, Candida albicans, Candida tropicalis, Candida parapsilopsis, Candida krusei, Cryptococcus neoformans, Coccidioides immitis, Exophalia dermatiditis, Fusarium oxysporum, Histoplasma capsulatum, Pneumocystis carinii, Trichosporon beigelii, Rhizopus arrhizus, Mucor rouxii, Rhizomucorpusillus, Absidia corymbigera, Botrytis cinerea, Erysiphe graminis, Magnaporthe grisea, Puccinia recodita, Septoria triticii, Tilletia controversa, and Ustilago maydis.

6. The method of claim 1 or 2, wherein the gene corresponds to an open reading frame selected from the group consisting of SEQ ID NOs: 6001-6932.

7. The method of claim 1 or 2, wherein the method further comprises
(c) introducing a nucleotide sequence encoding a transactivation fusion protein that is expressible in the diploid fungal cell, said transactivation fusion protein comprising a DNA binding domain and a transcription activation domain; and wherein the heterologous promoter in the promoter replacement fragment comprises at least one copy of a nucleotide sequence which is bound by the DNA binding domain of the transactivation fusion protein, such that binding of the transactivation fusion protein increases transcription from the heterologous promoter.

8. The method of claim 7, wherein the promoter replacement fragment further comprises a selectable marker.

9. The method of claim 8, wherein the selectable marker is selected from the group consisting of CaHIS3, CaSAT1, CaBSR1, CaURA3, CaLEU2, CaTRP1, and combinations thereof.

10. A strain of diploid fungal cells comprising modified alleles of a gene, wherein the first allele of the gene is inactivated by a gene disruption cassette comprising a nucleotide sequence encoding an expressible selectable marker; and the expression of the second allele of the gene is regulated by a heterologous promoter that is operably linked to the coding region of the second allele of the gene, and wherein the gene encodes a polypeptide consisting essentially of an amino acid sequence selected from the group consisting of SEQ ID NOs: 7001 to 7932.

11. The diploid fungal cells of claim 10 further comprising a nucleotide sequence encoding a transactivation fusion protein that is expressible in the diploid fungal cell, said transactivation fusion protein comprising a DNA binding domain and a transcription activation domain; and wherein the heterologous promoter in the promoter replacement fragment comprises at least one copy of a nucleotide sequence which is bound by the DNA binding domain of the transactivation fusion protein, such that binding of the transactivation fusion protein modulates transcription from the heterologous promoter.

12. The strain of diploid fungal cells of claim 10 or 11, wherein the gene is a gene essential for the growth and/or survival of the cells; or contributes to the virulence and/or pathogenicity of the fungal cells against a host organism.

13. The strain of diploid fungal cells of claim 10 or 11, wherein the gene corresponds to an open reading frame selected from the group consisting of SEQ ID NOs: 6001-6932.

14. A collection of diploid fungal strains comprising diploid strains of claim 10, wherein substantially all the different genes that encode the amino acid sequences of SEQ ID NOs: 7001 to 7932 are modified and are present in different diploid strains in the collection.

15. A collection of diploid fungal strains of claim 10 each comprising the modified alleles of a different gene, wherein each gene is essential for the growth and/or survival of the cells, and wherein each gene encodes a polypeptide consisting essentially of an amino acid sequence selected from the group consisting of SEQ ID NOs: 7001 to 7932.

16. The collection of diploid fungal strains of claim 15, wherein each of the genes corresponds to an open reading frame selected from the group consisting of SEQ ID NO: 6001-6932.

17. A collection of diploid fungal strains of claim 10 each strain comprising the modified alleles of a different gene, wherein each gene contributes to the virulence and/or pathogenicity of the cells to a host organism.

18. The collection of diploid fungal strains of claim 17, wherein substantially all of the genes in the genome of the diploid fungus that contribute to the virulence and/or pathogenicity of the fungal cells against a host organism are modified and present in the collection.

19. The collection of diploid fungal strains of claim 14, wherein the essential genes present in the collection all share a characteristic selected from the group consisting of: similar biological activity, similar intracellular localization, structural homology, sequence homology, cidal terminal phenotype, static terminal phenotype, sequence homology to human genes, and exclusivity with respect to the organism.

20. The collection of diploid fungal strains of claim 14, 15, 17, or 19 wherein the cells of each strain further comprise at least one molecular tag of about 20 nucleotides, the sequence of which is unique to each strain.

21. The collection of claim 20, wherein the molecular tag is disposed within the gene disruption cassette.

22. A nucleic acid molecule microarray comprising a plurality of nucleic acid molecules, wherein each nucleic acid molecule comprises a nucleotide sequence that is hybridizable to a target nucleotide sequence selected from the group consisting of SEQ ID NO: 6001 through to SEQ ID NO: 6932.

23. A nucleic acid molecule microarray comprising a plurality of nucleic acid molecules, wherein each nucleic acid molecule comprises a nucleotide sequence that is hybridizable to the nucleotide sequence of a gene that is either essential to the growth of a diploid fungal cell or contributes to the virulence and/or pathogenicity of the diploid fungal cells against a host organism, and wherein each of the gene encodes a polypeptide consisting essentially of an amino acid sequence selected from the group consisting of SEQ ID NOs: 7001 to 7932.

24. A method for identifying a gene that is essential to the survival of a fungus comprising the steps of:
(a) culturing the diploid fungal cells of claim 10 under conditions wherein the second allele of the gene is substantially underexpressed or not expressed; and
(b) determining viability of the cells; whereby a loss or reduction of viability as compared to a control indicates that the modified gene is essential to the survival of the fungus.

25. A method for identifying a gene that is essential to the growth of a fungus comprising the steps of:
(a) culturing the diploid fungal cells of claim 10 under conditions wherein the second allele of the gene is substantially underexpressed or not expressed; and
(b) determining growth of the cells; whereby a loss or reduction of growth of the cells as compared to a control indicates that the modified gene is essential to the growth of the fungus.

26. A method for identifying a gene that contributes to the virulence and/or pathogenicity of a fungus comprising the steps of:
(a) culturing diploid fungal cells of claim 10 or 11 under conditions wherein the second allele of the gene is substantially underexpressed or not expressed; and
(b) determining the virulence and/or pathogenicity of the cells toward a host cell or organism; whereby a reduction of virulence and/or pathogenicity as compared to a control indicates that the modified gene contributes to the virulence and/or pathogenicity of the fungus.

27. A method for identifying a gene that contributes to the resistance of a diploid fungus to an antifungal agent comprising the steps of:
(a) culturing the diploid fungal cells of claim 10 under conditions wherein the second allele is substantially overexpressed and in the presence of the antifungal agent; and
(b) determining the viability of the cells; whereby an increase in viability as compared to a control indicates that the modified gene contributes to the resistance of the diploid fungus to the antifungal agent.

28. A method for identifying an antifungal agent that inhibits the growth of a diploid fungus comprising the steps of:
(a) providing diploid fungal cells of claim 12; and
(b) culturing the diploid fungal cells under conditions wherein the second allele of the gene is underexpressed and in the presence of a test compound;
whereby a loss or reduction of growth of the diploid fungal cells as compared to a control indicates that the test compound is an antifungal agent.

29. A method for identifying a therapeutic agent for treatment of a mammalian disease, the method comprising the steps of:
(a) providing diploid cells of claim 10, wherein the modified gene in the diploid cells is an essential gene and displays sequence homology to a mammalian gene associated with the disease;
(b) culturing diploid fungal cells under conditions wherein the second allele of the gene is overexpressed or underexpressed and in the presence of a test compound;
whereby a difference in growth of the diploid fungal cells as compared to a control indicates that the test compound is a therapeutic agent.

30. A method for correlating changes in the levels of proteins with the inhibition of growth or proliferation of a diploid fungal cell, the method comprising the steps of:
(a) generating a first protein expression profile for a control diploid fungal cell which comprises two wild type alleles of the gene;
(b) culturing diploid fungal cells of claim 12 under conditions wherein the second allele of the gene is substantially underexpressed, not expressed or overexpressed, and generating a second protein expression profile for the cultured cells; and
(c) comparing the first protein expression profile with the second protein expression profile to identify changes in the levels of proteins.

31. A method for correlating changes in the levels of gene transcripts with the inhibition of growth or proliferation of a diploid fungal cell, the method comprising the steps of:
(a) generating a transcription profile for a control diploid fungal cell which comprises two wild type alleles of the gene;
(b) culturing diploid fungal cells of claim 12 under conditions wherein the second allele of the gene is substantially underexpressed, not expressed or overexpressed and generating a second transcription profile for the cultured cells; and
(c) comparing the first transcription profile with the second transcription profile to identify changes in the levels of gene transcripts.

32. A purified or isolated nucleic acid molecule comprises a nucleotide sequence encoding a gene product required for proliferation of Candida albicans, wherein said gene product consisting essentially of an amino acid sequence of one of SEQ ID NOs: 7001 to 7932.

33. The nucleic acid molecule of claim 32, wherein said nucleotide sequence is one of SEQ ID NOs: 6001 to 6932.

34. A nucleic acid molecule comprising a fragment of one of SEQ ID NOs: 6001 to 6932, said fragment selected from the group consisting of fragments comprising at least 10, at least 20, at least 25, at least 30, at least 50 and at least 100 consecutive nucleotides of one of SEQ ID NOs: 6001 to 6932.

35. A nucleic acid molecule comprising a nucleotide sequence that hybridizes under stringent condition to a second nucleic acid molecule consisting of (a) a nucleotide sequence selected from the group consisting of one of SEQ ID NOs: 6001 to 6932, or (b) a nucleotide sequence that encodes a polypeptide consisting of an amino acid sequence selected from the group consisting of one of SEQ ID NOs: 7001 to 7932; wherein said stringent condition comprises hybridization to filter-bound DNA in 6× sodium chloride/sodium citrate (SSC) at about 45° C. followed by one or more washes in 0.2×SSC/0.1% SDS at about 50-65° C.

36. The nucleic acid molecule of claim 34 or 35, which consists of the nucleotide sequence selected from the group consisting of one of SEQ ID NOs: 4001 to 4932, and 5001 to 5932.

37. A purified or isolated nucleic acid molecule obtained from an organism other than Candida albicans or Saccharomyces cerevisiae comprising a nucleotide sequence having at least 30% identity to a sequence selected from the group consisting of SEQ ID NOs: 6001-6932, fragments comprising at least 25 consecutive nucleotides of SEQ ID NOs: 6001-6932, the sequences complementary to SEQ ID NOs: 6001-6932 and the sequences complementary to fragments comprising at least 25 consecutive nucleotides of SEQ ID NOs: 6001-6932, as determined using BLASTN version 2.0 with the default parameters.

38. The purified or isolated nucleic acid molecule of claim 37, wherein said organism is selected from the group consisting of Absidia corymbigera, Aspergillus flavis, Aspergillus fumigatus, Aspergillus niger, Botrytis cinerea, Candida albicans, Candida dublinensis, Candida glabrata, Candida krusei, Candida parapsilopsis, Candida tropicalis, Coccidioides immitis, Cryptococcus neoformans, Erysiphe graminis, Exophalia dermatiditis, Fusarium oxysporum, Histoplasma capsulatum, Magnaporthe grisea, Mucor rouxii, Pneumocystis carinii, Puccinia graminis, Puccinia recodita, Puccinia striiformis, Rhizomucor pusillus, Rhizopus arrhizus, Septoria avenae, Septoria nodorum, Septoria triticii, Tilletia controversa, Tilletia triticii, Trichosporon beigelii, and Ustilago maydis.

39. A vector comprising a promoter operably linked to the nucleic acid molecule of claim 32, 33, 34, 35, or 37.

40. The vector of claim 39, wherein said promoter is regulatable.

41. The vector of claim 39, wherein said promoter is active in an organism selected from the group consisting of Absidia corymbigera, Aspergillus flavis, Aspergillus fumigatus, Aspergillus niger, Botrytis cinerea, Candida albicans, Candida dublinensis, Candida glabrata, Candida krusei, Candida parapsilopsis, Candida tropicalis, Coccidioides immitis, Cryptococcus neoformans, Erysiphe graminis, Exophalia dermatiditis, Fusarium oxysporum, Histoplasma capsulatum, Magnaporthe grisea, Mucor rouxii, Pneumocystis carinii, Puccinia graminis, Puccinia recodita, Puccinia striiformis, Rhizomucor pusillus, Rhizopus arrhizus, Septoria avenae, Septoria nodorum, Septoria triticii, Tilletia controversa, Tilletia triticii, Trichosporon beigelii, and Ustilago maydis.

42. A host cell containing the vector of claim 39.

43. A purified or isolated polypeptide comprising an amino acid sequence selected from the group consisting of one of SEQ ID NOs: 63 to 123.

44. A purified or isolated polypeptide obtained from an organism other than Candida albicans or Saccharomyces cerevisiae comprising an amino acid sequence having at least 30% similarity to an amino acid sequence selected from the group consisting of one of SEQ ID NOs: 7001 to 7932 as determined using FASTA version 3.0t78 with the default parameters.

45. The polypeptide of claim 44, wherein said organism is selected from the group consisting of Absidia corymbigera, Aspergillus flavis, Aspergillus fumigatus, Aspergillus niger, Botrytis cinerea, Candida albicans, Candida dublinensis, Candida glabrata, Candida krusei, Candida parapsilopsis, Candida tropicalis, Coccidioides immitis, Cryptococcus neoformans, Erysiphe graminis, Exophalia dermatiditis, Fusarium oxysporum, Histoplasma capsulatum, Magnaporthe grisea, Mucor rouxii, Pneumocystis carinii, Puccinia graminis, Puccinia recodita, Puccinia striiformis, Rhizomucor pusillus, Rhizopus arrhizus, Septoria avenae, Septoria nodorum, Septoria triticii, Tilletia controversa, Tilletia triticii, Trichosporon beigelii, and Ustilago maydis.

46. A fusion protein comprising a fragment of a first polypeptide fused to a second polypeptide, said fragment consisting of at least 6 consecutive residues of an amino acid sequence selected from one of SEQ ID NOs: 7001 to 7932.

47. A method of producing a polypeptide, said method comprises introducing into a cell, a vector comprising a promoter operably linked to a nucleotide sequence encoding a polypeptide consisting of an amino acid sequence selected from the group consisting of one of SEQ ID NOs: 7001 to 7932; and culturing the cell such that the nucleotide sequence is expressed.

48. A method of producing a polypeptide, said method comprising providing a cell which comprises a heterologous promoter operably linked to a nucleotide sequence encoding a polypeptide consisting of an amino acid sequence selected from the group consisting of one of SEQ ID NOs: 7001 to 7932; and culturing the cell such that the nucleotide sequence is expressed.

49. A method for identifying a compound which modulates the activity of a gene product encoded by a nucleic acid comprising a nucleotide sequence selected from the group consisting of one of SEQ ID NOs: 6001 to 6932, said method comprising:
(a) contacting said gene product with a compound; and
(b) determining whether said compound modulates the activity of said gene product.

50. The method of claim 49, wherein the activity of the gene product is inhibited.

51. The method of claim 49, wherein said gene product is a polypeptide and said activity is selected from the group consisting of an enzymatic activity, carbon compound catabolism activity, a biosynthetic activity, a transporter activity, a transcriptional activity, a translational activity, a signal transduction activity, a DNA replication activity, and a cell division activity.

52. A method of eliciting an immune response in an animal, comprising introducing into the animal a composition comprising an isolated polypeptide, the amino acid sequence of which comprises at least 6 consecutive residues of one of SEQ ID NOs: 7001 to 7932.

53. A strain of Candida albicans wherein a first allele of a gene comprising a nucleotide sequence selected from the group consisting of one of SEQ ID NOs: 6001 to 6932 is inactive and a second allele of the gene is under the control of a heterologous promoter.

54. A strain of Candida albicans comprising a nucleic acid molecule comprising a nucleotide sequence selected from one of SEQ ID NOs: 6001 to 6932 under the control of a heterologous promoter.

55. The strain of claim 53 or 54, wherein said heterologous promoter is regulatable.

56. A method of identifying a compound or binding partner that binds to a polypeptide comprising an amino acid sequence selected from the group consisting of one of SEQ ID NOs: 7001 to 7932 or a fragment thereof said method comprising:
(a) contacting the polypeptide or fragment thereof with a plurality of compounds or a preparation comprising one or more binding partners; and
(b) identifying a compound or binding partner that binds to the polypeptide or fragment thereof.

57. A method for identifying a compound having the ability to inhibit growth or proliferation of Candida albicans, said method comprising the steps of:
(a) reducing the level or activity of a gene product encoded by a nucleic acid selected from the group consisting of SEQ ID NOs: 6001 to 6932 in a Candida albicans cell relative to a wild type cell, wherein said reduced level is not lethal to said cell;
(b) contacting said cell with a compound; and
(c) determining whether said compound inhibits the growth or proliferation of said cell.

58. The method of claim 57, wherein said step of reducing the level or activity of said gene product comprises transcribing a nucleotide sequence encoding said gene product from a regulatable promoter under conditions in which said gene product is expressed at said reduced level.

59. The method of claim 58, wherein said gene product is a polypeptide comprising a sequence selected from the group consisting of polypeptides encoded by SEQ ID NOs: 7001 to 7932.

60. A method for inhibiting growth or proliferation of Candida albicans cells comprising contacting the cells with a compound that (i) reduce the level of or inhibit the activity of a nucleotide sequence selected from the group consisting of SEQ ID NOs: 6001 to 6932, or (ii) reduce the level of or inhibit the activity of a gene product encoded by a nucleotide sequence selected from the group consisting of SEQ ID NOs: 6001 to 6932.

61. The method of claim 60, wherein said gene product is a polypeptide comprising an amino acid sequence selected from the group consisting of polypeptides encoded by SEQ ID NOs: 7001 to 7932.

62. The method of claim 60, wherein the compound is an antibody, a fragment of an antibody, an antisense nucleic acid molecule, or a ribozyme.

63. A method for manufacturing an antimycotic compound comprising the steps of:
(a) screening a pluralities of candidate compounds to identify a compound that reduces the activity or level of a gene product encoded by a nucleotide sequence selected from the group consisting of SEQ ID NOs: 6001 to 6932; and
(b) manufacturing the compound so identified.

64. The method of claim 63, wherein said gene product is a polypeptide comprising an amino acid sequence selected from the group consisting of polypeptides encoded by SEQ ID NOs: 6001 to 6932.

65. A method for treating an infection of a subject by Candida albicans comprising administering a pharmaceutical composition comprising a therapeutically effective amount of a compound that reduces the activity or level of a gene product encoded by a nucleic acid comprising a sequence selected from the group consisting of SEQ ID NOs: 6001 to 6932 and a pharmaceutically acceptable carrier, to said subject.

66. The method of claim 65, wherein the compound is an antibody, a fragment of an antibody, an antisense nucleic acid molecule, or a ribozyme.

67. A method for preventing or containing contamination of an object by Candida albicans comprising contacting the object with a composition comprising an effective amount of a compound that reduces the activity or level of a gene product encoded by a nucleic acid comprising a sequence selected from the group consisting of SEQ ID NOs: 6001 to 6932.

68. A method for preventing or inhibiting formation on a surface of a biofilm comprising Candida albicans, said method comprising contacting the surface with a composition comprising an effective amount of a compound that reduces the activity or level of a gene product encoded by a nucleic acid comprising a sequence selected from the group consisting of SEQ ID NOs: 6001 to 6932.

69. A pharmaceutical composition comprising a therapeutically effective amount of an agent which reduces the activity or level of a gene product encoded by a nucleic acid selected from the group consisting of SEQ ID NOs: 6001 to 6932 in a pharmaceutically acceptable carrier.

70. The method of claim 65, wherein said subject is selected from the group consisting of a plant, a vertebrate, a mammal, an avian, and a human.

71. An antibody preparation which binds the polypeptide of claim 43 or 44.

72. The antibody preparation of claim 71 which comprises a monoclonal antibody.

73. A method for evaluating a compound against a target gene product encoded by a nucleotide sequence comprising one of SEQ ID NOs: 6001 to 6932, said method comprising the steps of:
(a) contacting wild type diploid fungal cells with the compound and generating a first protein expression profile;
(b) determining the protein expression profile of diploid fungal cells of claim 12 which have been cultured under conditions wherein the second allele of the target gene is substantially underexpressed, not expressed or overexpressed and generating a second protein expression profile for the cultured cells; and
(c) comparing the first protein expression profile with the second protein expression profile to identify similarities in the profiles.

74. A method for evaluating a compound against a target gene product encoded by a nucleotide sequence comprising one of SEQ ID NOs: 6001 to 6932, said method comprising the steps of:
(a) contacting wild type diploid fungal cells with the compound and generating a first transcription profile;
(b) determining the transcription profile of diploid fungal cells of claim 12 which have been cultured under conditions wherein the second allele of the target gene is substantially underexpressed, not expressed or overexpressed and generating a second transcription profile for the cultured cells; and
(c) comparing the first transcription profile with the second transcription profile to identify similarities in the profiles.

75. A method for identifying an antimycotic compound comprising screening a plurality of compounds to identify a compound that reduces the activity or level of a gene product, said gene product being encoded by a nucleotide sequence that is naturally occurring in Saccharomyces cerevisiae and that is the ortholog of a gene having a nucleotide sequence selected from the group consisting of SEQ ID NOs: 6001 to 6932.

76. A computer or a computer readable medium that comprises at least one nucleotide sequence selected from the group consisting of SEQ ID NOs: 1-932, 1001-1932, 2001-2932, 3001-3932, 4001-4932, 5001-5932, and 6001-6932, or at least one amino acid sequence selected from the group consisting of SEQ ID NOs: 7001-7932.

77. A method assisted by a computer for identifying a putatively essential gene of a fungus, comprising detecting sequence homology between a fungal nucleotide sequence or fungal amino acid sequence with at least one nucleotide sequence selected from the group consisting of SEQ ID NOs: 6001-6932, or at least one amino acid sequence selected from the group consisting of SEQ ID NOs: 7001-7932.
